(19) United States
(12) Patent Application Publication
(10) Pub. No.: US 2023/0212172 A1
(43) Pub. Date: Jul. 6, 2023

(54) ARYLACETAMIDE ANALOGS OF PIPERAZINE-[1,2,4]TRIAZOLO[4,3-B]PYRIDAZINES

(71) Applicants: University of Vermont and State Agricultural College, Burlington, VT (US); Saint Louis University, St. Louis, MO (US)

(72) Inventors: Christopher D. Huston, Burlington, VT (US); Erin E. Stebbins, Burlington, VT (US); Marvin J. Meyers, Wentzville, MO (US); Edmund Oboh, St. Louis, MO (US); Tanner Schubert, St. Louis, MO (US)

(21) Appl. No.: 18/001,148

(22) PCT Filed: Jun. 8, 2021
(86) PCT No.: PCT/US2021/036424

§ 371 Date: Dec. 8, 2022

Related U.S. Application Data
(60) Provisional application No. 63/036,390, filed on Jun. 8, 2020.

(51) Int. Cl. C07D 487/04; A61P 33/14

(52) U.S. Cl. CPC C07D 487/04; A61P 33/14

(57) ABSTRACT

Provided are compounds with the following structure: Formula (I), and methods of making and using same. The methods of using the compounds may be methods for treating or prophylaxis of a cryptosporidium infection.

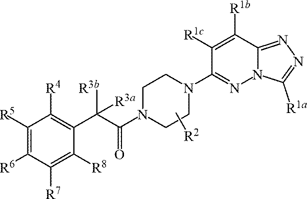

### CROSS REFERENCE TO RELATED APPLICATION

This application claims priority to U.S. Provisional Application No. 63/036,390, filed Jun. 8, 2020, the entire disclosure of which is incorporated herein by reference.

### STATEMENT REGARDING FEDERALLY SPONSORED RESEARCH

This invention was made with government support under contract no. R01AI143951 awarded by the National Institutes of Health. The government has certain rights in the invention.

### BACKGROUND OF THE DISCLOSURE

The apicomplexan parasites Cryptosporidium parvum and Cryptosporidium hominis are major etiologic agents of cryptosporidiosis in humans. Infection is typically self-limited in immunocompetent adults, but it can lead to chronic and fulminant disease in immunocompromised patients, as well as malnutrition and stunting in children. Nitazoxanide is the current standard of care for cryptosporidiosis, but the drug only exhibits partial efficacy in children and is no more effective than placebo in AIDS patients. Unfortunately, the development of novel therapeutics for cryptosporidiosis has proven to be extremely difficult as a result of the financial obstacles that plague drug discovery for diseases that disproportionately affect the developing world, as well as technical limitations associated with the laboratory study of Cryptosporidium parasites.

While cryptosporidiosis is a significant cause of self-limited diarrhea in immunocompetent individuals who may be exposed to parasites through contaminated municipal and recreational water supplies or through occupational exposures, the burden of cryptosporidiosis is even more substantial in immunocompromised and pediatric populations. Immunodeficient individuals, including patients maintained on immunosuppressive regimens following organ transplantation and AIDS patients, in particular, risk developing chronic, fulminant, and sometimes fatal disease (especially when CD-4⁺ T-cell counts drop below 50 cells/mm³). Diarrhea is also a leading cause of death in children under 5 years of age, and the recent Global Enteric Multicenter Study (GEMS) identified Cryptosporidium as a major cause of life-threatening diarrhea during the first two years of life. Moreover, cryptosporidiosis has been associated with malnutrition and persistent deficits in development in this population.

### SUMMARY OF THE DISCLOSURE

The present disclosure relates to compounds and methods for treating or prophylaxis of a cryptosporidium infection. For example, the method comprises administering to an individual diagnosed with or suspected of having a cryptosporidium infection or at risk of having cryptosporidium infection, a composition comprising a therapeutically effective or a prophylactically effective amount of a compound disclosed herein. The present disclosure also provides pharmaceutical compositions for alleviating the symptoms of, for treating, or for preventing the occurrence of cryptosporidium infection. In one embodiment, the pharmaceutical compositions are suitable for oral administration. The present disclosure also provides kits for alleviating the symptoms of, for treating, or for preventing the occurrence of cryptosporidium infection. The kits comprise one or more compounds of the present disclosure (such as in an oral composition) and instructions for use, storage and the like.

### BRIEF DESCRIPTION OF THE FIGURES

For a fuller understanding of the nature and objects of the disclosure, reference should be made to the following detailed description taken in conjunction with the accompanying drawings.

FIG. 1 shows results for treatment of C. parvum infected NOD SCID gamma mice with SLU-0002633 by oral gavage at 50 mg/kg twice daily.

FIG. 2 shows treatment with SLU-0002633 by oral gavage at 1, 5, 15, and 50 mg/kg twice daily.

FIG. 3 shows treatment with SLU-0002633 by oral gavage at 1, 5, 15, and 50 mg/kg twice daily.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

The present disclosure provides compounds and methods for treating a Cryptosporidium infection or a method for prophylaxis and/or treatment comprising administering to an individual a therapeutically effective amount of a compound having a [1,2,4]triazolo[4,3-b]pyridazine (henceforth referred to as “triazolopyridazine”) scaffold.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative examples of groups include:

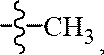

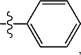

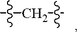

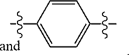

As used herein, unless otherwise indicated, the term “alkyl” or “alkyl group” refers to branched or unbranched, linear saturated hydrocarbon groups and/or cyclic hydrocarbon groups. Examples of alkyl groups include, but are not limited to, methyl groups, ethyl groups, propyl groups, butyl groups, isopropyl groups, tert-butyl groups, cyclopropyl groups, cyclopentyl groups, cyclohexyl groups, and the like. Alkyl groups are saturated groups, unless it is a cyclic group. For example, an alkyl group is a C₁ to C₄₀ alkyl group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, and C₄₀). The alkyl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof.

As used herein, the term “cycloalkyl” or “cycloalkyl group” refers to a cyclic hydrocarbon group, e.g., cyclopropyl, cyclobutyl, cyclohexyl, and cyclopentyl groups. Cycloalkyl groups can be saturated or partially unsaturated ring systems optionally substituted with, for example, one to three substituents. Each substituent is independently chosen from alkyl, —NH₂, oxo (═O), phenyl, haloalkyl (e.g., —CF₃), halo (e.g., —F, —Cl, —Br, —I), alkoxy, and —OH groups. Additionally, alkyl substituents may be substituted with various other functional groups. Additional non-limiting examples include aliphatic groups (e.g., alkyl groups, alkenyl groups, alkynyl groups, and the like), halogenated aliphatic groups (e.g., trifluoromethyl group), aryl groups, halogenated aryl groups, alkoxide groups, nitro groups, carboxylate groups, carboxylic acids, ether groups, alkyne groups (e.g., acetylenyl groups and the like), and the like, and combinations thereof.

As used herein, unless otherwise indicated, the term “aryl” or “aryl group” refers to C₅ to C₃₀ aromatic or partially aromatic carbocyclic groups, including all integer numbers of carbons and ranges of numbers of carbons therebetween (e.g., C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, and C₃₀). An aryl group may also be referred to as an aromatic group. The aryl groups may comprise polyaryl groups such as, for example, fused rings, biaryl groups, or a combination thereof. The aryl group may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), aryl groups, alkoxides, carboxylates, carboxylic acids, ether groups, and the like, and combinations thereof. Examples of aryl groups include, but are not limited to, phenyl groups, biaryl groups (e.g., biphenyl groups and the like), fused ring groups (e.g., naphthyl groups and the like), hydroxybenzyl groups, tolyl groups, xylyl groups, and the like.

As used herein, the term “heteroaryl” or “hereteroaryl” refers to a monocyclic or bicyclic ring system comprising one or two aromatic rings and containing at least one nitrogen or oxygen atom in an aromatic ring. Unless otherwise indicated, a heteroaryl group can be unsubstituted or substituted with one or more, and in particular one or two, substituents. Non-limiting examples of substituents include halogens (—F, —Cl, —Br, and —I), aliphatic groups (e.g., alkyl groups, alkenyl groups, alkynyl groups, and the like), halogenated aliphatic groups (e.g., trifluoromethyl group), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof. Examples of heteroaryl groups include, benzofuranyl, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl groups, and substituents analogs of any of the foregoing heteroaryl groups.

As used herein, unless otherwise indicated, the term “alkoxy” or “alkoxy group” refers to

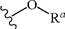

where R^{a} is a linear, branched or cyclic C₁-C₆ alkyl group, including all integer numbers of carbons and ranges of numbers of carbons therebetween. For example, suitable alkoxy groups include methoxy, ethoxy, propoxy, iso-propoxy, butoxy, sec-butoxy, tert-butoxy, and hexoxy groups. Additionally, alkyl substituents can be substituted with various other functional groups, e.g. functional groups disclosed herein.

As used herein, unless otherwise indicated, the term “amino” or “amino group” refers to

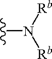

where each R^{b} is selected independently from the group consisting of hydrogen atom, substituted or unsubstituted C₁-C₁₀ alkyl, including all integer numbers of carbons and ranges of numbers of carbons therebetween, substituted or unsubstituted phenyl, substituted or unsubstituted heteroaryl, substituted carbonyl, substituted sulfonyl, haloalkyl, and substituted or unsubstituted benzyl groups.

As used herein, unless otherwise indicated, the term “benzyl” or “benzyl group” refers to

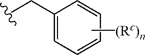

where R^{c} is a substituent on the phenyl ring and n is from 0 to 5. The substituents can be the same or different. For example, the substituents on the benzyl group include substituted or unsubstituted alkyl, —NH₂, phenyl, haloalkyl (e.g., —CF₃), halo (e.g., —F, —Cl, —Br, —I), alkoxy (e.g., —OMe), and —OH groups.

As used herein, unless otherwise indicated, halogen means fluorine, chlorine, bromine, and iodine, and halo means fluoro, chloro, bromo, and iodo.

As used herein, unless otherwise indicated, the term “phenoxy” or “phenoxy group” (—OPh) refers to

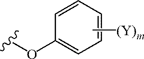

where each Y is independently selected from the group consisting of F, Cl, Br, and I and m can be 0, 1 or 2.

As used herein, unless otherwise indicated, the term “phenyl” or “phenyl group” means

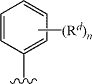

where each R^{d} is an independent substituent on the phenyl group and n is from 0 to 5. The substituents at different occurrences can be the same or different. For example, the substituents on the phenyl group include substituted or unsubstituted C₁-C₆ alkyl, including all integer numbers of carbons and ranges of numbers of carbons therebetween, substituted or unsubstituted amino, haloalkyl (e.g., —CF₃), halo (e.g., —F, —Cl, —Br, —I), substituted or unsubstituted alkoxy (e.g., —OMe), and sulfonyl group. In certain instances, two adjacent R groups can be connected through to form a dioxolyl group.

In an aspect, the present disclosure provides compounds. The compounds of the present disclosure may be suitable in treating an individual diagnosed with or suspected of having a Cryptosporidium infection.

A compound of the present disclosure can have the following structure:

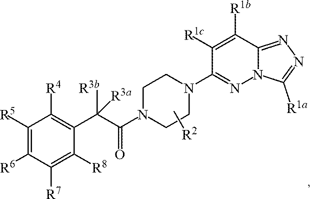

where R^{1a}, R^{1b}, and R^{1c} are independently chosen from H, substituted alkyl groups (e.g., haloalkyl groups, such as, for example, trifluoromethyl), unsubstituted alkyl groups, substituted aryl groups, unsubstituted aryl groups, substituted aralkyl groups, and unsubstituted aralkyl groups; R² is chosen from H, substituted alkyl groups, and unsubstituted alkyl groups; R^{3a} and R^{3b} are independently chosen from H, substituted alkyl groups, unsubstituted alkyl groups, halogen, —OH, —NH₂, and R^{3a} and R^{3b} may combine to form a ring (e.g., cyclopropyl

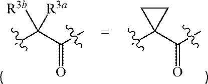

and the like) or a ═O; R⁴ and R⁸ are independently chosen from H, halogens, substituted alkyl groups, and unsubstituted alkyl groups; R⁵, R⁶, and R⁷ are independently chosen from H, halogens, substituted alkyl groups (e.g., haloalkyl groups; alkylamino groups ((CH₂)ₙNH₂, where n is 1, 2, 3, or 4; (CH₂)ₙNR′R″, where n is 1, 2, 3, or 4); (CH2)ₙOH groups, where n is 1, 2, 3, or 4, and the like), unsubstituted alkyl groups, substituted cycloalkyl groups, unsubstituted cycloalkyl groups, substituted alkoxy groups, unsubstituted alkoxy groups, substituted aryl groups, unsubstituted aryl groups, substituted aralkyl groups, unsubstituted aralkyl groups, SO₂NR′R″, (CH₂)ₙ-alkoxy groups (where n is 1, 2, 3, or 4), (CH₂)ₙNHCO-alkyl (where alkyl is substituted or unsubstituted, and n is 1, 2, 3, or 4), nitro groups, alcohol groups, —SF₅ groups, and CONR′R″, where R′ and R″ are independently chosen from H, substituted alkyl groups (e.g., methoxyethyl), unsubstituted alkyl groups (e.g., methyl), Cl, and F. In various examples, R⁴ and R⁵, R⁵ and R⁶, R⁶ and R⁷, or R⁷ and R⁸ may combine to form a ring. The ring may be aromatic or non-aromatic.

R¹ groups (e.g., R^{1a}, R^{1b}, and/or R^{1c} groups) may be various substituents. R^{1a}, R^{1b}, and R^{1c} are independently chosen from H, substituted alkyl groups (e.g., haloalkyl groups, such as, for example, trifluoromethyl), unsubstituted alkyl groups, substituted aryl groups, unsubstituted aryl groups, substituted aralkyl groups, and unsubstituted aralkyl groups. In various examples, R^{1a}, R^{1b}, and R^{1c} are independently chosen from —H, methyl, trifluoromethyl, phenyl, benzyl, cyclopropyl, and

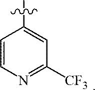

In various examples, R^{1a} is chosen from —H, methyl, trifluoromethyl, phenyl, benzyl, cyclopropyl, and

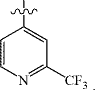

In various examples, R^{1b} is chosen from —H and methyl. In various examples, R^{1c} is chosen from -H and methyl.

R² groups may be various substituents. In various examples, R² is chosen from H, substituted alkyl groups, and unsubstituted alkyl groups. In various examples, R² is chosen from —H and methyl.

R³ groups (e.g., R^{3a} and/or R^{3b} groups) may be various substituents. In various examples, R^{3a} and R^{3b} are independently chosen from H, substituted alkyl groups, unsubstituted alkyl groups, halogen, OH, NH₂, and R^{3a} and R^{3b} may combine to form a ring (e.g., cyclopropyl

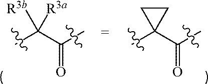

and the like) or a ═O. In various examples, R^{3a} and R^{3b} are independently chosen from —H, —F, methyl, and combinations thereof. In various examples, R^{3a} and R^{3b} are joined to form a carbonyl or cyclopropyl group.

R⁴ groups may be various substituents. In various examples, R⁴ is chosen from H, halogens, substituted alkyl groups, and unsubstituted alkyl groups. In various examples, R⁴ is chosen from —H, —Cl, and —F. In various examples, R⁴ is joined with R⁵ to form a ring, such as, for example a phenyl ring, as seen in, such as, for example, compound SLU-0002257. In various examples, the formed ring is aromatic or non-aromatic.

R⁵, R⁶, and R⁷ groups may be various substituents. In various examples, are independently chosen from H, halogens, substituted alkyl groups (e.g., haloalkyl groups; alkylamino groups ((CH₂)ₙNH₂, where n is 1, 2, 3, or 4; (CH₂)ₙNR′R″, where n is 1, 2, 3, or 4); (CH₂)ₙOH groups, where n is 1, 2, 3, or 4, and the like), unsubstituted alkyl groups, substituted cycloalkyl groups, unsubstituted cycloalkyl groups, substituted alkoxy groups, unsubstituted alkoxy groups, substituted aryl groups, unsubstituted aryl groups, substituted aralkyl groups, unsubstituted aralkyl groups, SO₂NR′R″, (CH₂)ₙ-alkoxy groups (where n is 1, 2, 3, or 4), (CH₂)ₙNHCO-alkyl (where alkyl is substituted or unsubstituted, and n is 1, 2, 3, or 4), nitro groups, alcohol groups, —SF₅ groups, and CONR′R″, where R′ and R″ are independently chosen from H, substituted alkyl groups (e.g., methoxyethyl), unsubstituted alkyl groups (e.g., methyl), Cl, and F. In various examples, R⁵ and R⁶ may be joined to form a ring. In various examples, R⁶ and R⁷ may be joined to form a ring. The formed ring may be aromatic or non-aromatic. In various examples, R⁵, R⁶, and R⁷ are independently chosen from —H, —F, —Cl, —Br, —OCH₃, —SF₅, —CF₃, —CH₃, t-butyl, ethyl, isopropyl, cyclopropyl, —CONH₂, —SO₂NH₂, —SO₂CH₃, phenyl, benzyl, phenoxy, —CH₂OH, —CCH, —CHF₂, —CH2F, —CN, —NO₂, —OCF₃, —CF₂CH₃, —CF₂CF₃, —CCCH₃, —CH₂OCH₃, —CO₂H (and salts and deprotonated forms thereof), —CONHCH₃, —SO₂NHCH₃,

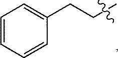

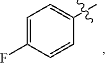

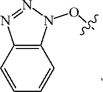

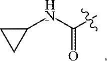

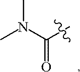

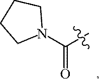

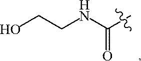

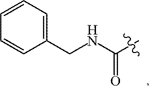

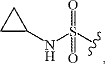

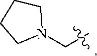

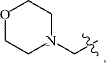

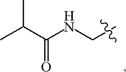

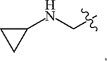

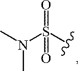

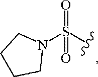

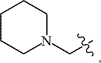

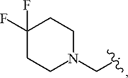

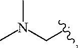

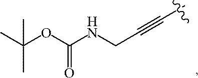

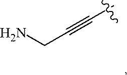

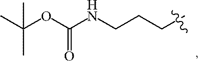

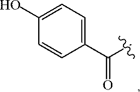

and combinations thereof. In various examples, R⁵ is chosen from —H, —F, —Cl, —Br, —OCH₃, —SF₅, —CF₃, —CH₃, t-butyl, ethyl, isopropyl, cyclopropyl, —CONH₂, —SO₂NH₂, —SO₂CH₃, phenyl, benzyl, phenoxy, —CH₂OH, —CCH, —CHF₂, —CH₂F, —CN, —NO₂, —OCF₃, —CF₂CH₃, —CF₂CF₃, —CCCH₃, —CH₂OCH₃, —CO₂H (and salts and deprotonated forms thereof), —CONHCH3, —SO₂NHCH₃,

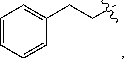

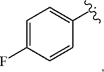

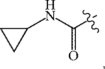

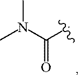

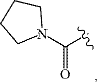

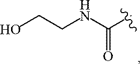

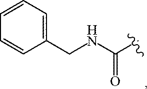

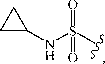

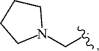

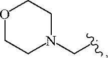

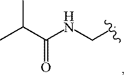

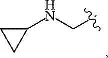

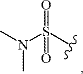

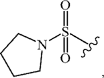

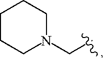

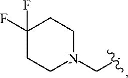

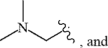

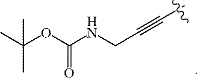

In various examples, R⁶ is chosen from —H, —F, —Cl, —Br, —OCH₃, —SF₅, —CF₃, —CH₃, t-butyl, ethyl, —CONH₂, —SO₂NH₂, —SO₂CH₃, phenyl, benzyl, phenoxy, —CH₂OH, —CCH, —CN, —NO₂, —CO₂H (and salts and deprotonated forms thereof),

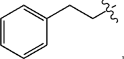

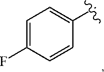

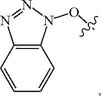

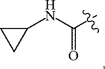

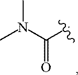

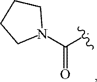

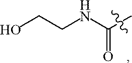

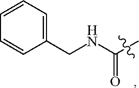

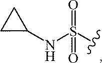

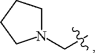

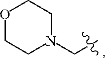

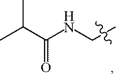

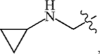

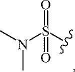

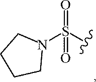

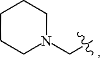

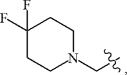

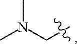

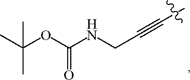

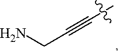

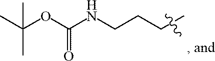

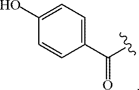

In various examples, R⁷ is chosen from —H, —Cl, —F, and —Br. In various examples, R⁵ and R⁶ are joined to form a ring, such as, for example, to form a phenyl group, a cyclopentyl, or a methylenedioxy group.

R⁸ groups may be various substituents. In various examples, R⁷ is chosen from H, halogens, substituted alkyl groups, and unsubstituted alkyl groups. In various examples, R⁴ is chosen from —H, —Cl, and —F. In various examples, R⁸ is joined with R⁷ to form a ring, such as, for example a phenyl ring. In various examples, the formed ring is aromatic or non-aromatic.

In various examples, a compound of the present disclosure has the following structure:

| | | |
|---|---|---|
| Structure | IUPAC | Example No. (May be simply referred to by the last four digits, e.g., SLU-0002178 may be referred to as 2178 or as SLU-2178) |
| 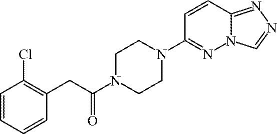 [SMILES: O=C(Cc1ccccc1Cl)N1CCN(c2ccc3nncn3n2)CC1] | 2-(2-chlorophenyl)-1-(4-{[1,2,4]triazolo[4,3-b]pyridazin-6-yl}piperazin-1-yl)ethan-1-one | SLU-0002178 |
| 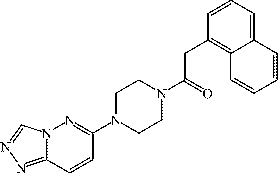 [SMILES: O=C(Cc1cccc2ccccc12)N1CCN(c2ccc3nncn3n2)CC1] | 2-(naphthalen-1-yl)-1-(4-{[1,2,4]triazolo[4,3-b]pyridazin-6-yl}piperazin-1-yl)ethan-1-one | SLU-0002257 |
| 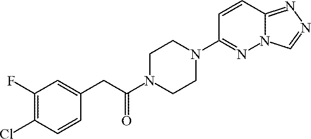 [SMILES: O=C(Cc1ccc(Cl)c(F)c1)N1CCN(c2ccc3nncn3n2)CC1] | 2-(4-chloro-3-fluorophenyl)-1-(4-{[1,2,4]triazolo[4,3-b]pyridazin-6-yl}piperazin-1-yl)ethan-1-one | SLU-0002631 |
| 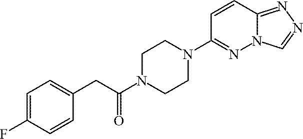 [SMILES: O=C(Cc1ccc(F)cc1)N1CCN(c2ccc3nncn3n2)CC1] | 2-(4-fluorophenyl)-1-(4-{[1,2,4]triazolo[4,3-b]pyridazin-6-yl}piperazin-1-yl)ethan-1-one | SLU-0002632 |
| 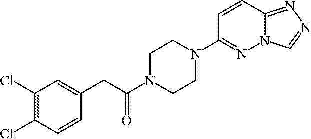 [SMILES: O=C(Cc1ccc(Cl)c(Cl)c1)N1CCN(c2ccc3nncn3n2)CC1] | 2-(3,4-dichlorophenyl)-1-(4-{[1,2,4]triazolo[4,3-b]pyridazin-6-yl}piperazin-1-yl)ethan-1-one | SLU-0002633 |
| 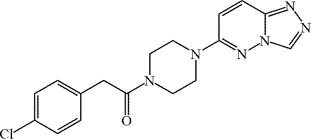 [SMILES: O=C(Cc1ccc(Cl)cc1)N1CCN(c2ccc3nncn3n2)CC1] | 2-(4-chlorophenyl)-1-(4-{[1,2,4]triazolo[4,3-b]pyridazin-6-yl}piperazin-1-yl)ethan-1-one | SLU-0002634 |
| 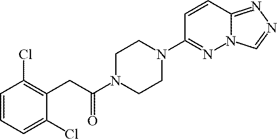 [SMILES: O=C(Cc1c(Cl)cccc1Cl)N1CCN(c2ccc3nncn3n2)CC1] | 2-(2,6-dichlorophenyl)-1-(4-{[1,2,4]triazolo[4,3-b]pyridazin-6-yl}piperazin-1-yl)ethan-1-one | SLU-0002720 |
| 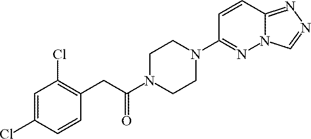 [SMILES: O=C(Cc1ccc(Cl)cc1Cl)N1CCN(c2ccc3nncn3n2)CC1] | 2-(2,4-dichlorophenyl)-1-(4-{[1,2,4]triazolo[4,3-b]pyridazin-6-yl}piperazin-1-yl)ethan-1-one | SLU-0002721 |
| 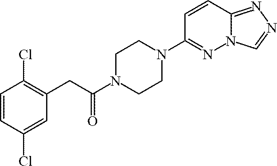 [SMILES: O=C(Cc1cc(Cl)ccc1Cl)N1CCN(c2ccc3nncn3n2)CC1] | 2-(2,5-dichlorophenyl)-1-(4-{[1,2,4]triazolo[4,3-b]pyridazin-6-yl}piperazin-1-yl)ethan-1-one | SLU-0002746 |
| 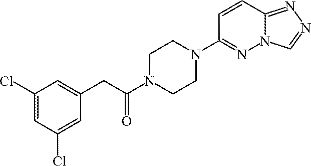 [SMILES: O=C(Cc1cc(Cl)cc(Cl)c1)N1CCN(c2ccc3nncn3n2)CC1] | 2-(3,5-dichlorophenyl)-1-(4-{[1,2,4]triazolo[4,3-b]pyridazin-6-yl}piperazin-1-yl)ethan-1-one | SLU-0002747 |
| 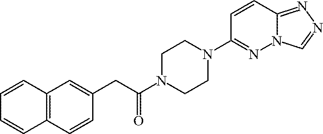 [SMILES: O=C(Cc1ccc2ccccc2c1)N1CCN(c2ccc3nncn3n2)CC1] | 2-(naphthalen-2-yl)-1-(4-{[1,2,4]triazolo[4,3-b]pyridazin-6-yl}piperazin-1-yl)ethan-1-one | SLU-0002749 |
| 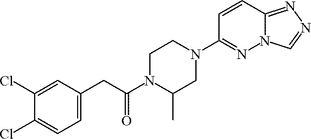 [SMILES: CC1CN(c2ccc3nncn3n2)CCN1C(=O)Cc1ccc(Cl)c(Cl)c1] | 2-(3,4-dichlorophenyl)-1-(2-methyl-4-{[1,2,4]triazolo[4,3-b]pyridazin-6-yl}piperazin-1-yl)ethan-1-one | SLU-0002752 |
| 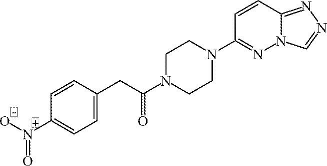 | 2-(4-nitrophenyl)-1-(4-{[1,2,4]triazolo[4,3-b]pyridazin-6-yl}piperazin-1-yl)ethan-1-one | SLU-0002755 |
| 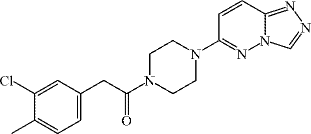 [SMILES: Cc1ccc(CC(=O)N2CCN(c3ccc4nncn4n3)CC2)cc1Cl] | 2-(3-chloro-4-methylphenyl)-1-(4-{[1,2,4]triazolo[4,3-b]pyridazin-6-yl}piperazin-1-yl)ethan-1-one | SLU-0002756 |
| 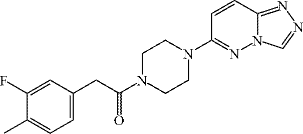 [SMILES: Cc1ccc(CC(=O)N2CCN(c3ccc4nncn4n3)CC2)cc1F] | 2-(3-fluoro-4-methylphenyl)-1-(4-{[1,2,4]triazolo[4,3-b]pyridazin-6-yl}piperazin-1-yl)ethan-1-one | SLU-0002757 |
| 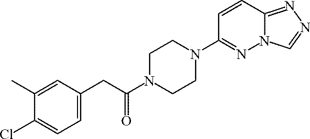 [SMILES: Cc1cc(CC(=O)N2CCN(c3ccc4nncn4n3)CC2)ccc1Cl] | 2-(4-chloro-3-methylphenyl)-1-(4-{[1,2,4]triazolo[4,3-b]pyridazin-6-yl}piperazin-1-yl)ethan-1-one | SLU-0002758 |
| 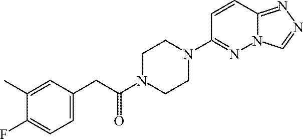 [SMILES: Cc1cc(CC(=O)N2CCN(c3ccc4nncn4n3)CC2)ccc1F] | 2-(4-fluoro-3 -methylphenyl)-1-(4-{[1,2,4]triazolo[4,3-b]pyridazin-6-yl}piperazin-1-yl)ethan-1-one | SLU-0002759 |
| 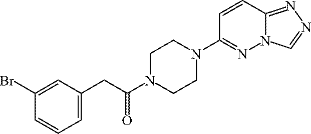 [SMILES: O=C(Cc1cccc(Br)c1)N1CCN(c2ccc3nncn3n2)CC1] | 2-(3-bromophenyl)-1-(4-{[1,2,4]triazolo[4,3-b]pyridazin-6-yl}piperazin-1-yl)ethan-1-one | SLU-0002760 |
| 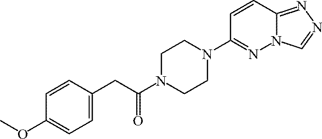 [SMILES: COc1ccc(CC(=O)N2CCN(c3ccc4nncn4n3)CC2)cc1] | 2-(4-methoxyphenyl)-1-(4-{[1,2,4]triazolo[4,3-b]pyridazin-6-yl}piperazin-1-yl)ethan-1-one | SLU-0002761 |
| 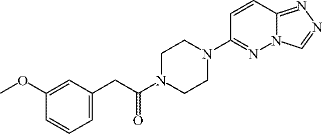 [SMILES: COc1cccc(CC(=O)N2CCN(c3ccc4nncn4n3)CC2)c1] | 2-(3-methoxyphenyl)-1-(4-{[1,2,4]triazolo[4,3-b]pyridazin-6-yl}piperazin-1-yl)ethan-1-one | SLU-0002762 |
| 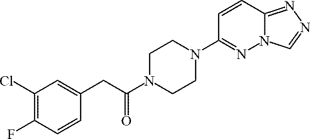 [SMILES: O=C(Cc1ccc(F)c(Cl)c1)N1CCN(c2ccc3nncn3n2)CC1] | 2-(3-chloro-4-fluorophenyl)-1-(4-{[1,2,4]triazolo[4,3-b]pyridazin-6-yl}piperazin-1-yl)ethan-1-one | SLU-0002763 |
| 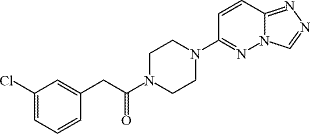 [SMILES: O=C(Cc1cccc(Cl)c1)N1CCN(c2ccc3nncn3n2)CC1] | 2-(3 -chlorophenyl)-1-(4-{[1,2,4]triazolo[4,3-b]pyridazin-6-yl}piperazin-1-yl)ethan-1-one | SLU-0002764 |
| 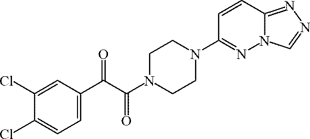 [SMILES: O=C(C(=O)N1CCN(c2ccc3nncn3n2)CC1)c1ccc(Cl)c(Cl)c1] | 1-(3,4-dichlorophenyl)-2-(4-{ [1,2,4]triazolo[4,3-b]pyridazin-6-yl}piperazin-1-yl)ethane-1,2-dione | SLU-0002765 |
| 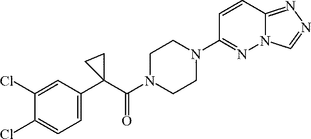 [SMILES: O=C(N1CCN(c2ccc3nncn3n2)CC1)C1(c2ccc(Cl)c(Cl)c2)CC1] | 1-[1-(3,4-dichlorophenyl)cyclopro panecarbonyl]-4-{[1,2,4]triazolo[4,3-b]pyridazin-6-yl}piperazine | SLU-0002766 |
| 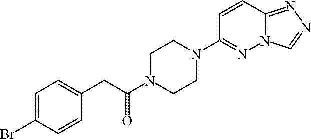 [SMILES: O=C(Cc1ccc(Br)cc1)N1CCN(c2ccc3nncn3n2)CC1] | 2-(4-bromophenyl)-1-(4-{[1,2,4]triazolo[4,3-b]pyridazin-6-yl}piperazin-1-yl)ethan-1-one | SLU-0002767 |
| 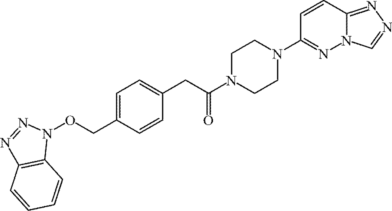 [SMILES: O=C(Cc1ccc(COn2nnc3ccccc32)cc1)N1CCN(c2ccc3nncn3n2)CC1] | 2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one | SLU-0002796 |
| 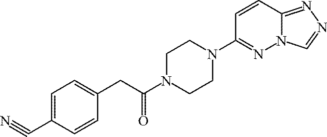 [SMILES: N#Cc1ccc(CC(=O)N2CCN(c3ccc4nncn4n3)CC2)cc1] | 4-[2-oxo-2-(4-{[1,2,4]triazolo[4,3-b]pyridazin-6-yl}piperazin-1-yl)ethyl]benzonitrile | SLU-0002797 |
| 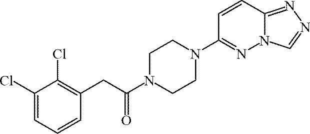 [SMILES: O=C(Cc1cccc(Cl)c1Cl)N1CCN(c2ccc3nncn3n2)CC1] | 2-(2,3 -dichlorophenyl)-1-(4-{[1,2,4]triazolo[4,3-b]pyridazin-6-yl}piperazin-1-yl)ethan-1-one | SLU-0002798 |
| 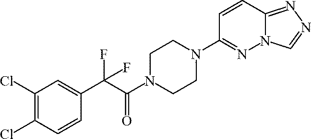 [SMILES: O=C(N1CCN(c2ccc3nncn3n2)CC1)C(F)(F)c1ccc(Cl)c(Cl)c1] | 2-(3,4-dichlorophenyl)-2,2-difluoro-1-(4-{[1,2,4]triazolo[4,3-b]pyridazin-6-yl}piperazin-1-yl)ethan-1-one | SLU-0002799 |
| 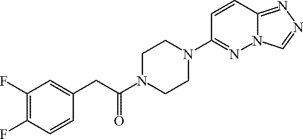 [SMILES: O=C(Cc1ccc(F)c(F)c1)N1CCN(c2ccc3nncn3n2)CC1] | 2-(3,4-difluorophenyl)-1-(4-{[1,2,4]triazolo[4,3-b]pyridazin-6-yl}piperazin-1-yl)ethan-1-one | SLU-0002800 |
| 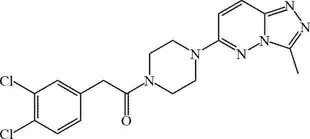 [SMILES: Cc1nnc2ccc(N3CCN(C(=O)Cc4ccc(Cl)c(Cl)c4)CC3)nn12] | 2-(3,4-dichlorophenyl)-1-(4-{3-methyl-[1,2,4]triazolo[4,3-b]pyridazin-6-yl}piperazin-1-yl)ethan-1-one | SLU-0002808 |
| 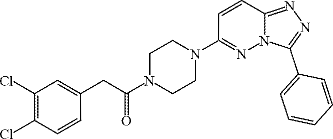 [SMILES: O=C(Cc1ccc(Cl)c(Cl)c1)N1CCN(c2ccc3nnc(-c4ccccc4)n3n2)CC1] | 2-(3,4-dichlorophenyl)-1-(4-{3-phenyl-[1,2,4]triazolo[4,3-b]pyridazin-6-yl}piperazin-1-yl)ethan-1-one | SLU-0002807 |
| 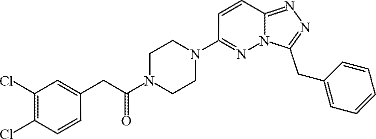 [SMILES: O=C(Cc1ccc(Cl)c(Cl)c1)N1CCN(c2ccc3nnc(Cc4ccccc4)n3n2)CC1] | 1-(4-{3-benzyl-[1,2,4]triazolo[4,3-b]pyridazin-6-yl}piperazin-1 -yl)-2-(3,4-dichlorophenyl)ethan-1-one | SLU-0010249 |
| 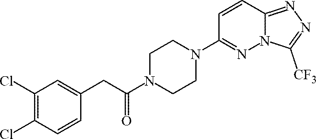 [SMILES: O=C(Cc1ccc(Cl)c(Cl)c1)N1CCN(c2ccc3nnc(C(F)(F)F)n3n2)CC1] | 2-(3,4-dichlorophenyl)-1-{4-[3-(trifluoromethyl)-[1,2,4]triazolo[4,3-b]pyridazin-6-yl]piperazin-1-yl}ethan-1-one | SLU-0002810 |
| 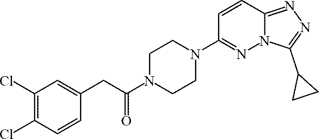 [SMILES: O=C(Cc1ccc(Cl)c(Cl)c1)N1CCN(c2ccc3nnc(C4CC4)n3n2)CC1] | 1-(4-{3-cyclopropyl-[1,2,4]triazolo[4,3-b]pyridazin-6-yl}piperazin-1-yl)-2-(3,4-dichlorophenyl)ethan-1-one | |
| 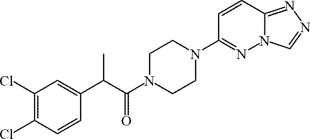 [SMILES: CC(C(=O)N1CCN(c2ccc3nncn3n2)CC1)c1ccc(Cl)c(Cl)c1] | 2-(3,4-dichlorophenyl)-1-(4-{[1,2,4]triazolo[4,3-b]pyridazin-6-yl}piperazin-1-yl)propan-1-one | |
| 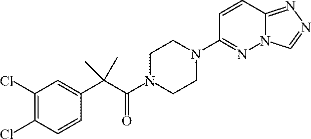 [SMILES: CC(C)(C(=O)N1CCN(c2ccc3nncn3n2)CC1)c1ccc(Cl)c(Cl)c1] | 2-(3,4-dichlorophenyl)-2-methyl-1-(4-{[1,2,4]triazolo[4,3-b]pyridazin-6-yl}piperazin-1-yl)propan-1-one | |
| 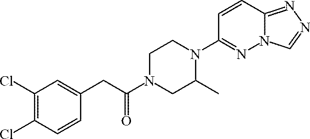 [SMILES: CC1CN(C(=O)Cc2ccc(Cl)c(Cl)c2)CCN1c1ccc2nncn2n1] | 2-(3,4-dichlorophenyl)-1-(3-methyl-4-{[1,2,4]triazolo[4,3-b]pyridazin-6-yl}piperazin-1-yl)ethan-1-one | |
| 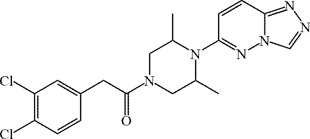 [SMILES: CC1CN(C(=O)Cc2ccc(Cl)c(Cl)c2)CC(C)N1c1ccc2nncn2n1] | 2-(3,4-dichlorophenyl)-1-(3,5-dimethyl-4-{[1,2,4]triazolo[4,3-b]pyridazin-6-yl}piperazin-1-yl)ethan-1-one | |
| 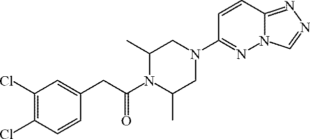 [SMILES: CC1CN(c2ccc3nncn3n2)CC(C)N1C(=O)Cc1ccc(Cl)c(Cl)c1] | 2-(3,4-dichlorophenyl)-1-(2,6-dimethyl-4-{[1,2,4]triazolo[4,3-b]pyridazin-6-yl}piperazin-1-yl)ethan-1-one | |
| 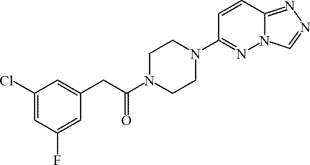 [SMILES: O=C(Cc1cc(F)cc(Cl)c1)N1CCN(c2ccc3nncn3n2)CC1] | 2-(3-chloro-5-fluorophenyl)-1-(4-{[1,2,4]triazolo[4,3-b]pyridazin-6-yl}piperazin-1-yl)ethan-1-one | SLU-0010483 |
| 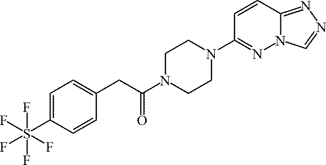 [SMILES: O=C(Cc1ccc(S(F)(F)(F)(F)F)cc1)N1CCN(c2ccc3nncn3n2)CC1] | 2-[4-(pentafluoro-λ⁶- sulfanyl)phenyl]-1-(4-{[1,2,4]triazolo[4,3-b]pyridazin-6-yl}piperazin-1-yl)ethan-1-one | SLU-0010441 |
| 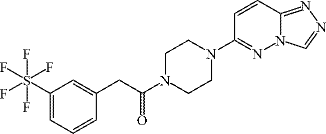 [SMILES: O=C(Cc1cccc(S(F)(F)(F)(F)F)c1)N1CCN(c2ccc3nncn3n2)CC1] | 2-[3-(pentafluoro-λ⁶- sulfanyl)phenyl]-1-(4-{[1,2,4]triazolo[4,3-b]pyridazin-6-yl}piperazin-1-yl)ethan-1-one | SLU-0010442 |
| 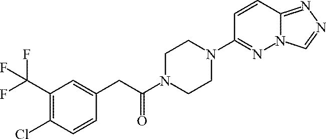 [SMILES: O=C(Cc1ccc(Cl)c(C(F)(F)F)c1)N1CCN(c2ccc3nncn3n2)CC1] | 2-[4-chloro-3-(trifluoromethyl)phenyl] -1-(4-{[1,2,4]triazolo[4,3-b]pyridazin-6-yl}piperazin-1-yl)ethan-1-one | SLU-0010481 |
| 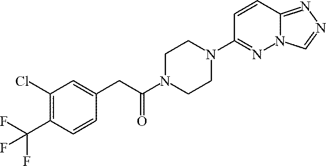 [SMILES: O=C(Cc1ccc(C(F)(F)F)c(Cl)c1)N1CCN(c2ccc3nncn3n2)CC1] | 2-[3-chloro-4-(trifluoromethyl)phenyl] -1-(4-{[1,2,4]triazolo[4,3-b]pyridazin-6- | SLU-0010551 |
| | yl}piperazin-1-yl)ethan-1-one | |
| 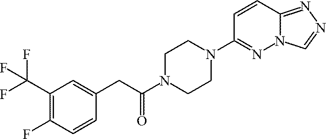 [SMILES: O=C(Cc1ccc(F)c(C(F)(F)F)c1)N1CCN(c2ccc3nncn3n2)CC1] | 2-[4-fluoro-3-(trifluoromethyl)phenyl] -1-(4-{[1,2,4]triazolo[4,3-b]pyridazin-6-yl}piperazin-1-yl)ethan-1-one | SLU-0010482 |
| 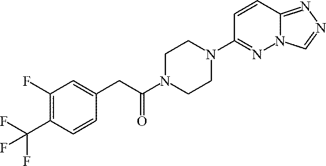 [SMILES: O=C(Cc1ccc(C(F)(F)F)c(F)c1)N1CCN(c2ccc3nncn3n2)CC1] | 2-[3-fluoro-4-(trifluoromethyl)phenyl] -1-(4-{[1,2,4]triazolo[4,3-b]pyridazin-6-yl}piperazin-1-yl)ethan-1-one | SLU-0010480 |
| 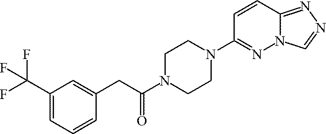 [SMILES: O=C(Cc1cccc(C(F)(F)F)c1)N1CCN(c2ccc3nncn3n2)CC1] | 1-(4-{[1,2,4]triazolo[4,3-b]pyridazin-6-yl}piperazin-1-yl)-2-[3-(trifluoromethyl)phenyl] ethan-1-one | SLU-0010549 |
| 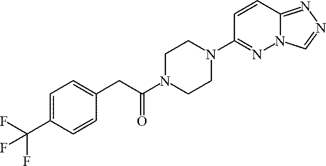 [SMILES: O=C(Cc1ccc(C(F)(F)F)cc1)N1CCN(c2ccc3nncn3n2)CC1] | 1-(4-{[1,2,4]triazolo[4,3-b]pyridazin-6-yl}piperazin-1-yl)-2-[4-(trifluoromethyl)phenyl] ethan-1-one | SLU-0010484 |
| 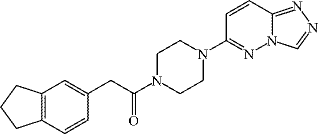 [SMILES: O=C(Cc1ccc2c(c1)CCC2)N1CCN(c2ccc3nncn3n2)CC1] | 2-(2,3-dihydro-1H-inden-5-yl)-1-(4-{[1,2,4]triazolo[4,3-b]pyridazin-6-yl}piperazin-1-yl)ethan-1-one | |
| 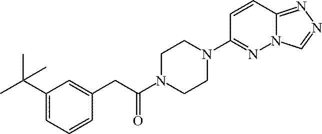 [SMILES: CC(C)(C)c1cccc(CC(=O)N2CCN(c3ccc4nncn4n3)CC2)c1] | 2-(3-tert-butylphenyl)-1-(4-{[1,2,4]triazolo[4,3-b]pyridazin-6-yl}piperazin-1-yl)ethan-1-one | |
| 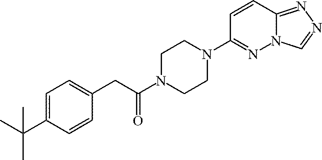 [SMILES: CC(C)(C)c1ccc(CC(=O)N2CCN(c3ccc4nncn4n3)CC2)cc1] | 2-(4-tert-butylphenyl)-1-(4-{[1,2,4]triazolo[4,3-b]pyridazin-6-yl}piperazin-1-yl)ethan-1-one | |
| 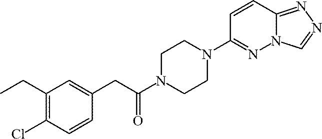 [SMILES: CCc1cc(CC(=O)N2CCN(c3ccc4nncn4n3)CC2)ccc1Cl] | 2-(4-chloro-3-ethylphenyl)-1-(4-{[1,2,4]triazolo[4,3-b]pyridazin-6-yl}piperazin-1-yl)ethan-1-one | |
| 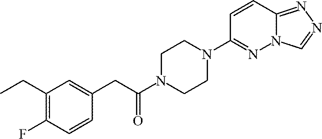 [SMILES: CCc1cc(CC(=O)N2CCN(c3ccc4nncn4n3)CC2)ccc1F] | 2-(3-ethyl-4-fluorophenyl)-1-(4-{[1,2,4]triazolo[4,3-b]pyridazin-6-yl}piperazin-1-yl)ethan-1-one | |
| 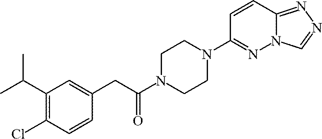 [SMILES: CC(C)c1cc(CC(=O)N2CCN(c3ccc4nncn4n3)CC2)ccc1Cl] | 2- [4-chloro-3 -(propan-2-yl)phenyl]-1-(4-{[1,2,4]triazolo[4,3-b]pyridazin-6-yl}piperazin-1-yl)ethan-1-one | |
| 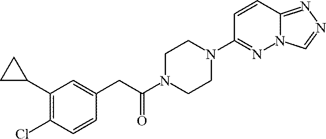 [SMILES: O=C(Cc1ccc(Cl)c(C2CC2)c1)N1CCN(c2ccc3nncn3n2)CC1] | 2-(4-chloro-3-cyclopropylphenyl)-1 -(4-{[1,2,4]triazolo[4,3-b]pyridazin-6-yl}piperazin-1-yl)ethan-1-one | |
| 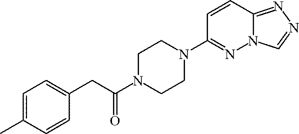 [SMILES: Cc1ccc(CC(=O)N2CCN(c3ccc4nncn4n3)CC2)cc1] | 2-(4-methylphenyl)-1-(4-{[1,2,4]triazolo[4,3-b]pyridazin-6-yl}piperazin-1-yl)ethan-1-one | SLU-0010505 |
| 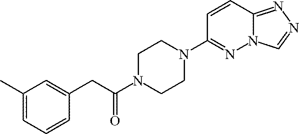 [SMILES: Cc1cccc(CC(=O)N2CCN(c3ccc4nncn4n3)CC2)c1] | 2-(3-methylphenyl)-1-(4-{[1,2,4]triazolo[4,3-b]pyridazin-6-yl}piperazin-1-yl)ethan-1-one | SLU-0010504 |
| 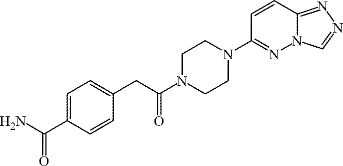 [SMILES: NC(=O)c1ccc(CC(=O)N2CCN(c3ccc4nncn4n3)CC2)cc1] | 4-[2-oxo-2-(4-{[1,2,4]triazolo[4,3-b]pyridazin-6-yl}piperazin-1-yl)ethyl]benzamide | |
| 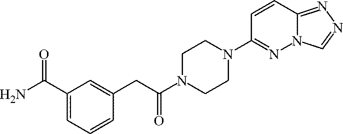 [SMILES: NC(=O)c1cccc(CC(=O)N2CCN(c3ccc4nncn4n3)CC2)c1] | 3-[2-oxo-2-(4-{[1,2,4]triazolo[4,3-b]pyridazin-6-yl}piperazin-1-yl)ethyl]benzamide | |
| 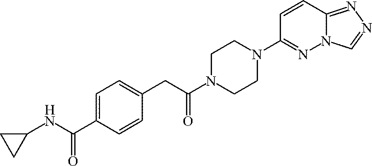 [SMILES: O=C(NC1CC1)c1ccc(CC(=O)N2CCN(c3ccc4nncn4n3)CC2)cc1] | N-cyclopropyl-4-[2-oxo-2-(4-{[1,2,4]triazolo[4,3-b]pyridazin-6-yl}piperazin-1-yl)ethyl]benzamide | |
| 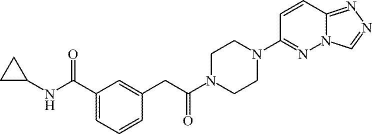 [SMILES: O=C(NC1CC1)c1cccc(CC(=O)N2CCN(c3ccc4nncn4n3)CC2)c1] | N-cyclopropyl-3-[2-oxo-2-(4-{[1,2,4]triazolo[4,3-b]pyridazin-6-yl}piperazin-1-yl)ethyl]benzamide | |
| 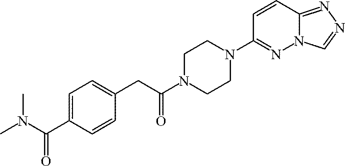 [SMILES: CN(C)C(=O)c1ccc(CC(=O)N2CCN(c3ccc4nncn4n3)CC2)cc1] | N,N-dimethyl-4-[2-oxo-2-(4-{[1,2,4]triazolo[4,3-b]pyridazin-6-yl}piperazin-1-yl)ethyl]benzamide | |
| 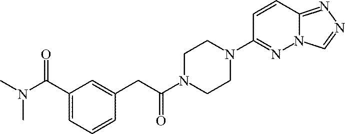 [SMILES: CN(C)C(=O)c1cccc(CC(=O)N2CCN(c3ccc4nncn4n3)CC2)c1] | N,N-dimethyl-3-[2-oxo-2-(4-{[1,2,4]triazolo[4,3-b]pyridazin-6-yl}piperazin-1-yl)ethyl]benzamide | |
| 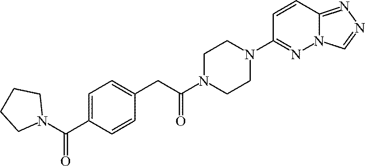 [SMILES: O=C(Cc1ccc(C(=O)N2CCCC2)cc1)N1CCN(c2ccc3nncn3n2)CC1] | 2-[4-(pyrrolidine-1-carbonyl)phenyl]-1-(4-{[1,2,4]triazolo[4,3-b]pyridazin-6-yl}piperazin-1-yl)ethan-1-one | |
| 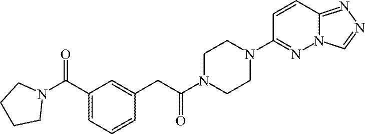 [SMILES: O=C(Cc1cccc(C(=O)N2CCCC2)c1)N1CCN(c2ccc3nncn3n2)CC1] | 2-[3-(pyrrolidine-1-carbonyl)phenyl]-1-(4-{[1,2,4]triazolo[4,3-b]pyridazin-6-yl}piperazin-1-yl)ethan-1-one | |
| 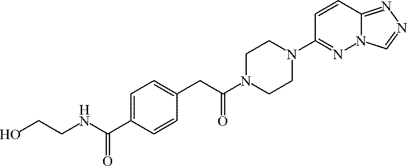 [SMILES: O=C(NCCO)c1ccc(CC(=O)N2CCN(c3ccc4nncn4n3)CC2)cc1] | N-(2-hydroxyethyl)-4-[2-oxo-2-(4-{[1,2,4]triazolo[4,3-b]pyridazin-6-yl(piperazin-1-yl)ethyl]benzamide | |
| 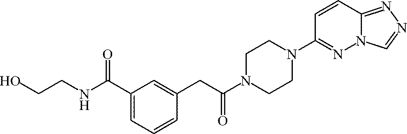 [SMILES: O=C(NCCO)c1cccc(CC(=O)N2CCN(c3ccc4nncn4n3)CC2)c1] | N-(2-hydroxyethyl)-3-[2-oxo-2-(4-{[1,2,4]triazolo[4,3-b]pyridazin-6-yl}piperazin-1-yl)ethyl]benzamide | |
| 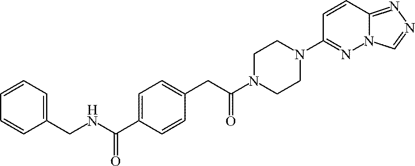 [SMILES: O=C(NCc1ccccc1)c1ccc(CC(=O)N2CCN(c3ccc4nncn4n3)CC2)cc1] | N-benzyl-4-[2-oxo-2-(4-{[1,2,4]triazolo[4,3-b]pyridazin-6-yl}piperazin-1-yl)ethyl]benzamide | |
| 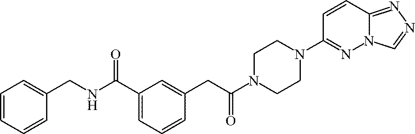 [SMILES: O=C(NCc1ccccc1)c1cccc(CC(=O)N2CCN(c3ccc4nncn4n3)CC2)c1] | N-benzyl-3-[2-oxo-2-(4-{[1,2,4]triazolo[4,3-b]pyridazin-6-yl}piperazin-1-yl)ethyl]benzamide | |
| 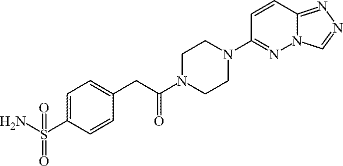 [SMILES: NS(=O)(=O)c1ccc(CC(=O)N2CCN(c3ccc4nncn4n3)CC2)cc1] | 4-[2-oxo-2-(4-{[1,2,4]triazolo[4,3-b]pyridazin-6-yl}piperazin-1-yl)ethyl]benzene-1-sulfonamide | |
| 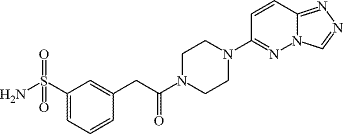 [SMILES: NS(=O)(=O)c1cccc(CC(=O)N2CCN(c3ccc4nncn4n3)CC2)c1] | 3-[2-oxo-2-(4-{[1,2,4]triazolo[4,3-b]pyridazin-6-yl}piperazin-1-yl)ethyl]benzene-1-sulfonamide | |
| 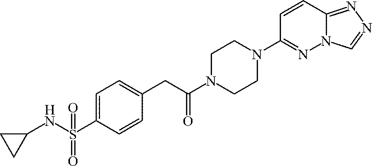 [SMILES: O=C(Cc1ccc(S(=O)(=O)NC2CC2)cc1)N1CCN(c2ccc3nncn3n2)CC1] | N-cyclopropyl-4-[2-oxo-2-(4-{[1,2,4]triazolo[4,3-b]pyridazin-6-yl}piperazin-1-yl)ethyl]benzene-1-sulfonamide | |
| 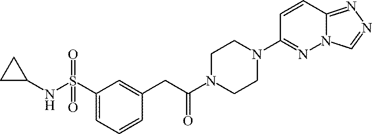 [SMILES: O=C(Cc1cccc(S(=O)(=O)NC2CC2)c1)N1CCN(c2ccc3nncn3n2)CC1] | N-cyclopropyl-3-[2-oxo-2-(4-{[1,2,4]triazolo[4,3-b]pyridazin-6-yl}piperazin-1-yl)ethyl]benzene-1-sulfonamide | |
| 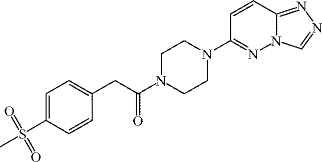 [SMILES: CS(=O)(=O)c1ccc(CC(=O)N2CCN(c3ccc4nncn4n3)CC2)cc1] | 2-(4-methanesulfonylphenyl) -1-(4-{[1,2,4]triazolo[4,3-b]pyridazin-6-yl}piperazin-1-yl)ethan-1-one | |
| 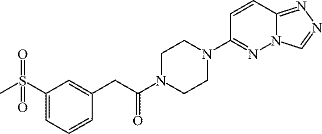 [SMILES: CS(=O)(=O)c1cccc(CC(=O)N2CCN(c3ccc4nncn4n3)CC2)c1] | 2-(3-methanesulfonylphenyl) -1-(4-{[1,2,4]triazolo[4,3-b]pyridazin-6-yl}piperazin-1-yl)ethan-1-one | |
| 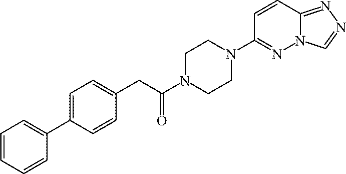 [SMILES: O=C(Cc1ccc(-c2ccccc2)cc1)N1CCN(c2ccc3nncn3n2)CC1] | 2-{[1,1′-biphenyl]-4-yl}-1-(4-{[1,2,4]triazolo[4,3-b]pyridazin-6-yl}piperazin-1-yl)ethan-1-one | SLU-0002809 |
| 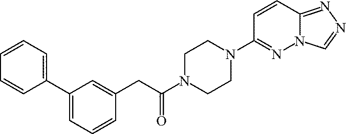 [SMILES: O=C(Cc1cccc(-c2ccccc2)c1)N1CCN(c2ccc3nncn3n2)CC1] | 2-{[1,1′-biphenyl]-3-yl}-1-(4-{[1,2,4]triazolo[4,3-b]pyridazin-6-yl}piperazin-1-yl)ethan-1-one | |
| 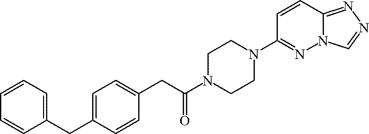 [SMILES: O=C(Cc1ccc(Cc2ccccc2)cc1)N1CCN(c2ccc3nncn3n2)CC1] | 2-(4-benzylphenyl)-1-(4-{[1,2,4]triazolo[4,3-b]pyridazin-6-yl}piperazin-1-yl)ethan-1-one | |
| 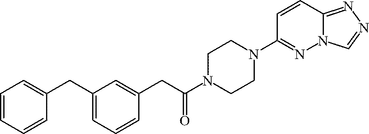 [SMILES: O=C(Cc1cccc(Cc2ccccc2)c1)N1CCN(c2ccc3nncn3n2)CC1] | 2-(3-benzylphenyl)-1-(4-{[1,2,4]triazolo[4,3-b]pyridazin-6-yl}piperazin-1-yl)ethan-1-one | |
| 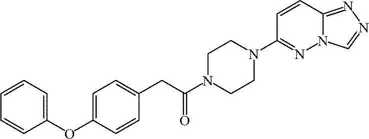 [SMILES: O=C(Cc1ccc(Oc2ccccc2)cc1)N1CCN(c2ccc3nncn3n2)CC1] | 2-(4-phenoxyphenyl)-1-(4-{[1,2,4]triazolo[4,3-b]pyridazin-6-yl}piperazin-1-yl)ethan-1-one | |
| 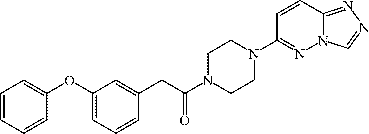 [SMILES: O=C(Cc1cccc(Oc2ccccc2)c1)N1CCN(c2ccc3nncn3n2)CC1] | 2-(3-phenoxyphenyl)-1-(4-{[1,2,4]triazolo[4,3-b]pyridazin-6-yl}piperazin-1-yl)ethan-1-one | |
| 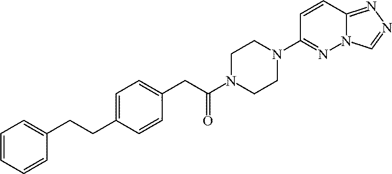 [SMILES: O=C(Cc1ccc(CCc2ccccc2)cc1)N1CCN(c2ccc3nncn3n2)CC1] | 2-[4-(2-phenylethyl)phenyl]-1-(4-{ [1,2,4]triazolo[4,3-b]pyridazin-6-yl}piperazin-1-yl)ethan-1-one | |
| 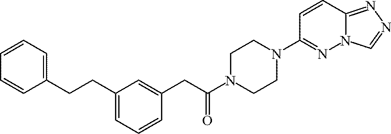 [SMILES: O=C(Cc1cccc(CCc2ccccc2)c1)N1CCN(c2ccc3nncn3n2)CC1] | 2-[3-(2-phenylethyl)phenyl]-1-(4-{ [1,2,4]triazolo[4,3-b]pyridazin-6-yl}piperazin-1-yl)ethan-1-one | |
| 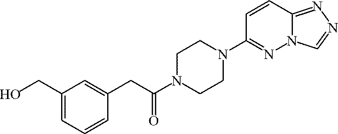 [SMILES: O=C(Cc1cccc(CO)c1)N1CCN(c2ccc3nncn3n2)CC1] | 2-[3-(hydroxymethyl)phenyl] -1-(4-{[1,2,4]triazolo[4,3-b]pyridazin-6-yl}piperazin-1-yl)ethan-1-one | |
| 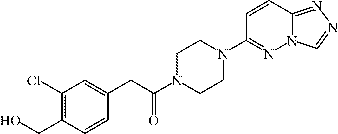 [SMILES: O=C(Cc1ccc(CO)c(Cl)c1)N1CCN(c2ccc3nncn3n2)CC1] | 2-[3-chloro-4-(hydroxymethyl)phenyl] -1-(4-{[1,2,4]triazolo[4,3-b]pyridazin-6-yl}piperazin-1-yl)ethan-1-one | |
| 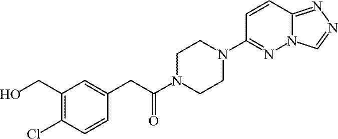 [SMILES: O=C(Cc1ccc(Cl)c(CO)c1)N1CCN(c2ccc3nncn3n2)CC1] | 2-[4-chloro-3-(hydroxymethyl)phenyl] -1-(4-{[1,2,4]triazolo[4,3-b]pyridazin-6-yl}piperazin-1-yl)ethan-1-one | |
| 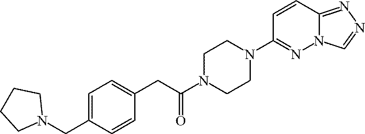 [SMILES: O=C(Cc1ccc(CN2CCCC2)cc1)N1CCN(c2ccc3nncn3n2)CC1] | 2-{4-[(pyrrolidin-1-yl)methyl]phenyl}-1-(4-{[1,2,4]triazolo[4,3-b]pyridazin-6-yl}piperazin-1-yl)ethan-1-one | |
| 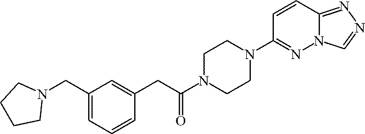 [SMILES: O=C(Cc1cccc(CN2CCCC2)c1)N1CCN(c2ccc3nncn3n2)CC1] | 2-{3-[(pyrrolidin-1-yl)methyl]phenyl}-1-(4-{[1,2,4]triazolo[4,3-b]pyridazin-6-yl}piperazin-1-yl)ethan-1-one | |
| 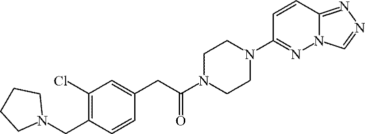 [SMILES: O=C(Cc1ccc(CN2CCCC2)c(Cl)c1)N1CCN(c2ccc3nncn3n2)CC1] | 2-{3-chloro-4-[(pyrrolidin-1-yl)methyl]phenyl}-1-(4-{[1,2,4]triazolo[4,3-b]pyridazin-6-yl}piperazin-1-yl)ethan-1-one | |
| 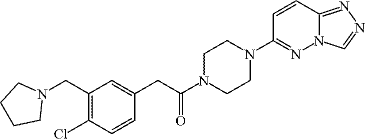 [SMILES: O=C(Cc1ccc(Cl)c(CN2CCCC2)c1)N1CCN(c2ccc3nncn3n2)CC1] | 2-{4-chloro-3-[(pyrrolidin-1-yl)methyl]phenyl}-1-(4-{[1,2,4]triazolo[4,3-b]pyridazin-6-yl}piperazin-1-yl)ethan-1-one | |
| 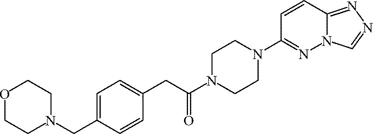 [SMILES: O=C(Cc1ccc(CN2CCOCC2)cc1)N1CCN(c2ccc3nncn3n2)CC1] | 2-{4-[(morpholin-4-yl)methyl]phenyl}-1-(4-{[1,2,4]triazolo[4,3-b]pyridazin-6-yl}piperazin-1-yl)ethan-1-one | SLU-0002817 |
| 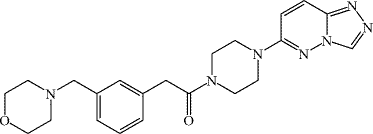 [SMILES: O=C(Cc1cccc(CN2CCOCC2)c1)N1CCN(c2ccc3nncn3n2)CC1] | 2-{3-[(morpholin-4-yl)methyl]phenyl}-1-(4-{[1,2,4]triazolo[4,3-b]pyridazin-6-yl}piperazin-1-yl)ethan-1-one | |
| 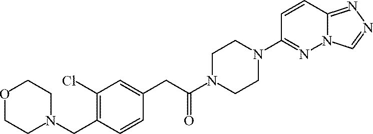 [SMILES: O=C(Cc1ccc(CN2CCOCC2)c(Cl)c1)N1CCN(c2ccc3nncn3n2)CC1] | 2-{3-chloro-4-[(morpholin-4-yl)methyl]phenyl}-1-(4-{[1,2,4]triazolo[4,3-b]pyridazin-6-yl}piperazin-1-yl)ethan-1-one | |
| 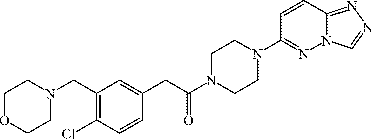 [SMILES: O=C(Cc1ccc(Cl)c(CN2CCOCC2)c1)N1CCN(c2ccc3nncn3n2)CC1] | 2-{4-chloro-3-[(morpholin-4-yl)methyl]phenyl}-1-(4-{[1,2,4]triazolo[4,3-b]pyridazin-6-yl}piperazin-1-yl)ethan-1-one | |
| 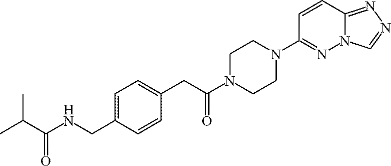 [SMILES: CC(C)C(=O)NCc1ccc(CC(=O)N2CCN(c3ccc4nncn4n3)CC2)cc1] | 2-methyl-N-({4-[2-oxo-2-(4-{[1,2,4]triazolo[4,3-b]pyridazin-6-yl}piperazin-1-yl)ethyl]phenyl}methyl) propanamide | |
| 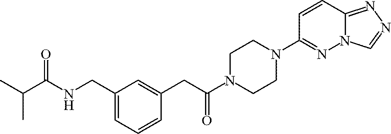 [SMILES: CC(C)C(=O)NCc1cccc(CC(=O)N2CCN(c3ccc4nncn4n3)CC2)c1] | 2-methyl-N-({3-[2-oxo-2-(4-{[1,2,4]triazolo[4,3-b]pyridazin-6-yl}piperazin-1-yl)ethyl]phenyl}methyl) propanamide | |
| 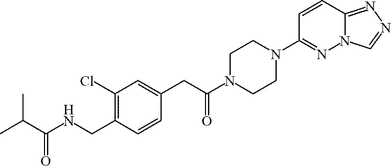 [SMILES: CC(C)C(=O)NCc1ccc(CC(=O)N2CCN(c3ccc4nncn4n3)CC2)cc1Cl] | N-({ 2-chloro-4-[2-oxo-2-(4-{ [1,2,4]triazolo[4,3-b]pyridazin-6-yl}piperazin-1-yl)ethyl]phenyl}methyl) -2-methylpropanamide | |
| 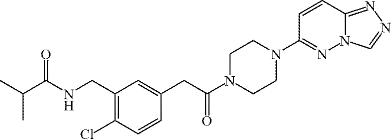 [SMILES: CC(C)C(=O)NCc1cc(CC(=O)N2CCN(c3ccc4nncn4n3)CC2)ccc1Cl] | N-({ 2-chloro-5-[2-oxo-2-(4-{ [1,2,4]triazolo[4,3-b]pyridazin-6-yl}piperazin-1-yl)ethyl]phenyl}methyl) -2-methylpropanamide | |
| 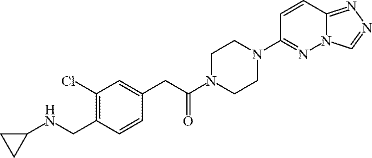 [SMILES: O=C(Cc1ccc(CNC2CC2)c(Cl)c1)N1CCN(c2ccc3nncn3n2)CC1] | 2-{3-chloro-4-[(cyclopropylamino)met hyl]phenyl}-1-(4-{[1,2,4]triazolo[4,3-b]pyridazin-6-yl}piperazin-1-yl)ethan-1-one | |
| 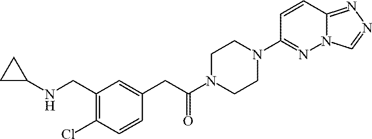 [SMILES: O=C(Cc1ccc(Cl)c(CNC2CC2)c1)N1CCN(c2ccc3nncn3n2)CC1] | 2-{4-chloro-3-[(cyclopropylamino)met hyl]phenyl}-1-(4-{[1,2,4]triazolo[4,3-b]pyridazin-6-yl}piperazin-1-yl)ethan-1-one | |
| 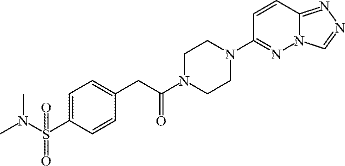 [SMILES: CN(C)S(=O)(=O)c1ccc(CC(=O)N2CCN(c3ccc4nncn4n3)CC2)cc1] | N,N-dimethyl-4-[2-oxo-2-(4-{[1,2,4]triazolo[4,3-b]pyridazin-6-yl}piperazin-1-yl)ethyl]benzene-1-sulfonamide | |
| 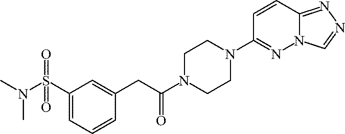 [SMILES: CN(C)S(=O)(=O)c1cccc(CC(=O)N2CCN(c3ccc4nncn4n3)CC2)c1] | N,N-dimethyl-3-[2-oxo-2-(4-{[1,2,4]triazolo[4,3-b]pyridazin-6-yl}piperazin-1-yl)ethyl]benzene-1-sulfonamide | |
| 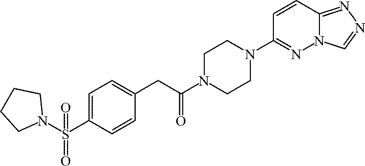 [SMILES: O=C(Cc1ccc(S(=O)(=O)N2CCCC2)cc1)N1CCN(c2ccc3nncn3n2)CC1] | 2-[4-(pyrrolidine-1-sulfonyl)phenyl]-1-(4-{[1,2,4]triazolo[4,3-b]pyridazin-6-yl}piperazin-1-yl)ethan-1-one | |
| 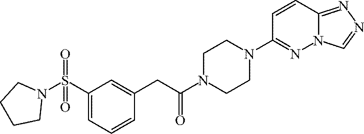 [SMILES: O=C(Cc1cccc(S(=O)(=O)N2CCCC2)c1)N1CCN(c2ccc3nncn3n2)CC1] | 2-[3-(pyrrolidine-1-sulfonyl)phenyl]-1-(4-{[1,2,4]triazolo[4,3-b]pyridazin-6-yl}piperazin-1-yl)ethan-1-one | |
| 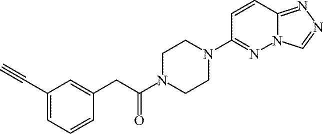 [SMILES: C#Cc1cccc(CC(=O)N2CCN(c3ccc4nncn4n3)CC2)c1] | 2-(3-ethynylphenyl)-1-(4-{[1,2,4]triazolo[4,3-b]pyridazin-6-yl}piperazin-1-yl)ethan-1-one | |
| 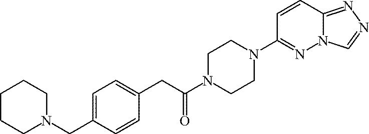 [SMILES: O=C(Cc1ccc(CN2CCCCC2)cc1)N1CCN(c2ccc3nncn3n2)CC1] | 2-{4-[(piperidin-1-yl)methyl]phenyl}-1-(4-{[1,2,4]triazolo[4,3-b]pyridazin-6-yl}piperazin-1-yl)ethan-1-one | |
| 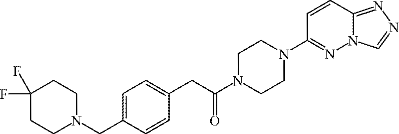 [SMILES: O=C(Cc1ccc(CN2CCC(F)(F)CC2)cc1)N1CCN(c2ccc3nncn3n2)CC1] | 2-{4-[(4,4-difluoropiperidin-1-yl)methyl]phenyl}-1-(4-{[1,2,4]triazolo[4,3-b]pyridazin-6-yl}piperazin-1-yl)ethan-1-one | |
| 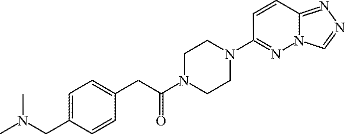 [SMILES: CN(C)Cc1ccc(CC(=O)N2CCN(c3ccc4nncn4n3)CC2)cc1] | 2-{4-[(dimethylamino)methyl ]phenyl}-1-(4-{[1,2,4]triazolo[4,3-b]pyridazin-6-yl}piperazin-1-yl)ethan-1-one | |
| 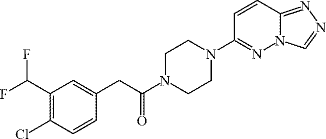 [SMILES: O=C(Cc1ccc(Cl)c(C(F)F)c1)N1CCN(c2ccc3nncn3n2)CC1] | 2-[4-chloro-3-(difluoromethyl)phenyl] -1-(4-{[1,2,4]triazolo[4,3-b]pyridazin-6-yl}piperazin-1-yl)ethan-1-one | |
| 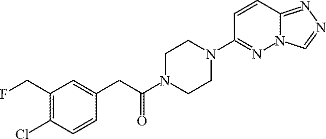 [SMILES: O=C(Cc1ccc(Cl)c(CF)c1)N1CCN(c2ccc3nncn3n2)CC1] | 2-[4-chloro-3-(fluoromethyl)phenyl]-1-(4-{[1,2,4]triazolo[4,3-b]pyridazin-6-yl}piperazin-1-yl)ethan-1-one | |
| 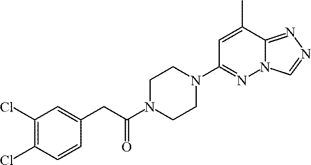 [SMILES: Cc1cc(N2CCN(C(=O)Cc3ccc(Cl)c(Cl)c3)CC2)nn2cnnc12] | 2-(3,4-dichlorophenyl)-1-(4-{8-methyl-[1,2,4]triazolo[4,3-b]pyridazin-6-yl}piperazin-1-yl)ethan-1-one | |
| 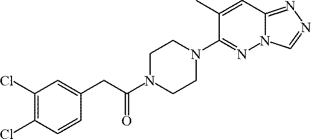 [SMILES: Cc1cc2nncn2nc1N1CCN(C(=O)Cc2ccc(Cl)c(Cl)c2)CC1] | 2-(3,4-dichlorophenyl)-1-(4-{7-methyl-[1,2,4]triazolo[4,3-b]pyridazin-6-yl}piperazin-1-yl)ethan-1-one | |
| 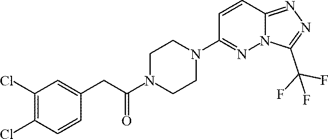 [SMILES: O=C(Cc1ccc(Cl)c(Cl)c1)N1CCN(c2ccc3nnc(C(F)(F)F)n3n2)CC1] | 2-(3,4-dichlorophenyl)-1 -{ 4-[3-(trifluoromethyl)-[1,2,4]triazolo[4,3-b]pyridazin-6-yl]piperazin-1 -yl} ethan-1 -one | SLU-0002810 |
| 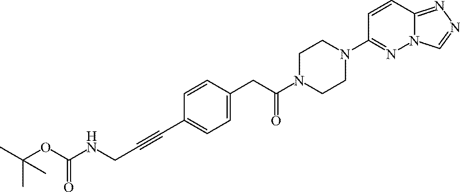 [SMILES: CC(C)(C)OC(=O)NCC#Cc1ccc(CC(=O)N2CCN(c3ccc4nncn4n3)CC2)cc1] | tert-butyl N-(3-{4-[2-oxo-2-(4-{[1,2,4]triazolo[4,3-b]pyridazin-6-yl}piperazin-1-yl)ethyl]phenyl}prop-2-yn-1-yl)carbamate | SLU-0002811 |
| 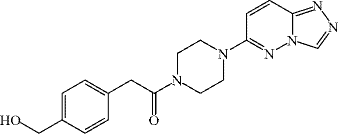 [SMILES: O=C(Cc1ccc(CO)cc1)N1CCN(c2ccc3nncn3n2)CC1] | 2-[4-(hydroxymethyl)phenyl] -1-(4-{[1,2,4]triazolo[4,3-b]pyridazin-6-yl }piperazin-1 -yl)ethan-1 -one | SLU-0002818 |
| 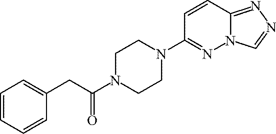 [SMILES: O=C(Cc1ccccc1)N1CCN(c2ccc3nncn3n2)CC1] | 2-phenyl-1-(4-{[1,2,4]triazolo[4,3-b]pyridazin-6-yl}piperazin-1-yl)ethan-1-one | SLU-0002819 |
| 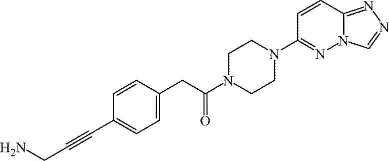 [SMILES: NCC#Cc1ccc(CC(=O)N2CCN(c3ccc4nncn4n3)CC2)cc1] | 2-[4-(3-aminoprop-1-yn-1-yl)phenyl]-1-(4-{[1,2,4]triazolo[4,3-b]pyridazin-6-yl}piperazin-1-yl)ethan-1-one | SLU-0010037 |
| 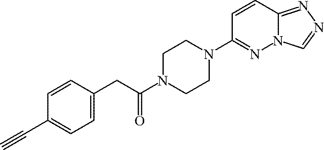 [SMILES: C#Cc1ccc(CC(=O)N2CCN(c3ccc4nncn4n3)CC2)cc1] | 2-(4-ethynylphenyl)-1-(4-{[1,2,4]triazolo[4,3-b]pyridazin-6-yl}piperazin-1-yl)ethan-1-one | SLU-0010046 |
| 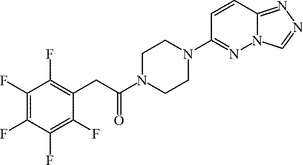 [SMILES: O=C(Cc1c(F)c(F)c(F)c(F)c1F)N1CCN(c2ccc3nncn3n2)CC1] | 2-(2,3,4,5,6-pentafluorophenyl)-1-(4-{[1,2,4]triazolo[4,3-b]pyridazin-6-yl}piperazin-1-yl)ethan-1-one | SLU-0010090 |
| 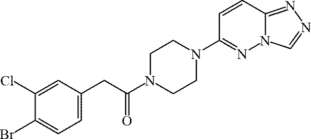 [SMILES: O=C(Cc1ccc(Br)c(Cl)c1)N1CCN(c2ccc3nncn3n2)CC1] | 2-(4-bromo-3-chlorophenyl)-1-(4-{[1,2,4]triazolo[4,3-b]pyridazin-6-yl}piperazin-1-yl)ethan-1-one | SLU-0010238 |
| 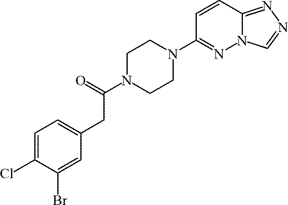 [SMILES: O=C(Cc1ccc(Cl)c(Br)c1)N1CCN(c2ccc3nncn3n2)CC1] | 2-(3-bromo-4-chlorophenyl)-1-(4-{[1,2,4]triazolo[4,3-b]pyridazin-6-yl}piperazin-1-yl)ethan-1-one | SLU-0010239 |
| 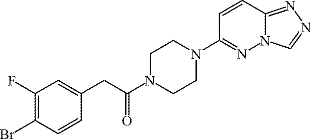 [SMILES: O=C(Cc1ccc(Br)c(F)c1)N1CCN(c2ccc3nncn3n2)CC1] | 2-(4-bromo-3-fluorophenyl)-1-(4-{[1,2,4]triazolo[4,3-b]pyridazin-6-yl }piperazin-1 -yl)ethan-1 -one | SLU-0010240 |
| 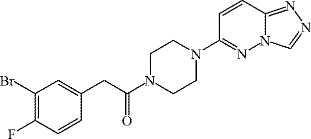 [SMILES: O=C(Cc1ccc(F)c(Br)c1)N1CCN(c2ccc3nncn3n2)CC1] | 2-(3-bromo-4-fluorophenyl)-1-(4-{[1,2,4]triazolo[4,3-b]pyridazin-6-yl }piperazin-1 -yl)ethan-1 -one | SLU-0010241 |
| 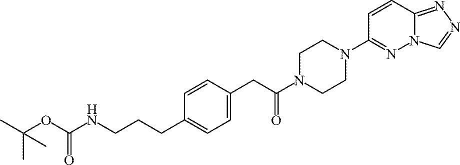 [SMILES: CC(C)(C)OC(=O)NCCCc1ccc(CC(=O)N2CCN(c3ccc4nncn4n3)CC2)cc1] | tert-butyl N-(3-{4-[2-oxo-2-(4-{[1,2,4]triazolo[4,3-b]pyridazin-6-yl}piperazin-1-yl)ethyl]phenyl}propyl)car bamate | SLU-0010242 |
| 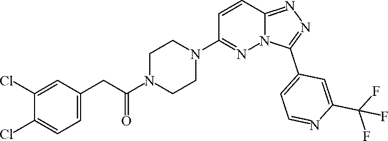 [SMILES: O=C(Cc1ccc(Cl)c(Cl)c1)N1CCN(c2ccc3nnc(-c4ccnc(C(F)(F)F)c4)n3n2)CC1] | 2-(3,4-dichlorophenyl)-1-(4-{3-[2-(trifluoromethyl)pyridin-4-yl]-[1,2,4]triazolo[4,3-b]pyridazin-6-yl}piperazin-1-yl)ethan-1-one | SLU-0010262 |
| 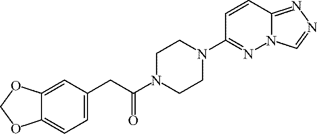 [SMILES: O=C(Cc1ccc2c(c1)OCO2)N1CCN(c2ccc3nncn3n2)CC1] | 2-(2H-1,3-benzodioxol-5-yl)-1-(4-{[1,2,4]triazolo[4,3-b]pyridazin-6-yl }piperazin-1 -yl)ethan-1 -one | SLU-0010422 |
| 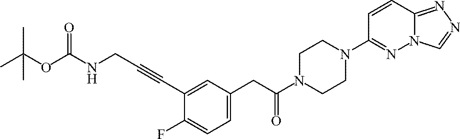 [SMILES: CC(C)(C)OC(=O)NCC#Cc1cc(CC(=O)N2CCN(c3ccc4nncn4n3)CC2)ccc1F] | tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate | SLU-0010443 |
| 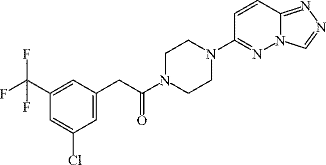 [SMILES: O=C(Cc1cc(Cl)cc(C(F)(F)F)c1)N1CCN(c2ccc3nncn3n2)CC1] | 2-[3-chloro-5-(trifluoromethyl)phenyl] -1-(4-{[1,2,4]triazolo[4,3-b]pyridazin-6-yl }piperazin-1 -yl)ethan-1 -one | SLU-0010479 |
| 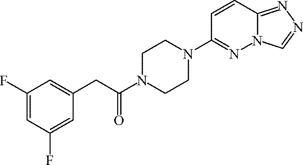 [SMILES: O=C(Cc1cc(F)cc(F)c1)N1CCN(c2ccc3nncn3n2)CC1] | 2-(3,5-difluorophenyl)-1-(4-{[1,2,4]triazolo[4,3-b]pyridazin-6-yl }piperazin-1 -yl)ethan-1 -one | SLU-0010485 |
| 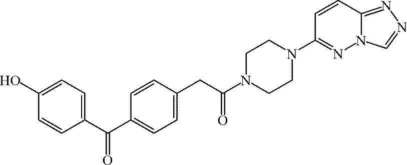 [SMILES: O=C(c1ccc(O)cc1)c1ccc(CC(=O)N2CCN(c3ccc4nncn4n3)CC2)cc1] | 2-[4-(4-hydroxybenzoyl)phenyl] -1-(4-{[1,2,4]triazolo[4,3-b]pyridazin-6-yl }piperazin-1 -yl)ethan-1 -one | SLU-0010500 |
| 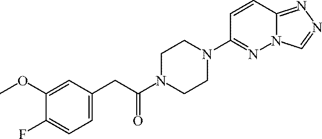 [SMILES: COc1cc(CC(=O)N2CCN(c3ccc4nncn4n3)CC2)ccc1F] | 2-(4-fluoro-3-methoxyphenyl)-1-(4-{[1,2,4]triazolo[4,3-b]pyridazin-6-yl }piperazin-1 -yl)ethan-1 -one | SLU-0010502 |
| 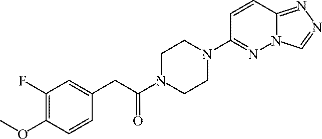 [SMILES: COc1ccc(CC(=O)N2CCN(c3ccc4nncn4n3)CC2)cc1F] | 2-(3-fluoro-4-methoxyphenyl)-1-(4-{[1,2,4]triazolo[4,3-b]pyridazin-6-yl }piperazin-1 -yl)ethan-1 -one | SLU-0010503 |
| 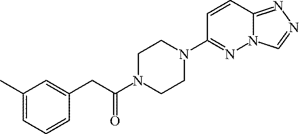 [SMILES: Cc1cccc(CC(=O)N2CCN(c3ccc4nncn4n3)CC2)c1] | 2-(3-methylphenyl)-1-(4-{[1,2,4]triazolo[4,3-b]pyridazin-6-yl }piperazin-1 -yl)ethan-1 -one | SLU-0010504 |
| 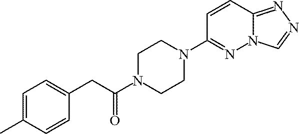 [SMILES: Cc1ccc(CC(=O)N2CCN(c3ccc4nncn4n3)CC2)cc1] | 2-(4-methylphenyl)-1-(4-{[1,2,4]triazolo[4,3-b]pyridazin-6-yl }piperazin-1 -yl)ethan-1 -one | SLU-0010505 |
| 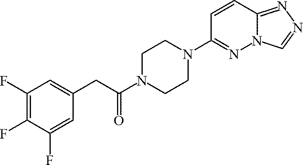 [SMILES: O=C(Cc1cc(F)c(F)c(F)c1)N1CCN(c2ccc3nncn3n2)CC1] | 1-(4-{[1,2,4]triazolo[4,3-b]pyridazin-6-yl }piperazin-1 -yl)-2-(3,4,5-trifluorophenyl)ethan-1 -one | SLU-0010550 |
| 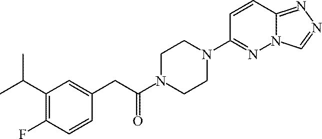 [SMILES: CC(C)c1cc(CC(=O)N2CCN(c3ccc4nncn4n3)CC2)ccc1F] | 2-[4-fluoro-3-(propan-2-yl)phenyl]-1-(4-{[1,2,4]triazolo[4,3-b]pyridazin-6-yl }piperazin-1 -yl)ethan-1 -one | |
| 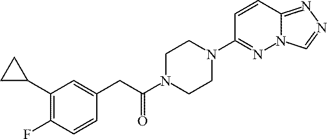 [SMILES: O=C(Cc1ccc(F)c(C2CC2)c1)N1CCN(c2ccc3nncn3n2)CC1] | 2-(3-cyclopropyl-4-fluorophenyl)-1-(4-{[1,2,4]triazolo[4,3-b]pyridazin-6-yl }piperazin-1 -yl)ethan-1 -one | |
| 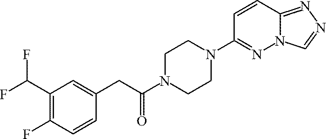 [SMILES: O=C(Cc1ccc(F)c(C(F)F)c1)N1CCN(c2ccc3nncn3n2)CC1] | 2-[3-(difluoromethyl)-4-fluorophenyl] -1-(4-{[1,2,4]triazolo[4,3-b]pyridazin-6-yl }piperazin-1 -yl)ethan-1 -one | |
| 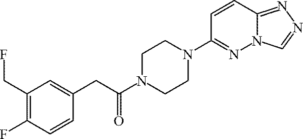 [SMILES: O=C(Cc1ccc(F)c(CF)c1)N1CCN(c2ccc3nncn3n2)CC1] | 2-[4-fluoro-3-(fluoromethyl)phenyl]-1-(4-{[1,2,4]triazolo[4,3-b]pyridazin-6-yl }piperazin-1 -yl)ethan-1 -one | |
| 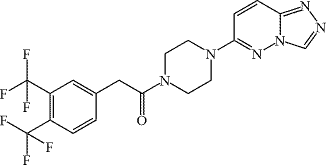 [SMILES: O=C(Cc1ccc(C(F)(F)F)c(C(F)(F)F)c1)N1CCN(c2ccc3nncn3n2)CC1] | 2-[3,4-bis(trifluoromethyl)phenyl ]-1-(4-{[1,2,4]triazolo[4,3-b]pyridazin-6-yl }piperazin-1 -yl)ethan-1 -one | |
| 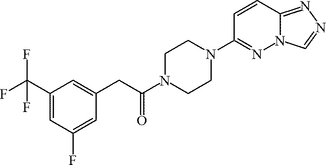 [SMILES: O=C(Cc1cc(F)cc(C(F)(F)F)c1)N1CCN(c2ccc3nncn3n2)CC1] | 2-[3-fluoro-5-(trifluoromethyl)phenyl]-1-(4-{[1,2,4]triazolo[4,3-b]pyridazin-6-yl }piperazin-1 -yl)ethan-1 -one | |
| 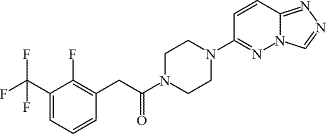 [SMILES: O=C(Cc1cccc(C(F)(F)F)c1F)N1CCN(c2ccc3nncn3n2)CC1] | 2-[2-fluoro-3-(trifluoromethyl)phenyl]-1-(4-{[1,2,4]triazolo[4,3-b]pyridazin-6-yl }piperazin-1 -yl)ethan-1 -one | |
| 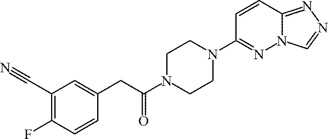 [SMILES: N#Cc1cc(CC(=O)N2CCN(c3ccc4nncn4n3)CC2)ccc1F] | 2-fluoro-5-[2-oxo-2-(4-{[1,2,4]triazolo[4,3-b]pyridazin-6-yl}piperazin-1-yl)ethyl]benzonitrile | |
| 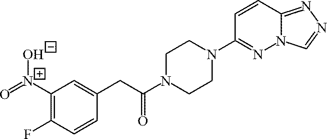 | 2-(4-fluoro-3-nitrophenyl)-1-(4-{[1,2,4]triazolo[4,3-b]pyridazin-6-yl }piperazin-1 -yl)ethan-1 -one | |
| 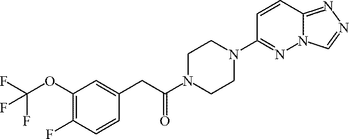 [SMILES: O=C(Cc1ccc(F)c(OC(F)(F)F)c1)N1CCN(c2ccc3nncn3n2)CC1] | 2-[4-fluoro-3-(trifluoromethoxy)phenyl] -1-(4-{[1,2,4]triazolo[4,3-b]pyridazin-6-yl }piperazin-1 -yl)ethan-1 -one | |
| 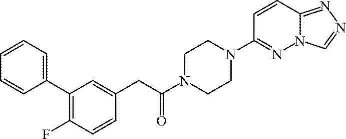 [SMILES: O=C(Cc1ccc(F)c(-c2ccccc2)c1)N1CCN(c2ccc3nncn3n2)CC1] | 2-{6-fluoro-[1,1′-biphenyl]-3-yl}-1-(4-{[1,2,4]triazolo[4,3-b]pyridazin-6-yl }piperazin-1 -yl)ethan-1 -one | |
| 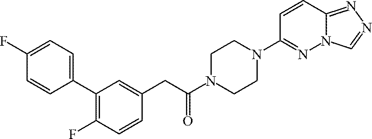 [SMILES: O=C(Cc1ccc(F)c(-c2ccc(F)cc2)c1)N1CCN(c2ccc3nncn3n2)CC1] | 2-{4′,6-difluoro-[1,1′-biphenyl]-3-yl}-1-(4-{[1,2,4]triazolo[4,3-b]pyridazin-6-yl }piperazin-1 -yl)ethan-1 -one | |
| 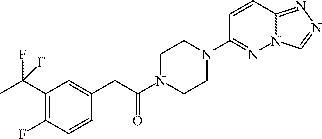 [SMILES: CC(F)(F)c1cc(CC(=O)N2CCN(c3ccc4nncn4n3)CC2)ccc1F] | 2-[3-(1,1-difluoroethyl)-4-fluorophenyl]-1-(4-{[1,2,4]triazolo[4,3-b]pyridazin-6-yl }piperazin-1 -yl)ethan-1 -one | |
| 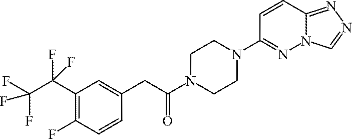 [SMILES: O=C(Cc1ccc(F)c(C(F)(F)C(F)(F)F)c1)N1CCN(c2ccc3nncn3n2)CC1] | 2-[4-fluoro-3-(1,1,2,2,2-pentafluoroethyl)phenyl]-1-(4-{[1,2,4]triazolo[4,3-b]pyridazin-6-yl }piperazin-1 -yl)ethan-1 -one | |
| 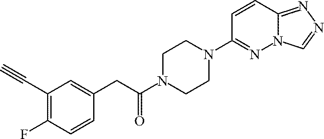 [SMILES: C#Cc1cc(CC(=O)N2CCN(c3ccc4nncn4n3)CC2)ccc1F] | 2-(3-ethynyl-4-fluorophenyl)-1-(4-{[1,2,4]triazolo[4,3-b]pyridazin-6-yl }piperazin-1 -yl)ethan-1 -one | |
| 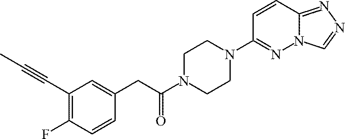 [SMILES: CC#Cc1cc(CC(=O)N2CCN(c3ccc4nncn4n3)CC2)ccc1F] | 2-[4-fluoro-3-(prop-1-yn-1-yl)phenyl]-1-(4-{[1,2,4]triazolo[4,3-b]pyridazin-6-yl }piperazin-1 -yl)ethan-1 -one | |
| 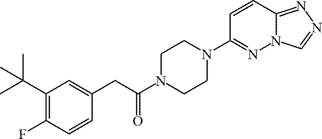 [SMILES: CC(C)(C)c1cc(CC(=O)N2CCN(c3ccc4nncn4n3)CC2)ccc1F] | 2-(3-tert-butyl-4-fluorophenyl)-1-(4-{[1,2,4]triazolo[4,3-b]pyridazin-6-yl}piperazin-1-yl)ethan-1-one | |
| 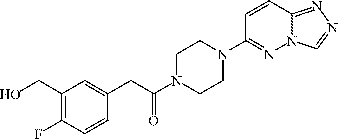 [SMILES: O=C(Cc1ccc(F)c(CO)c1)N1CCN(c2ccc3nncn3n2)CC1] | 2-[4-fluoro-3-(hydroxymethyl)phenyl]-1-(4-{[1,2,4]triazolo[4,3-b]pyridazin-6-yl}piperazin-1-yl)ethan-1-one | |
| 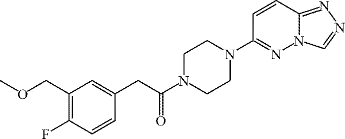 [SMILES: COCc1cc(CC(=O)N2CCN(c3ccc4nncn4n3)CC2)ccc1F] | 2-[4-fluoro-3-(methoxymethyl)phenyl]-1-(4-{[1,2,4]triazolo[4,3-b]pyridazin-6-yl}piperazin-1-yl)ethan-1-one | |
| 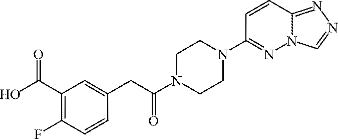 [SMILES: O=C(O)c1cc(CC(=O)N2CCN(c3ccc4nncn4n3)CC2)ccc1F] | 2-fluoro-5-[2-oxo-2-(4-{[1,2,4]triazolo[4,3-b]pyridazin-6-yl}piperazin-1-yl)ethyl]benzoic acid | |
| 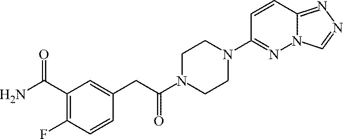 [SMILES: NC(=O)c1cc(CC(=O)N2CCN(c3ccc4nncn4n3)CC2)ccc1F] | 2-fluoro-5-[2-oxo-2-(4-{[1,2,4]triazolo[4,3-b]pyridazin-6-yl}piperazin-1-yl)ethyl]benzamide | |
| 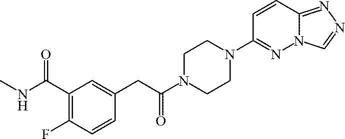 [SMILES: CNC(=O)c1cc(CC(=O)N2CCN(c3ccc4nncn4n3)CC2)ccc1F] | 2-fluoro-N-methyl-5-[2-oxo-2-(4-{[1,2,4]triazolo[4,3-b]pyridazin-6-yl}piperazin-1-yl)ethyl]benzamide | |
| 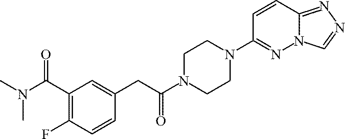 [SMILES: CN(C)C(=O)c1cc(CC(=O)N2CCN(c3ccc4nncn4n3)CC2)ccc1F] | 2-fluoro-N,N-dimethyl-5-[2-oxo-2-(4-{[1,2,4]triazolo[4,3-b]pyridazin-6-yl}piperazin-1-yl)ethyl]benzamide | |
| 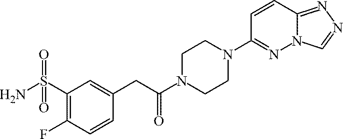 [SMILES: NS(=O)(=O)c1cc(CC(=O)N2CCN(c3ccc4nncn4n3)CC2)ccc1F] | 2-fluoro-5-[2-oxo-2-(4-{[1,2,4]triazolo[4,3-b]pyridazin-6-yl}piperazin-1-yl)ethyl]benzene-1-sulfonamide | |
| 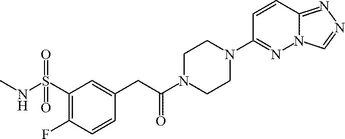 [SMILES: CNS(=O)(=O)c1cc(CC(=O)N2CCN(c3ccc4nncn4n3)CC2)ccc1F] | 2-fluoro-N-methyl-5-[2-oxo-2-(4-{[1,2,4]triazolo[4,3-b]pyridazin-6-yl}piperazin-1-yl)ethyl]benzene-1-sulfonamide | |
| 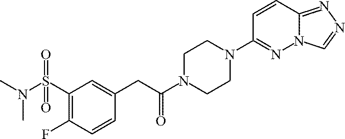 [SMILES: CN(C)S(=O)(=O)c1cc(CC(=O)N2CCN(c3ccc4nncn4n3)CC2)ccc1F] | 2-fluoro-N,N-dimethyl-5-[2-oxo-2-(4-{[1,2,4]triazolo[4,3-b]pyridazin-6-yl}piperazin-1-yl)ethyl]benzene-1-sulfonamide | |
| 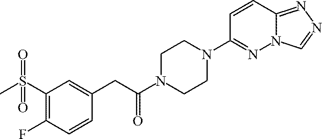 [SMILES: CS(=O)(=O)c1cc(CC(=O)N2CCN(c3ccc4nncn4n3)CC2)ccc1F] | 2-(4-fluoro-3-methanesulfonylphenyl)-1-(4-{[1,2,4]triazolo[4,3-b]pyridazin-6-yl}piperazin-1-yl)ethan-1-one | |
| 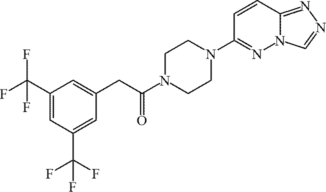 [SMILES: O=C(Cc1cc(C(F)(F)F)cc(C(F)(F)F)c1)N1CCN(c2ccc3nncn3n2)CC1] | 2-[3,5-bis(trifluoromethyl)phenyl ]-1-(4-{[1,2,4]triazolo[4,3-b]pyridazin-6-yl}piperazin-1-yl)ethan-1-one | SLU-0010495 |

In certain embodiments, a compound of the present disclosure does not have certain substituents at R^{1a}, R^{1b}, R^{1c}, R², R^{3a}, R^{3b}, R⁴, R⁵, R⁶, R⁷, or R⁸. In various embodiments, R^{1a} is not ethyl, a heteroaryl group

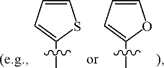

or an alkylheteroaryl group

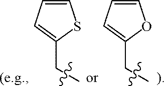

In various other examples, R^{3a} or R^{3b} is not —NH₂, —CH₂NH₂, or phenyl. In various examples, R^{3a} and R^{3b} do not join together to form

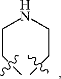

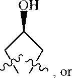

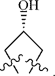

In various examples, R^{3a} and/or R^{3b} do not join with R⁴ and/or R⁸ to form one or more rings. In various examples, R⁴-R⁸ is/are not —NH₂ or —OH. In various examples, R⁵ and R⁶ do not join together to form

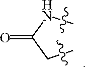

In various examples, a compound of the present disclosure does not have the following structure:

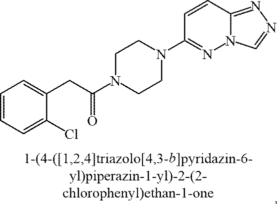

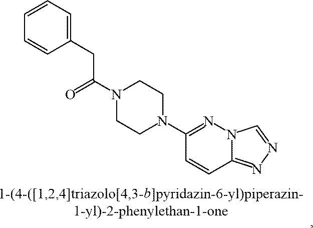

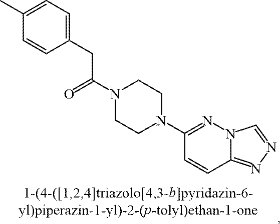

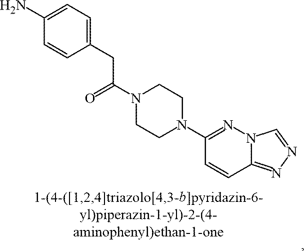

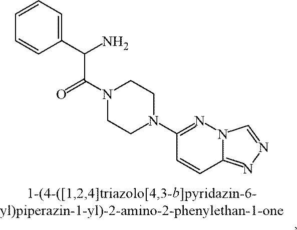

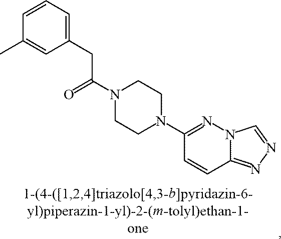

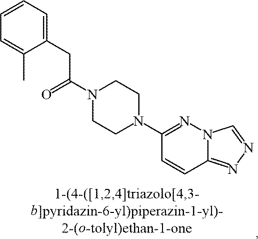

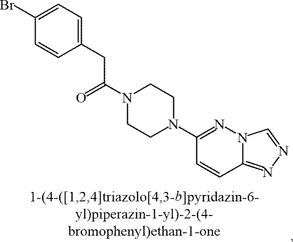

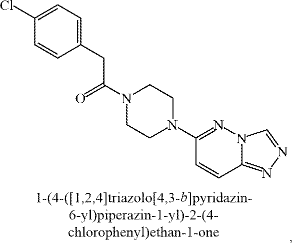

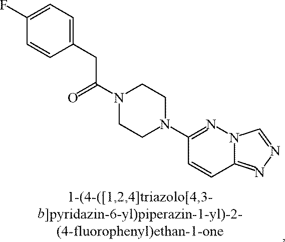

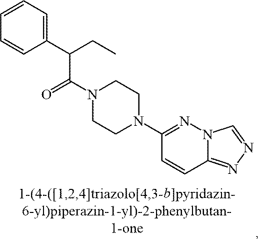

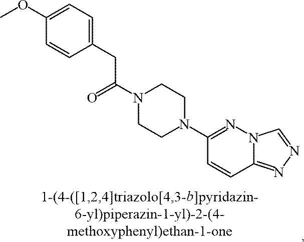

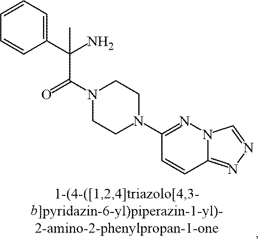

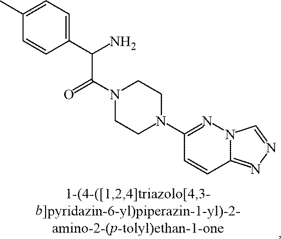

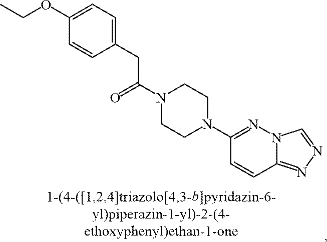

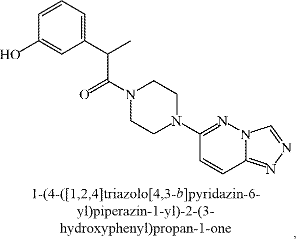

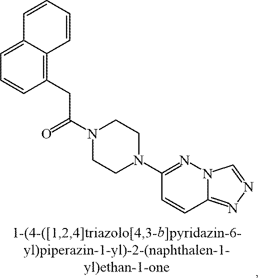

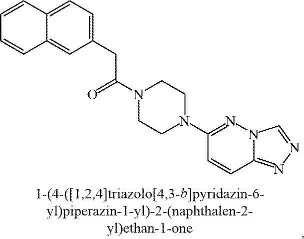

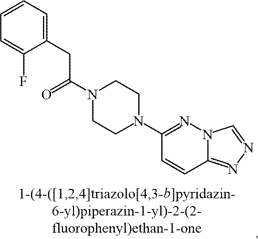

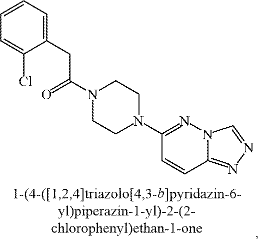

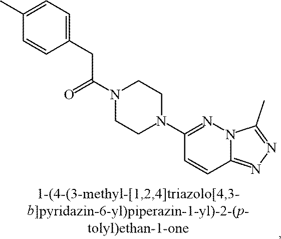

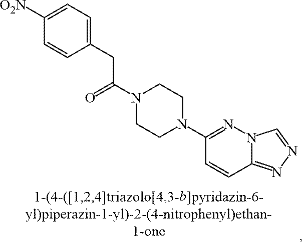

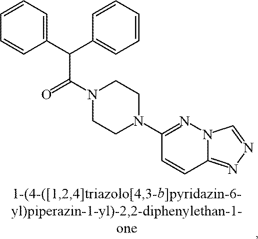

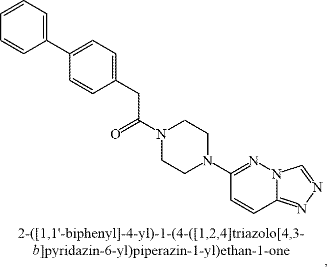

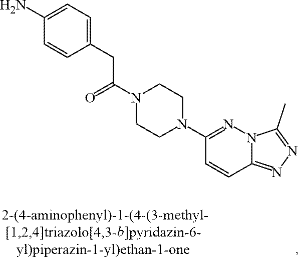

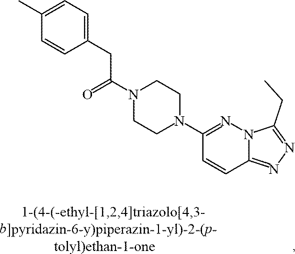

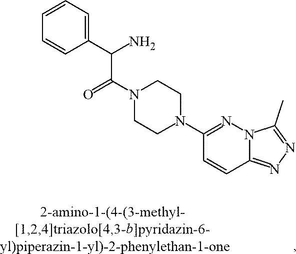

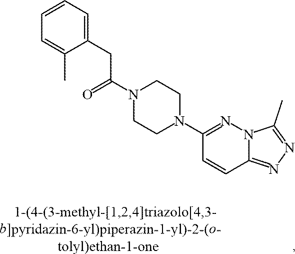

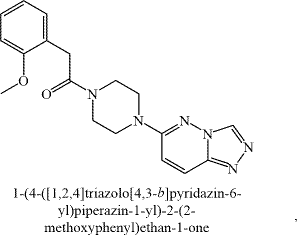

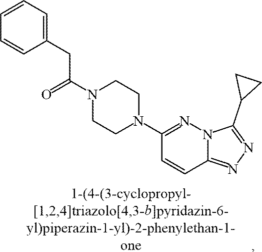

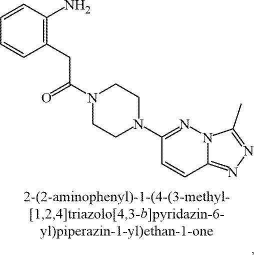

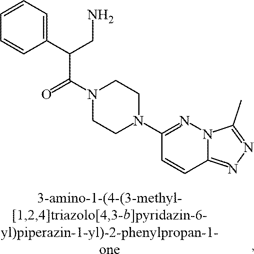

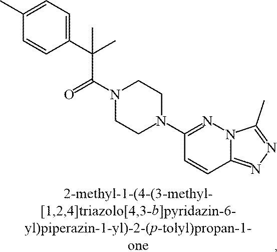

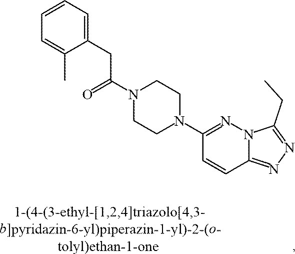

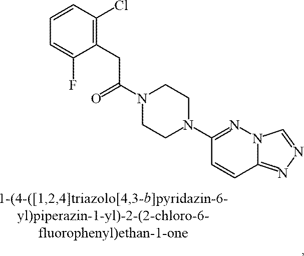

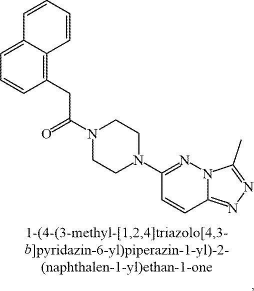

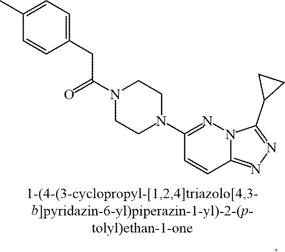

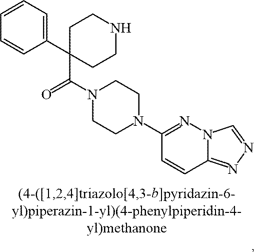

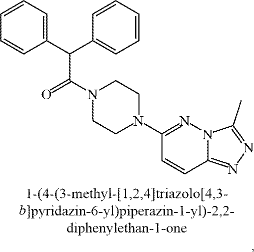

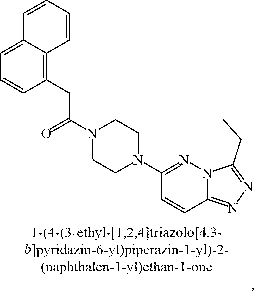

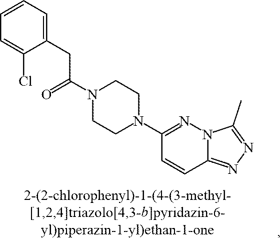

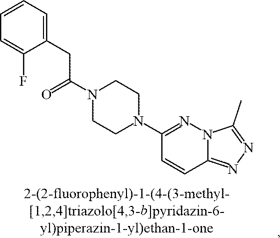

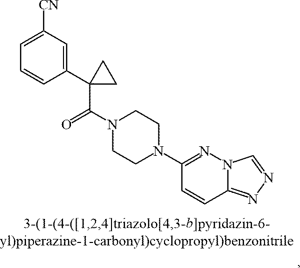

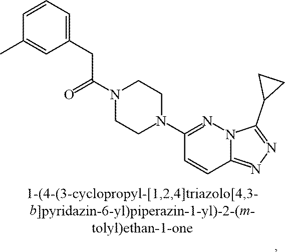

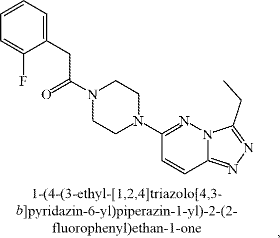

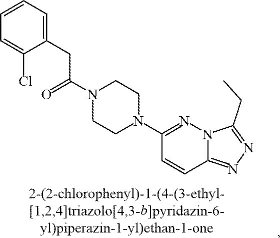

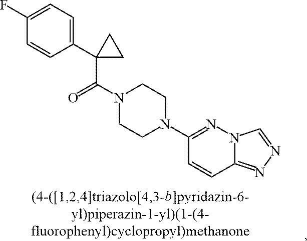

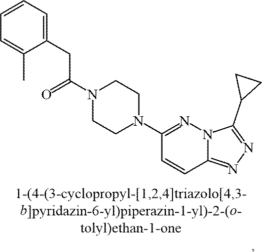

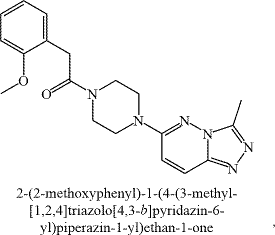

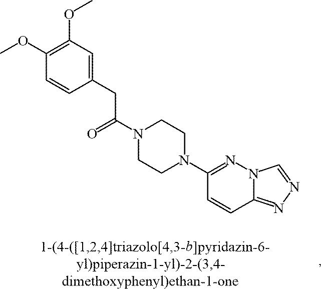

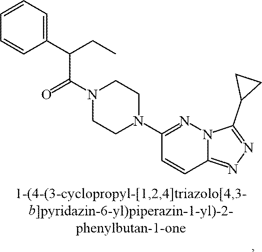

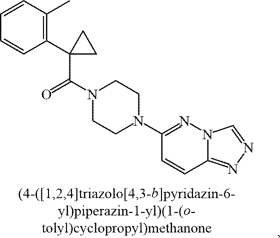

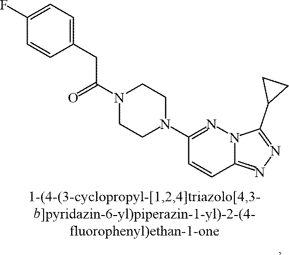

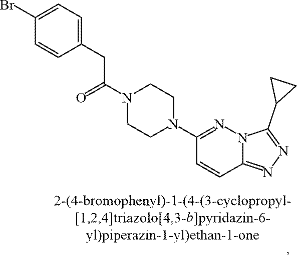

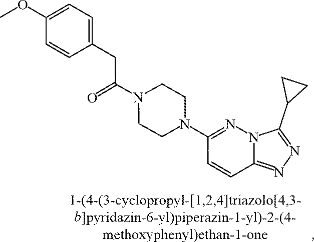

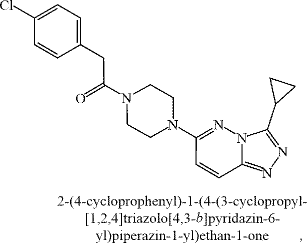

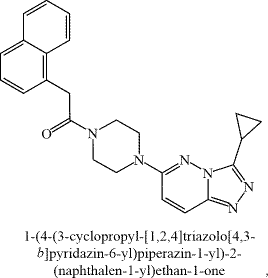

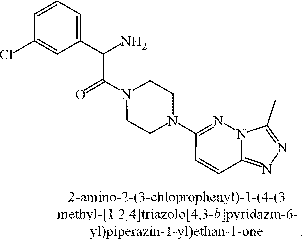

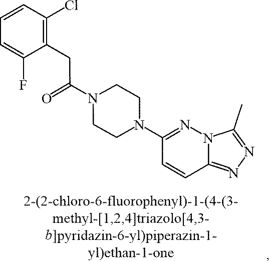

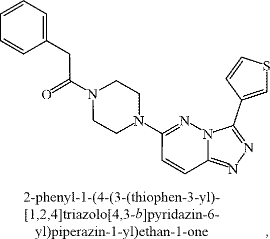

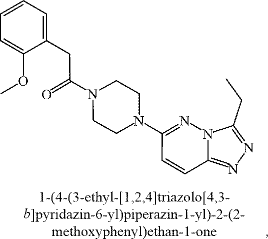

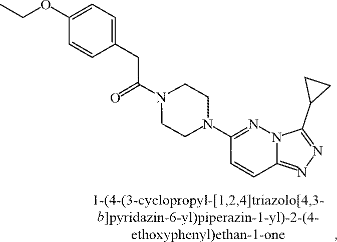

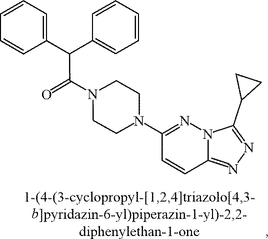

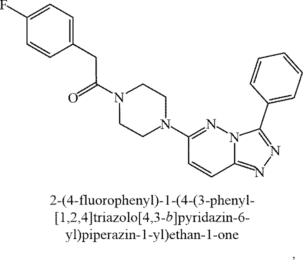

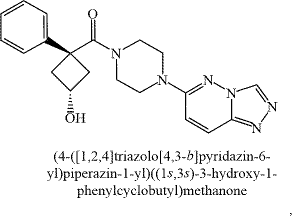

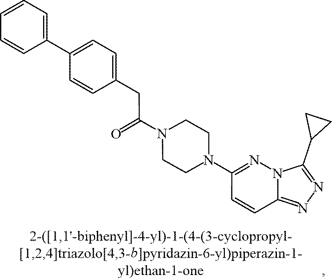

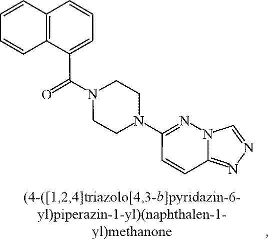

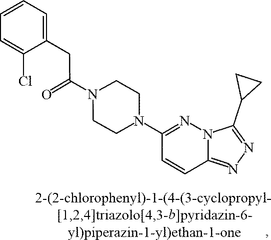

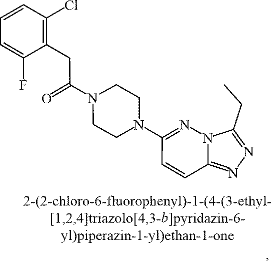

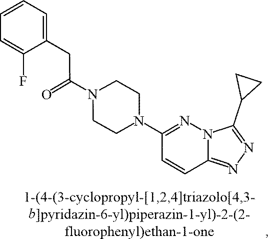

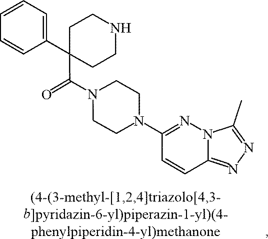

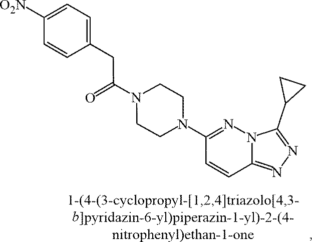

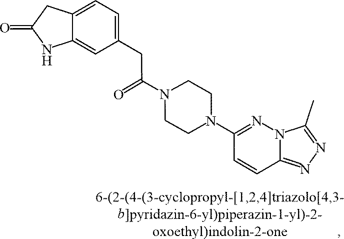

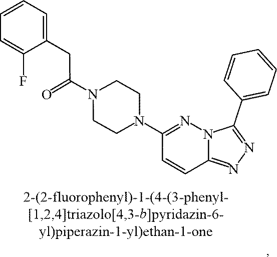

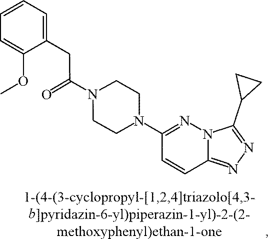

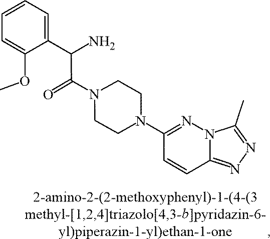

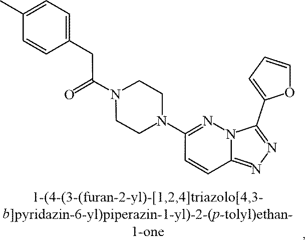

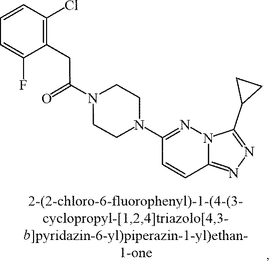

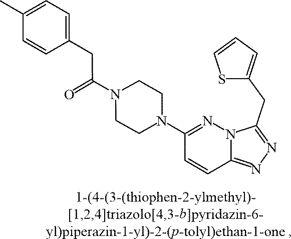

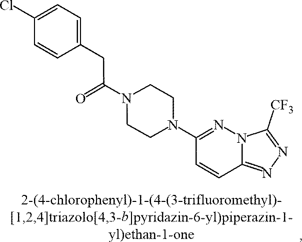

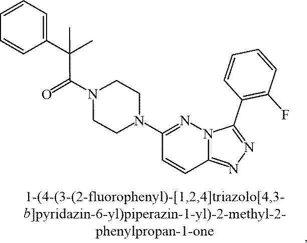

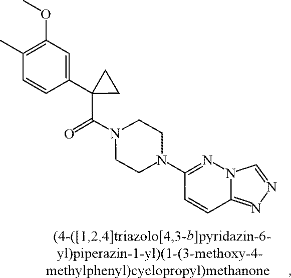

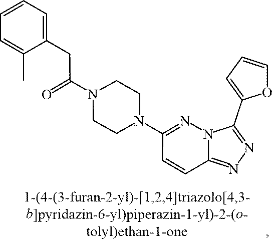

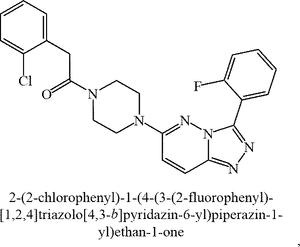

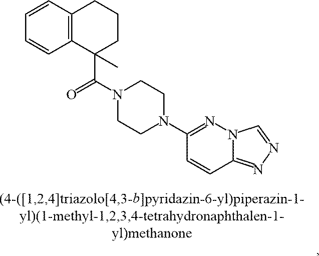

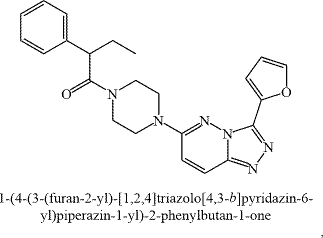

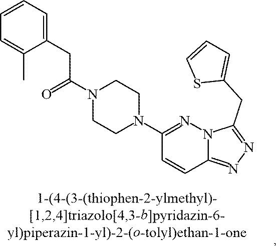

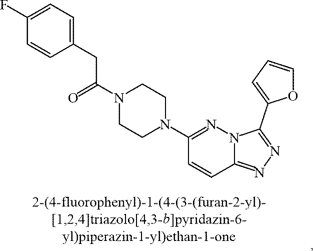

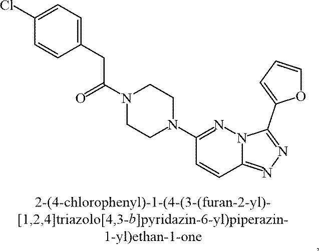

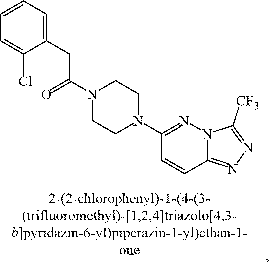

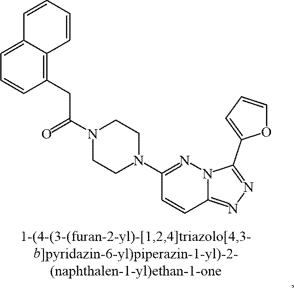

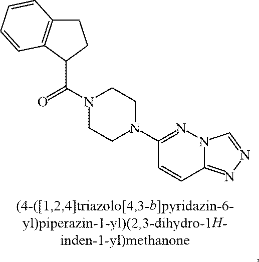

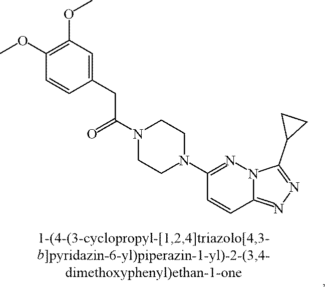

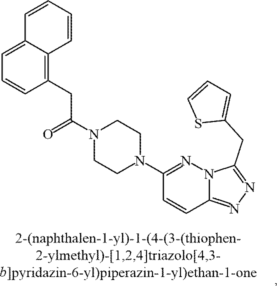

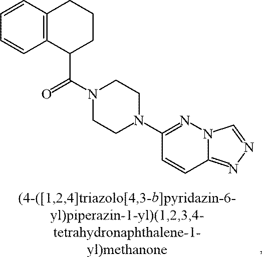

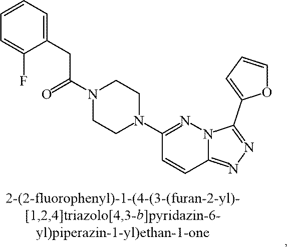

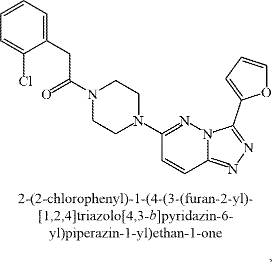

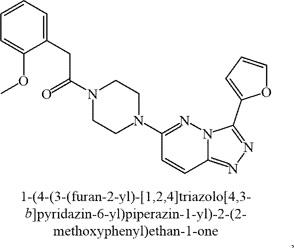

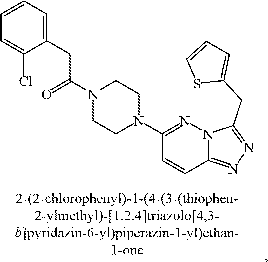

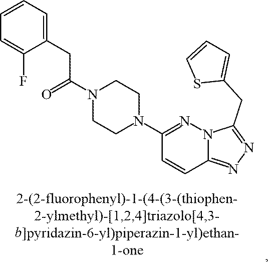

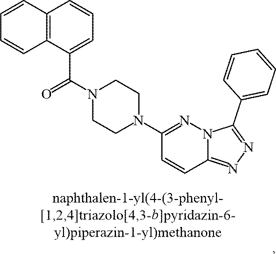

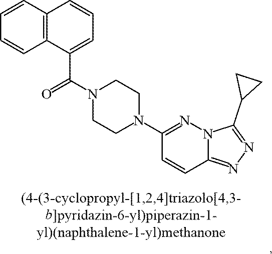

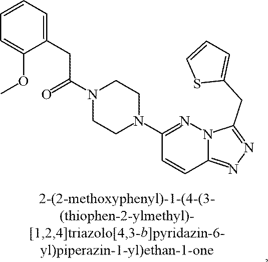

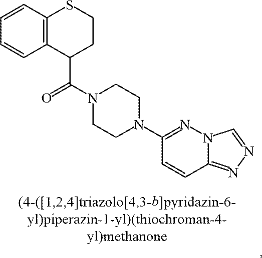

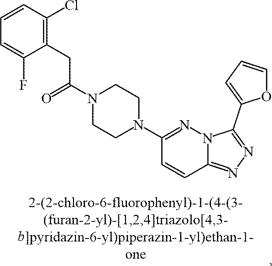

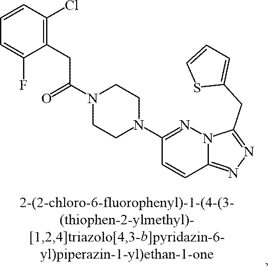

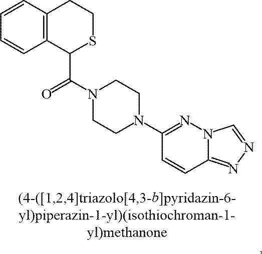

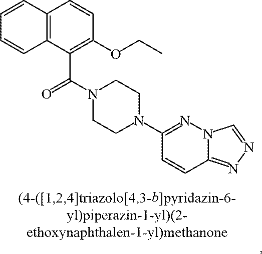

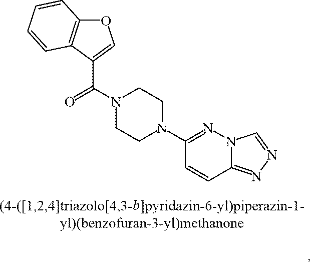

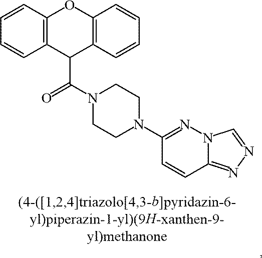

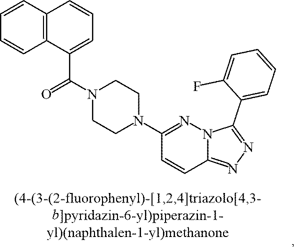

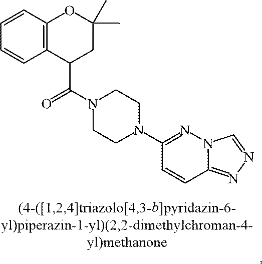

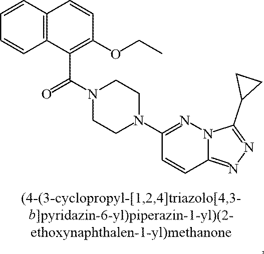

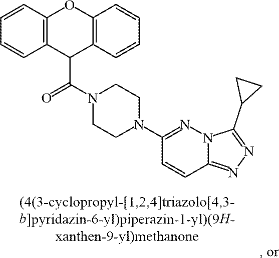

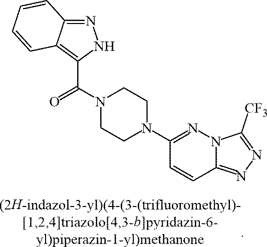

The present disclosure includes all possible stereoisomers and geometric isomers of a compound of the present disclosure. The present disclosure includes both racemic compounds and optically active isomers. When a compound of the present disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of a compound of the present disclosure are possible, the present disclosure is intended to include all tautomeric forms of the compounds.

Prodrugs of a compound of the present disclosure also can be used as the compound in a method of the present disclosure. It is well established that a prodrug approach, wherein a compound is derivatized into a form suitable for formulation and/or administration, then released as a drug in vivo, has been successfully employed to transiently (e.g., bioreversibly) alter the physicochemical properties of the compound (see, H. Bundgaard, Ed., “Design of Prodrugs,” Elsevier, Amsterdam, (1985); R.B. Silverman, “The Organic Chemistry of Drug Design and Drug Action,” Academic Press, San Diego, chapter 8, (1992); K.M. Hillgren et al., Med. Res. Rev., 15, 83 (1995)).

Compounds of the present disclosure can contain one or more functional groups. The functional groups, if desired or necessary, can be modified to provide a prodrug. Suitable prodrugs include, for example, acid derivatives, such as amides and esters. It also is appreciated by those skilled in the art that N-oxides can be used as a prodrug.

Compounds of the disclosure may exist as salts. Pharmaceutically acceptable salts of the compounds of the disclosure generally are preferred in the methods of the disclosure. As used herein, the term “pharmaceutically acceptable salts” refers to salts or zwitterionic forms of a compound of the present disclosure. Salts of compounds of the present disclosure can be prepared during the final isolation and purification of the compounds or separately by reacting the compound with an acid having a suitable cation. The pharmaceutically acceptable salts of a compound of the present disclosure are acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the disclosure can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present disclosure appearing herein is intended to include a compound of the present disclosure as well as pharmaceutically acceptable salts, hydrates, or prodrugs thereof.

The compounds may exhibit wide variability in pharmacokinetic and physicochemical properties while still retaining desirable biological activity as described herein. For example solubility, e.g., log P, is variable while still retaining desirable biological activity as described herein.

In an aspect, the present disclosure provides compositions comprising one or more compound(s) of the present disclosure. The compositions may further comprise one or more pharmaceutically acceptable carrier(s).

The compositions may include one or more pharmaceutically acceptable carrier(s). Non-limiting examples of compositions include solutions, suspensions, emulsions, solid injectable compositions that are dissolved or suspended in a solvent before use, and the like. Injections may be prepared by dissolving, suspending, or emulsifying one or more of the active ingredient(s) in a diluent. Non-limiting examples of diluents include distilled water (e.g., for injection), physiological saline, vegetable oil, alcohol, and the like, and combinations thereof. Injections may contain, for example, stabilizers, solubilizers, suspending agents, emulsifiers, soothing agents, buffers, preservatives, and the like, and combinations thereof. Injections may be sterilized in the final formulation step or prepared by sterile procedure. A pharmaceutical composition of the disclosure may also be formulated into a sterile solid preparation, for example, by freeze-drying, and may be used after sterilized or dissolved in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, such as, for example, sodium carboxymethyl cellulose, ethyl cellulose, and cellulose acetate; powdered tragacanth; malt; gelatin; talc; excipients, such as, for example, cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example, glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer’s solution; ethyl alcohol; phosphate buffer solutions; other non-toxic compatible substances employed in pharmaceutical formulations, and the like, and combinations thereof. Non-limiting examples of pharmaceutically acceptable carriers are found in: Remington: The Science and Practice of Pharmacy (2005) 21st Edition, Philadelphia, PA. Lippincott Williams & Wilkins.

Compositions of the disclosure can comprise more than one pharmaceutical agent. For example, a first composition comprising a compound of the disclosure and a first pharmaceutical agent can be separately prepared from a composition which comprises the same compound of the disclosure and a second pharmaceutical agent, and such preparations can be mixed to provide a two-pronged (or more) approach to achieving the desired prophylaxis or therapy in an individual. Further, compositions of the disclosure can be prepared using mixed preparations of any of the compounds disclosed herein.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of a compound of the present disclosure include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to a compound of the disclosure, the composition may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The composition may be for administration to an individual in need of treatment.

In various examples, a composition comprises a pH sensitive polymer.

In an embodiment the composition comprises a compound of the present disclosure encapsulated in a pH sensitive polymer suitable for release of a compound of the disclosure in the small intestines, distal small intestine, or colon.

In an embodiment, the pH sensitive polymer suitable for such a release can be a synthetic anionic polymer based on a monomer such as acrylic acid, methacrylic acid, propionic acid, 2-acrylmido-2-methylpropylsulfonic acid, 2-methacryloxyethylsulfonic acid, 3-methacryloxy-2-hydroxypropylsulfonic acid, ethylenesulfonic acid, styrenesulfonic acid, sulfoxyethyl methacrylate, or a combination thereof. In another embodiment, the anionic polymer can be a natural anionic polymer such as hyaluronic acid, alginic acid, carboxymethyl cellulose, carboxymethyl dextran, poly(aspartic acid), or heparin. Several commercial pH sensitive polymers are available. For example, Eudragit L, Eudragit S from Röhm Pharma GmBH (based on methacrylic acid and methyl methacrylate) or CMEC from Freund Sangyo Co., Ltd; CAP from Wako Pure Chemicals Ltd.; or HP-50 and ASM from Shin-Etsu Chemical Co., Ltd. (derived from cellulose) can be used.

Without intending to be bound by any particular theory it is considered that compounds with adequate solubility to access the parasites within intestinal epithelial cells are desired. In an embodiment, the compounds are largely retained at the site of infection within the gut. Consistent with the ability of achieving this goal, the SAR studies conducted demonstrate that formulations of each of these chemical scaffolds with widely divergent solubility (and, therefore, systemic absorption) have retained potency against Cryptosporidium within intestinal epithelial cells. In essence, compounds for treatment of cryptosporidiosis should break several of Lipinski’s rules for selection of drug leads. Thus, formulations of each scaffold that are optimized for treatment of cryptosporidiosis will be distinct from formulations appropriate for treatment of a systemic infection such as malaria. Drug exposure (both the ability to penetrate the cell and parasitophorous vacuole, and retention at the site of infection rather than oral absorption) will be equivalent. In an embodiment, the compounds are not released systemically but are rather retained in the gastrointestinal tract. In an embodiment, the compounds of the method are taken up by the cells of the lumen of the gastrointestinal tract. In another embodiment, systemic exposure may be necessary in certain cases. For example, in severely immunocompromised people such as those with AIDS, infection can involve the biliary tree and, rarely, even the lungs. In these circumstances, a drug or formulation that favors systemic absorption, and/or enterohepatic recirculation may be desirable.

In an embodiment, a formulation comprising a compound of the disclosure is formulated in a manner such that an extended release in the small intestines, distal small intestine, or colon is achieved. For example, pH sensitive polymers can be used as described herein.

In an aspect, the present disclosure provides a methods for treating an individual diagnosed with or suspected of having a cryptosporidium infection. Compounds of the present disclosure can be used in the methods.

In an embodiment, a method for treating an individual diagnosed with or suspected of having a cryptosporidium infection comprises administering to the individual a therapeutically effective amount of a compound or composition of the present disclosure.

The compounds may be used to treat a cryptosporidium infection caused by any species of Cryptosporidium. Examples of Cryptosporidium species include, Cryptosporidium parvum or Cryptosporidium hominis.

Cryptosporidium infection is defined as detection of Cryptosporidium in the feces, by any standard means such as microscopic parasite exam, antigen detection, and polymerase chain reaction. In some instances, such individuals may be suffering from diarrhea, but may be asymptomatic where there are indications to treat for prevention of long-term sequelae (such as malnutrition and growth stunting) and for prevention of spreading infection to others. In areas where Cryptosporidium infection are highly endemic, treatment for cryptosporidiosis may be provided (alone or in combination with treatment for other enteric infections) for suspected infection without microbiologic confirmation.

The individual to be treated by the method of the disclosure may be human or non-human (e.g., mammal). Non-human animals include ungulates such as bovines. Additional on-limiting examples of non-human mammals include pigs, mice, rats, rabbits, cats, dogs, or other agricultural mammals, pet, or service animals, and the like.

Compositions comprising a compound of the disclosure and a pharmaceutical agent can be prepared at a patient’s bedside, or by a pharmaceutical manufacture. In the latter case, the compositions can be provided in any suitable container, such as a sealed sterile vial or ampoule, and may be further packaged to include instruction documents for use by a pharmacist, physician or other health care provider. The compositions can be provided as a liquid, or as a lyophilized or powder form that can be reconstituted if necessary when ready for use. In particular, the compositions can be provided in combination with any suitable delivery form or vehicle, examples of which include, for example, liquids, caplets, capsules, tablets, inhalants or aerosol, etc. The delivery devices may comprise components that facilitate release of the pharmaceutical agents over certain time periods and/or intervals, and can include compositions that enhance delivery of the pharmaceuticals, such as nanoparticle, microsphere or liposome formulations, a variety of which are known in the art and are commercially available.

The dose of the composition comprising a compound of the present disclosure and a pharmaceutical agent generally depends upon the needs of the individual to whom the composition of the disclosure is to be administered. These factors include, for example, the weight, age, sex, medical history, and nature and stage of the disease for which a therapeutic or prophylactic effect is desired. The compositions can be used in conjunction with any other conventional treatment modality designed to improve the disorder for which a desired therapeutic or prophylactic effect is intended, non-limiting examples of which include surgical interventions and radiation therapies. The compositions can be administered once, or over a series of administrations at various intervals determined using ordinary skill in the art, and given the benefit of the present disclosure.

It is envisioned, therefore, that a compound of the present disclosure is useful in the treatment of a cryptosporidium infection. Thus, the present disclosure concerns the use of a compound of the present disclosure, or a pharmaceutically acceptable salt thereof, or a pharmaceutical composition containing either a compound of the present disclosure, or a pharmaceutically acceptable salt thereof, for the manufacture of a medicament for the treatment of such conditions and diseases.

The compounds of the present disclosure can be therapeutically administered as the neat chemical, but it is preferred to administer a compound of the present disclosure as a pharmaceutical composition or formulation. Thus, the present disclosure provides a pharmaceutical composition comprising a compound of the present disclosure together with a pharmaceutically acceptable diluent or carrier therefor. Also provided is a process of preparing a pharmaceutical composition comprising admixing a compound of the present disclosure with a pharmaceutically acceptable diluent or carrier therefor.

In one embodiment, the pharmaceutically-acceptable formulation is such that it provides sustained delivery of a compound of the present disclosure to an individual for at least 12 hours, 24 hours, 36 hours, 48 hours, one week, two weeks, three weeks, or four weeks after the pharmaceutically-acceptable formulation is administered to the individual.

In certain embodiments, these pharmaceutical compositions are suitable for oral administration to an individual. In other embodiments, as described in detail below, the pharmaceutical compositions of the present disclosure may be specially formulated for administration in solid or liquid form, including those adapted for the following: oral administration, for example, drenches (aqueous or non-aqueous solutions or suspensions), tablets, boluses, powders, granules, and pastes.

The compositions may conveniently be presented in unit dosage form and may be prepared by any methods well known in the art of pharmacy. The amount of active ingredient which can be combined with a carrier material to produce a single dosage form will vary depending upon the individual being treated, the particular mode of administration. The amount of active ingredient which can be combined with a carrier material to produce a single dosage form will generally be that amount of a compound of the present disclosure which produces a therapeutic effect. Generally, out of one hundred per cent, this amount will range from about 1 per cent to about ninety-nine percent of active ingredient, preferably from about 5 per cent to about 70 per cent, more preferably from about 10 per cent to about 30 per cent.

Methods of preparing these compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

When a compound of the present disclosure are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

In certain embodiments, the methods of the disclosure include administering to an individual a therapeutically effective amount of a compound of the present disclosure in combination with another pharmaceutically active ingredient. Pharmaceutically active ingredients that may be used can be found in Harrison’s Principles of Internal Medicine, Thirteenth Edition, Eds. T.R. Harrison et al. McGraw-Hill N.Y., NY; and the Physicians Desk Reference 50th Edition 1997, Oradell New Jersey, Medical Economics Co., the complete contents of which are expressly incorporated herein by reference. A compound of the present disclosure and the pharmaceutically active ingredient may be administered to the individual in the same pharmaceutical composition or in different pharmaceutical compositions (at the same time or at different times).

Methods delineated herein include those wherein the individual is identified as in need of a particular stated treatment. Identifying an individual in need of such treatment can be in the judgment of an individual or a health care professional and can be subjective (e.g. opinion) or objective (e.g. measurable by a test or diagnostic method). In other methods, the individual is prescreened or identified as in need of such treatment by assessment for a relevant marker or indicator of suitability for such treatment.

The compounds and compositions disclosed in the present disclosure can also be used for prophylaxis in an individual who is at risk of being exposed to cryptosporidium. A prophylactic use may be useful, for example, in an individual who is about to undertake a journey to a region where an outbreak of cryptosporidium has been reported or is known to occur. For the prophylactic use, one or more doses of a composition comprising the compounds of the present disclosure may be administered. Dosing for prophylactic treatment may vary compared to dosing of known cryptosporidium infections. In one embodiment treatment may be given 1-3 times a day for 1 to 10 days or longer. In one embodiment, treatment is given for up to three times (such as 1 to 3 times) daily for up to 10 days (such as 1-10 days) in immunocompetent hosts. For immmuosuppressed hosts such as those with AIDS, long-term suppressive treatment may be warranted.

The identification of those patients who are in need of prophylactic treatment for a Cryptosporidium infection can readily identify such candidate patients, by the use of, for example, clinical tests, physical examination and medical/family history. The individual may have a Cryptosporidium infection, may be at risk of developing a Cryptosporidium infection, or may need prophylactic treatment prior to anticipated or unanticipated exposure to a condition(s) capable of increasing susceptibility to a Cryptosporidium infection. In an embodiment, those in need of prophylactic treatment for a Cryptosporidium infection can take a therapeutically effective amount of a compound of the present disclosure from 1 to 30 days prior to an anticipated or unanticipated exposure to a condition(s) capable of increasing susceptibility to a Cryptosporidium infection. In another embodiment, those in need of prophylactic treatment for a Cryptosporidium infection can take a therapeutically effective amount of a compound of the present disclosure from 1 to 24 hours prior to an anticipated or unanticipated exposure to a condition(s) capable of increasing susceptibility to a Cryptosporidium infection. In an embodiment, an individual can take a therapeutically effective amount of a compound of the present disclosure shortly after exposure to Cryptosporidium infection.

In another aspect, the disclosure provides a packaged composition including a therapeutically effective amount of a compound of the present disclosure and a pharmaceutically acceptable carrier or diluent. The composition may be formulated for treating an individual suffering from or susceptible to a Cryptosporidium infection, and packaged with instructions to treat an individual suffering from or susceptible to a Cryptosporidium infection.

In one aspect, the disclosure provides a kit for treating a Cryptosporidium infection in an individual is provided and includes a compound of the present disclosure, pharmaceutically acceptable esters, salts, and prodrugs thereof, and instructions for use. In certain embodiments, the disclosure provides: a kit for treating a Cryptosporidium infection, in an individual, the kit comprising a compound of the present disclosure. The kit may also include instructions for administration of the compound or composition. The instructions may include details on one or more of the following: dosage, frequency, number of administrations to be carried out (such as number of tablets to be consumed), whether the composition needs to be taken with food, water etc., storage of the composition, and the like.

For veterinary use, a compound of the present disclosure, or a pharmaceutically acceptable salt or prodrug, is administered as a suitably acceptable formulation in accordance with normal veterinary practice. The veterinarian can readily determine the dosing regimen and route of administration that is most appropriate for a particular animal. Animals treatable by the present compounds and methods include, but are not limited to, bovines or ungulates.

When administered in combination with other therapeutics, a present compound may be administered at relatively lower dosages. In addition, the use of targeting agents may allow the necessary dosage to be relatively low. Certain compounds may be administered at relatively high dosages due to factors including, but not limited to, low toxicity and high clearance.

For human use, a compound of the present disclosure can be administered alone, but generally is administered in admixture with a pharmaceutical carrier selected with regard to the intended route of administration and standard pharmaceutical practice. Pharmaceutical compositions for use in accordance with the present disclosure can be formulated in a conventional manner using one or more physiologically acceptable carrier comprising excipients and auxiliaries that facilitate processing of a compound of the present disclosure into pharmaceutical preparations.

In an aspect, a compound as described herein exhibits activity against Cryptosporidium. In certain embodiments, the Cryptosporidium infection is from the Cryptosporidium parvum, Cryptosporidium hominis, Cryptosporidium andersoni, or a combination thereof. In various embodiments, the compounds of the disclosure are those which display in vitro EC₅₀ values less than or equal to 10 µM, less than or equal to 9 µM, less than or equal to 8 µM, less than or equal to 7 µM, less than or equal to 6 µM, less than or equal to 5 µM, less than or equal to 4 µM, less than or equal to 3 µM, or less than or equal to 2 µM against Cryptosporidium parvum.

As appreciated by persons skilled in the art, additional active or ancillary agents can be used in the methods described herein. Reference herein to treatment also extends to prophylaxis, as well as to treatment of established diseases or symptoms.

A method of the present disclosure be applied to cell populations ex vivo. For example, the present compounds can be used ex vivo to determine the optimal schedule and/or dosing of administration of the present compound for a given indication, cell type, patient, and other parameter. Information gleaned from such use can be used for experimental purposes or in the clinic to set protocol for in vivo treatment. Other ex vivo uses for which the disclosure is suited are apparent to those skilled in the art.

The steps of the method described in the various embodiments and examples disclosed herein are sufficient to treat an individual diagnosed with or suspected of having a cryptosporidium infection or a method for prophylaxis in an individual diagnosed with or suspected of having a cryptosporidium infection. Thus, in an embodiment, the method consists essentially of a combination of the steps of the method disclosed herein. In another embodiment, the method consists of such steps.

In various embodiments, a compound of the present disclosure has the following structure:

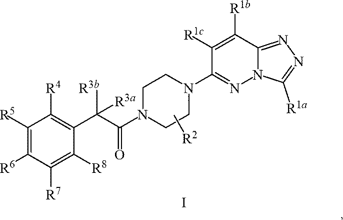

where R^{1a}, R^{1b}, and R^{1c} are independently chosen from H, substituted alkyl groups (e.g., haloalkyl groups, such as, for example, trifluoromethyl), unsubstituted alkyl groups, substituted aryl groups, unsubstituted aryl groups, substituted aralkyl groups, and unsubstituted aralkyl groups; R² is chosen from H, substituted alkyl groups, and unsubstituted alkyl groups; R^{3a} and R^{3b} are independently chosen from H, substituted alkyl groups, unsubstituted alkyl groups, halogen, —OH, —NH₂, and R^{3a} and R^{3b} may combine to form a ring (e.g., cyclopropyl

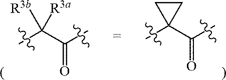

and the like) or a ═O; R⁴ and R⁸ are independently chosen from H, halogens, substituted alkyl groups, and unsubstituted alkyl groups; R⁵, R⁶, and R⁷ are independently chosen from H, halogens, substituted alkyl groups (e.g., haloalkyl groups; alkylamino groups ((CH₂)ₙNH₂, where n is 1, 2, 3, or 4; (CH₂)ₙNR′R″, where n is 1, 2, 3, or 4); (CH₂)ₙOH groups, where n is 1, 2, 3, or 4, and the like), unsubstituted alkyl groups, substituted cycloalkyl groups, unsubstituted cycloalkyl groups, substituted alkoxy groups, unsubstituted alkoxy groups, substituted aryl groups, unsubstituted aryl groups, substituted aralkyl groups, unsubstituted aralkyl groups, SO₂NR′R″, (CH₂)ₙ-alkoxy groups (where n is 1, 2, 3, or 4), (CH₂)ₙNHCO-alkyl (where alkyl is substituted or unsubstituted, and n is 1, 2, 3, or 4), nitro groups, alcohol groups, -SF₅ groups, and CONR′R″, where R′ and R″ are independently chosen from H, substituted alkyl groups (e.g., methoxyethyl), unsubstituted alkyl groups (e.g., methyl), Cl, and F and, R⁴ and R⁵, R⁵ and R⁶, R⁶ and R⁷, or R⁷ and R⁸ may combine to form a ring, where with the proviso compound is not:

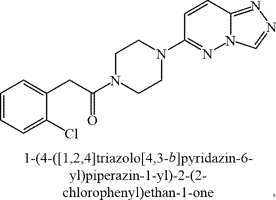

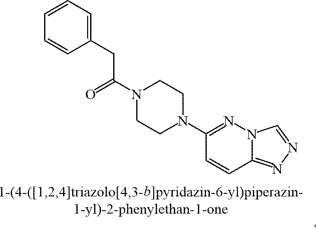

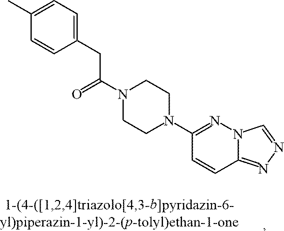

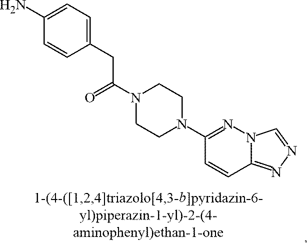

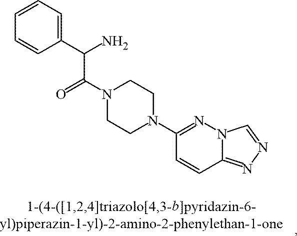

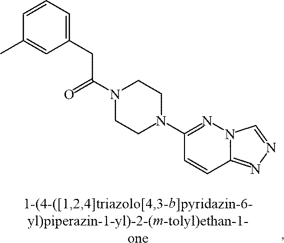

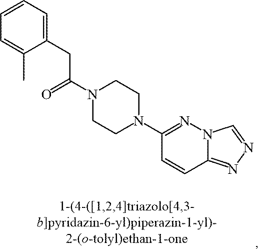

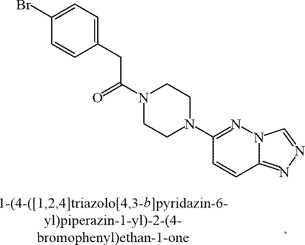

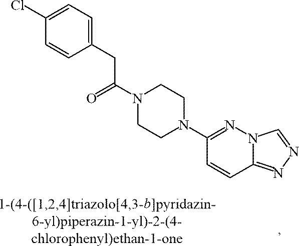

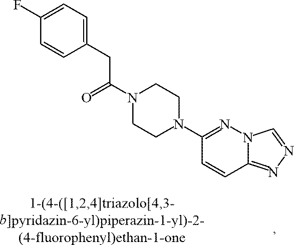

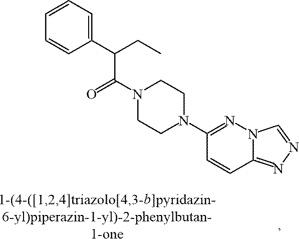

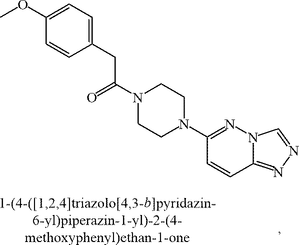

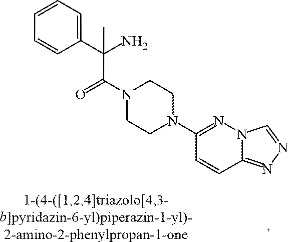

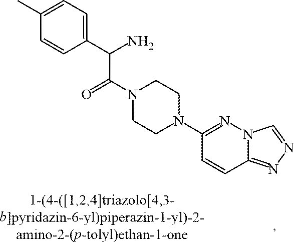

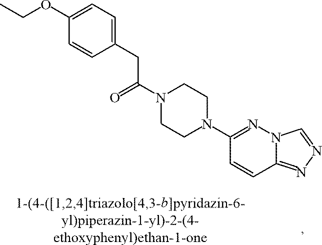

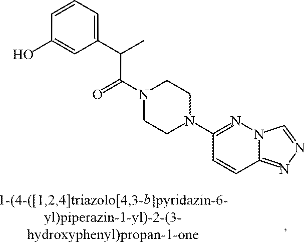

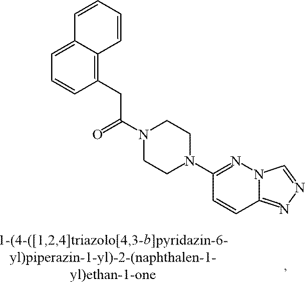

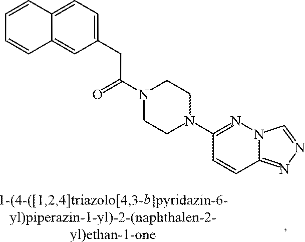

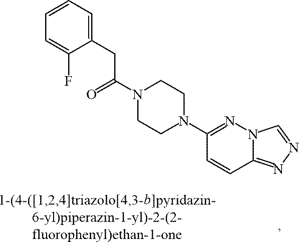

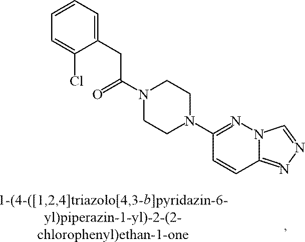

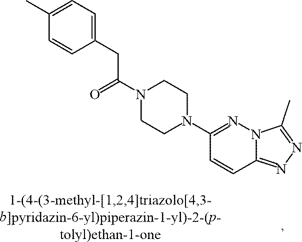

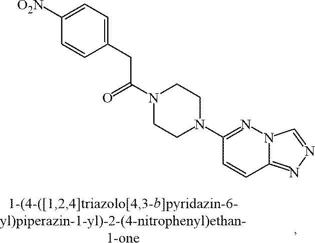

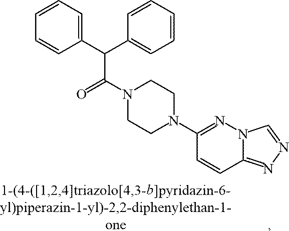

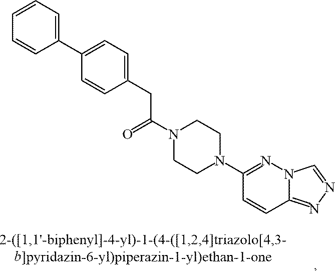

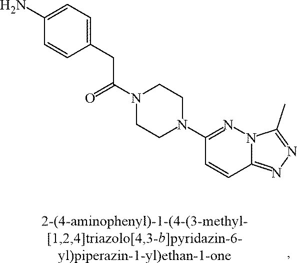

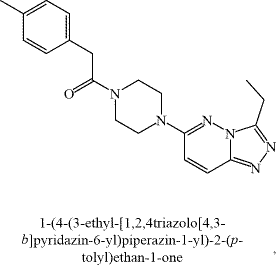

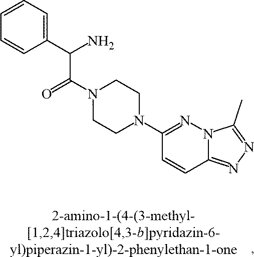

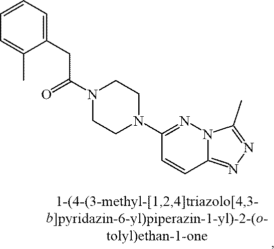

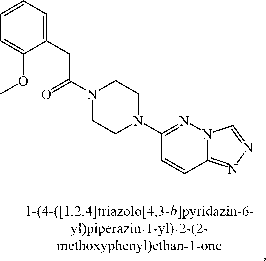

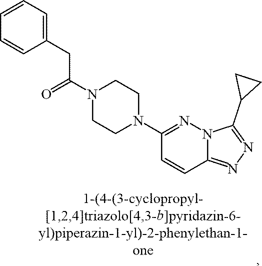

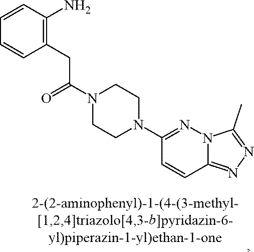

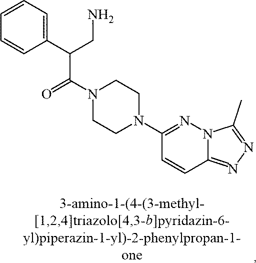

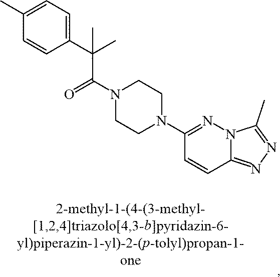

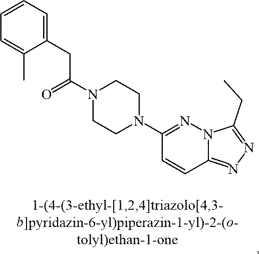

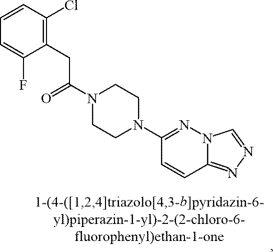

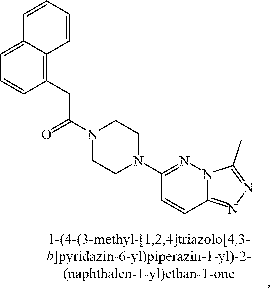

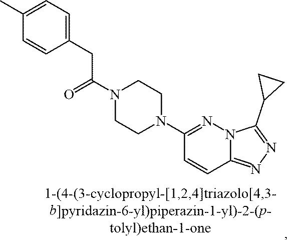

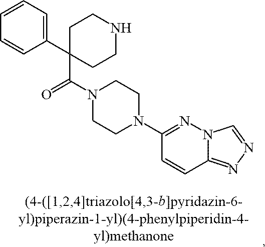

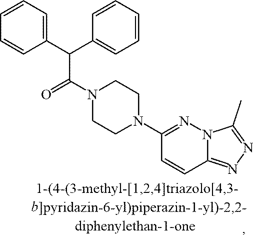

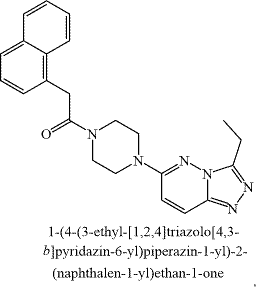

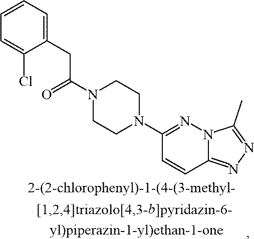

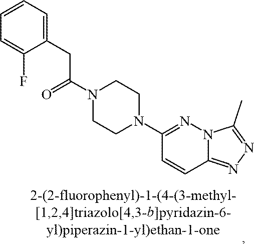

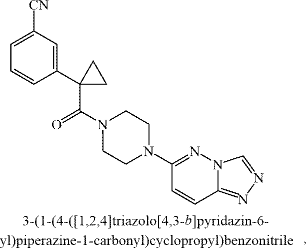

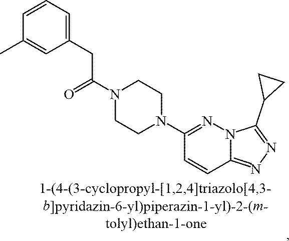

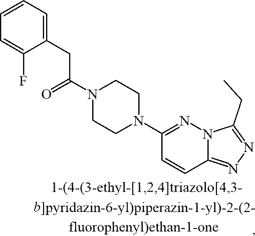

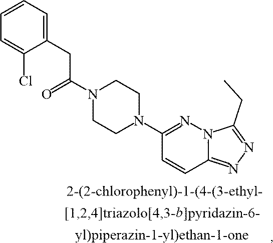

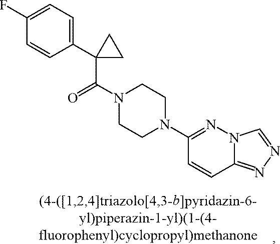

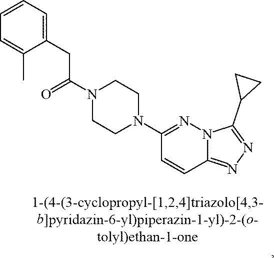

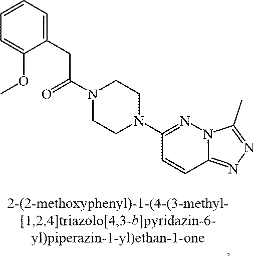

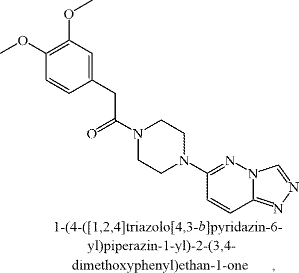

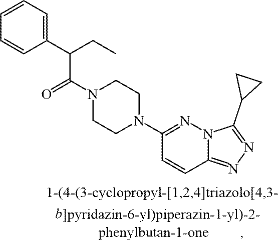

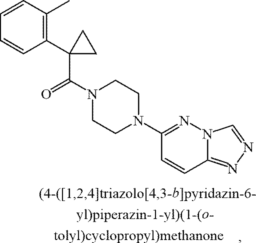

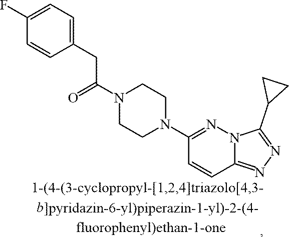

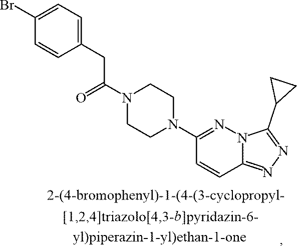

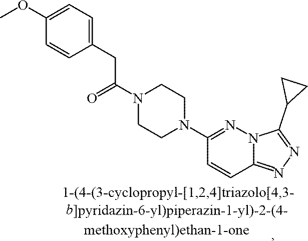

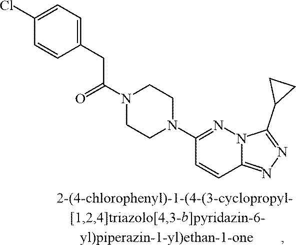

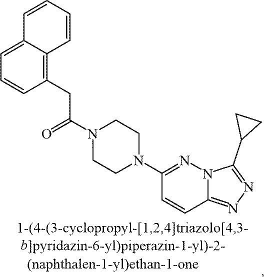

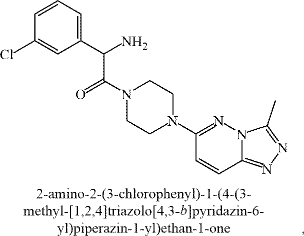

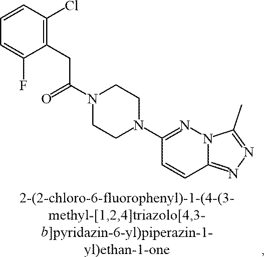

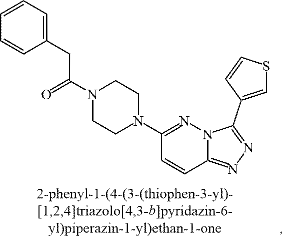

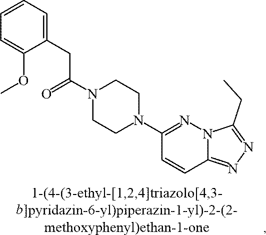

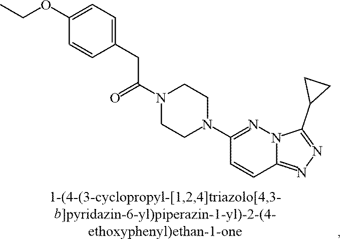

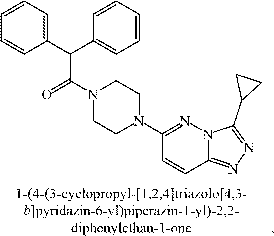

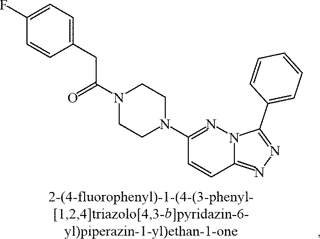

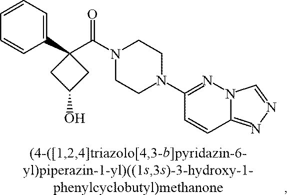

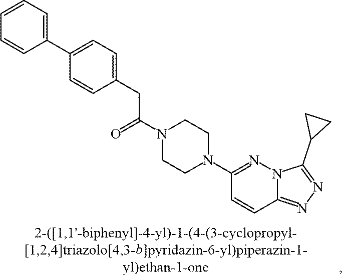

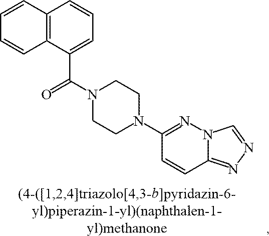

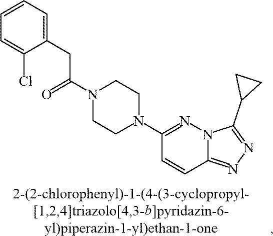

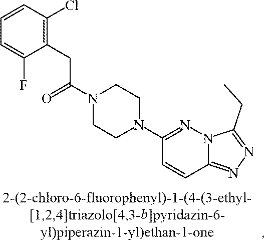

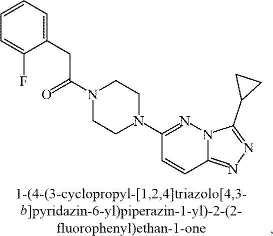

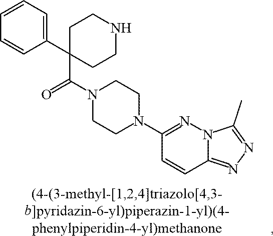

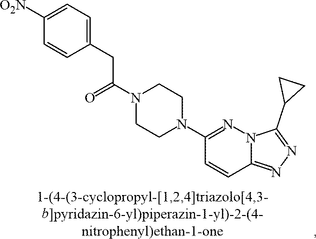

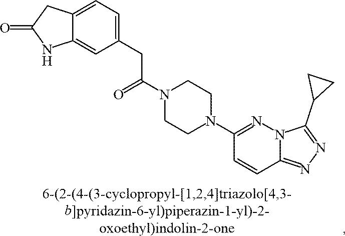

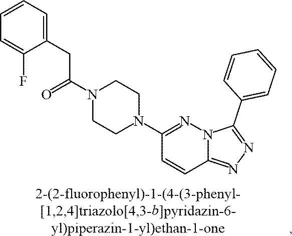

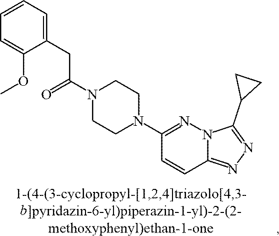

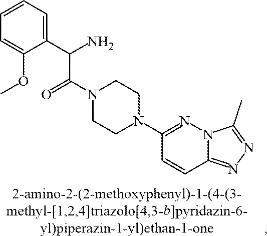

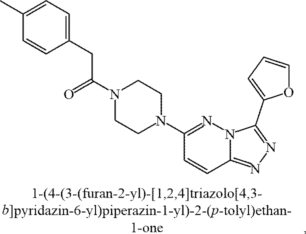

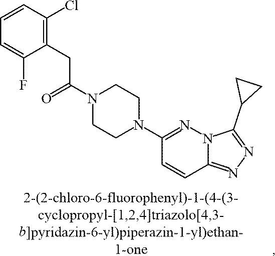

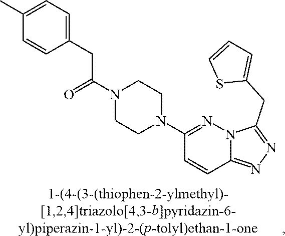

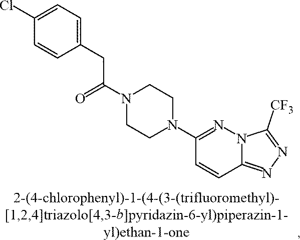

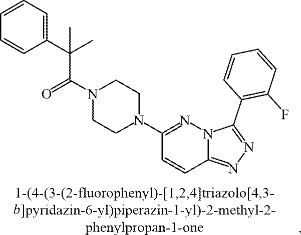

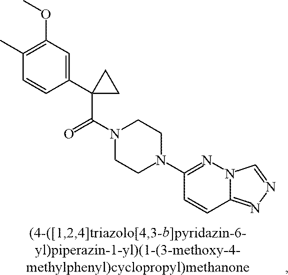

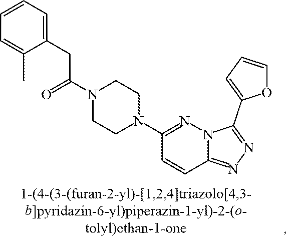

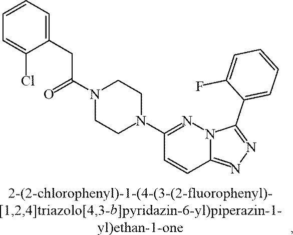

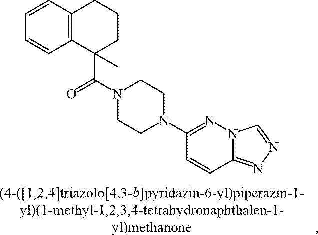

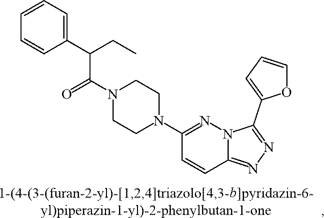

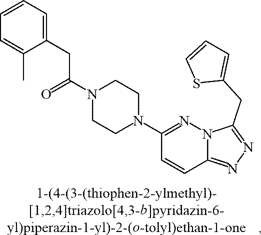

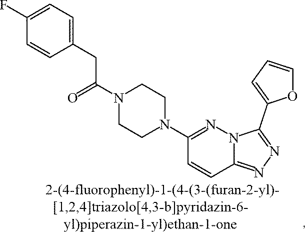

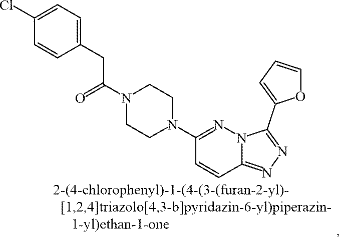

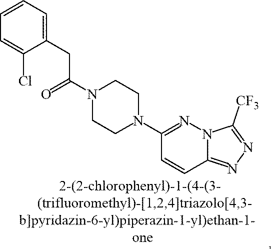

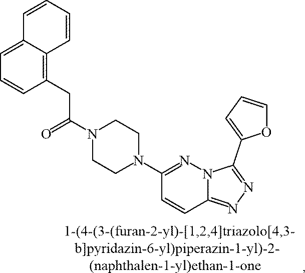

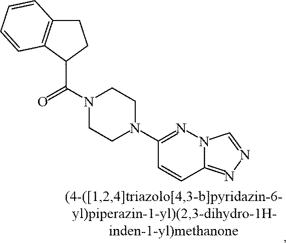

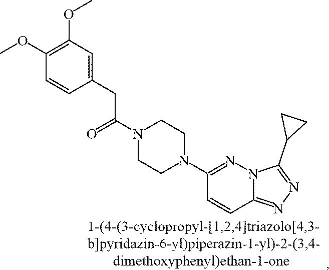

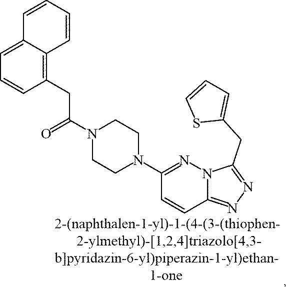

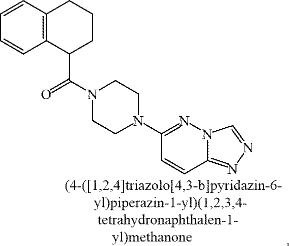

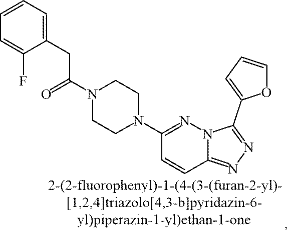

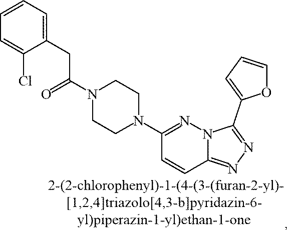

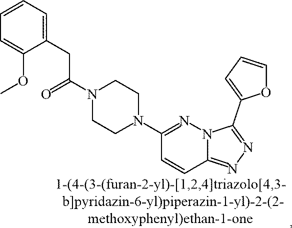

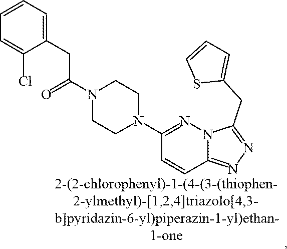

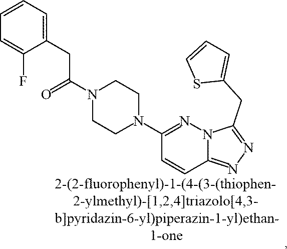

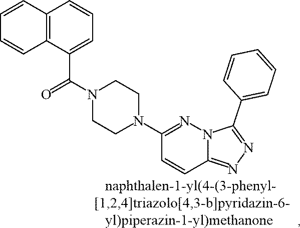

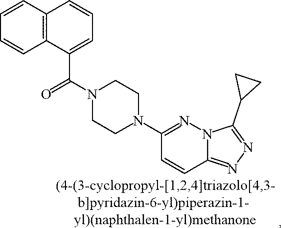

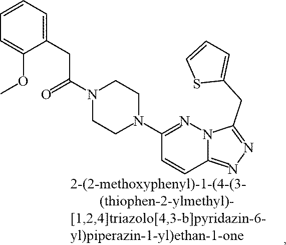

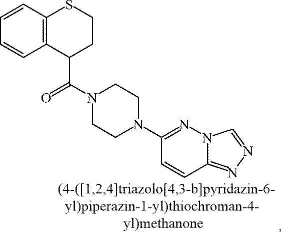

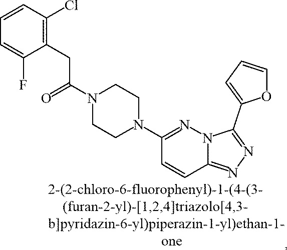

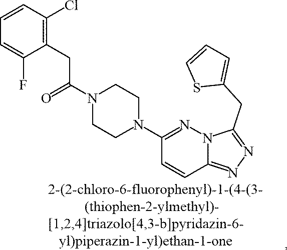

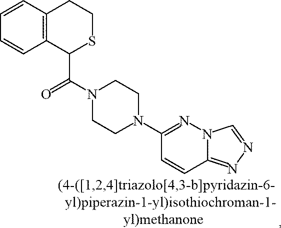

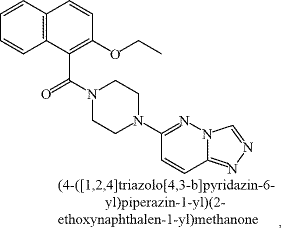

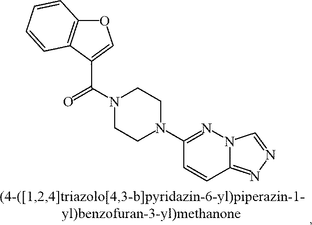

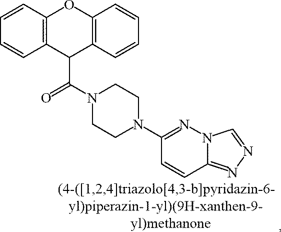

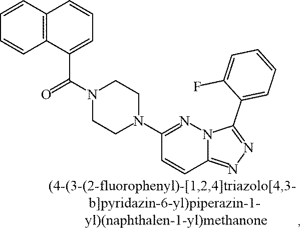

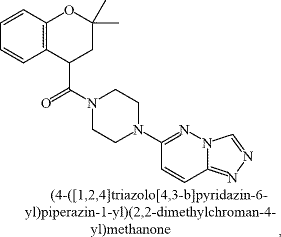

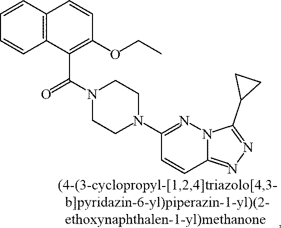

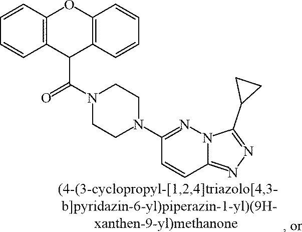

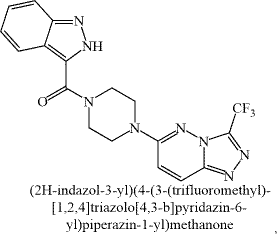

In such embodiments, R^{1a} may be chosen from —H, methyl, trifluoromethyl, phenyl, benzyl, cyclopropyl, and

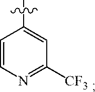

R^{1b} may be chosen from —H and methyl; and R^{1c} may be chosen from —H and methyl. In such embodiments, each R² may be independently chosen from -H and methyl. In such embodiments, R⁴ may be chosen from —H, —Cl, and —F. In such embodiments, R⁵, R⁶, and R⁷ may be independently chosen from —H, —F, —Cl, —Br, —OCH₃, —SF₅, —CF₃, —CH₃, t-butyl, ethyl, isopropyl, cyclopropyl, —CONH2, —SO₂NH₂, —SO₂CH₃, phenyl, benzyl, phenoxy, —CH₂OH, —CCH, —CHF₂, —CH₂F, —CN, —NO₂, —OCF₃, —CF₂CH₃, —CF₂CF₃, —CCCH₃, —CH₂OCH₃, —CO₂H (and salts and deprotonated forms thereof), —CONHCH₃, —SO₂NHCH₃,

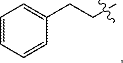

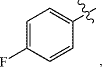

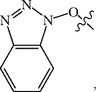

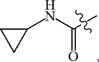

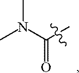

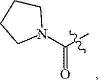

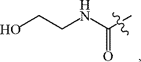

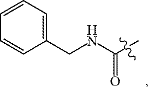

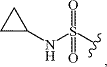

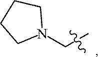

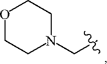

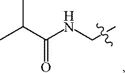

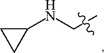

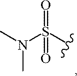

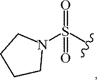

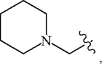

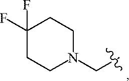

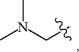

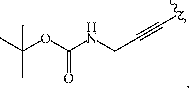

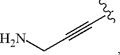

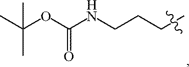

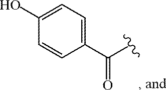

combinations thereof. In such embodiments, R⁸ may be chosen from —H, —Cl, and —F. In various embodiments, R⁴ and R⁵ may be joined to form a ring, R⁵ and R⁶ may be joined to form a ring, R⁶ and R⁷ may be joined to form a ring, or R⁷ and R⁸ may be joined to form a ring. In embodiments of the present paragraph, a compound of the present disclosure may be 2-(2-chlorophenyl)-1-(4-{[1,2,4]triazolo[4,3-b]pyridazin-6-yl}piperazin-1-yl)ethan-1-one; 2-(4-chloro-3-fluorophenyl)-1-(4-{[1,2,4]triazolo[4,3-b]pyridazin-6-yl}piperazin-1-yl)ethan-1-one; 2-(3,4-dichlorophenyl)-1-(4-{[1,2,4]triazolo[4,3-b]pyridazin-6-yl}piperazin-1-yl)ethan-1-one; 2-(2,6-dichlorophenyl)-1-(4-{[1,2,4]triazolo[4,3-b]pyridazin-6-yl}piperazin-1-yl)ethan-1-one; 2-(2,4-dichlorophenyl)-1-(4-{[1,2,4]triazolo[4,3-b]pyridazin-6-yl}piperazin-1-yl)ethan-1-one; 2-(2,5-dichlorophenyl)-1-(4-{[1,2,4]triazolo[4,3-b]pyridazin-6-yl}piperazin-1-yl)ethan-1-one; 2-(3,5-dichlorophenyl)-1-(4-{[1,2,4]triazolo[4,3-b]pyridazin-6-yl}piperazin-1-yl)ethan-1-one; 2-(3,4-dichlorophenyl)-1-(2-methyl-4-{[1,2,4]triazolo[4,3-b]pyridazin-6-yl }piperazin-1-yl)ethan-1-one; 2-(3-chloro-4-methylphenyl)-1-(4-{[1,2,4]triazolo[4,3-b]pyridazin-6-yl}piperazin-1-yl)ethan-1-one; 2-(3-fluoro-4-methylphenyl)-1-(4-{[1,2,4]triazolo[4,3-b]pyridazin-6-yl}piperazin-1-yl)ethan-1-one; 2-(4-chloro-3-methylphenyl)-1-(4-{[1,2,4]triazolo[4,3-b]pyridazin-6-yl}piperazin-1-yl)ethan-1-one; 2-(4-fluoro-3-methylphenyl)-1-(4-{[1,2,4]triazolo[4,3-b]pyridazin-6-yl}piperazin-1-yl)ethan-1-one; 2-(3-bromophenyl)-1-(4-{[1,2,4]triazolo[4,3-b]pyridazin-6-yl}piperazin-1-yl)ethan-1-one; 2-(3-methoxyphenyl)-1-(4-{[1,2,4]triazolo[4,3-b]pyridazin-6-yl}piperazin-1-yl)ethan-1-one; 2-(3-chloro-4-fluorophenyl)-1-(4-{[1,2,4]triazolo[4,3-b]pyridazin-6-yl}piperazin-1-yl)ethan-1-one; 2-(3-chlorophenyl)-1-(4-{[1,2,4]triazolo[4,3-b]pyridazin-6-yl}piperazin-1-yl)ethan-1-one; 1-(3,4-dichlorophenyl)-2-(4-{[1,2,4]triazolo[4,3-b]pyridazin-6-yl}piperazin-1-yl)ethane-1,2-dione; 1-[1-(3,4-dichlorophenyl)cyclopropanecarbonyl]-4-{[1,2,4]triazolo[4,3-b]pyridazin-6-yl}piperazine; 2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one; 4-[2-oxo-2-(4-{[1,2,4]triazolo[4,3-b]pyridazin-6-yl}piperazin-1-yl)ethyl]benzonitrile; 2-(2,3-dichlorophenyl)-1-(4-{[1,2,4]triazolo[4,3-b]pyridazin-6-yl}piperazin-1-yl)ethan-1-one; 2-(3,4-dichlorophenyl)-2,2-difluoro-1-(4-{[1,2,4]triazolo[4,3-b]pyridazin-6-yl}piperazin-1-yl)ethan-1-one; 2-(3,4-difluorophenyl)-1-(4-{[1,2,4]triazolo[4,3-b]pyridazin-6-yl}piperazin-1-yl)ethan-1-one; 2-(3,4-dichlorophenyl)-1-(4-{3-methyl-[1,2,4]triazolo[4,3-b]pyridazin-6-yl}piperazin-1-yl)ethan-1-one; 2-(3,4-dichlorophenyl)-1-(4-{3-phenyl-[1,2,4]triazolo[4,3-b]pyridazin-6-yl}piperazin-1-yl)ethan-1-one; 1-(4-{3-benzyl-[1,2,4]triazolo[4,3-b]pyridazin-6-yl}piperazin-1-yl)-2-(3,4-dichlorophenyl)ethan-1-one; 2-(3,4-dichlorophenyl)-1-{4-[3-(trifluoromethyl)-[1,2,4]triazolo[4,3-b]pyridazin-6-yl]piperazin-1-yl}ethan-1-one; 1-(4-{3-cyclopropyl-[1,2,4]triazolo[4,3-b]pyridazin-6-yl}piperazin-1-yl)-2-(3,4-dichlorophenyl)ethan-1-one; 2-(3,4-dichlorophenyl)-1-(4-{[1,2,4]triazolo[4,3-b]pyridazin-6-yl}piperazin-1-yl)propan-1-one; 2-(3,4-dichlorophenyl)-2-methyl-1-(4-{[1,2,4]triazolo[4,3-b]pyridazin-6-yl}piperazin-1-yl)propan-1-one; 2-(3,4-dichlorophenyl)-1-(3-methyl-4-{[1,2,4]triazolo[4,3-b]pyridazin-6-yl}piperazin-1-yl)ethan-1-one; 2-(3,4-dichlorophenyl)-1-(3,5-dimethyl-4-{[1,2,4]triazolo[4,3-b]pyridazin-6-yl}piperazin-1-yl)ethan-1-one; 2-(3,4-dichlorophenyl)-1-(2,6-dimethyl-4-{[1,2,4]triazolo[4,3-b]pyridazin-6-yl}piperazin-1-yl)ethan-1-one; 2-(3-chloro-5-fluorophenyl)-1-(4-{[1,2,4]triazolo[4,3-b]pyridazin-6-yl}piperazin-1-yl)ethan-1-one; 2-[4-(pentafluoro-λ⁶-sulfanyl)phenyl]-1-(4-{[1,2,4]triazolo[4,3-b]pyridazin-6-yl}piperazin-1-yl)ethan-1-one; 2-[3-(pentafluoro-λ⁶-sulfanyl)phenyl]-1-(4-{[1,2,4]triazolo[4,3-b]pyridazin-6-yl}piperazin-1-yl)ethan-1-one; 2-[4-chloro-3-(trifluoromethyl)phenyl]-1-(4-{[1,2,4]triazolo[4,3-b]pyridazin-6-yl}piperazin-1-yl)ethan-1-one; 2-[3-chloro-4-(trifluoromethyl)phenyl]-1-(4-{[1,2,4]triazolo[4,3-b]pyridazin-6-yl}piperazin-1-yl)ethan-1-one; 2-[4-fluoro-3-(trifluoromethyl)phenyl]-1-(4-{[1,2,4]triazolo[4,3-b]pyridazin-6-yl}piperazin-1-yl)ethan-1-one; 2-[3-fluoro-4-(trifluoromethyl)phenyl]-1-(4-{[1,2,4]triazolo[4,3-b]pyridazin-6-yl}piperazin-1-yl)ethan-1-one; 1-(4-{[1,2,4]triazolo[4,3-b]pyridazin-6-yl}piperazin-1-yl)-2-[3-(trifluoromethyl)phenyl]ethan-1-one; 1-(4-{[1,2,4]triazolo[4,3-b]pyridazin-6-yl}piperazin-1-yl)-2-[4-(trifluoromethyl)phenyl]ethan-1-one; 2-(2,3-dihydro-1H-inden-5-yl)-1-(4-{[1,2,4]triazolo[4,3-b]pyridazin-6-yl}piperazin-1-yl)ethan-1-one; 2-(3-tert-butylphenyl)-1-(4-{[1,2,4]triazolo[4,3-b]pyridazin-6-yl}piperazin-1-yl)ethan-1-one; 2-(4-tert-butylphenyl)-1-(4-{[1,2,4]triazolo[4,3-b]pyridazin-6-yl}piperazin-1-yl)ethan-1-one; 2-(4-chloro-3-ethylphenyl)-1-(4-{[1,2,4]triazolo[4,3-b]pyridazin-6-yl}piperazin-1-yl)ethan-1-one; 2-(3-ethyl-4-fluorophenyl)-1-(4-{[1,2,4]triazolo[4,3-b]pyridazin-6-yl}piperazin-1-yl)ethan-1-one; 2-[4-chloro-3-(propan-2-yl)phenyl]-1-(4-{[1,2,4]triazolo[4,3-b]pyridazin-6-yl}piperazin-1-yl)ethan-1-one; 2-(4-chloro-3-cyclopropylphenyl)-1-(4-{[1,2,4]triazolo[4,3-b]pyridazin-6-yl}piperazin-1-yl)ethan-1-one; 2-(4-methylphenyl)-1-(4-{[1,2,4]triazolo[4,3-b]pyridazin-6-yl}piperazin-1-yl)ethan-1-one; 2-(3-methylphenyl)-1-(4-{[1,2,4]triazolo[4,3-b]pyridazin-6-yl}piperazin-1-yl)ethan-1-one; 4-[2-oxo-2-(4-{[1,2,4]triazolo[4,3-b]pyridazin-6-yl}piperazin-1-yl)ethyl]benzamide; 3-[2-oxo-2-(4-{[1,2,4]triazolo[4,3-b]pyridazin-6-yl}piperazin-1-yl)ethyl]benzamide; N-cyclopropyl-4-[2-oxo-2-(4-{[1,2,4]triazolo[4,3-b]pyridazin-6-yl}piperazin-1-yl)ethyl]benzamide; N-cyclopropyl-3-[2-oxo-2-(4-{[1,2,4]triazolo[4,3-b]pyridazin-6-yl}piperazin-1-yl)ethyl]benzamide; N,N-dimethyl-4-[2-oxo-2-(4-{[1,2,4]triazolo[4,3-b]pyridazin-6-yl}piperazin-1-yl)ethyl]benzamide; N,N-dimethyl-3-[2-oxo-2-(4-{[1,2,4]triazolo[4,3-b]pyridazin-6-yl(piperazin-1-yl)ethyl]benzamide; 2-[4-(pyrrolidine-1-carbonyl)phenyl]-1-(4-{[1,2,4]triazolo[4,3-b]pyridazin-6-yl}piperazin-1-yl)ethan-1-one; 2-[3-(pyrrolidine-1-carbonyl)phenyl]-1-(4-{[1,2,4]triazolo[4,3-b]pyridazin-6-yl}piperazin-1-yl)ethan-1-one; N-(2-hydroxyethyl)-4-[2-oxo-2-(4-{[1,2,4]triazolo[4,3-b]pyridazin-6-yl}piperazin-1-yl)ethyl]benzamide; N-(2-hydroxyethyl)-3-[2-oxo-2-(4-{[1,2,4]triazolo[4,3-b]pyridazin-6-yl}piperazin-1-yl)ethyl]benzamide; N-benzyl-4-[2-oxo-2-(4-{[1,2,4]triazolo[4,3-b]pyridazin-6-yl}piperazin-1-yl)ethyl]benzamide;N-benzyl-3-[2-oxo-2-(4-{[1,2,4]triazolo[4,3-b]pyridazin-6-yl}piperazin-1-yl)ethyl]benzamide; 4-[2-oxo-2-(4-{[1,2,4]triazolo[4,3-b]pyridazin-6-yl}piperazin-1-yl)ethyl]benzene-1-sulfonamide; 3-[2-oxo-2-(4-{[1,2,4]triazolo[4,3-b]pyridazin-6-yl}piperazin-1-yl)ethyl]benzene-1-sulfonamide; N-cyclopropyl-4-[2-oxo-2-(4-{[1,2,4]triazolo[4,3-b]pyridazin-6-yl}piperazin-1-yl)ethyl]benzene-1-sulfonamide; N-cyclopropyl-3-[2-oxo-2-(4-{[1,2,4]triazolo[4,3-b]pyridazin-6-yl}piperazin-1-yl)ethyl]benzene-1-sulfonamide; 2-(4-methanesulfonylphenyl)-1-(4-{[1,2,4]triazolo[4,3-b]pyridazin-6-yl}piperazin-1-yl)ethan-1-one; 2-(3-methanesulfonylphenyl)-1-(4-{[1,2,4]triazolo[4,3-b]pyridazin-6-yl}piperazin-1-yl)ethan-1-one; 2-{[1,1′-biphenyl]-3-yl}-1-(4-{[1,2,4]triazolo[4,3-b]pyridazin-6-yl}piperazin-1-yl)ethan-1-one; 2-(4-benzylphenyl)-1-(4-{[1,2,4]triazolo[4,3-b]pyridazin-6-yl}piperazin-1-yl)ethan-1-one; 2-(3-benzylphenyl)-1-(4-{[1,2,4]triazolo[4,3-b]pyridazin-6-yl}piperazin-1-yl)ethan-1-one; 2-(4-phenoxyphenyl)-1-(4-{[1,2,4]triazolo[4,3-b]pyridazin-6-yl}piperazin-1-yl)ethan-1-one; 2-(3-phenoxyphenyl)-1-(4-{[1,2,4]triazolo[4,3-b]pyridazin-6-yl}piperazin-1-yl)ethan-1-one; 2-[4-(2-phenylethyl)phenyl]-1-(4-{[1,2,4]triazolo[4,3-b]pyridazin-6-yl}piperazin-1-yl)ethan-1-one; 2-[3-(2-phenylethyl)phenyl]-1-(4-{[1,2,4]triazolo[4,3-b]pyridazin-6-yl(piperazin-1-yl)ethan-1-one; 2-[3-(hydroxymethyl)phenyl]-1-(4-{[1,2,4]triazolo[4,3-b]pyridazin-6-yl}piperazin-1-yl)ethan-1-one; 2-[3-chloro-4-(hydroxymethyl)phenyl]-1-(4-{[1,2,4]triazolo[4,3-b]pyridazin-6-yl}piperazin-1-yl)ethan-1-one; 2-[4-chloro-3-(hydroxymethyl)phenyl]-1-(4-{[1,2,4]triazolo[4,3-b]pyridazin-6-yl}piperazin-1-yl)ethan-1-one; 2-{4-[(pyrrolidin-1-yl)methyl]phenyl}-1-(4-{[1,2,4]triazolo[4,3-b]pyridazin-6-yl}piperazin-1-yl)ethan-1-one; 2-{3-[(pyrrolidin-1-yl)methyl]phenyl}-1-(4-{[1,2,4]triazolo[4,3-b]pyridazin-6-yl}piperazin-1-yl)ethan-1-one; 2-{3-chloro-4-[(pyrrolidin-1-yl)methyl]phenyl}-1-(4-{[1,2,4]triazolo[4,3-b]pyridazin-6-yl}piperazin-1-yl)ethan-1-one; 2-{4-chloro-3-[(pyrrolidin-1-yl)methyl]phenyl}-1-(4-{[1,2,4]triazolo[4,3-b]pyridazin-6-yl}piperazin-1-yl)ethan-1-one; 2-{4-[(morpholin-4-yl)methyl]phenyl{-1-(4-{[1,2,4]triazolo[4,3-b]pyridazin-6-yl{piperazin-1-yl)ethan-1-one; 2-{3-[(morpholin-4-yl)methyl]phenyl}-1-(4-{[1,2,4]triazolo[4,3-b]pyridazin-6-yl}piperazin-1-yl)ethan-1-one; 2-{3-chloro-4-[(morpholin-4-yl)methyl]phenyl}-1-(4-{[1,2,4]triazolo[4,3-b]pyridazin-6-yl}piperazin-1-yl)ethan-1-one; 2-{4-chloro-3-[(morpholin-4-yl)methyl]phenyl}-1-(4-{[1,2,4]triazolo[4,3-b]pyridazin-6-yl}piperazin-1-yl)ethan-1-one; 2-methyl-N-({4-[2-oxo-2-(4-{[1,2,4]triazolo[4,3-b]pyridazin-6-yl}piperazin-1-yl)ethyl]phenyl}methyl)propanamide; 2-methyl-N-({3-[2-oxo-2-(4-{[1,2,4]triazolo[4,3-b]pyridazin-6-yl}piperazin-1-yl)ethyl]phenyl}methyl)propanamide; N-({2-chloro-4-[2-oxo-2-(4-{[1,2,4]triazolo[4,3-b]pyridazin-6-yl}piperazin-1-yl)ethyl]phenyl}methyl)-2-methylpropanamide; N-({2-chloro-5-[2-oxo-2-(4-{[1,2,4]triazolo[4,3-b]pyridazin-6-yl}piperazin-1-yl)ethyl]phenyl}methyl)-2-methylpropanamide; 2-{3-chloro-4-[(cyclopropylamino)methyl]phenyl}-1-(4-{[1,2,4]triazolo[4,3-b]pyridazin-6-yl}piperazin-1-yl)ethan-1-one; 2-{4-chloro-3-[(cyclopropylamino)methyl]phenyl}-1-(4-{[1,2,4]triazolo[4,3-b]pyridazin-6-yl}piperazin-1-yl)ethan-1-one; N,N-dimethyl-4-[2-oxo-2-(4-{[1,2,4]triazolo[4,3-b]pyridazin-6-yl}piperazin-1-yl)ethyl]benzene-1-sulfonamide;N,N-dimethyl-3-[2-oxo-2-(4-{[1,2,4]triazolo[4,3-b]pyridazin-6-yl}piperazin-1-yl)ethyl]benzene-1-sulfonamide; 2-[4-(pyrrolidine-1-sulfonyl)phenyl]-1-(4-{[1,2,4]triazolo[4,3-b]pyridazin-6-yl}piperazin-1-yl)ethan-1-one; 2-[3-(pyrrolidine-1-sulfonyl)phenyl]-1-(4-{[1,2,4]triazolo[4,3-b]pyridazin-6-yl}piperazin-1-yl)ethan-1-one; 2-(3-ethynylphenyl)-1-(4-{[1,2,4]triazolo[4,3-b]pyridazin-6-yl}piperazin-1-yl)ethan-1-one; 2-{4-[(piperidin-1-yl)methyl]phenyl}-1-(4-{[1,2,4]triazolo[4,3-b]pyridazin-6-yl}piperazin-1-yl)ethan-1-one; 2-{4-[(4,4-difluoropiperidin-1-yl)methyl]phenyl}-1-(4-{[1,2,4]triazolo[4,3-b]pyridazin-6-yl}piperazin-1-yl)ethan-1-one; 2-{4-[(dimethylamino)methyl]phenyl}-1-(4-{[1,2,4]triazolo[4,3-b]pyridazin-6-yl}piperazin-1-yl)ethan-1-one; 2-[4-chloro-3-(difluoromethyl)phenyl]-1-(4-{[1,2,4]triazolo[4,3-b]pyridazin-6-yl}piperazin-1-yl)ethan-1-one; 2-[4-chloro-3-(fluoromethyl)phenyl]-1-(4-{[1,2,4]triazolo[4,3-b]pyridazin-6-yl}piperazin-1-yl)ethan-1-one; 2-(3,4-dichlorophenyl)-1-(4-{8-methyl-[1,2,4]triazolo[4,3-b]pyridazin-6-yl}piperazin-1-yl)ethan-1-one; 2-(3,4-dichlorophenyl)-1-(4-{7-methyl-[1,2,4]triazolo[4,3-b]pyridazin-6-yl}piperazin-1-yl)ethan-1-one; 2-(3,4-dichlorophenyl)-1-{4-[3-(trifluoromethyl)-[1,2,4]triazolo[4,3-b]pyridazin-6-yl]piperazin-1-yl}ethan-1-one; tert-butyl N-(3-{4-[2-oxo-2-(4-{[1,2,4]triazolo[4,3-b]pyridazin-6-yl}piperazin-1-yl)ethyl]phenyl}prop-2-yn-1-yl)carbamate; 2-[4-(hydroxymethyl)phenyl]-1-(4-{[1,2,4]triazolo[4,3-b]pyridazin-6-yl}piperazin-1-yl)ethan-1-one; 2-[4-(3-aminoprop-1-yn-1-yl)phenyl]-1-(4-{[1,2,4]triazolo[4,3-b]pyridazin-6-yl}piperazin-1-yl)ethan-1-one; 2-(4-ethynylphenyl)-1-(4-{[1,2,4]triazolo[4,3-b]pyridazin-6-yl}piperazin-1-yl)ethan-1-one; 2-(2,3,4,5,6-pentafluorophenyl)-1-(4-{[1,2,4]triazolo[4,3-b]pyridazin-6-yl}piperazin-1-yl)ethan-1-one; 2-(4-bromo-3-chlorophenyl)-1-(4-{[1,2,4]triazolo[4,3-b]pyridazin-6-yl}piperazin-1-yl)ethan-1-one; 2-(3-bromo-4-chlorophenyl)-1-(4-{[1,2,4]triazolo[4,3-b]pyridazin-6-yl}piperazin-1-yl)ethan-1-one; 2-(4-bromo-3-fluorophenyl)-1-(4-{[1,2,4]triazolo[4,3-b]pyridazin-6-yl}piperazin-1-yl)ethan-1-one; 2-(3-bromo-4-fluorophenyl)-1-(4-{[1,2,4]triazolo[4,3-b]pyridazin-6-yl}piperazin-1-yl)ethan-1-one; tert-butyl N-(3-{4-[2-oxo-2-(4-{[1,2,4]triazolo[4,3-b]pyridazin-6-yl}piperazin-1-yl)ethyl]phenyl}propyl)carbamate; 2-(3,4-dichlorophenyl)-1-(4-{3-[2-(trifluoromethyl)pyridin-4-yl]-[1,2,4]triazolo[4,3-b]pyridazin-6-yl}piperazin-1-yl)ethan-1-one; 2-(2H-1,3-benzodioxol-5-yl)-1-(4-{[1,2,4]triazolo[4,3-b]pyridazin-6-yl}piperazin-1-yl)ethan-1-one; tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate; 2-[3-chloro-5-(trifluoromethyl)phenyl]-1-(4-{[1,2,4]triazolo[4,3-b]pyridazin-6-yl}piperazin-1-yl)ethan-1-one; 2-(3,5-difluorophenyl)-1-(4-{[1,2,4]triazolo[4,3-b]pyridazin-6-yl}piperazin-1-yl)ethan-1-one; 2-[4-(4-hydroxybenzoyl)phenyl]-1-(4-{[1,2,4]triazolo[4,3-b]pyridazin-6-yl}piperazin-1-yl)ethan-1-one; 2-(4-fluoro-3-methoxyphenyl)-1-(4-{[1,2,4]triazolo[4,3-b]pyridazin-6-yl}piperazin-1-yl)ethan-1-one; 2-[3,5-bis(trifluoromethyl)phenyl]-1-(4-{[1,2,4]triazolo[4,3-b]pyridazin-6-yl}piperazin-1-yl)ethan-1-one; 2-(3-fluoro-4-methoxyphenyl)-1-(4-{[1,2,4]triazolo[4,3-b]pyridazin-6-yl}piperazin-1-yl)ethan-1-one; 1-(4-{[1,2,4]triazolo[4,3-b]pyridazin-6-yl}piperazin-1-yl)-2-(3,4,5-trifluorophenyl)ethan-1-one; 2-[4-fluoro-3-(propan-2-yl)phenyl]-1-(4-{[1,2,4]triazolo[4,3-b]pyridazin-6-yl}piperazin-1-yl)ethan-1-one; 2-(3-cyclopropyl-4-fluorophenyl)-1-(4-{[1,2,4]triazolo[4,3-b]pyridazin-6-yl}piperazin-1-yl)ethan-1-one; 2-[3-(difluoromethyl)-4-fluorophenyl]-1-(4-{[1,2,4]triazolo[4,3-b]pyridazin-6-yl}piperazin-1-yl)ethan-1-one; 2-[4-fluoro-3-(fluoromethyl)phenyl]-1-(4-{[1,2,4]triazolo[4,3-b]pyridazin-6-yl}piperazin-1-yl)ethan-1-one; 2-[3,4-bis(trifluoromethyl)phenyl]-1-(4-{[1,2,4]triazolo[4,3-b]pyridazin-6-yl}piperazin-1-yl)ethan-1-one; 2-[3-fluoro-5-(trifluoromethyl)phenyl]-1-(4-{[1,2,4]triazolo[4,3-b]pyridazin-6-yl}piperazin-1-yl)ethan-1-one; 2-[2-fluoro-3-(trifluoromethyl)phenyl]-1-(4-{[1,2,4]triazolo[4,3-b]pyridazin-6-yl}piperazin-1-yl)ethan-1-one; 2-fluoro-5-[2-oxo-2-(4-{[1,2,4]triazolo[4,3-b]pyridazin-6-yl}piperazin-1-yl)ethyl]benzonitrile; 2-(4-fluoro-3-nitrophenyl)-1-(4-{[1,2,4]triazolo[4,3-b]pyridazin-6-yl}piperazin-1-yl)ethan-1-one; 2-[4-fluoro-3-(trifluoromethoxy)phenyl]-1-(4-{[1,2,4]triazolo[4,3-b]pyridazin-6-yl}piperazin-1-yl)ethan-1-one; 2-{6-fluoro-[1,1′-biphenyl]-3-yl}-1-(4-{[1,2,4]triazolo[4,3-b]pyridazin-6-yl}piperazin-1-yl)ethan-1-one; 2-{4′,6-difluoro-[1,1′-biphenyl]-3-yl}-1-(4-{[1,2,4]triazolo[4,3-b]pyridazin-6-yl}piperazin-1-yl)ethan-1-one; 2-[3-(1,1-difluoroethyl)-4-fluorophenyl]-1-(4-{[1,2,4]triazolo[4,3-b]pyridazin-6-yl}piperazin-1-yl)ethan-1-one; 2-[4-fluoro-3-(1,1,2,2,2-pentafluoroethyl)phenyl]-1-(4-{[1,2,4]triazolo[4,3-b]pyridazin-6-yl}piperazin-1-yl)ethan-1-one; 2-(3-ethynyl-4-fluorophenyl)-1-(4-{[1,2,4]triazolo[4,3-b]pyridazin-6-yl}piperazin-1-yl)ethan-1-one; 2-[4-fluoro-3-(prop-1-yn-1-yl)phenyl]-1-(4-{[1,2,4]triazolo[4,3-b]pyridazin-6-yl}piperazin-1-yl)ethan-1-one; 2-(3-tert-butyl-4-fluorophenyl)-1-(4-{[1,2,4]triazolo[4,3-b]pyridazin-6-yl}piperazin-1-yl)ethan-1-one; 2-[4-fluoro-3-(hydroxymethyl)phenyl]-1-(4-{[1,2,4]triazolo[4,3-b]pyridazin-6-yl}piperazin-1-yl)ethan-1-one; 2-[4-fluoro-3-(methoxymethyl)phenyl]-1-(4-{[1,2,4]triazolo[4,3-b]pyridazin-6-yl}piperazin-1-yl)ethan-1-one; 2-fluoro-5-[2-oxo-2-(4-{[1,2,4]triazolo[4,3-b]pyridazin-6-yl{piperazin-1-yl)ethyl]benzoic acid; 2-fluoro-5-[2-oxo-2-(4-{[1,2,4]triazolo[4,3-b]pyridazin-6-yl}piperazin-1-yl)ethyl]benzamide; 2-fluoro-N-methyl-5-[2-oxo-2-(4-{[1,2,4]triazolo[4,3-b]pyridazin-6-yl}piperazin-1-yl)ethyl]benzamide; 2-fluoro-N,N-dimethyl-5-[2-oxo-2-(4-{[1,2,4]triazolo[4,3-b]pyridazin-6-yl}piperazin-1-yl)ethyl]benzamide; 2-fluoro-5-[2-oxo-2-(4-{[1,2,4]triazolo[4,3-b]pyridazin-6-yl}piperazin-1-yl)ethyl]benzene-1-sulfonamide; 2-fluoro-N-methyl-5-[2-oxo-2-(4-{[1,2,4]triazolo[4,3-b]pyridazin-6-yl}piperazin-1-yl)ethyl]benzene-1-sulfonamide; 2-fluoro-N,N-dimethyl-5-[2-oxo-2-(4-{[1,2,4]triazolo[4,3-b]pyridazin-6-yl}piperazin-1-yl)ethyl]benzene-1-sulfonamide; or 2-(4-fluoro-3-methanesulfonylphenyl)-1-(4-{[1,2,4]triazolo[4,3-b]pyridazin-6-yl}piperazin-1-yl)ethan-1-one. In such embodiments, the compound may be 2-[4-fluoro-3-(trifluoromethyl)phenyl]-1-(4-{[1,2,4]triazolo[4,3-b]pyridazin-6-yl}piperazin-1-yl)ethan-1-one; 2-(3-bromo-4-chlorophenyl)-1-(4-{[1,2,4]triazolo[4,3-b]pyridazin-6-yl}piperazin-1-yl)ethan-1-one; 2-(3,4-dichlorophenyl)-1-(4-{[1,2,4]triazolo[4,3-b]pyridazin-6-yl}piperazin-1-yl)ethan-1-one; 2-(3-bromo-4-fluorophenyl)-1-(4-{[1,2,4]triazolo[4,3-b]pyridazin-6-yl}piperazin-1-yl)ethan-1-one; 2-(3-chloro-4-fluorophenyl)-1-(4-{[1,2,4]triazolo[4,3-b]pyridazin-6-yl}piperazin-1-yl)ethan-1-one; 2-(4-bromo-3-chlorophenyl)-1-(4-{[1,2,4]triazolo[4,3-b]pyridazin-6-yl}piperazin-1-yl)ethan-1-one; 2-[4-chloro-3-(trifluoromethyl)phenyl]-1-(4-{[1,2,4]triazolo[4,3-b]pyridazin-6-yl}piperazin-1-yl)ethan-1-one; 2-(4-chloro-3-methylphenyl)-1-(4-{[1,2,4]triazolo[4,3-b]pyridazin-6-yl}piperazin-1-yl)ethan-1-one; 2-[3,5-bis(trifluoromethyl)phenyl]-1-(4-{[1,2,4]triazolo[4,3-b]pyridazin-6-yl}piperazin-1-yl)ethan-1-one; 2-(4-fluoro-3-methylphenyl)-1-(4-{[1,2,4]triazolo[4,3-b]pyridazin-6-yl}piperazin-1-yl)ethan-1-one; 2-[3-chloro-5-(trifluoromethyl)phenyl]-1-(4-{[1,2,4]triazolo[4,3-b]pyridazin-6-yl}piperazin-1-yl)ethan-1-one; 2-(4-chloro-3-fluorophenyl)-1-(4-{[1,2,4]triazolo[4,3-b]pyridazin-6-yl}piperazin-1-yl)ethan-1-one; 2-(3,4-difluorophenyl)-1-(4-{[1,2,4]triazolo[4,3-b]pyridazin-6-yl}piperazin-1-yl)ethan-1-one; 2-(4-bromo-3-fluorophenyl)-1-(4-{[1,2,4]triazolo[4,3-b]pyridazin-6-yl}piperazin-1-yl)ethan-1-one; 2-(3-chloro-5-fluorophenyl)-1-(4-{[1,2,4]triazolo[4,3-b]pyridazin-6-yl}piperazin-1-yl)ethan-1-one; 2-(4-ethynylphenyl)-1-(4-{[1,2,4]triazolo[4,3-b]pyridazin-6-yl}piperazin-1-yl)ethan-1-one; or 2-(3,5-dichlorophenyl)-1-(4-{[1,2,4]triazolo[4,3-b]pyridazin-6-yl}piperazin-1-yl)ethan-1-one.

In an embodiment, a composition of the present disclosure comprises one or more compounds of the present disclosure and a pharmaceutically acceptable carrier. In such an embodiment, the composition is for oral administration and the one or more compounds are encapsulated in a pH sensitive polymer suitable for release of the compound in the small intestines, distal small intestine, or colon. In such an embodiment, the one or more compounds are chosen from: 2-(2-chlorophenyl)-1-(4-{[1,2,4]triazolo[4,3-b]pyridazin-6-yl}piperazin-1-yl)ethan-1-one; 2-(naphthalen-1-yl)-1-(4-{[1,2,4]triazolo[4,3-b]pyridazin-6-yl}piperazin-1-yl)ethan-1-one; 2-(4-chloro-3-fluorophenyl)-1-(4-{[1,2,4]triazolo[4,3-b]pyridazin-6-yl}piperazin-1-yl)ethan-1-one; 2-(4-fluorophenyl)-1-(4-{[1,2,4]triazolo[4,3-b]pyridazin-6-yl}piperazin-1-yl)ethan-1-one; 2-(3,4-dichlorophenyl)-1-(4-{[1,2,4]triazolo[4,3-b]pyridazin-6-yl}piperazin-1-yl)ethan-1-one; 2-(4-chlorophenyl)-1-(4-{[1,2,4]triazolo[4,3-b]pyridazin-6-yl}piperazin-1-yl)ethan-1-one; 2-(2,6-dichlorophenyl)-1-(4-{[1,2,4]triazolo[4,3-b]pyridazin-6-yl}piperazin-1-yl)ethan-1-one; 2-(2,4-dichlorophenyl)-1-(4-{[1,2,4]triazolo[4,3-b]pyridazin-6-yl}piperazin-1-yl)ethan-1-one; 2-(2,5-dichlorophenyl)-1-(4-{[1,2,4]triazolo[4,3-b]pyridazin-6-yl}piperazin-1-yl)ethan-1-one; 2-(3,5-dichlorophenyl)-1-(4-{[1,2,4]triazolo[4,3-b]pyridazin-6-yl}piperazin-1-yl)ethan-1-one; 2-(naphthalen-2-yl)-1-(4-{[1,2,4]triazolo[4,3-b]pyridazin-6-yl}piperazin-1-yl)ethan-1-one; 2-(3,4-dichlorophenyl)-1-(2-methyl-4-{[1,2,4]triazolo[4,3-b]pyridazin-6-yl}piperazin-1-yl)ethan-1-one; 2-(4-nitrophenyl)-1-(4-{[1,2,4]triazolo[4,3-b]pyridazin-6-yl}piperazin-1-yl)ethan-1-one; 2-(3-chloro-4-methylphenyl)-1-(4-{[1,2,4]triazolo[4,3-b]pyridazin-6-yl}piperazin-1-yl)ethan-1-one; 2-(3-fluoro-4-methylphenyl)-1-(4-{[1,2,4]triazolo[4,3-b]pyridazin-6-yl}piperazin-1-yl)ethan-1-one; 2-(4-chloro-3-methylphenyl)-1-(4-{[1,2,4]triazolo[4,3-b]pyridazin-6-yl}piperazin-1-yl)ethan-1-one; 2-(4-fluoro-3-methylphenyl)-1-(4-{[1,2,4]triazolo[4,3-b]pyridazin-6-yl}piperazin-1-yl)ethan-1-one; 2-(3-bromophenyl)-1-(4-{[1,2,4]triazolo[4,3-b]pyridazin-6-yl}piperazin-1-yl)ethan-1-one; 2-(4-methoxyphenyl)-1-(4-{[1,2,4]triazolo[4,3-b]pyridazin-6-yl}piperazin-1-yl)ethan-1-one; 2-(3-methoxyphenyl)-1-(4-{[1,2,4]triazolo[4,3-b]pyridazin-6-yl}piperazin-1-yl)ethan-1-one; 2-(3-chloro-4-fluorophenyl)-1-(4-{[1,2,4]triazolo[4,3-b]pyridazin-6-yl}piperazin-1-yl)ethan-1-one; 2-(3-chlorophenyl)-1-(4-{[1,2,4]triazolo[4,3-b]pyridazin-6-yl}piperazin-1-yl)ethan-1-one; 1-(3,4-dichlorophenyl)-2-(4-{[1,2,4]triazolo[4,3-b]pyridazin-6-yl}piperazin-1-yl)ethane-1,2-dione; 1-[1-(3,4-dichlorophenyl)cyclopropanecarbonyl]-4-{[1,2,4]triazolo[4,3-b]pyridazin-6-yl}piperazine; 2-(4-bromophenyl)-1-(4-{[1,2,4]triazolo[4,3-b]pyridazin-6-yl}piperazin-1-yl)ethan-1-one; 2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one; 4-[2-oxo-2-(4-{[1,2,4]triazolo[4,3-b]pyridazin-6-yl}piperazin-1-yl)ethyl]benzonitrile; 2-(2,3-dichlorophenyl)-1-(4-{[1,2,4]triazolo[4,3-b]pyridazin-6-yl}piperazin-1-yl)ethan-1-one; 2-(3,4-dichlorophenyl)-2,2-difluoro-1-(4-{[1,2,4]triazolo[4,3-b]pyridazin-6-yl}piperazin-1-yl)ethan-1-one; 2-(3,4-difluorophenyl)-1-(4-{[1,2,4]triazolo[4,3-b]pyridazin-6-yl}piperazin-1-yl)ethan-1-one; 2-(3,4-dichlorophenyl)-1-(4-{3-methyl-[1,2,4]triazolo[4,3-b]pyridazin-6-yl}piperazin-1-yl)ethan-1-one; 2-(3,4-dichlorophenyl)-1-(4-{3-phenyl-[1,2,4]triazolo[4,3-b]pyridazin-6-yl}piperazin-1-yl)ethan-1-one; 1-(4-{3-benzyl-[1,2,4]triazolo[4,3-b]pyridazin-6-yl}piperazin-1-yl)-2-(3,4-dichlorophenyl)ethan-1-one; 2-(3,4-di chlorophenyl)-1-{4-[3-(trifluoromethyl)-[1,2,4]triazolo[4,3-b]pyridazin-6-yl]piperazin-1-yl}ethan-1-one; 1-(4-{3-cyclopropyl-[1,2,4]triazolo[4,3-b]pyridazin-6-yl}piperazin-1-yl)-2-(3,4-dichlorophenyl)ethan-1-one; 2-(3,4-dichlorophenyl)-1-(4-{[1,2,4]triazolo[4,3-b]pyridazin-6-yl}piperazin-1-yl)propan-1-one; 2-(3,4-dichlorophenyl)-2-methyl-1-(4-{[1,2,4]triazolo[4,3-b]pyridazin-6-yl}piperazin-1-yl)propan-1-one; 2-(3,4-dichlorophenyl)-1-(3-methyl-4-{[1,2,4]triazolo[4,3-b]pyridazin-6-yl}piperazin-1-yl)ethan-1-one; 2-(3,4-dichlorophenyl)-1-(3,5-dimethyl-4-{[1,2,4]triazolo[4,3-b]pyridazin-6-yl}piperazin-1-yl)ethan-1-one; 2-(3,4-dichlorophenyl)-1-(2,6-dimethyl-4-{[1,2,4]triazolo[4,3-b]pyridazin-6-yl}piperazin-1-yl)ethan-1-one; 2-(3-chloro-5-fluorophenyl)-1-(4-{[1,2,4]triazolo[4,3-b]pyridazin-6-yl}piperazin-1-yl)ethan-1-one; 2-[4-(pentafluoro-λ⁶-sulfanyl)phenyl]-1-(4-{[1,2,4]triazolo[4,3-b]pyridazin-6-yl}piperazin-1-yl)ethan-1-one; 2-[3-(pentafluoro-λ⁶-sulfanyl)phenyl]-1-(4-{[1,2,4]triazolo[4,3-b]pyridazin-6-yl}piperazin-1-yl)ethan-1-one; 2-[4-chloro-3-(trifluoromethyl)phenyl]-1-(4-{[1,2,4]triazolo[4,3-b]pyridazin-6-yl}piperazin-1-yl)ethan-1-one; 2-[3-chloro-4-(trifluoromethyl)phenyl]-1-(4-{[1,2,4]triazolo[4,3-b]pyridazin-6-yl} piperazin-1-yl)ethan-1-one; 2-[4-fluoro-3-(trifluoromethyl)phenyl]-1-(4-{[1,2,4]triazolo[4,3-b]pyridazin-6-yl}piperazin-1-yl)ethan-1-one; 2-[3-fluoro-4-(trifluoromethyl)phenyl]-1-(4-{[1,2,4]triazolo[4,3-b]pyridazin-6-yl}piperazin-1-yl)ethan-1-one; 1-(4-{[1,2,4]triazolo[4,3-b]pyridazin-6-yl}piperazin-1-yl)-2-[3-(trifluoromethyl)phenyl]ethan-1-one; 1-(4-{[1,2,4]triazolo[4,3-b]pyridazin-6-yl}piperazin-1-yl)-2-[4-(trifluoromethyl)phenyl]ethan-1-one; 2-(2,3-dihydro-1H-inden-5-yl)-1-(4-{[1,2,4]triazolo[4,3-b]pyridazin-6-yl}piperazin-1-yl)ethan-1-one; 2-(3-tert-butylphenyl)-1-(4-{[1,2,4]triazolo[4,3-b]pyridazin-6-yl}piperazin-1-yl)ethan-1-one; 2-(4-tert-butylphenyl)-1-(4-{[1,2,4]triazolo[4,3-b]pyridazin-6-yl}piperazin-1-yl)ethan-1-one; 2-(4-chloro-3-ethylphenyl)-1-(4-{[1,2,4]triazolo[4,3-b]pyridazin-6-yl}piperazin-1-yl)ethan-1-one; 2-(3-ethyl-4-fluorophenyl)-1-(4-{[1,2,4]triazolo[4,3-b]pyridazin-6-yl}piperazin-1-yl)ethan-1-one; 2-[4-chloro-3-(propan-2-yl)phenyl]-1-(4-{[1,2,4]triazolo[4,3-b]pyridazin-6-yl}piperazin-1-yl)ethan-1-one; 2-(4-chloro-3-cyclopropylphenyl)-1-(4-{[1,2,4]triazolo[4,3-b]pyridazin-6-yl}piperazin-1-yl)ethan-1-one; 2-(4-methylphenyl)-1-(4-{[1,2,4]triazolo[4,3-b]pyridazin-6-yl}piperazin-1-yl)ethan-1-one; 2-(3-methylphenyl)-1-(4-{[1,2,4]triazolo[4,3-b]pyridazin-6-yl}piperazin-1-yl)ethan-1-one; 4-[2-oxo-2-(4-{[1,2,4]triazolo[4,3-b]pyridazin-6-yl}piperazin-1-yl)ethyl]benzamide; 3-[2-oxo-2-(4-{[1,2,4]triazolo[4,3-b]pyridazin-6-yl}piperazin-1-yl)ethyl]benzamide; N-cyclopropyl-4-[2-oxo-2-(4-{[1,2,4]triazolo[4,3-b]pyridazin-6-yl}piperazin-1-yl)ethyl]benzamide; N-cyclopropyl-3-[2-oxo-2-(4-{[1,2,4]triazolo[4,3-b]pyridazin-6-yl}piperazin-1-yl)ethyl]benzamide; N,N-dimethyl-4-[2-oxo-2-(4-{[1,2,4]triazolo[4,3-b]pyridazin-6-yl}piperazin-1-yl)ethyl]benzamide; N,N-dimethyl-3-[2-oxo-2-(4-{[1,2,4]triazolo[4,3-b]pyridazin-6-yl}piperazin-1-yl)ethyl]benzamide; 2-[4-(pyrrolidine-1-carbonyl)phenyl]-1-(4-{[1,2,4]triazolo[4,3-b]pyridazin-6-yl}piperazin-1-yl)ethan-1-one; 2-[3-(pyrrolidine-1-carbonyl)phenyl]-1-(4-{[1,2,4]triazolo[4,3-b]pyridazin-6-yl}piperazin-1-yl)ethan-1-one; N-(2-hydroxyethyl)-4-[2-oxo-2-(4-{[1,2,4]triazolo[4,3-b]pyridazin-6-yl}piperazin-1-yl)ethyl]benzamide; N-(2-hydroxyethyl)-3-[2-oxo-2-(4-{[1,2,4]triazolo[4,3-b]pyridazin-6-yl}piperazin-1-yl)ethyl]benzamide; N-benzyl-4-[2-oxo-2-(4-{[1,2,4]triazolo[4,3-b]pyridazin-6-yl}piperazin-1-yl)ethyl]benzamide; N-benzyl-3-[2-oxo-2-(4-{[1,2,4]triazolo[4,3-b]pyridazin-6-yl}piperazin-1-yl)ethyl]benzamide; 4-[2-oxo-2-(4-{[1,2,4]triazolo[4,3-b]pyridazin-6-yl}piperazin-1-yl)ethyl]benzene-1-sulfonamide; 3-[2-oxo-2-(4-{[1,2,4]triazolo[4,3-b]pyridazin-6-yl}piperazin-1-yl)ethyl]benzene-1-sulfonamide; N-cyclopropyl-4-[2-oxo-2-(4-{[1,2,4]triazolo[4,3-b]pyridazin-6-yl}piperazin-1-yl)ethyl]benzene-1-sulfonamide; N-cyclopropyl-3-[2-oxo-2-(4-{[1,2,4]triazolo[4,3-b]pyridazin-6-yl}piperazin-1-yl)ethyl]benzene-1-sulfonamide; 2-(4-methanesulfonylphenyl)-1-(4-{[1,2,4]triazolo[4,3-b]pyridazin-6-yl}piperazin-1-yl)ethan-1-one; 2-(3-methanesulfonylphenyl)-1-(4-{[1,2,4]triazolo[4,3-b]pyridazin-6-yl}piperazin-1-yl)ethan-1-one; 2-{[1,1′-biphenyl]-4-yl}-1-(4-{[1,2,4]triazolo[4,3-b]pyridazin-6-yl}piperazin-1-yl)ethan-1-one; 2-{[1,1′-biphenyl]-3-yl}-1-(4-{[1,2,4]triazolo[4,3-b]pyridazin-6-yl}piperazin-1-yl)ethan-1-one; 2-(4-benzylphenyl)-1-(4-{[1,2,4]triazolo[4,3-b]pyridazin-6-yl}piperazin-1-yl)ethan-1-one; 2-(3-benzylphenyl)-1-(4-{[1,2,4]triazolo[4,3-b]pyridazin-6-yl}piperazin-1-yl)ethan-1-one; 2-(4-phenoxyphenyl)-1-(4-{[1,2,4]triazolo[4,3-b]pyridazin-6-yl}piperazin-1-yl)ethan-1-one; 2-(3-phenoxyphenyl)-1-(4-{[1,2,4]triazolo[4,3-b]pyridazin-6-yl}piperazin-1-yl)ethan-1-one; 2-[4-(2-phenylethyl)phenyl]-1-(4-{[1,2,4]triazolo[4,3-b]pyridazin-6-yl}piperazin-1-yl)ethan-1-one; 2-[3-(2-phenylethyl)phenyl]-1-(4-{[1,2,4]triazolo[4,3-b]pyridazin-6-yl}piperazin-1-yl)ethan-1-one; 2-[3-(hydroxymethyl)phenyl]-1-(4-{[1,2,4]triazolo[4,3-b]pyridazin-6-yl}piperazin-1-yl)ethan-1-one; 2-[3-chloro-4-(hydroxymethyl)phenyl]-1-(4-{[1,2,4]triazolo[4,3-b]pyridazin-6-yl}piperazin-1-yl)ethan-1-one; 2-[4-chloro-3-(hydroxymethyl)phenyl]-1-(4-{[1,2,4]triazolo[4,3-b]pyridazin-6-yl}piperazin-1-yl)ethan-1-one; 2-{4-[(pyrrolidin-1-yl)methyl]phenyl}-1-(4-{[1,2,4]triazolo[4,3-b]pyridazin-6-yl}piperazin-1-yl)ethan-1-one; 2-{3-[(pyrrolidin-1-yl)methyl]phenyl}-1-(4-{[1,2,4]triazolo[4,3-b]pyridazin-6-yl}piperazin-1-yl)ethan-1-one; 2-{3-chloro-4-[(pyrrolidin-1-yl)methyl]phenyl}-1-(4-{[1,2,4]triazolo[4,3-b]pyridazin-6-yl}piperazin-1-yl)ethan-1-one; 2-{4-chloro-3-[(pyrrolidin-1-yl)methyl]phenyl}-1-(4-{[1,2,4]triazolo[4,3-b]pyridazin-6-yl}piperazin-1-yl)ethan-1-one; 2-{4-[(morpholin-4-yl)methyl]phenyl}-1-(4-{[1,2,4]triazolo[4,3-b]pyridazin-6-yl}piperazin-1-yl)ethan-1-one; 2-{3-[(morpholin-4-yl)methyl]phenyl}-1-(4-{[1,2,4]triazolo[4,3-b]pyridazin-6-yl}piperazin-1-yl)ethan-1-one; 2-{3-chloro-4-[(morpholin-4-yl)methyl]phenyl}-1-(4-{[1,2,4]triazolo[4,3-b]pyridazin-6-yl}piperazin-1-yl)ethan-1-one; 2-{4-chloro-3-[(morpholin-4-yl)methyl]phenyl}-1-(4-{[1,2,4]triazolo[4,3-b]pyridazin-6-yl}piperazin-1-yl)ethan-1-one; 2-methyl-N-({4-[2-oxo-2-(4-{[1,2,4]triazolo[4,3-b]pyridazin-6-yl}piperazin-1-yl)ethyl]phenyl}methyl)propanamide; 2-methyl-N-({3-[2-oxo-2-(4-{[1,2,4]triazolo[4,3-b]pyridazin-6-yl}piperazin-1-yl)ethyl]phenyl}methyl)propanamide;N-({2-chloro-4-[2-oxo-2-(4-{[1,2,4]triazolo[4,3-b]pyridazin-6-yl}piperazin-1-yl)ethyl]phenyl}methyl)-2-methylpropanamide;N-({2-chloro-5-[2-oxo-2-(4-{[1,2,4]triazolo[4,3-b]pyridazin-6-yl}piperazin-1-yl)ethyl]phenyl}methyl)-2-methylpropanamide; 2-{3-chloro-4-[(cyclopropylamino)methyl]phenyl}-1-(4-{[1,2,4]triazolo[4,3-b]pyridazin-6-yl}piperazin-1-yl)ethan-1-one; 2-{4-chloro-3-[(cyclopropylamino)methyl]phenyl}-1-(4-{[1,2,4]triazolo[4,3-b]pyridazin-6-yl}piperazin-1-yl)ethan-1-one; N,N-dimethyl-4-[2-oxo-2-(4-{[1,2,4]triazolo[4,3-b]pyridazin-6-yl}piperazin-1-yl)ethyl]benzene-1-sulfonamide; N,N-dimethyl-3-[2-oxo-2-(4-{[1,2,4]triazolo[4,3-b]pyridazin-6-yl}piperazin-1-yl)ethyl]benzene-1-sulfonamide; 2-[4-(pyrrolidine-1-sulfonyl)phenyl]-1-(4-{[1,2,4]triazolo[4,3-b]pyridazin-6-yl}piperazin-1-yl)ethan-1-one; 2-[3-(pyrrolidine-1-sulfonyl)phenyl]-1-(4-{[1,2,4]triazolo[4,3-b]pyridazin-6-yl}piperazin-1-yl)ethan-1-one; 2-(3-ethynylphenyl)-1-(4-{[1,2,4]triazolo[4,3-b]pyridazin-6-yl}piperazin-1-yl)ethan-1-one; 2-{4- [(piperidin-1-yl)methyl]phenyl}-1-(4-{[1,2,4]triazolo[4,3-b]pyridazin-6-yl(piperazin-1-yl)ethan-1-one; 2-{4-[(4,4-difluoropiperidin-1-yl)methyl]phenyl}-1-(4-{[1,2,4]triazolo[4,3-b]pyridazin-6-yl(piperazin-1-yl)ethan-1-one; 2-{4-[(dimethylamino)methyl]phenyl}-1-(4-{[1,2,4]triazolo[4,3-b]pyridazin-6-yl}piperazin-1-yl)ethan-1-one; 2-[4-chloro-3-(difluoromethyl)phenyl]-1-(4-{[1,2,4]triazolo[4,3-b]pyridazin-6-yl}piperazin-1-yl)ethan-1-one; 2-[4-chloro-3-(fluoromethyl)phenyl]-1-(4-{[1,2,4]triazolo[4,3-b]pyridazin-6-yl}piperazin-1-yl)ethan-1-one; 2-(3,4-dichlorophenyl)-1-(4-{8-methyl-[1,2,4]triazolo[4,3-b]pyridazin-6-yl}piperazin-1-yl)ethan-1-one; 2-(3,4-dichlorophenyl)-1-(4-{7-methyl-[1,2,4]triazolo[4,3-b]pyridazin-6-yl}piperazin-1-yl)ethan-1-one; 2-(3,4-dichlorophenyl)-1-{4-[3-(trifluoromethyl)-[1,2,4]triazolo[4,3-b]pyridazin-6-yl]piperazin-1-yl}ethan-1-one; tert-butyl N-(3-{4-[2-oxo-2-(4-{[1,2,4]triazolo[4,3-b]pyridazin-6-yl}piperazin-1-yl)ethyl]phenyl}prop-2-yn-1-yl)carbamate; 2-[4-(hydroxymethyl)phenyl]-1-(4-{[1,2,4]triazolo[4,3-b]pyridazin-6-yl}piperazin-1-yl)ethan-1-one; 2-phenyl-1-(4-{[1,2,4]triazolo[4,3-b]pyridazin-6-yl}piperazin-1-yl)ethan-1-one; 2-[4-(3-aminoprop-1-yn-1-yl)phenyl]-1-(4-{[1,2,4]triazolo[4,3-b]pyridazin-6-yl}piperazin-1-yl)ethan-1-one; 2-(4-ethynylphenyl)-1-(4-{[1,2,4]triazolo[4,3-b]pyridazin-6-yl}piperazin-1-yl)ethan-1-one; 2-(2,3,4,5,6-pentafluorophenyl)-1-(4-{[1,2,4]triazolo[4,3-b]pyridazin-6-yl}piperazin-1-yl)ethan-1-one; 2-(4-bromo-3-chlorophenyl)-1-(4-{[1,2,4]triazolo[4,3-b]pyridazin-6-yl}piperazin-1-yl)ethan-1-one; 2-(3-bromo-4-chlorophenyl)-1-(4-{[1,2,4]triazolo[4,3-b]pyridazin-6-yl}piperazin-1-yl)ethan-1-one; 2-(4-bromo-3-fluorophenyl)-1-(4-{[1,2,4]triazolo[4,3-b]pyridazin-6-yl}piperazin-1-yl)ethan-1-one; 2-(3-bromo-4-fluorophenyl)-1-(4-{[1,2,4]triazolo[4,3-b]pyridazin-6-yl}piperazin-1-yl)ethan-1-one; tert-butyl-N-(3-{4-[2-oxo-2-(4-{[1,2,4]triazolo[4,3-b]pyridazin-6-yl(piperazin-1-yl)ethyl]phenyl}propyl)carbamate; 2-(3,4-dichlorophenyl)-1-(4-{3-[2-(trifluoromethyl)pyridin-4-yl]-[1,2,4]triazolo[4,3-b]pyridazin-6-yl}piperazin-1-yl)ethan-1-one; 2-(2H-1,3-benzodioxol-5-yl)-1-(4-{[1,2,4]triazolo[4,3-b]pyridazin-6-yl}piperazin-1-yl)ethan-1-one; tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate; 2-[3-chloro-5-(trifluoromethyl)phenyl]-1-(4-{[1,2,4]triazolo[4,3-b]pyridazin-6-yl}piperazin-1-yl)ethan-1-one; 2-(3,5-difluorophenyl)-1-(4-{[1,2,4]triazolo[4,3-b]pyridazin-6-yl}piperazin-1-yl)ethan-1-one; 2-[4-(4-hydroxybenzoyl)phenyl]-1-(4-{[1,2,4]triazolo[4,3-b]pyridazin-6-yl}piperazin-1-yl)ethan-1-one; 2-(4-fluoro-3-methoxyphenyl)-1-(4-{[1,2,4]triazolo[4,3-b]pyridazin-6-yl}piperazin-1-yl)ethan-1-one; 2-[3,5-bis(trifluoromethyl)phenyl]-1-(4-{[1,2,4]triazolo[4,3-b]pyridazin-6-yl}piperazin-1-yl)ethan-1-one; 2-(3-fluoro-4-methoxyphenyl)-1-(4-{[1,2,4]triazolo[4,3-b]pyridazin-6-yl}piperazin-1-yl)ethan-1-one; 2-(3-methylphenyl)-1-(4-{[1,2,4]triazolo[4,3-b]pyridazin-6-yl}piperazin-1-yl)ethan-1-one; 2-(4-methylphenyl)-1-(4-{[1,2,4]triazolo[4,3-b]pyridazin-6-yl}piperazin-1-yl)ethan-1-one; 1-(4-{[1,2,4]triazolo[4,3-b]pyridazin-6-yl}piperazin-1-yl)-2-(3,4,5-trifluorophenyl)ethan-1-one; 2-[4-fluoro-3-(propan-2-yl)phenyl]-1-(4-{[1,2,4]triazolo[4,3-b]pyridazin-6-yl}piperazin-1-yl)ethan-1-one; 2-(3-cyclopropyl-4-fluorophenyl)-1-(4-{[1,2,4]triazolo[4,3-b]pyridazin-6-yl}piperazin-1-yl)ethan-1-one; 2-[3-(difluoromethyl)-4-fluorophenyl]-1-(4-{[1,2,4]triazolo[4,3-b]pyridazin-6-yl}piperazin-1-yl)ethan-1-one; 2-[4-fluoro-3-(fluoromethyl)phenyl]-1-(4-{[1,2,4]triazolo[4,3-b]pyridazin-6-yl}piperazin-1-yl)ethan-1-one; 2-[3,4-bis(trifluoromethyl)phenyl]-1-(4-{[1,2,4]triazolo[4,3-b]pyridazin-6-yl}piperazin-1-yl)ethan-1-one; 2-[3-fluoro-5-(trifluoromethyl)phenyl]-1-(4-{[1,2,4]triazolo[4,3-b]pyridazin-6-yl}piperazin-1-yl)ethan-1-one; 2-[2-fluoro-3-(trifluoromethyl)phenyl]-1-(4-{[1,2,4]triazolo[4,3-b]pyridazin-6-yl}piperazin-1-yl)ethan-1-one; 2-fluoro-5-[2-oxo-2-(4-{[1,2,4]triazolo[4,3-b]pyridazin-6-yl}piperazin-1-yl)ethyl]benzonitrile; 2-(4-fluoro-3-nitrophenyl)-1-(4-{[1,2,4]triazolo[4,3-b]pyridazin-6-yl}piperazin-1-yl)ethan-1-one; 2-[4-fluoro-3-(trifluoromethoxy)phenyl]-1-(4-{[1,2,4]triazolo[4,3-b]pyridazin-6-yl}piperazin-1-yl)ethan-1-one; 2-{6-fluoro-[1,1′-biphenyl]-3-yl}-1-(4-{[1,2,4]triazolo[4,3-b]pyridazin-6-yl}piperazin-1-yl)ethan-1-one; 2-{4′,6-difluoro-[1,1′-biphenyl]-3-yl}-1-(4-{[1,2,4]triazolo[4,3-b]pyridazin-6-yl}piperazin-1-yl)ethan-1-one; 2-[3-(1,1-difluoroethyl)-4-fluorophenyl]-1-(4-{[1,2,4]triazolo[4,3-b]pyridazin-6-yl}piperazin-1-yl)ethan-1-one; 2-[4-fluoro-3-(1, 1,2,2,2-pentafluoroethyl)phenyl]-1-(4-{[1,2,4]triazolo[4,3-b]pyridazin-6-yl}piperazin-1-yl)ethan-1-one; 2-(3-ethynyl-4-fluorophenyl)-1-(4-{[1,2,4]triazolo[4,3-b]pyridazin-6-yl}piperazin-1-yl)ethan-1-one; 2-[4-fluoro-3-(prop-1-yn-1-yl)phenyl]-1-(4-{[1,2,4]triazolo[4,3-b]pyridazin-6-yl}piperazin-1-yl)ethan-1-one; 2-(3-tert-butyl-4-fluorophenyl)-1-(4-{[1,2,4]triazolo[4,3-b]pyridazin-6-yl}piperazin-1-yl)ethan-1-one; 2-[4-fluoro-3-(hydroxymethyl)phenyl]-1-(4-{[1,2,4]triazolo[4,3-b]pyridazin-6-yl}piperazin-1-yl)ethan-1-one; 2-[4-fluoro-3-(methoxymethyl)phenyl]-1-(4-{[1,2,4]triazolo[4,3-b]pyridazin-6-yl}piperazin-1-yl)ethan-1-one; 2-fluoro-5-[2-oxo-2-(4-{[1,2,4]triazolo[4,3-b]pyridazin-6-yl(piperazin-1-yl)ethyl]benzoic acid; 2-fluoro-5-[2-oxo-2-(4-{[1,2,4]triazolo[4,3-b]pyridazin-6-yl}piperazin-1-yl)ethyl]benzamide; 2-fluoro-N-methyl-5-[2-oxo-2-(4-{[1,2,4]triazolo[4,3-b]pyridazin-6-yl}piperazin-1-yl)ethyl]benzamide; 2-fluoro-N,N-dimethyl-5-[2-oxo-2-(4-{[1,2,4]triazolo[4,3-b]pyridazin-6-yl}piperazin-1-yl)ethyl]benzamide; 2-fluoro-5-[2-oxo-2-(4-{[1,2,4]triazolo[4,3-b]pyridazin-6-yl}piperazin-1-yl)ethyl]benzene-1-sulfonamide; 2-fluoro-N-methyl-5-[2-oxo-2-(4-{[1,2,4]triazolo[4,3-b]pyridazin-6-yl}piperazin-1-yl)ethyl]benzene-1-sulfonamide; 2-fluoro-N,N-dimethyl-5-[2-oxo-2-(4-{[1,2,4]triazolo[4,3-b]pyridazin-6-yl}piperazin-1-yl)ethyl]benzene-1-sulfonamide; 2-(4-fluoro-3-methanesulfonylphenyl)-1-(4-{[1,2,4]triazolo[4,3-b]pyridazin-6-yl}piperazin-1-yl)ethan-1-one; and combinations thereof. In such embodiments, the one or more compounds may be chosen from: 2-[4-fluoro-3-(trifluoromethyl)phenyl]-1-(4-{[1,2,4]triazolo[4,3-b]pyridazin-6-yl}piperazin-1-yl)ethan-1-one; 2-(3-bromo-4-chlorophenyl)-1-(4-{[1,2,4]triazolo[4,3-b]pyridazin-6-yl}piperazin-1-yl)ethan-1-one; 2-(3,4-dichlorophenyl)-1-(4-{[1,2,4]triazolo[4,3-b]pyridazin-6-yl}piperazin-1-yl)ethan-1-one; 2-(3-bromo-4-fluorophenyl)-1-(4-{[1,2,4]triazolo[4,3-b]pyridazin-6-yl}piperazin-1-yl)ethan-1-one; 2-(3-chloro-4-fluorophenyl)-1-(4-{[1,2,4]triazolo[4,3-b]pyridazin-6-yl}piperazin-1-yl)ethan-1-one; 2-(4-bromo-3-chlorophenyl)-1-(4-{[1,2,4]triazolo[4,3-b]pyridazin-6-yl}piperazin-1-yl)ethan-1-one; 2-[4-chloro-3-(trifluoromethyl)phenyl]-1-(4-f[1,2,4]triazolo[4,3-b]pyridazin-6-yl}piperazin-1-yl)ethan-1-one; 2-(4-chloro-3-methylphenyl)-1-(4-{[1,2,4]triazolo[4,3-b]pyridazin-6-yl}piperazin-1-yl)ethan-1-one; 2-[3,5-bis(trifluoromethyl)phenyl]-1-(4-{[1,2,4]triazolo[4,3-b]pyridazin-6-yl}piperazin-1-yl)ethan-1-one; 2-(4-fluoro-3-methylphenyl)-1-(4-{[1,2,4]triazolo[4,3-b]pyridazin-6-yl}piperazin-1-yl)ethan-1-one; 2-[3-chloro-5-(trifluoromethyl)phenyl]-1-(4-{[1,2,4]triazolo[4,3-b]pyridazin-6-yl}piperazin-1-yl)ethan-1-one; 2-(4-chloro-3-fluorophenyl)-1-(4-{[1,2,4]triazolo[4,3-b]pyridazin-6-yl}piperazin-1-yl)ethan-1-one; 2-(3,4-difluorophenyl)-1-(4-{[1,2,4]triazolo[4,3-b]pyridazin-6-yl}piperazin-1-yl)ethan-1-one; 2-(4-bromo-3-fluorophenyl)-1-(4-{[1,2,4]triazolo[4,3-b]pyridazin-6-yl}piperazin-1-yl)ethan-1-one; 2-(4-chlorophenyl)-1-(4-{[1,2,4]triazolo[4,3-b]pyridazin-6-yl}piperazin-1-yl)ethan-1-one; 2-(3-chloro-5-fluorophenyl)-1-(4-{[1,2,4]triazolo[4,3-b]pyridazin-6-yl}piperazin-1-yl)ethan-1-one; 2-(4-ethynylphenyl)-1-(4-{[1,2,4]triazolo[4,3-b]pyridazin-6-yl}piperazin-1-yl)ethan-1-one; 2-(3,5-dichlorophenyl)-1-(4-{[1,2,4]triazolo[4,3-b]pyridazin-6-yl}piperazin-1-yl)ethan-1-one; and combinations thereof.

In an embodiment, a method of the present disclosure for treating an individual diagnosed with or suspected of having a Cryptosporidium infection or prophylaxis in an individual who is at risk of having a Cryptosporidium infection may comprise administering one or more compounds of the present disclosure or a composition comprising the one or more compounds in a therapeutically effective or prophylactically effective amount. In such an embodiment, the individual is a human or non-human animal. In such an embodiment, the one or more compounds may be chosen from: 2-(2-chlorophenyl)-1-(4-{[1,2,4]triazolo[4,3-b]pyridazin-6-yl}piperazin-1-yl)ethan-1-one; 2-(naphthalen-1-yl)-1-(4-{[1,2,4]triazolo[4,3-b]pyridazin-6-yl}piperazin-1-yl)ethan-1-one; 2-(4-chloro-3-fluorophenyl)-1-(4-{[1,2,4]triazolo[4,3-b]pyridazin-6-yl}piperazin-1-yl)ethan-1-one; 2-(4-fluorophenyl)-1-(4-{[1,2,4]triazolo[4,3-b]pyridazin-6-yl}piperazin-1-yl)ethan-1-one; 2-(3,4-dichlorophenyl)-1-(4-{[1,2,4]triazolo[4,3-b]pyridazin-6-yl}piperazin-1-yl)ethan-1-one; 2-(4-chlorophenyl)-1-(4-{[1,2,4]triazolo[4,3-b]pyridazin-6-yl}piperazin-1-yl)ethan-1-one; 2-(2,6-dichlorophenyl)-1-(4-{[1,2,4]triazolo[4,3-b]pyridazin-6-yl}piperazin-1-yl)ethan-1-one; 2-(2,4-dichlorophenyl)-1-(4-{[1,2,4]triazolo[4,3-b]pyridazin-6-yl}piperazin-1-yl)ethan-1-one; 2-(2,5-dichlorophenyl)-1-(4-{[1,2,4]triazolo[4,3-b]pyridazin-6-yl}piperazin-1-yl)ethan-1-one; 2-(3,5-dichlorophenyl)-1-(4-{[1,2,4]triazolo[4,3-b]pyridazin-6-yl}piperazin-1-yl)ethan-1-one; 2-(naphthalen-2-yl)-1-(4-{[1,2,4]triazolo[4,3-b]pyridazin-6-yl}piperazin-1-yl)ethan-1-one; 2-(3,4-dichlorophenyl)-1-(2-methyl-4-{[1,2,4]triazolo[4,3-b]pyridazin-6-yl}piperazin-1-yl)ethan-1-one; 2-(4-nitrophenyl)-1-(4-{[1,2,4]triazolo[4,3-b]pyridazin-6-yl}piperazin-1-yl)ethan-1-one; 2-(3-chloro-4-methylphenyl)-1-(4-{[1,2,4]triazolo[4,3-b]pyridazin-6-yl}piperazin-1-yl)ethan-1-one; 2-(3-fluoro-4-methylphenyl)-1-(4-{[1,2,4]triazolo[4,3-b]pyridazin-6-yl}piperazin-1-yl)ethan-1-one; 2-(4-chloro-3-methylphenyl)-1-(4-{[1,2,4]triazolo[4,3-b]pyridazin-6-yl}piperazin-1-yl)ethan-1-one; 2-(4-fluoro-3-methylphenyl)-1-(4-{[1,2,4]triazolo[4,3-b]pyridazin-6-yl}piperazin-1-yl)ethan-1-one; 2-(3-bromophenyl)-1-(4-[{1,2,4]triazolo[4,3-b]pyridazin-6-yl}piperazin-1-yl)ethan-1-one; 2-(4-methoxyphenyl)-1-(4-{[1,2,4]triazolo[4,3-b]pyridazin-6-yl}piperazin-1-yl)ethan-1-one; 2-(3-methoxyphenyl)-1-(4-{[1,2,4]triazolo[4,3-b]pyridazin-6-yl}piperazin-1-yl)ethan-1-one; 2-(3-chloro-4-fluorophenyl)-1-(4-{[1,2,4]triazolo[4,3-b]pyridazin-6-yl}piperazin-1-yl)ethan-1-one; 2-(3-chlorophenyl)-1-(4-{[1,2,4]triazolo[4,3-b]pyridazin-6-yl}piperazin-1-yl)ethan-1-one; 1-(3,4-dichlorophenyl)-2-(4-{[1,2,4]triazolo[4,3-b]pyridazin-6-yl}piperazin-1-yl)ethane-1,2-dione; 1-[1-(3,4-dichlorophenyl)cyclopropanecarbonyl]-4-{[1,2,4]triazolo[4,3-b]pyridazin-6-yl}piperazine; 2-(4-bromophenyl)-1-(4-{[1,2,4]triazolo[4,3-b]pyridazin-6-yl}piperazin-1-yl)ethan-1-one; 2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one; 4-[2-oxo-2-(4-{[1,2,4]triazolo[4,3-b]pyridazin-6-yl}piperazin-1-yl)ethyl]benzonitrile; 2-(2,3-dichlorophenyl)-1-(4-{[1,2,4]triazolo[4,3-b]pyridazin-6-yl}piperazin-1-yl)ethan-1-one; 2-(3,4-dichlorophenyl)-2,2-difluoro-1-(4-{[1,2,4]triazolo[4,3-b]pyridazin-6-yl}piperazin-1-yl)ethan-1-one; 2-(3,4-difluorophenyl)-1-(4-{[1,2,4]triazolo[4,3-b]pyridazin-6-yl}piperazin-1-yl)ethan-1-one; 2-(3,4-dichlorophenyl)-1-(4-{3-methyl-[1,2,4]triazolo[4,3-b]pyridazin-6-yl}piperazin-1-yl)ethan-1-one; 2-(3,4-dichlorophenyl)-1-(4-{3-phenyl-[1,2,4]triazolo[4,3-b]pyridazin-6-yl}piperazin-1-yl)ethan-1-one; 1-(4-{3-benzyl-[1,2,4]triazolo[4,3-b]pyridazin-6-yl}piperazin-1-yl)-2-(3,4-dichlorophenyl)ethan-1-one; 2-(3,4-dichlorophenyl)-1-{4-[3-(trifluoromethyl)-[1,2,4]triazolo[4,3-b]pyridazin-6-yl]piperazin-1-yl}ethan-1-one; 1-(4-{3-cyclopropyl-[1,2,4]triazolo[4,3-b]pyridazin-6-yl}piperazin-1-yl)-2-(3,4-dichlorophenyl)ethan-1-one; 2-(3,4-dichlorophenyl)-1-(4-{[1,2,4]triazolo[4,3-b]pyridazin-6-yl}piperazin-1-yl)propan-1-one; 2-(3,4-dichlorophenyl)-2-methyl-1-(4-{[1,2,4]triazolo[4,3-b]pyridazin-6-yl}piperazin-1-yl)propan-1-one; 2-(3,4-dichlorophenyl)-1-(3-methyl-4-{[1,2,4]triazolo[4,3-b]pyridazin-6-yl}piperazin-1-yl)ethan-1-one; 2-(3,4-dichlorophenyl)-1-(3,5-dimethyl-4-{[1,2,4]triazolo[4,3-b]pyridazin-6-yl}piperazin-1-yl)ethan-1-one; 2-(3,4-dichlorophenyl)-1-(2,6-dimethyl-4-{[1,2,4]triazolo[4,3-b]pyridazin-6-yl}piperazin-1-yl)ethan-1-one; 2-(3-chloro-5-fluorophenyl)-1-(4-{[1,2,4]triazolo[4,3-b]pyridazin-6-yl}piperazin-1-yl)ethan-1-one; 2-[4-(pentafluoro-λ⁶-sulfanyl)phenyl]-1-(4-{[1,2,4]triazolo[4,3-b]pyridazin-6-yl}piperazin-1-yl)ethan-1-one; 2-[3-(pentafluoro-λ⁶-sulfanyl)phenyl]-1-(4-{[1,2,4]triazolo[4,3-b]pyridazin-6-yl}piperazin-1-yl)ethan-1-one; 2-[4-chloro-3-(trifluoromethyl)phenyl]-1-(4-{[1,2,4]triazolo[4,3-b]pyridazin-6-yl}piperazin-1-yl)ethan-1-one; 2-[3-chloro-4-(trifluoromethyl)phenyl]-1-(4-{[1,2,4]triazolo[4,3-b]pyridazin-6-yl}piperazin-1-yl)ethan-1-one; 2-[4-fluoro-3-(trifluoromethyl)phenyl]-1-(4-{[1,2,4]triazolo[4,3-b]pyridazin-6-yl}piperazin-1-yl)ethan-1-one; 2-[3-fluoro-4-(trifluoromethyl)phenyl]-1-(4-{[1,2,4]triazolo[4,3-b]pyridazin-6-yl}piperazin-1-yl)ethan-1-one; 1-(4-{[1,2,4]triazolo[4,3-b]pyridazin-6-yl}piperazin-1-yl)-2-[3-(trifluoromethyl)phenyl]ethan-1-one; 1-(4-{[1,2,4]triazolo[4,3-b]pyridazin-6-yl}piperazin-1-yl)-2-[4-(trifluoromethyl)phenyl]ethan-1-one; 2-(2,3-dihydro-1H-inden-5-yl)-1-(4-{[1,2,4]triazolo[4,3-b]pyridazin-6-yl}piperazin-1-yl)ethan-1-one; 2-(3-tert-butylphenyl)-1-(4-{[1,2,4]triazolo[4,3-b]pyridazin-6-yl}piperazin-1-yl)ethan-1-one; 2-(4-tert-butylphenyl)-1-(4-{[1,2,4]triazolo[4,3-b]pyridazin-6-yl}piperazin-1-yl)ethan-1-one; 2-(4-chloro-3-ethylphenyl)-1-(4-{[1,2,4]triazolo[4,3-b]pyridazin-6-yl}piperazin-1-yl)ethan-1-one; 2-(3-ethyl-4-fluorophenyl)-1-(4-{[1,2,4]triazolo[4,3-b]pyridazin-6-yl}piperazin-1-yl)ethan-1-one; 2-[4-chloro-3-(propan-2-yl)phenyl]-1-(4-{[1,2,4]triazolo[4,3-b]pyridazin-6-yl}piperazin-1-yl)ethan-1-one; 2-(4-chloro-3-cyclopropylphenyl)-1-(4-{[1,2,4]triazolo[4,3-b]pyridazin-6-yl}piperazin-1-yl)ethan-1-one; 2-(4-methylphenyl)-1-(4-{[1,2,4]triazolo[4,3-b]pyridazin-6-yl}piperazin-1-yl)ethan-1-one; 2-(3-methylphenyl)-1 -(4-{[1,2,4]triazolo[4,3-b]pyridazin-6-yl}piperazin-1-yl)ethan-1-one; 4-[2-oxo-2-(4-{[1,2,4]triazolo[4,3-b]pyridazin-6-yl}piperazin-1-yl)ethyl]benzamide; 3-[2-oxo-2-(4-{[1,2,4]triazolo[4,3-b]pyridazin-6-yl}piperazin-1-yl)ethyl]benzamide; N-cyclopropyl-4-[2-oxo-2-(4-{[1,2,4]triazolo[4,3-b]pyridazin-6-yl}piperazin-1-yl)ethyl]benzamide; N-cyclopropyl-3-[2-oxo-2-(4-{[1,2,4]triazolo[4,3-b]pyridazin-6-yl}piperazin-1-yl)ethyl]benzamide; N,N-dimethyl-4-[2-oxo-2-(4-{[1,2,4]triazolo[4,3-b]pyridazin-6-yl}piperazin-1-yl)ethyl]benzamide; N,N-dimethyl-3-[2-oxo-2-(4-{[1,2,4]triazolo[4,3-b]pyridazin-6-yl}piperazin-1-yl)ethyl]benzamide; 2-[4-(pyrrolidine-1-carbonyl)phenyl]-1-(4-{[1,2,4]triazolo[4,3-b]pyridazin-6-yl}piperazin-1-yl)ethan-1-one; 2-[3-(pyrrolidine-1-carbonyl)phenyl]-1-(4-{[1,2,4]triazolo[4,3-b]pyridazin-6-yl}piperazin-1-yl)ethan-1-one; N-(2-hydroxyethyl)-4-[2-oxo-2-(4-{[1,2,4]triazolo[4,3-b]pyridazin-6-yl}piperazin-1-yl)ethyl]benzamide; N-(2-hydroxyethyl)-3-[2-oxo-2-(4-{[1,2,4]triazolo[4,3-b]pyridazin-6-yl}piperazin-1-yl)ethyl]benzamide; N-benzyl-4-[2-oxo-2-(4-{[1,2,4]triazolo[4,3-b]pyridazin-6-yl}piperazin-1-yl)ethyl]benzamide; N-benzyl-3-[2-oxo-2-(4-{[1,2,4]triazolo[4,3-b]pyridazin-6-yl}piperazin-1-yl)ethyl]benzamide; 4-[2-oxo-2-(4-{[1,2,4]triazolo[4,3-b]pyridazin-6-yl}piperazin-1-yl)ethyl]benzene-1-sulfonamide; 3-[2-oxo-2-(4-{[1,2,4]triazolo[4,3-b]pyridazin-6-yl}piperazin-1-yl)ethyl]benzene-1-sulfonamide; N-cyclopropyl-4-[2-oxo-2-(4-{[1,2,4]triazolo[4,3-b]pyridazin-6-yl}piperazin-1-yl)ethyl]benzene-1-sulfonamide; N-cyclopropyl-3-[2-oxo-2-(4-{[1,2,4]triazolo[4,3-b]pyridazin-6-yl}piperazin-1-yl)ethyl]benzene-1-sulfonamide; 2-(4-methanesulfonylphenyl)-1-(4-{[1,2,4]triazolo[4,3-b]pyridazin-6-yl}piperazin-1-yl)ethan-1-one; 2-(3-methanesulfonylphenyl)-1-(4-{[1,2,4]triazolo[4,3-b]pyridazin-6-yl}piperazin-1-yl)ethan-1-one; 2-{[1,1′-biphenyl]-4-yl}-1-(4-{[1,2,4]triazolo[4,3-b]pyridazin-6-yl}piperazin-1-yl)ethan-1-one; 2-{[1,1′-biphenyl]-3-yl}-1-(4-{[1,2,4]triazolo[4,3-b]pyridazin-6-yl}piperazin-1-yl)ethan-1-one; 2-(4-benzylphenyl)-1-(4-{[1,2,4]triazolo[4,3-b]pyridazin-6-yl}piperazin-1-yl)ethan-1-one; 2-(3-benzylphenyl)-1-(4-{[1,2,4]triazolo[4,3-b]pyridazin-6-yl}piperazin-1-yl)ethan-1-one; 2-(4-phenoxyphenyl)-1-(4-{[1,2,4]triazolo[4,3-b]pyridazin-6-yl}piperazin-1-yl)ethan-1-one; 2-(3-phenoxyphenyl)-1-(4-{[1,2,4]triazolo[4,3-b]pyridazin-6-yl}piperazin-1-yl)ethan-1-one; 2-[4-(2-phenylethyl)phenyl]-1-(4-{[1,2,4]triazolo[4,3-b]pyridazin-6-yl}piperazin-1-yl)ethan-1-one; 2-[3-(2-phenylethyl)phenyl]-1-(4-{[1,2,4]triazolo[4,3-b]pyridazin-6-yl}piperazin-1-yl)ethan-1-one; 2-[3-(hydroxymethyl)phenyl]-1-(4-{[1,2,4]triazolo[4,3-b]pyridazin-6-yl}piperazin-1-yl)ethan-1-one; 2-[3-chloro-4-(hydroxymethyl)phenyl]-1-(4-{[1,2,4]triazolo[4,3-b]pyridazin-6-yl}piperazin-1-yl)ethan-1-one; 2-[4-chloro-3-(hydroxymethyl)phenyl]-1-(4-{[1,2,4]triazolo[4,3-b]pyridazin-6-yl}piperazin-1-yl)ethan-1-one; 2-{4-[(pyrrolidin-1-yl)methyl]phenyl}-1-(4-{[1,2,4]triazolo[4,3-b]pyridazin-6-yl}piperazin-1-yl)ethan-1-one; 2-{3-[(pyrrolidin-1-yl)methyl]phenyl}-1-(4-{[1,2,4]triazolo[4,3-b]pyridazin-6-yl}piperazin-1-yl)ethan-1-one; 2-{3-chloro-4-[(pyrrolidin-1-yl)methyl]phenyl}-1-(4-{[1,2,4]triazolo[4,3-b]pyridazin-6-yl}piperazin-1-yl)ethan-1-one; 2-{4-chloro-3-[(pyrrolidin-1-yl)methyl]phenyl}-1-(4-{[1,2,4]triazolo[4,3-b]pyridazin-6-yl}piperazin-1-yl)ethan-1-one; 2-{4-[(morpholin-4-yl)methyl]phenyl}-1-(4-{[1,2,4]triazolo[4,3-b]pyridazin-6-yl}piperazin-1-yl)ethan-1-one; 2-{3-[(morpholin-4-yl)methyl]phenyl}-1-(4-{[1,2,4]triazolo[4,3-b]pyridazin-6-yl}piperazin-1-yl)ethan-1-one; 2-{3-chloro-4-[(morpholin-4-yl)methyl]phenyl}-1-(4-{[1,2,4]triazolo[4,3-b]pyridazin-6-yl}piperazin-1-yl)ethan-1-one; 2-{4-chloro-3-[(morpholin-4-yl)methyl]phenyl}-1-(4-{[1,2,4]triazolo[4,3-b]pyridazin-6-yl}piperazin-1-yl)ethan-1-one; 2-methyl-N-({4-[2-oxo-2-(4{[1,2,4]triazolo[4,3-b]pyridazin-6-yl}piperazin-1-yl)ethyl]phenyl}methyl)propanamide; 2-methyl-N-({3-[2-oxo-2-(4-{[1,2,4]triazolo[4,3-b]pyridazin-6-yl}piperazin-1-yl)ethyl]phenyl}methyl)propanamide; N-({2-chloro-4-[2-oxo-2-(4-{[1,2,4]triazolo[4,3-b]pyridazin-6-yl}piperazin-1-yl)ethyl]phenyl}methyl)-2-methylpropanamide; N-({2-chloro-5-[2-oxo-2-(4-{[1,2,4]triazolo[4,3-b]pyridazin-6-yl}piperazin-1-yl)ethyl]phenyl}methyl)-2-methylpropanamide; 2-{3-chloro-4-[(cyclopropylamino)methyl]phenyl}-1-(4-{[1,2,4]triazolo[4,3-b]pyridazin-6-yl}piperazin-1-yl)ethan-1-one; 2-{4-chloro-3-[(cyclopropylamino)methyl]phenyl}-1-(4-{[1,2,4]triazolo[4,3-b]pyridazin-6-yl}piperazin-1-yl)ethan-1-one; N,N-dimethyl-4-[2-oxo-2-(4-{[1,2,4]triazolo[4,3-b]pyridazin-6-yl}piperazin-1-yl)ethyl]benzene-1-sulfonamide; N,N-dimethyl-3-[2-oxo-2-(4-{[1,2,4]triazolo[4,3-b]pyridazin-6-yl}piperazin-1-yl)ethyl]benzene-1-sulfonamide; 2-[4-(pyrrolidine-1-sulfonyl)phenyl]-1-(4-{[1,2,4]triazolo[4,3-b]pyridazin-6-yl}piperazin-1-yl)ethan-1-one; 2-[3-(pyrrolidine-1-sulfonyl)phenyl]-1-(4-{ [1,2,4]triazolo[4,3-b]pyridazin-6-yl}piperazin-1-yl)ethan-1-one; 2-(3-ethynylphenyl)-1-(4-{[1,2,4]triazolo[4,3-b]pyridazin-6-yl}piperazin-1-yl)ethan-1-one; 2-{ 4-[(piperidin-1-yl)methyl]phenyl}-1-(4-{[1,2,4]triazolo[4,3-b]pyridazin-6-yl}piperazin-1-yl)ethan-1-one; 2-{4-[(4,4-difluoropiperidin-1-yl)methyl]phenyl}-1-(4-{[1,2,4]triazolo[4,3-b]pyridazin-6-yl}piperazin-1-yl)ethan-1-one; 2-{4-[(dimethylamino)methyl]phenyl}-1-(4-{[1,2,4]triazolo[4,3-b]pyridazin-6-yl}piperazin-1-yl)ethan-1-one; 2-[4-chloro-3-(difluoromethyl)phenyl]-1-(4-{[1,2,4]triazolo[4,3-b]pyridazin-6-yl}piperazin-1-yl)ethan-1-one; 2-[4-chloro-3-(fluoromethyl)phenyl]-1-(4-{[1,2,4]triazolo[4,3-b]pyridazin-6-yl}piperazin-1-yl)ethan-1-one; 2-(3,4-dichlorophenyl)-1-(4-{8-methyl-[1,2,4]triazolo[4,3-b]pyridazin-6-yl}piperazin-1-yl)ethan-1-one; 2-(3,4-dichlorophenyl)-1-(4-{7-methyl-[1,2,4]triazolo[4,3-b]pyridazin-6-yl}piperazin-1-yl)ethan-1-one; 2-(3,4-dichlorophenyl)-1-{4-[3-(trifluoromethyl)-[1,2,4]triazolo[4,3-b]pyridazin-6-yl]piperazin-1-yl}ethan-1-one; tert-butyl N-(3-{4-[2-oxo-2-(4-{[1,2,4]triazolo[4,3-b]pyridazin-6-yl}piperazin-1-yl)ethyl]phenyl}prop-2-yn-1-yl)carbamate; 2-[4-(hydroxymethyl)phenyl]-1-(4-{[1,2,4]triazolo[4,3-b]pyridazin-6-yl}piperazin-1-yl)ethan-1-one; 2-phenyl-1-(4-{[1,2,4]triazolo[4,3-b]pyridazin-6-yl}piperazin-1-yl)ethan-1-one; 2-[4-(3-aminoprop-1-yn-1-yl)phenyl]-1-(4-{[1,2,4]triazolo[4,3-b]pyridazin-6-yl}piperazin-1-yl)ethan-1-one; 2-(4-ethynylphenyl)-1-(4-{[1,2,4]triazolo[4,3-b]pyridazin-6-yl}piperazin-1-yl)ethan-1-one; 2-(2,3,4,5,6-pentafluorophenyl)-1-(4-{[1,2,4]triazolo[4,3-b]pyridazin-6-yl}piperazin-1-yl)ethan-1-one; 2-(4-bromo-3-chlorophenyl)-1-(4-{[1,2,4]triazolo[4,3-b]pyridazin-6-yl}piperazin-1-yl)ethan-1-one; 2-(3-bromo-4-chlorophenyl)-1-(4-{[1,2,4]triazolo[4,3-b]pyridazin-6-yl}piperazin-1-yl)ethan-1-one; 2-(4-bromo-3-fluorophenyl)-1-(4-{[1,2,4]triazolo[4,3-b]pyridazin-6-yl}piperazin-1-yl)ethan-1-one; 2-(3-bromo-4-fluorophenyl)-1-(4-{[1,2,4]triazolo[4,3-b]pyridazin-6-yl}piperazin-1-yl)ethan-1-one; tert-butyl N-(3-{4-[2-oxo-2-(4-{[1,2,4]triazolo[4,3-b]pyridazin-6-yl}piperazin-1-yl)ethyl]phenyl}propyl)carbamate; 2-(3,4-dichlorophenyl)-1-(4-{3-[2-(trifluoromethyl)pyridin-4-yl]-[1,2,4]triazolo[4,3-b]pyridazin-6-yl}piperazin-1-yl)ethan-1-one; 2-(2H-1,3-benzodioxol-5-yl)-1-(4-{[1,2,4]triazolo[4,3-b]pyridazin-6-yl}piperazin-1-yl)ethan-1-one; tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate; 2-[3-chloro-5-(trifluoromethyl)phenyl]-1-(4-{[1,2,4]triazolo[4,3-b]pyridazin-6-yl}piperazin-1-yl)ethan-1-one; 2-(3,5-difluorophenyl)-1-(4-{[1,2,4]triazolo[4,3-b]pyridazin-6-yl}piperazin-1-yl)ethan-1-one; 2-[4-(4-hydroxybenzoyl)phenyl]-1-(4-{[1,2,4]triazolo[4,3-b]pyridazin-6-yl}piperazin-1-yl)ethan-1-one; 2-(4-fluoro-3-methoxyphenyl)-1-(4-{[1,2,4]triazolo[4,3-b]pyridazin-6-yl}piperazin-1-yl)ethan-1-one; 2-[3,5-bis(trifluoromethyl)phenyl]-1-(4-{[1,2,4]triazolo[4,3-b]pyridazin-6-yl}piperazin-1-yl)ethan-1-one; 2-(3-fluoro-4-methoxyphenyl)-1-(4-{[1,2,4]triazolo[4,3-b]pyridazin-6-yl}piperazin-1-yl)ethan-1-one; 2-(3-methylphenyl)-1-(4-{[1,2,4]triazolo[4,3-b]pyridazin-6-yl}piperazin-1-yl)ethan-1-one; 2-(4-methylphenyl)-1-(4-{[1,2,4]triazolo[4,3-b]pyridazin-6-yl}piperazin-1-yl)ethan-1-one; 1-(4-{[1,2,4]triazolo[4,3-b]pyridazin-6-yl}piperazin-1-yl)-2-(3,4,5-trifluorophenyl)ethan-1-one; 2-[4-fluoro-3-(propan-2-yl)phenyl]-1-(4-{[1,2,4]triazolo[4,3-b]pyridazin-6-yl}piperazin-1-yl)ethan-1-one; 2-(3-cyclopropyl-4-fluorophenyl)-1-(4-{[1,2,4]triazolo[4,3-b]pyridazin-6-yl}piperazin-1-yl)ethan-1-one; 2-[3-(difluoromethyl)-4-fluorophenyl]-1-(4-{[1,2,4]triazolo[4,3-b]pyridazin-6-yl}piperazin-1-yl)ethan-1-one; 2-[4-fluoro-3-(fluoromethyl)phenyl]-1-(4-{[1,2,4]triazolo[4,3-b]pyridazin-6-yl}piperazin-1-yl)ethan-1-one; 2-[3,4-bis(trifluoromethyl)phenyl]-1-(4-{[1,2,4]triazolo[4,3-b]pyridazin-6-yl}piperazin-1-yl)ethan-1-one; 2-[3-fluoro-5-(trifluoromethyl)phenyl]-1-(4-{[1,2,4]triazolo[4,3-b]pyridazin-6-yl}piperazin-1-yl)ethan-1-one; 2-[2-fluoro-3-(trifluoromethyl)phenyl]-1-(4-{[1,2,4]triazolo[4,3-b]pyridazin-6-yl}piperazin-1-yl)ethan-1-one; 2-fluoro-5-[2-oxo-2-(4-{[1,2,4]triazolo[4,3-b]pyridazin-6-yl}piperazin-1-yl)ethyl]benzonitrile; 2-(4-fluoro-3-nitrophenyl)-1-(4-{[1,2,4]triazolo[4,3-b]pyridazin-6-yl}piperazin-1-yl)ethan-1-one; 2-[4-fluoro-3-(trifluoromethoxy)phenyl]-1-(4-{[1,2,4]triazolo[4,3-b]pyridazin-6-yl}piperazin-1-yl)ethan-1-one; 2-{6-fluoro-[1,1′-biphenyl]-3-yl}-1-(4-{[1,2,4]triazolo[4,3-b]pyridazin-6-yl}piperazin-1-yl)ethan-1-one; 2-{4′,6-difluoro-[1,1′-biphenyl]-3-yl}-1-(4-{[1,2,4]triazolo[4,3-b]pyridazin-6-yl}piperazin-1-yl)ethan-1-one; 2-[3-(1,1-difluoroethyl)-4-fluorophenyl]-1-(4-{[1,2,4]triazolo[4,3-b]pyridazin-6-yl}piperazin-1-yl)ethan-1-one; 2-[4-fluoro-3-(1,1,2,2,2-pentafluoroethyl)phenyl]-1-(4-{[1,2,4]triazolo[4,3-b]pyridazin-6-yl}piperazin-1-yl)ethan-1-one; 2-(3-ethynyl-4-fluorophenyl)-1-(4-{[1,2,4]triazolo[4,3-b]pyridazin-6-yl(piperazin-1-yl)ethan-1-one; 2-[4-fluoro-3-(prop-1-yn-1-yl)phenyl]-1-(4-{[1,2,4]triazolo[4,3-b]pyridazin-6-yl}piperazin-1-yl)ethan-1-one; 2-(3-tert-butyl-4-fluorophenyl)-1-(4-{[1,2,4]triazolo[4,3-b]pyridazin-6-yl}piperazin-1-yl)ethan-1-one; 2-[4-fluoro-3-(hydroxymethyl)phenyl]-1-(4-{[1,2,4]triazolo[4,3-b]pyridazin-6-yl}piperazin-1-yl)ethan-1-one; 2-[4-fluoro-3-(methoxymethyl)phenyl]-1-(4-{[1,2,4]triazolo[4,3-b]pyridazin-6-yl}piperazin-1-yl)ethan-1-one; 2-fluoro-5-[2-oxo-2-(4-{[1,2,4]triazolo[4,3-b]pyridazin-6-yl(piperazin-1-yl)ethyl]benzoic acid; 2-fluoro-5-[2-oxo-2-(4-{[1,2,4]triazolo[4,3-b]pyridazin-6-yl}piperazin-1-yl)ethyl]benzamide; 2-fluoro-N-methyl-5-[2-oxo-2-(4-{[1,2,4]triazolo[4,3-b]pyridazin-6-yl}piperazin-1-yl)ethyl]benzamide; 2-fluoro-N,N-dimethyl-5-[2-oxo-2-(4-{[1,2,4]triazolo[4,3-b]pyridazin-6-yl}piperazin-1-yl)ethyl]benzamide; 2-fluoro-5-[2-oxo-2-(4-{[1,2,4]triazolo[4,3-b]pyridazin-6-yl(piperazin-1-yl)ethyl]benzene-1-sulfonamide; 2-fluoro-N-methyl-5-[2-oxo-2-(4-{[1,2,4]triazolo[4,3-b]pyridazin-6-yl}piperazin-1-yl)ethyl]benzene-1-sulfonamide; 2-fluoro-N,N-dimethyl-5-[2-oxo-2-(4-{[1,2,4]triazolo[4,3-b]pyridazin-6-yl}piperazin-1-yl)ethyl]benzene-1-sulfonamide; 2-(4-fluoro-3-methanesulfonylphenyl)-1-(4-{[1,2,4]triazolo[4,3-b]pyridazin-6-yl}piperazin-1-yl)ethan-1-one; and combinations thereof. In such an embodiment, the one or more compounds may be chosen from 2-[4-fluoro-3-(trifluoromethyl)phenyl]-1-(4-{[1,2,4]triazolo[4,3-b]pyridazin-6-yl}piperazin-1-yl)ethan-1-one; 2-(3-bromo-4-chlorophenyl)-1-(4-{[1,2,4]triazolo[4,3-b]pyridazin-6-yl}piperazin-1-yl)ethan-1-one; 2-(3,4-dichlorophenyl)-1-(4-{ [1,2,4]triazolo[4,3-b]pyridazin-6-yl}piperazin-1-yl)ethan-1-one; 2-(3-bromo-4-fluorophenyl)-1-(4-{[1,2,4]triazolo[4,3 -b]pyridazin-6-yl}piperazin-1-yl)ethan-1-one; 2-(3-chloro-4-fluorophenyl)-1-(4-{[1,2,4]triazolo[4,3-b]pyridazin-6-yl}piperazin-1-yl)ethan-1-one; 2-(4-bromo-3-chlorophenyl)-1-(4-{[1,2,4]triazolo[4,3-b]pyridazin-6-yl{piperazin-1-yl)ethan-1-one; 2-[4-chloro-3-(trifluoromethyl)phenyl]-1-(4-{[1,2,4]triazolo[4,3-b]pyridazin-6-yl}piperazin-1-yl)ethan-1-one; 2-(4-chloro-3-methylphenyl)-1-(4-{[1,2,4]triazolo[4,3-b]pyridazin-6-yl}piperazin-1-yl)ethan-1-one; 2-[3,5-bis(trifluoromethyl)phenyl]-1-(4-{[1,2,4]triazolo[4,3-b]pyridazin-6-yl}piperazin-1-yl)ethan-1-one; 2-(4-fluoro-3-methylphenyl)-1-(4-{[1,2,4]triazolo[4,3-b]pyridazin-6-yl}piperazin-1-yl)ethan-1-one; 2-[3-chloro-5-(trifluoromethyl)phenyl]-1-(4-{[1,2,4]triazolo[4,3-b]pyridazin-6-yl}piperazin-1-yl)ethan-1-one; 2-(4-chloro-3-fluorophenyl)-1-(4-{[1,2,4]triazolo[4,3-b]pyridazin-6-yl}piperazin-1-yl)ethan-1-one; 2-(3,4-difluorophenyl)-1-(4-{[1,2,4]triazolo[4,3-b]pyridazin-6-yl}piperazin-1-yl)ethan-1-one; 2-(4-bromo-3-fluorophenyl)-1-(4-{[1,2,4]triazolo[4,3-b]pyridazin-6-yl}piperazin-1-yl)ethan-1-one; 2-(4-chlorophenyl)-1-(4-{[1,2,4]triazolo[4,3-b]pyridazin-6-yl}piperazin-1-yl)ethan-1-one; 2-(3-chloro-5-fluorophenyl)-1-(4-{[1,2,4]triazolo[4,3-b]pyridazin-6-yl}piperazin-1-yl)ethan-1-one; 2-(4-ethynylphenyl)-1-(4-{[1,2,4]triazolo[4,3-b]pyridazin-6-yl}piperazin-1-yl)ethan-1-one; 2-(3,5-dichlorophenyl)-1-(4-{[1,2,4]triazolo[4,3-b]pyridazin-6-yl}piperazin-1-yl)ethan-1-one; and combinations thereof.

In an embodiment, a kit of the present disclosure comprises one or more compounds of the present disclosure or a composition comprising the one or more compounds or materials to prepare the composition and instructions on administration details to an individual who has been diagnosed with or who is at risk of getting Cryptosporidium infection where the details comprise one or more of the following: dosage, frequency, and length of time for administration of the compound or the composition. In such an embodiment, the compound may be chosen from: 2-(2-chlorophenyl)-1-(4-{[1,2,4]triazolo[4,3-b]pyridazin-6-yl}piperazin-1-yl)ethan-1-one; 2-(naphthalen-1-yl)-1-(4-{[1,2,4]triazolo[4,3-b]pyridazin-6-yl}piperazin-1-yl)ethan-1-one; 2-(4-chloro-3-fluorophenyl)-1-(4-{[1,2,4]triazolo[4,3-b]pyridazin-6-yl}piperazin-1-yl)ethan-1-one; 2-(4-fluorophenyl)-1-(4-{[1,2,4]triazolo[4,3-b]pyridazin-6-yl}piperazin-1-yl)ethan-1-one; 2-(3,4-dichlorophenyl)-1-(4-{[1,2,4]triazolo[4,3-b]pyridazin-6-yl}piperazin-1-yl)ethan-1-one; 2-(4-chlorophenyl)-1-(4-{[1,2,4]triazolo[4,3-b]pyridazin-6-yl}piperazin-1-yl)ethan-1-one; 2-(2,6-dichlorophenyl)-1-(4-{[1,2,4]triazolo[4,3-b]pyridazin-6-yl}piperazin-1-yl)ethan-1-one; 2-(2,4-dichlorophenyl)-1-(4-{[1,2,4]triazolo[4,3-b]pyridazin-6-yl}piperazin-1-yl)ethan-1-one; 2-(2,5-dichlorophenyl)-1-(4-{[1,2,4]triazolo[4,3-b]pyridazin-6-yl}piperazin-1-yl)ethan-1-one; 2-(3,5-dichlorophenyl)-1-(4-{[1,2,4]triazolo[4,3-b]pyridazin-6-yl}piperazin-1-yl)ethan-1-one; 2-(naphthalen-2-yl)-1-(4-{[1,2,4]triazolo[4,3-b]pyridazin-6-yl}piperazin-1-yl)ethan-1-one; 2-(3,4-dichlorophenyl)-1-(2-methyl-4-{[1,2,4]triazolo[4,3-b]pyridazin-6-yl}piperazin-1-yl)ethan-1-one; 2-(4-nitrophenyl)-1-(4-{[1,2,4]triazolo[4,3-b]pyridazin-6-yl}piperazin-1-yl)ethan-1-one; 2-(3-chloro-4-methylphenyl)-1-(4-{[1,2,4]triazolo[4,3-b]pyridazin-6-yl}piperazin-1-yl)ethan-1-one; 2-(3-fluoro-4-methylphenyl)-1-(4-{[1,2,4]triazolo[4,3-b]pyridazin-6-yl}piperazin-1-yl)ethan-1-one; 2-(4-chloro-3-methylphenyl)-1-(4-{[1,2,4]triazolo[4,3-b]pyridazin-6-yl}piperazin-1-yl)ethan-1-one; 2-(4-fluoro-3-methylphenyl)-1-(4-{[1,2,4]triazolo[4,3-b]pyridazin-6-yl}piperazin-1-yl)ethan-1-one; 2-(3-bromophenyl)-1-(4-{[1,2,4]triazolo[4,3-b]pyridazin-6-yl}piperazin-1-yl)ethan-1-one; 2-(4-methoxyphenyl)-1-(4-{[1,2,4]triazolo[4,3-b]pyridazin-6-yl}piperazin-1-yl)ethan-1-one; 2-(3-methoxyphenyl)-1-(4-{[1,2,4]triazolo[4,3-b]pyridazin-6-yl}piperazin-1-yl)ethan-1-one; 2-(3-chloro-4-fluorophenyl)-1-(4-{[1,2,4]triazolo[4,3-b]pyridazin-6-yl}piperazin-1-yl)ethan-1-one; 2-(3-chlorophenyl)-1-(4-{[1,2,4]triazolo[4,3-b]pyridazin-6-yl }piperazin-1-yl)ethan-1-one; 1-(3,4-dichlorophenyl)-2-(4-{[1,2,4]triazolo[4,3-b]pyridazin-6-yl}piperazin-1-yl)ethane-1,2-dione; 1-[1-(3,4-dichlorophenyl)cyclopropanecarbonyl]-4-{[1,2,4]triazolo[4,3-b]pyridazin-6-yl}piperazine; 2-(4-bromophenyl)-1-(4-{[1,2,4]triazolo[4,3-b]pyridazin-6-yl}piperazin-1-yl)ethan-1-one; 2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one; 4-[2-oxo-2-(4-{[1,2,4]triazolo[4,3-b]pyridazin-6-yl}piperazin-1-yl)ethyl]benzonitrile; 2-(2,3-dichlorophenyl)-1-(4-{[1,2,4]triazolo[4,3-b]pyridazin-6-yl}piperazin-1-yl)ethan-1-one; 2-(3,4-dichlorophenyl)-2,2-difluoro-1-(4-{[1,2,4]triazolo[4,3-b]pyridazin-6-yl}piperazin-1-yl)ethan-1-one; 2-(3,4-difluorophenyl)-1-(4-{[1,2,4]triazolo[4,3-b]pyridazin-6-yl}piperazin-1-yl)ethan-1-one; 2-(3,4-dichlorophenyl)-1-(4-{3-methyl-[1,2,4]triazolo[4,3-b]pyridazin-6-yl}piperazin-1-yl)ethan-1-one; 2-(3,4-dichlorophenyl)-1-(4-{3-phenyl-[1,2,4]triazolo[4,3-b]pyridazin-6-yl}piperazin-1-yl)ethan-1-one; 1-(4-{3-benzyl-[1,2,4]triazolo[4,3-b]pyridazin-6-yl}piperazin-1-yl)-2-(3,4-dichlorophenyl)ethan-1-one; 2-(3,4-dichlorophenyl)-1-{4-[3-(trifluoromethyl)-[1,2,4]triazolo[4,3-b]pyridazin-6-yl]piperazin-1-yl}ethan-1-one; 1-(4-{3-cyclopropyl-[1,2,4]triazolo[4,3-b]pyridazin-6-yl}piperazin-1-yl)-2-(3,4-dichlorophenyl)ethan-1-one; 2-(3,4-dichlorophenyl)-1-(4-{[1,2,4]triazolo[4,3-b]pyridazin-6-yl}piperazin-1-yl)propan-1-one; 2-(3,4-dichlorophenyl)-2-methyl-1-(4-{[1,2,4]triazolo[4,3-b]pyridazin-6-yl}piperazin-1-yl)propan-1-one; 2-(3,4-dichlorophenyl)-1-(3-methyl-4-{[1,2,4]triazolo[4,3-b]pyridazin-6-yl}piperazin-1-yl)ethan-1-one; 2-(3,4-dichlorophenyl)-1-(3,5-dimethyl-4-{[1,2,4]triazolo[4,3-b]pyridazin-6-yl}piperazin-1-yl)ethan-1-one; 2-(3,4-dichlorophenyl)-1-(2,6-dimethyl-4-{[1,2,4]triazolo[4,3-b]pyridazin-6-yl}piperazin-1-yl)ethan-1-one; 2-(3-chloro-5-fluorophenyl)-1-(4-{[1,2,4]triazolo[4,3-b]pyridazin-6-yl}piperazin-1-yl)ethan-1-one; 2-[4-(pentafluoro-λ⁶-sulfanyl)phenyl]-1-(4-{[1,2,4]triazolo[4,3-b]pyridazin-6-yl}piperazin-1-yl)ethan-1-one; 2-[3-(pentafluoro-λ⁶-sulfanyl)phenyl]-1-(4-{[1,2,4]triazolo[4,3-b]pyridazin-6-yl}piperazin-1-yl)ethan-1-one; 2-[4-chloro-3-(trifluoromethyl)phenyl]-1-(4-{[1,2,4]triazolo[4,3-b]pyridazin-6-yl}piperazin-1-yl)ethan-1-one; 2-[3-chloro-4-(trifluoromethyl)phenyl]-1-(4-{[1,2,4]triazolo[4,3-b]pyridazin-6-yl}piperazin-1-yl)ethan-1-one; 2-[4-fluoro-3-(trifluoromethyl)phenyl]-1-(4-{[1,2,4]triazolo[4,3-b]pyridazin-6-yl}piperazin-1-yl)ethan-1-one; 2-[3-fluoro-4-(trifluoromethyl)phenyl]-1-(4-{[1,2,4]triazolo[4,3-b]pyridazin-6-yl}piperazin-1-yl)ethan-1-one; 1-(4-{[1,2,4]triazolo[4,3-b]pyridazin-6-yl}piperazin-1-yl)-2-[3-(trifluoromethyl)phenyl]ethan-1-one; 1-(4-{[1,2,4]triazolo[4,3-b]pyridazin-6-yl}piperazin-1-yl)-2-[4-(trifluoromethyl)phenyl]ethan-1-one; 2-(2,3-dihydro-1H-inden-5-yl)-1-(4-{[1,2,4]triazolo[4,3-b]pyridazin-6-yl}piperazin-1-yl)ethan-1-one; 2-(3-tert-butylphenyl)-1-(4-{[1,2,4]triazolo[4,3-b]pyridazin-6-yl}piperazin-1-yl)ethan-1-one; 2-(4-tert-butylphenyl)-1-(4-{[1,2,4]triazolo[4,3-b]pyridazin-6-yl}piperazin-1-yl)ethan-1-one; 2-(4-chloro-3-ethylphenyl)-1-(4-{[1,2,4]triazolo[4,3-b]pyridazin-6-yl}piperazin-1-yl)ethan-1-one; 2-(3-ethyl-4-fluorophenyl)-1-(4-{[1,2,4]triazolo[4,3-b]pyridazin-6-yl}piperazin-1-yl)ethan-1-one; 2-[4-chloro-3-(propan-2-yl)phenyl]-1-(4-{[1,2,4]triazolo[4,3-b]pyridazin-6-yl}piperazin-1-yl)ethan-1-one; 2-(4-chloro-3-cyclopropylphenyl)-1-(4-{[1,2,4]triazolo[4,3-b]pyridazin-6-yl}piperazin-1-yl)ethan-1-one; 2-(4-methylphenyl)-1-(4-{[1,2,4]triazolo[4,3-b]pyridazin-6-yl}piperazin-1-yl)ethan-1-one; 2-(3-methylphenyl)-1-(4-{[1,2,4]triazolo[4,3-b]pyridazin-6-yl}piperazin-1-yl)ethan-1-one; 4-[2-oxo-2-(4-{[1,2,4]triazolo[4,3-b]pyridazin-6-yl}piperazin-1-yl)ethyl]benzamide; 3-[2-oxo-2-(4-{[1,2,4]triazolo[4,3-b]pyridazin-6-yl}piperazin-1-yl)ethyl]benzamide; N-cyclopropyl-4-[2-oxo-2-(4-{[1,2,4]triazolo[4,3-b]pyridazin-6-yl}piperazin-1-yl)ethyl]benzamide; N-cyclopropyl-3-[2-oxo-2-(4-{[1,2,4]triazolo[4,3-b]pyridazin-6-yl}piperazin-1-yl)ethyl]benzamide; N,N-dimethyl-4-[2-oxo-2-(4-{[1,2,4]triazolo[4,3-b]pyridazin-6-yl}piperazin-1-yl)ethyl]benzamide; N,N-dimethyl-3-[2-oxo-2-(4-{[1,2,4]triazolo[4,3-b]pyridazin-6-yl}piperazin-1-yl)ethyl]benzamide; 2-[4-(pyrrolidine-1-carbonyl)phenyl]-1-(4-{[1,2,4]triazolo[4,3-b]pyridazin-6-yl}piperazin-1-yl)ethan-1-one; 2-[3-(pyrrolidine-1-carbonyl)phenyl]-1-(4-{[1,2,4]triazolo[4,3-b]pyridazin-6-yl}piperazin-1-yl)ethan-1-one; N-(2-hydroxyethyl)-4-[2-oxo-2-(4-{[1,2,4]triazolo[4,3-b]pyridazin-6-yl}piperazin-1-yl)ethyl]benzamide; N-(2-hydroxyethyl)-3-[2-oxo-2-(4-{[1,2,4]triazolo[4,3-b]pyridazin-6-yl}piperazin-1-yl)ethyl]benzamide; N-benzyl-4-[2-oxo-2-(4-{[1,2,4]triazolo[4,3-b]pyridazin-6-yl}piperazin-1-yl)ethyl]benzamide; N-benzyl-3-[2-oxo-2-(4-{[1,2,4]triazolo[4,3-b]pyridazin-6-yl}piperazin-1-yl)ethyl]benzamide; 4-[2-oxo-2-(4-{[1,2,4]triazolo[4,3-b]pyridazin-6-yl}piperazin-1-yl)ethyl]benzene-1-sulfonamide; 3-[2-oxo-2-(4-{[1,2,4]triazolo[4,3-b]pyridazin-6-yl}piperazin-1-yl)ethyl]benzene-1-sulfonamide; N-cyclopropyl-4-[2-oxo-2-(4-{[1,2,4]triazolo[4,3-b]pyridazin-6-yl}piperazin-1-yl)ethyl]benzene-1-sulfonamide; N-cyclopropyl-3-[2-oxo-2-(4-{[1,2,4]triazolo[4,3-b]pyridazin-6-yl}piperazin-1-yl)ethyl]benzene-1-sulfonamide; 2-(4-methanesulfonylphenyl)-1-(4-{[1,2,4]triazolo[4,3-b]pyridazin-6-yl }piperazin-1-yl)ethan-1-one; 2-(3-methanesulfonylphenyl)-1-(4-{[1,2,4]triazolo[4,3-b]pyridazin-6-yl }piperazin-1-yl)ethan-1-one; 2-{[1,1′-biphenyl]-4-yl}-1-(4-{[1,2,4]triazolo[4,3-b]pyridazin-6-yl}piperazin-1-yl)ethan-1-one; 2-{[1,1′-biphenyl]-3-yl}-1-(4-{[1,2,4]triazolo[4,3-b]pyridazin-6-yl}piperazin-1-yl)ethan-1-one; 2-(4-benzylphenyl)-1-(4-{[1,2,4]triazolo[4,3-b]pyridazin-6-yl}piperazin-1-yl)ethan-1-one; 2-(3-benzylphenyl)-1-(4-{[1,2,4]triazolo[4,3-b]pyridazin-6-yl}piperazin-1-yl)ethan-1-one; 2-(4-phenoxyphenyl)-1-(4-{[1,2,4]triazolo[4,3-b]pyridazin-6-yl}piperazin-1-yl)ethan-1-one; 2-(3-phenoxyphenyl)-1-(4-{[1,2,4]triazolo[4,3-b]pyridazin-6-yl}piperazin-1-yl)ethan-1-one; 2-[4-(2-phenylethyl)phenyl]-1-(4-{[1,2,4]triazolo[4,3-b]pyridazin-6-yl}piperazin-1-yl)ethan-1-one; 2-[3-(2-phenylethyl)phenyl]-1-(4-{[1,2,4]triazolo[4,3-b]pyridazin-6-yl }piperazin-1-yl)ethan-1-one; 2-[3-(hydroxymethyl)phenyl]-1-(4-{[1,2,4]triazolo[4,3-b]pyridazin-6-yl}piperazin-1-yl)ethan-1-one; 2-[3-chloro-4-(hydroxymethyl)phenyl]-1-(4-{[1,2,4]triazolo[4,3-b]pyridazin-6-yl}piperazin-1-yl)ethan-1-one; 2-[4-chloro-3-(hydroxymethyl)phenyl]-1-(4-{[1,2,4]triazolo[4,3-b]pyridazin-6-yl}piperazin-1-yl)ethan-1-one; 2-{4-[(pyrrolidin-1-yl)methyl]phenyl}-1-(4-{[1,2,4]triazolo[4,3-b]pyridazin-6-yl}piperazin-1-yl)ethan-1-one; 2-{3-[(pyrrolidin-1-yl)methyl]phenyl}-1-(4-{[1,2,4]triazolo[4,3-b]pyridazin-6-yl}piperazin-1-yl)ethan-1-one; 2-{3-chloro-4-[(pyrrolidin-1-yl)methyl]phenyl}-1-(4-{[1,2,4]triazolo[4,3-b]pyridazin-6-yl}piperazin-1-yl)ethan-1-one; 2-{4-chloro-3-[(pyrrolidin-1-yl)methyl]phenyl}-1-(4-{[1,2,4]triazolo[4,3-b]pyridazin-6-yl}piperazin-1-yl)ethan-1-one; 2-{4-[(morpholin-4-yl)methyl]phenyl}-1-(4-{[1,2,4]triazolo[4,3-b]pyridazin-6-yl}piperazin-1-yl)ethan-1-one; 2-{3-[(morpholin-4-yl)methyl]phenyl}-1-(4-{[1,2,4]triazolo[4,3-b]pyridazin-6-yl}piperazin-1-yl)ethan-1-one; 2-{3-chloro-4-[(morpholin-4-yl)methyl]phenyl}-1-(4-{[1,2,4]triazolo[4,3-b]pyridazin-6-yl}piperazin-1-yl)ethan-1-one; 2-{4-chloro-3-[(morpholin-4-yl)methyl]phenyl}-1-(4-{[1,2,4]triazolo[4,3-b]pyridazin-6-yl}piperazin-1-yl)ethan-1-one; 2-methyl-N-({4-[2-oxo-2-(4-{[1,2,4]triazolo[4,3-b]pyridazin-6-yl}piperazin-1-yl)ethyl]phenyl}methyl)propanamide; 2-methyl-N-({3-[2-oxo-2-(4-{[1,2,4]triazolo[4,3-b]pyridazin-6-yl}piperazin-1-yl)ethyl]phenyl}methyl)propanamide;N-({2-chloro-4-[2-oxo-2-(4-{[1,2,4]triazolo[4,3-b]pyridazin-6-yl}piperazin-1-yl)ethyl]phenyl}methyl)-2-methylpropanamide; N-({2-chloro-5-[2-oxo-2-(4-{[1,2,4]triazolo[4,3-b]pyridazin-6-yl}piperazin-1-yl)ethyl]phenyl}methyl)-2-methylpropanamide; 2-{3-chloro-4-[(cyclopropylamino)methyl]phenyl}-1-(4-{[1,2,4]triazolo[4,3-b]pyridazin-6-yl}piperazin-1-yl)ethan-1-one; 2-{4-chloro-3-[(cyclopropylamino)methyl]phenyl}-1-(4-{[1,2,4]triazolo[4,3-b]pyridazin-6-yl}piperazin-1-yl)ethan-1-one; N,N-dimethyl-4-[2-oxo-2-(4-{[1,2,4]triazolo[4,3-b]pyridazin-6-yl}piperazin-1-yl)ethyl]benzene-1-sulfonamide; N,N-dimethyl-3-[2-oxo-2-(4-{[1,2,4]triazolo[4,3-b]pyridazin-6-yl}piperazin-1-yl)ethyl]benzene-1-sulfonamide; 2-[4-(pyrrolidine-1-sulfonyl)phenyl]-1-(4-{[1,2,4]triazolo[4,3-b]pyridazin-6-yl}piperazin-1-yl)ethan-1-one; 2-[3-(pyrrolidine-1-sulfonyl)phenyl]-1-(4-{[1,2,4]triazolo[4,3-b]pyridazin-6-yl}piperazin-1-yl)ethan-1-one; 2-(3-ethynylphenyl)-1-(4-{[1,2,4]triazolo[4,3-b]pyridazin-6-yl}piperazin-1-yl)ethan-1-one; 2-{4-[(piperidin-1-yl)methyl]phenyl}-1-(4-{[1,2,4]triazolo[4,3-b]pyridazin-6-yl}piperazin-1-yl)ethan-1-one; 2-{4-[(4,4-difluoropiperidin-1-yl)methyl]phenyl}-1-(4-{[1,2,4]triazolo[4,3-b]pyridazin-6-yl(piperazin-1-yl)ethan-1-one; 2-{4-[(dimethylamino)methyl]phenyl}-1-(4-{[1,2,4]triazolo[4,3-b]pyridazin-6-yl}piperazin-1-yl)ethan-1-one; 2-[4-chloro-3-(difluoromethyl)phenyl]-1-(4-{[1,2,4]triazolo[4,3-b]pyridazin-6-yl}piperazin-1-yl)ethan-1-one; 2-[4-chloro-3-(fluoromethyl)phenyl]-1-(4-{[1,2,4]triazolo[4,3-b]pyridazin-6-yl}piperazin-1-yl)ethan-1-one; 2-(3,4-dichlorophenyl)-1-(4-{8-methyl-[1,2,4]triazolo[4,3-b]pyridazin-6-yl}piperazin-1-yl)ethan-1-one; 2-(3,4-dichlorophenyl)-1-(4-{7-methyl-[1,2,4]triazolo[4,3-b]pyridazin-6-yl}piperazin-1-yl)ethan-1-one; 2-(3,4-dichlorophenyl)-1-{4-[3-(trifluoromethyl)-[1,2,4]triazolo[4,3-b]pyridazin-6-yl]piperazin-1-yl}ethan-1-one; tert-butyl N-(3-{4-[2-oxo-2-(4-{[1,2,4]triazolo[4,3-b]pyridazin-6-yl}piperazin-1-yl)ethyl]phenyl }prop-2-yn-1-yl)carbamate; 2-[4-(hydroxymethyl)phenyl]-1-(4-{[1,2,4]triazolo[4,3-b]pyridazin-6-yl}piperazin-1-yl)ethan-1-one; 2-phenyl-1-(4-{[1,2,4]triazolo[4,3-b]pyridazin-6-yl}piperazin-1-yl)ethan-1-one; 2-[4-(3-aminoprop-1-yn-1-yl)phenyl]-1-(4-{[1,2,4]triazolo[4,3-b]pyridazin-6-yl}piperazin-1-yl)ethan-1-one; 2-(4-ethynylphenyl)-1-(4-{[1,2,4]triazolo[4,3-b]pyridazin-6-yl}piperazin-1-yl)ethan-1-one; 2-(2,3,4,5,6-pentafluorophenyl)-1-(4-{[1,2,4]triazolo[4,3-b]pyridazin-6-yl}piperazin-1-yl)ethan-1-one; 2-(4-bromo-3-chlorophenyl)-1-(4-{[1,2,4]triazolo[4,3-b]pyridazin-6-yl}piperazin-1-yl)ethan-1-one; 2-(3-bromo-4-chlorophenyl)-1-(4-{[1,2,4]triazolo[4,3-b]pyridazin-6-yl(piperazin-1-yl)ethan-1-one; 2-(4-bromo-3-fluorophenyl)-1-(4-{[1,2,4]triazolo[4,3-b]pyridazin-6-yl}piperazin-1-yl)ethan-1-one; 2-(3-bromo-4-fluorophenyl)-1-(4-{[1,2,4]triazolo[4,3-b]pyridazin-6-yl}piperazin-1-yl)ethan-1-one; tert-butyl N-(3-{4-[2-oxo-2-(4-{[1,2,4]triazolo[4,3-b]pyridazin-6-yl(piperazin-1-yl)ethyl]phenyl}propyl)carbamate; 2-(3,4-dichlorophenyl)-1-(4-{3-[2-(trifluoromethyl)pyridin-4-yl]-[1,2,4]triazolo[4,3-b]pyridazin-6-yl}piperazin-1-yl)ethan-1-one; 2-(2H-1,3-benzodioxol-5-yl)-1-(4-{[1,2,4]triazolo[4,3-b]pyridazin-6-yl}piperazin-1-yl)ethan-1-one; tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate; 2-[3-chloro-5-(trifluoromethyl)phenyl]-1-(4-{[1,2,4]triazolo[4,3-b]pyridazin-6-yl}piperazin-1-yl)ethan-1-one; 2-(3,5-difluorophenyl)-1-(4-{[1,2,4]triazolo[4,3-b]pyridazin-6-yl}piperazin-1-yl)ethan-1-one; 2-[4-(4-hydroxybenzoyl)phenyl]-1-(4-{[1,2,4]triazolo[4,3-b]pyridazin-6-yl}piperazin-1-yl)ethan-1-one; 2-(4-fluoro-3-methoxyphenyl)-1-(4-{[1,2,4]triazolo[4,3-b]pyridazin-6-yl}piperazin-1-yl)ethan-1-one; 2-[3,5-bis(trifluoromethyl)phenyl]-1-(4-{[1,2,4]triazolo[4,3-b]pyridazin-6-yl}piperazin-1-yl)ethan-1-one; 2-(3-fluoro-4-methoxyphenyl)-1-(4-{[1,2,4]triazolo[4,3-b]pyridazin-6-yl}piperazin-1-yl)ethan-1-one; 2-(3-methylphenyl)-1-(4-{[1,2,4]triazolo[4,3-b]pyridazin-6-yl}piperazin-1-yl)ethan-1-one; 2-(4-methylphenyl)-1-(4-{[1,2,4]triazolo[4,3-b]pyridazin-6-yl}piperazin-1-yl)ethan-1-one; 1-(4-{[1,2,4]triazolo[4,3-b]pyridazin-6-yl}piperazin-1-yl)-2-(3,4,5-trifluorophenyl)ethan-1-one; 2-[4-fluoro-3-(propan-2-yl)phenyl]-1-(4-{[1,2,4]triazolo[4,3-b]pyridazin-6-yl}piperazin-1-yl)ethan-1-one; 2-(3-cyclopropyl-4-fluorophenyl)-1-(4-{[1,2,4]triazolo[4,3-b]pyridazin-6-yl}piperazin-1-yl)ethan-1-one; 2-[3-(difluoromethyl)-4-fluorophenyl]-1-(4-{[1,2,4]triazolo[4,3-b]pyridazin-6-yl}piperazin-1-yl)ethan-1-one; 2-[4-fluoro-3-(fluoromethyl)phenyl]-1-(4-{[1,2,4]triazolo[4,3-b]pyridazin-6-yl }piperazin-1-yl)ethan-1-one; 2-[3,4-bis(trifluoromethyl)phenyl]-1-(4-{[1,2,4]triazolo[4,3-b]pyridazin-6-yl}piperazin-1-yl)ethan-1-one; 2-[3-fluoro-5-(trifluoromethyl)phenyl]-1-(4-{[1,2,4]triazolo[4,3-b]pyridazin-6-yl}piperazin-1-yl)ethan-1-one; 2-[2-fluoro-3-(trifluoromethyl)phenyl]-1-(4-{[1,2,4]triazolo[4,3-b]pyridazin-6-yl}piperazin-1-yl)ethan-1-one; 2-fluoro-5-[2-oxo-2-(4-{[1,2,4]triazolo[4,3-b]pyridazin-6-yl}piperazin-1-yl)ethyl]benzonitrile; 2-(4-fluoro-3-nitrophenyl)-1-(4-{[1,2,4]triazolo[4,3-b]pyridazin-6-yl}piperazin-1-yl)ethan-1-one; 2-[4-fluoro-3-(trifluoromethoxy)phenyl]-1-(4-{[1,2,4]triazolo[4,3-b]pyridazin-6-yl}piperazin-1-yl)ethan-1-one; 2-{6-fluoro-[1,1′-biphenyl]-3-yl}-1-(4-{[1,2,4]triazolo[4,3-b]pyridazin-6-yl}piperazin-1-yl)ethan-1-one; 2-{4′,6-difluoro-[1,1′-biphenyl]-3-yl}-1-(4-{[1,2,4]triazolo[4,3-b]pyridazin-6-yl}piperazin-1-yl)ethan-1-one; 2-[3-(1,1-difluoroethyl)-4-fluorophenyl]-1-(4-{[1,2,4]triazolo[4,3-b]pyridazin-6-yl}piperazin-1-yl)ethan-1-one; 2-[4-fluoro-3-(1,1,2,2,2-pentafluoroethyl)phenyl]-1-(4-{[1,2,4]triazolo[4,3-b]pyridazin-6-yl}piperazin-1-yl)ethan-1-one; 2-(3-ethynyl-4-fluorophenyl)-1-(4-{[1,2,4]triazolo[4,3-b]pyridazin-6-yl}piperazin-1-yl)ethan-1-one; 2-[4-fluoro-3-(prop-1-yn-1-yl)phenyl]-1-(4-{[1,2,4]triazolo[4,3-b]pyridazin-6-yl}piperazin-1-yl)ethan-1-one; 2-(3-tert-butyl-4-fluorophenyl)-1-(4-{[1,2,4]triazolo[4,3-b]pyridazin-6-yl}piperazin-1-yl)ethan-1-one; 2-[4-fluoro-3-(hydroxymethyl)phenyl]-1-(4-{[1,2,4]triazolo[4,3-b]pyridazin-6-yl}piperazin-1-yl)ethan-1-one; 2-[4-fluoro-3-(methoxymethyl)phenyl]-1-(4-{[1,2,4]triazolo[4,3-b]pyridazin-6-yl}piperazin-1-yl)ethan-1-one; 2-fluoro-5-[2-oxo-2-(4-{[1,2,4]triazolo[4,3-b]pyridazin-6-yl}piperazin-1-yl)ethyl]benzoic acid; 2-fluoro-5-[2-oxo-2-(4-{[1,2,4]triazolo[4,3-b]pyridazin-6-yl}piperazin-1-yl)ethyl]benzamide; 2-fluoro-N-methyl-5-[2-oxo-2-(4-{[1,2,4]triazolo[4,3-b]pyridazin-6-yl}piperazin-1-yl)ethyl]benzamide; 2-fluoro-N,N-dimethyl-5-[2-oxo-2-(4-{[1,2,4]triazolo[4,3-b]pyridazin-6-yl}piperazin-1-yl)ethyl]benzamide; 2-fluoro-5-[2-oxo-2-(4-{[1,2,4]triazolo[4,3-b]pyridazin-6-yl}piperazin-1-yl)ethyl]benzene-1-sulfonamide; 2-fluoro-N-methyl-5-[2-oxo-2-(4-{[1,2,4]triazolo[4,3-b]pyridazin-6-yl}piperazin-1-yl)ethyl]benzene-1-sulfonamide; 2-fluoro-N,N-dimethyl-5-[2-oxo-2-(4-{[1,2,4]triazolo[4,3-b]pyridazin-6-yl}piperazin-1-yl)ethyl]benzene-1-sulfonamide; 2-(4-fluoro-3-methanesulfonylphenyl)-1-(4-{[1,2,4]triazolo[4,3-b]pyridazin-6-yl}piperazin-1-yl)ethan-1-one; and combinations thereof. In such an embodiment, the compound may be chosen from: 2-[4-fluoro-3-(trifluoromethyl)phenyl]-1-(4-{[1,2,4]triazolo[4,3-b]pyridazin-6-yl}piperazin-1-yl)ethan-1-one; 2-(3-bromo-4-chlorophenyl)-1-(4-{[1,2,4]triazolo[4,3-b]pyridazin-6-yl}piperazin-1-yl)ethan-1-one; 2-(3,4-dichlorophenyl)-1-(4-{[1,2,4]triazolo[4,3-b]pyridazin-6-yl}piperazin-1-yl)ethan-1-one; 2-(3-bromo-4-fluorophenyl)-1-(4-{[1,2,4]triazolo[4,3-b]pyridazin-6-yl}piperazin-1-yl)ethan-1-one; 2-(3-chloro-4-fluorophenyl)-1-(4-{[1,2,4]triazolo[4,3-b]pyridazin-6-yl}piperazin-1-yl)ethan-1-one; 2-(4-bromo-3-chlorophenyl)-1-(4-{[1,2,4]triazolo[4,3-b]pyridazin-6-yl}piperazin-1-yl)ethan-1-one; 2-[4-chloro-3-(trifluoromethyl)phenyl]-1-(4-{[1,2,4]triazolo[4,3-b]pyridazin-6-yl}piperazin-1-yl)ethan-1-one; 2-(4-chloro-3-methylphenyl)-1-(4-{[1,2,4]triazolo[4,3-b]pyridazin-6-yl}piperazin-1-yl)ethan-1-one; 2-[3,5-bis(trifluoromethyl)phenyl]-1-(4-{[1,2,4]triazolo[4,3-b]pyridazin-6-yl}piperazin-1-yl)ethan-1-one; 2-(4-fluoro-3-methylphenyl)-1-(4-{[1,2,4]triazolo[4,3-b]pyridazin-6-yl}piperazin-1-yl)ethan-1-one; 2-[3-chloro-5-(trifluoromethyl)phenyl]-1-(4-{[1,2,4]triazolo[4,3-b]pyridazin-6-yl}piperazin-1-yl)ethan-1-one; 2-(4-chloro-3-fluorophenyl)-1-(4-f[1,2,4]triazolo[4,3-b]pyridazin-6-yl}piperazin-1-yl)ethan-1-one; 2-(3,4-difluorophenyl)-1-(4-{[1,2,4]triazolo[4,3-b]pyridazin-6-yl}piperazin-1-yl)ethan-1-one; 2-(4-bromo-3-fluorophenyl)-1-(4-{[1,2,4]triazolo[4,3-b]pyridazin-6-yl}piperazin-1-yl)ethan-1-one; 2-(4-chlorophenyl)-1-(4-{[1,2,4]triazolo[4,3-b]pyridazin-6-yl}piperazin-1-yl)ethan-1-one; 2-(3-chloro-5-fluorophenyl)-1-(4- {[1,2,4]triazolo[4,3-b]pyridazin-6-yl}piperazin-1-yl)ethan-1-one; 2-(4-ethynylphenyl)-1-(4-{[1,2,4]triazolo[4,3-b]pyridazin-6-yl}piperazin-1 -yl)ethan-1-one; 2-(3,5-dichlorophenyl)-1-(4-{[1,2,4]triazolo[4,3-b]pyridazin-6-yl}piperazin-1-yl)ethan-1-one; and combinations thereof.

The following example is presented to illustrate the present disclosure. It is not intended to be limiting in any manner.

### EXAMPLE

The following example describes compounds and synthesis of compounds of the present disclosure.

General Methods. Commercially available reagents and solvents were used without further purification unless stated otherwise. HPLC and LC-MS analyses were performed on an Agilent 1100 HPLC/MSD electrospray mass spectrometer in positive ion mode with scan range was 100-1000d. Preparative normal phase chromatography was performed on a CombiFlash Rf+ (Teledyne Isco) with SiliaFlash F60 40-63 µm (230-400 mesh) silica gel (SiliCycle Inc.). Preparative reverse phase HPLC was performed on a CombiFlash Rf+ (Teledyne Isco) equipped with RediSep Rf Gold pre-packed C18 cartridges and an acetonitrile/water gradient with or without 0.05% TFA as noted. NMR spectra were recorded on Bruker 400 MHz and 700 MHz spectrometers. The signal of the deuterated solvent was used as internal reference. Chemical shifts (δ) are given in ppm and are referenced to residual not fully deuterated solvent signal. Coupling constants (J) are given in Hz. All final compounds were purified to ≥95% as determined by HPLC UV absorbance unless noted otherwise.

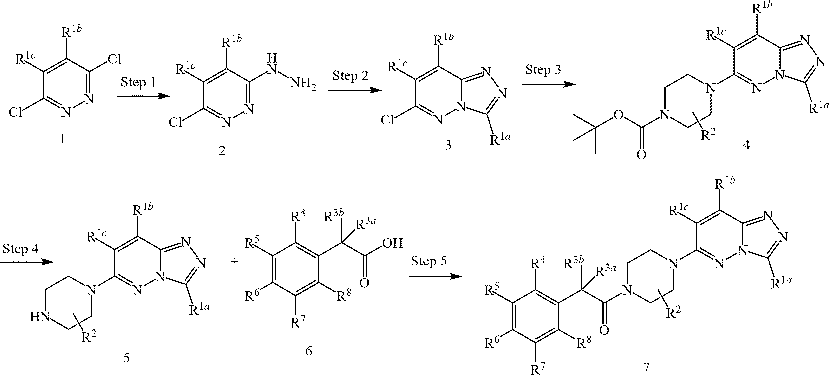

### Preparation of 3-chloro-6-hydrazinylpyridazine Hydrochloride (Scheme 1, Step 1, Intermediate 2)

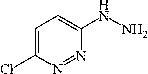

To a microwave vial was added 3,6-dichloropyridazine (1.00 g, 7.00 mmol) and hydrazine monohydrate (0.38 mL, 7.70 mmol). The mixture was dissolved in 10 mL of ethanol and microwaved for 2 h at 100° C. The crude reaction mixture was extracted three times with dichloromethane. The organic layer was dried with sodium sulfate and concentrated in vacuo to give the crude product (1.69 g). ¹H NMR (400 MHz, DMSO-d₆) δ ppm 7.08 (d, J=9.2, 1H), 7.42 (d, J=9.3, 1H), 8.05 (s, 4H), 8.17 - 8.23 (m, 1H). LC-MS: m/z 145.0, 147.0 (MH)⁺.

### Preparation of 6-chloro-[1,2,4]triazolo[4,3-b]pyridazine (Scheme 1, Step 2, Intermediate 3a).

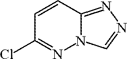

To a microwave vial was added 3-chloro-6-hydrazinopyridazine (3.24 g, 22.5 mmol). The starting material was dissolved in 15 mL formic acid, sealed, the heated to 100° C. in the microwave reactor for 2 h. The reaction mixture was neutralized with satd NaHCO₃. The aqueous layer was then extracted three times with dichloromethane. The organic layer was dried over sodium sulfate, filtered, and concentrated in vacuo to give 1.54 g of the crude title compound. ¹H NMR (400 MHz, D₂O) δ ppm 7.43 - 7.49 (m, 1H), 8.21 - 8.27 (m, 1H), 9.30 -9.34 (m, 1H). LC-MS: m/z 155.0, 157.0 (MH)⁺.

### Preparation of 6-chloro-3-(trifluoromethyl)-[1,2,4]triazolo[4,3-b]pyridazine (Scheme 1, Step 2, Intermediate 3b).

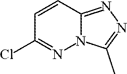

To a microwave vial was added 3-chloro-6-hydrazinopyridazine (100 mg, 0.7 mmol). The starting material was dissolved in 5 mL trifluoroacetic acid, sealed, the heated to 100° C. in the microwave reactor for 2 h. The reaction mixture was neutralized with satd NaHCO₃. The aqueous layer was then extracted three times with dichloromethane. The organic layer was dried over sodium sulfate, filtered, and concentrated in vacuo to give 107 mg of the crude title compound. LC-MS: m/z 168.0 (MH)⁺, 170.0.

### Preparation of 6-chloro-3-methyl-[1,2,4]triazolo[4,3-b]pyridazine (Scheme 1, Step 2, Intermediate 3c).

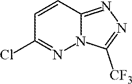 [SMILES: FC(F)(F)c1nnc2ccc(Cl)nn12]

To a microwave vial was added 3-chloro-6-hydrazinopyridazine (100 mg, 0.7 mmol). The starting material was dissolved in 5 mL acetic acid, sealed, the heated to 100° C. in the microwave reactor for 2 h. The reaction mixture was neutralized with satd NaHCO₃. The aqueous layer was then extracted three times with dichloromethane. The organic layer was dried over sodium sulfate, filtered, and concentrated in vacuo to give 94 mg of the crude title compound. LC-MS: m/z 222.0 (MH)⁺, 224.0.

### Preparation of 3-phenyl-6-chloro-[1,2,4]triazolo[4,3-b]pyridazine (Scheme 1, Step 2, Intermediate 3d).

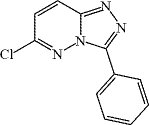 [SMILES: Clc1ccc2nnc(-c3ccccc3)n2n1]

To a round bottom flask was added 3-chloro-6-hydrazinopyridazine (100 mg, 0.70 mmol) and phenylacetaldehyde (0.1 mL, 0.76 mmol). The mixture was dissolved in ethanol, heated to 60° C., and stirred for 30 minutes. Me₄NBr (21 mg, 0.14 mmol) and Oxone (158 mg, 1.04 mmol) were added. The reaction mixture was then stirred overnight at 60° C. The crude mixture was diluted with water and extracted 3 times with DCM. The organic layers were dried over magnesium sulfate, filtered, and concentrated to give 67 mg of the crude product. LC-MS: m/z 230.0 (MH)⁺, 232.0.

### Preparation of 3-benzyl-6-chloro-[1,2,4]triazolo[4,3-b]pyridazine (Scheme 1, Step 2, Intermediate 3e).

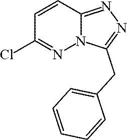 [SMILES: Clc1ccc2nnc(Cc3ccccc3)n2n1]

To a round bottom flask was added 3-chloro-6-hydrazinopyridazine (500 mg, 2.75 mmol), phenylacetic acid (415 mg, 3.05 mmol), EDC-HCl (1.07 g, 5.55 mmol), HOBt—H₂O (750 mg, 5.55 mmol), and triethylamine (1.25 mL, 8.35 mL). The mixture was dissolved in acetonitrile and stirred overnight at room temperature. The acetonitrile was removed in vacuo and the remaining mixture was dissolved in toluene and equipped with a Dean-Stark trap. The reaction mixture was heated to 200° C. overnight. The solvent was removed in vacuo and the product was extracted in ethyl acetate, rinsing twice with water and once with brine. The organic layer was dried over magnesium sulfate, filtered, and concentrated to give the title compound (138 mg, 20%). LC-MS: m/z 244.1 (MH)⁺, 246.0.

### Preparation of 6-chloro-3-(2-(trifluoromethyl)pyridin-4-yl)-[1,2,4]triazolo[4,3-b]pyridazine (Intermediate 3f).

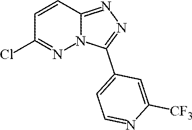 [SMILES: FC(F)(F)c1cc(-c2nnc3ccc(Cl)nn23)ccn1]

A well-dried round bottom flask was charged with Intermediate 2 (1.0 g, 7.0 mmol), isonicotinic acid (1.5 g, 7.9 mmol) and 15 mL of POCl₃. The mixture was then stirred and heated at 130° C. for 30 h. The reaction was then monitored with LCMS to completion. The resulting solution was allowed to cool to room temperature, concentrated and quenched with ice, after which was washed with NaHCO₃ and dried with Na₂SO₄. The organic layer was then concentrated to give white solid (1.2 g, 60%). m/z 300 (MH)⁺. ¹H NMR (400 MHz, DMSO-d₆) δ 9.22 (d, J = 0.73 Hz, 1H), 8.09 (s, 1H), 7.37 (d, J = 10.27 Hz, 1H), 7.22 (d, J = 9.54 Hz, 4H), 3.76 (s, 2H), 3.62 (br. s., 4H), 3.49 - 3.58 (m, 6H), 3.44 - 3.49 (m, 2H), 3.42 (s, 2H), 2.33 (d, J = 3.91 Hz, 4H).

### Preparation of Tert-butyl 4-{[1,2,4]triazolo[4,3-b]pyridazin-6-yl}piperazine-1-carboxylate (Scheme 1, Step 3, Intermediate 4a).

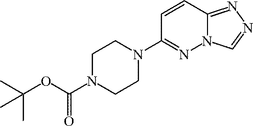 [SMILES: CC(C)(C)OC(=O)N1CCN(c2ccc3nncn3n2)CC1]

To a microwave vial was added 6-chloro[1,2,4]triazolo[4,3-b]pyridazine (1.54 g, 9.96 mmol), N-Boc-piperazine (2.04 g, 11.0 mmol), and DIEA (2.22 mL, 13.0 mmol). The starting materials were dissolved in ethanol. The vial was sealed and heated to 100° C. in the microwave reactor for 2 hours. Ethanol was removed in vacuo, water was added and the crude product was extracted with dichloromethane three times. The organic layer was dried over sodium sulfate, filtered, and concentrated in vacuo to give 2.16 g of crude tert-butyl 4-{[1,2,4]triazolo[4,3-b]pyridazin-6-yl}piperazine-1-carboxylate. ¹H NMR (400 MHz, CDCl₃) δ ppm 1.50 (s, 9H), 3.52 - 3.63 (m, 8H), 6.94 (d, J=10.2 Hz, 1H) 7.91 (d, J=10.2 Hz, 1H), 8.79 (s, 1H). LC-MS: m/z 305.1 (MH)⁺.

### Preparation of Tert-butyl 4-{3-benzyl-[1,2,4]triazolo[4,3-b]pyridazin-6-yl}piperazine-1-carboxylate (Scheme 1, Step 3, Intermediate 4e).

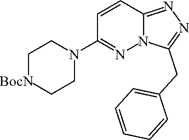 [SMILES: CC(C)(C)OC(=O)N1CCN(c2ccc3nnc(Cc4ccccc4)n3n2)CC1]

3-benzyl-6-chloro-[1,2,4]triazolo[4,3-b]pyridazine (138 mg, 0.57 mmol) was added to a microwave vial with N-Boc-piperazine (116 mg, 0.62 mmol) and DIEA (0.2 mL, 1.13 mmol), then dissolved in 5 mL of ethanol. The vial was sealed and microwaved at 100° C. for four hours. The solvent was removed in vacuo, and the remaining solid was purified via Reverse Phase Flash Chromatography (5➔95% CH₃CN/H₂O) to give the title compound (189 mg, 84%). LC-MS m/z 394.2 (M)⁺.

### Preparation of Tert-butyl 4-([1,2,4]triazolo[4,3-b]pyridazin-6-yl)-2-methylpiperazine-1-carboxylate (Scheme 1, Step 3, Intermediate 4 g).

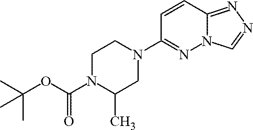 [SMILES: CC1CN(c2ccc3nncn3n2)CCN1C(=O)OC(C)(C)C]

6-chloro-[1,2,4]triazo[4,3-b]pyridazine (1.76 mmol), t-butyl-linker-1-carboxylate (1.93 mmol) and DIEA (0.39 mL) were dissolved in ethanol (4 mL) and heated in the microwave at 160° C. for 8 h. The crude mixture was extracted using EtOAc and water. The organic layer was then dried using Na₂SO₄ followed by concentration to give the title compound as a brown oil (392 mg, 95%). LC-MS m/z 319 (MH)⁺.

### Preparation of 1-{[1,2,4]triazolo[4,3-b]pyridazin-6-yl}piperazine hydrochloride (Scheme 1, Step 4, Intermediate 5a).

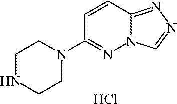 [SMILES: c1cc2nncn2nc1N1CCNCC1 ; Cl]

To a round-bottomed flask was added tert-butyl 4-{[1,2,4]triazolo[4,3-b]pyridazin-6-yl}piperazine-1-carboxylate (2.16 g, 7.10 mmol) and excess 4 M HCl in dioxane (12 mL). The reaction mixture was stirred at room temperature for 2 h. Dioxane was removed in vacuo, and the crude product was rinsed with DCM. The DCM was removed in vacuo, then the crude product was rinsed with diethyl ether, which was subsequently removed in vacuo. The crude product was rinsed again with diethyl ether. The diethyl ether was decanted, giving 1.76 g of 1-{[1,2,4]triazolo[4,3-b]pyridazin-6-yl}piperazine hydrochloride. ¹H NMR (400 MHz, D₂O) δ 3.38-3.44 (m,4H), 3.91-3.96 (m, 4H), 7.54-7.60 (m, 1H), 8.08-8.13 (m, 1H), 9.17-9.20 (m, 1H). LC-MS: m/z 205.1 (MH)⁺.

### Preparation of 1-{3-benzyl-[1,2,4]triazolo[4,3-b]pyridazin-6-yl}piperazine trifluoroacetate (Scheme 1, Step 4, Intermediate 5e)

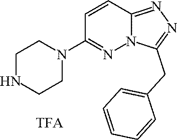 [SMILES: c1ccc(Cc2nnc3ccc(N4CCNCC4)nn23)cc1 ; "TFA"]

tert-butyl 4-{3-benzyl-[1,2,4]triazolo[4,3-b]pyridazin-6-yl}piperazine-1-carboxylate (189 mg, 0.48 mmol) was dissolved in 5 mL of trifluoroacetic acid and 5 mL of acetonitrile and stirred at room temperature for two hours. Solvent and excess TFA were removed in vacuo. The crude product was rinsed with DCM. The DCM was removed in vacuo, then the crude product was rinsed with diethyl ether, which was subsequently removed in vacuo. The crude product was rinsed again with diethyl ether. The diethyl ether was decanted, giving the title compound (228 mg, quant.). LC-MS m/z 294.2 (M)⁺.

### Preparation of 6-(piperazin-1-yl)-3-(2-(trifluoromethyl)pyridin-4-yl)-[1,2,4]triazolo[4,3-b]pyridazine hydrochloride (Intermediate 5f).

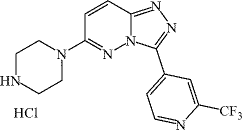 [SMILES: FC(F)(F)c1cc(-c2nnc3ccc(N4CCNCC4)nn23)ccn1 ; Cl]

The title compound was prepared from Intermediate 3f as described for the synthesis of Intermediates 4a and 5a. White solid (210 mg, 90%). m/z 349.8 (MH)⁺.

### Preparation of 6-(3-methylpiperazin-1-yl)-[1,2,4]triazolo[4,3-b]pyridazine hydrochloride (Scheme 1, Step 4, Intermediate 5 g)

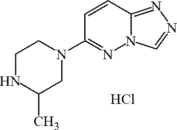 [SMILES: CC1CN(c2ccc3nncn3n2)CCN1 ; Cl]

Crude intermediate 3 was suspended in 4 M HCl in dioxane (8 mL) and stirred at room temperature for 1 hour. The gummy mixture slowly turned more heterogeneous white solid mixture. The suspension was concentrated and dried under vacuum to give the product. The LC-MS of the product was taken for the products formed. M/z 219, yield 98%, brown oily compound.

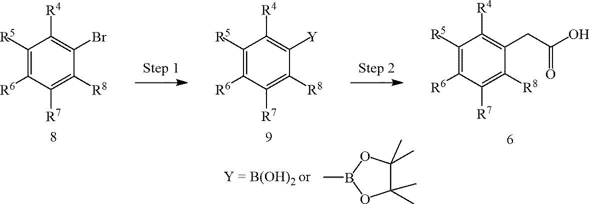

### Preparation of 2-(4-chloro-3-methylphenyl)-4,4,5,5-tetramethyl-1,3,2-dioxaborolane (Scheme 2, Step 1, Intermediate 9c).

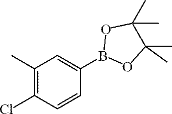

To a microwave vial was added 4-bromo-1-chloro-2-methylbenzene (0.322 mL, 2.45 mmol), B₂Pin₂ (685 mg, 2.70 mmol), PdCl₂(dppf) (90 mg, 0.12 mmol), and potassium acetate (482 mg, 4.9 mmol). The mixture was dissolved in anhydrous dioxane (15 mL), sealed, and degassed with argon for 20 minutes. The reaction mixture was heated to 90° C. overnight. The reaction mixture was filtered through Celite rinsing with DCM. The filtrate was concentrated in vacuo. The remaining oil was purified via flash chromatography eluting with 0-50% ethyl acetate/hexanes. Fractions of interest were combined to give 580 mg of 2-(4-chloro-3-methylphenyl)-4,4,5,5-tetramethyl-1,3,2-dioxaborolane.

### General Procedure for Preparation of Aryl Acetic Acid Derivatives (Scheme 2, Step 2, Intermediates 6a-h) (General Method A)

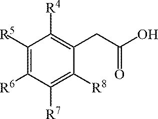

To a round-bottomed flask was added a boronic acid or ester 9(1 eq), α-bromo ethyl acetate (1.2 eq), K₃PO₄ (5 eq), Pd(OAc)₂ (0.03 eq), and P(o-tol)₃ or P(1-nap)₃ (0.09 eq). The mixture was dissolved in anhydrous THF, sealed, and degassed with argon for 20 minutes. The reaction mixture was stirred at room temperature overnight. To the reaction mixture, 2-5 mL of 1N LiOH was added. The reaction mixture was stirred overnight at room temperature. THF was removed in vacuo, and the remaining solution was filtered through Celite rinsing with DCM. The aqueous layer was acidified to pH 2 with 6N HCl, then extracted with DCM 3 times. The organic layers were combined, dried over Na₂SO₄, and concentrated in vacuo to give the corresponding aryl acetic acid. See Table 1 for compound characterization.

TABLE 1

Characterization of Intermediates 6a-h

| Int. | Structure | Yield | ¹H NMR (400 MHz) |
|---|---|---|---|
| 6a | 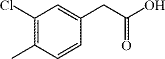 | 31% | (DMSO-d₆) δ 2.30 (s, 3H), 3.56 (s, 2H), 7.12 (d, J=7.6, 1H), 7.28 (d, J=7.7, 1H), 7.31 (s, 1H), 12.30 (br.s., 1H) |
| 6b | 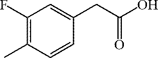 | 11% | (CDCl₃) □ ppm 2.26 (s, 3H), 3.51 (s, 2H), 6.95 (s, 1H), 6.97 (d, J=3.7, 2H), 7.15 (t, J=7.8, 1H), 8.39 (s, 1H) |
| 6c | 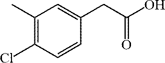 | 66% | (DMSO-d₆) δ 2.30 (s, 3H), 3.53 (s, 2H), 7.10 (d, J=8.2, 1H), 7.23 (s, 1H), 7.33 (d, J=8.1, 1H), 12.16-12.57 (br.s., 1H) |
| 6d | 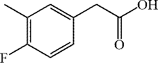 | 69% | (DMSO-d₆) δ ppm 2.21 (d, J=1.83 Hz, 3H), 3.51 (s, 2H), 7.00-7.12 (m, 2H), 7.15 (d, J=7.58 Hz, 1H), 12.30(s, 1H) |
| 6e | 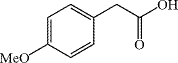 [SMILES: COc1ccc(CC(=O)O)cc1] | 90% | (DMSO-d₆) δ ppm 3.48 (s, 2 H) 3.73 (s, 3 H) 6.86 (d, J=8.56 Hz, 2 H) 7.16 (d, J=8.56 Hz, 2 H) 12.21 (s, 1 H) |
| 6f | 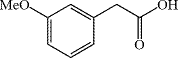 [SMILES: COc1cccc(CC(=O)O)c1] | 68% | (DMSO-d₆) δ ppm 3.53 (s, 2 H) 3.73 (s, 3 H) 6.77 -6.87 (m, 3 H) 7.17 - 7.27 (m, 1 H) 12.28 (br. s., 1 H) |
| 6g | 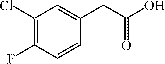 [SMILES: O=C(O)Cc1ccc(F)c(Cl)c1] | 19% | (DMSO-d₆) δ 3.61 (s, 2H), 7.23-7.31 (m, 1H), 7.31-7.39 (m, 1H), 7.49 (dd, J=7.3, 1.9, 1H), 12.44 (br.s., 1H) |
| 6h | 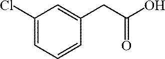 [SMILES: O=C(O)Cc1cccc(Cl)c1] | 59% | (DMSO-d₆) δ ppm 3.61 (s, 2 H) 7.20 - 7.26 (m, 1 H) 7.29 - 7.38 (m, 3 H) 12.41 (br. s., 1 H) |

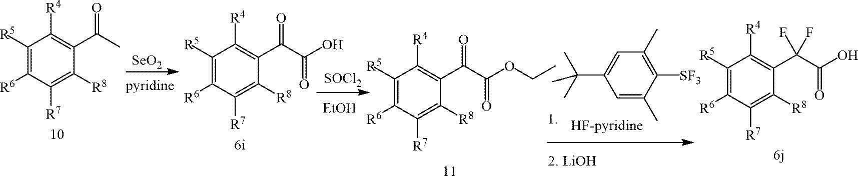

Preparation of 3,4-dichlorophenylglyoxylic acid (6i). To a round bottom flask, with a stirring bar was added 3,4-dichloroacetophenone (100 mg, 0.53 mmol) and selenium dioxide (88 mg, 0.79 mmol), then 5 mL of anhydrous pyridine was quickly added. The reaction mixture was then stirred and heated under a nitrogen atmosphere for 110° C. for 1 h. The temperature was lowered to 90° C. for another 4 h. The reaction mixture was allowed overnight, after which was filtered and concentrated, 20 mL of 2N NaOH was added and later, 30 mL of 2 N HCl was added. The product was filtered from the mixture. The residue was dissolved in EtOAc and concentrated under vacuum. Crude product was used for the next reaction. Yield 95%, white to pinkish solid compound.

Preparation of Ethyl-3,4-dichlorophenylglyoxyloate (11). 50 mL round bottom flask was charged with 3,4-dichlorophenylglyoxylic acid (400 mg, 1.83 mmol) and which dissolved with 30 mL of EtOH. The reaction vessel was cooled to 0° C. in water bath. Followed by addition of 2 mL of thionyl chloride (27 mmol). The reaction vessel was then refluxed at 80° C. for overnight. The reaction mixture was concentrated under vacuum and extracted using EtOAc and water and washed with NaHCO₃. The organic extract was then dried using NaSO₄, filtered and concentrated to give a yellow oily compound, crude yield 85%.

Preparation of 2-(3,4-dichlorophenyl)-2,2-difluoroacetic acid (6j). To a plastic tube was then dried by flashing N₂(_{g}) through and charged with 6ml of anhydrous DCM, followed by addition of Ethyl-3,4-dichlorophenylglyoxyloate (457 mg, 1.85 mmol) and addition of 4-tert-Butyl-2,6-dimethylphenyl sulfur trifluoride (694.7 mg, 2.77 mmol) and allowed to str for about 5 minutes. ~44 µL of HF-Py was added lastly. The reaction mixture was stirred for ~10 minutes after which the temperature was increased to 45° C. for 3 hours after which it was allowed to stir at room temperature for 16 h. The reaction was quenched with NaHCO₃ and extracted with EtOAc and water. The crude brown oil compound was then subjected to hydrolysis by stirring the crude product in 10 mL of LiOH with MeOH for overnight, after which the mixture was neutralized with 2N HCl and extracted with EtOAc, dried with Na₂SO₄ and concentrated to the title compound (brown solid, crude yield 67%).

General Procedure for Amide Couplings to Prepare Examples 2756-2764; 2767; 10090, 10238-10241, 10441-10442, 10502-10505 (Scheme 1, Step 5) (General Method B).

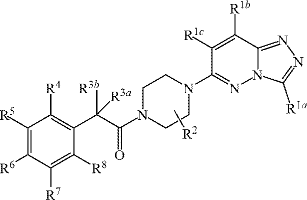

To a round-bottomed flask was added an aryl acetic acid 6 (1 eq), 1-{[1,2,4]triazolo[4,3-b]pyridazin-6-yl}piperazine (1.2 eq), EDC-HCl (2 eq), HOBt—H₂O (2 eq), and TEA (3 eq). The mixture was dissolved in DMF and stirred overnight at room temperature. DMF was removed in vacuo and the product was extracted with ethyl acetate, rinsing twice with water and once with brine. The organic layer was dried over Na₂SO₄, filtered, and concentrated in vacuo. The remaining oil was purified via flash chromatography. Fractions containing the desired product were combined to give the corresponding amide. See Table 2 for MS and NMR characterization.

TABLE 2

Characterization of Examples 2756-2764; 2767; 10090, 10238-10241, 10441-10442, 10502-10505

| Ex. | Structure | Yield | ¹H NMR (400 MHz) | LC-MS m/z (MH)⁺ |
|---|---|---|---|---|
| 2756 | 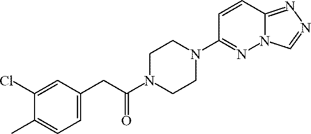 [SMILES: Cc1ccc(CC(=O)N2CCN(c3ccc4nncn4n3)CC2)cc1Cl] | Yellow solid, 39 mg, 38% | (DMSO-d₆) δ 2.29 (s, 3H), 3.54 (d, J=4.4, 4H), 3.61 (br.s., 2H), 3.65 (br.s., 2H), 3.76 (s, 2H), 7.12 (s, 1H), 7.28 (d, J=10.8, 2H), 7.39 (d, J=10.2, 1H), 8.11 (d, J=10.0, 1H), 9.23 (s, 1H) | 371, 373 |
| 2757 | 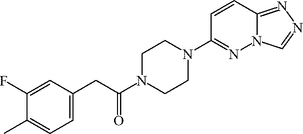 [SMILES: Cc1ccc(CC(=O)N2CCN(c3ccc4nncn4n3)CC2)cc1F] | Yellow solid, 225 mg, quant. | (DMSO-d₆) δ 2.20 (s, 3H), 3.54 (d, J=11.3, 4H), 3.59-3.68 (m, 4H), 3.76 (s, 2H), 6.97 (d, J=7.6, 1H), 7.02 (s, 1H), 7.20 (s, 1H), 7.38 (d, J=10.3, 1H), 8.10 (d, J=10.3, 1H), 9.22 (s, 1H) | 355 |
| 2758 | 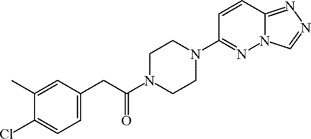 [SMILES: Cc1cc(CC(=O)N2CCN(c3ccc4nncn4n3)CC2)ccc1Cl] | Off-white solid, 132 mg, 66% | (DMSO-d₆) δ 2.30 (s, 3H), 3.53 (br.s., 4H), 3.57 - 3.68 (m, 4H), 3.75 (s, 2H), 7.06 -7.11 (m, 1H), 7.19 -7.23 (m, 1H), 7.33 (s, 1H) 7.40 (s, 1H), 8.08-8.14 (m, 1H), 9.23 (d, J=0.7, 1H) | 371 |
| 2759 | 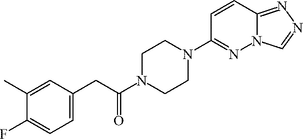 [SMILES: Cc1cc(CC(=O)N2CCN(c3ccc4nncn4n3)CC2)ccc1F] | Brown solid, 158 mg, 75% | (DMSO-d₆) δ 2.21 (s, 3H), 3.53 (br.s., 4H), 3.57-3.68 (m, 4H), 3.72 (s, 2H), 7.04-7.09 (m, 2H), 7.14 (d, J=7.7, 1H), 7.40 (d, J=10.1, 1H), 8.11 (d, J=10.1, 1H), 9.25 (s, 1H) | 355 |
| 2760 | 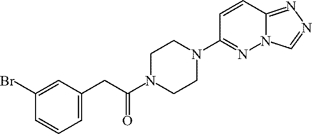 [SMILES: O=C(Cc1cccc(Br)c1)N1CCN(c2ccc3nncn3n2)CC1] | Yellow solid, 122 mg, 66% | (DMSO-d₆) δ 3.51-3.58 (m, 4H), 3.58-3.71 (m, 4H), 3.81 (s, 2H), 7.25-7.29 (m, 1H), 7.39 (d, J=10.3, 1H), 7.41-7.45 (m, 1H), 7.46 (s, 1H), 8.08 - 8.14 (m, 1H), 9.23 (s, 1H) | 401, 403 |
| 2761 | 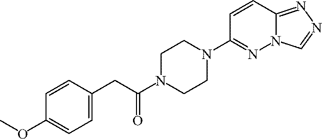 [SMILES: COc1ccc(CC(=O)N2CCN(c3ccc4nncn4n3)CC2)cc1] | Yellow solid, 114 mg, 54% | (DMSO-d₆) δ 3.49 (d, J=18.8, 4H), 3.62 (br.s., 4H), 3.67 -3.76 (m, 6H), 6.87 (d, J=8.2, 2H), 7.16 (d, J=7.8, 2H), 7.37 (d, J=10.0, 1H), 8.10 (d, J=10.0, 1H), 9.22 (s, 1H) | 353 |
| 2762 | 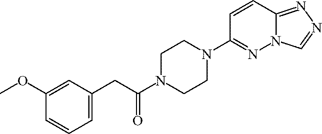 [SMILES: COc1cccc(CC(=O)N2CCN(c3ccc4nncn4n3)CC2)c1] | Yellow solid, 102 mg, 48% | (DMSO-d₆) δ 3.49 (d, J=18.8, 4H), 3.62 (br.s., 4H), 3.67 -3.76 (m, 6H), 6.87 (d, J=8.2, 2H), 7.16 (d, J=7.8, 2H), 7.37 (d, J=10.0, 1H), 8.10 (d, J=10.0, 1H), 9.22 (s, 1H) | 353 |
| 2763 | 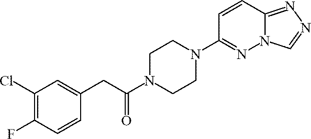 [SMILES: O=C(Cc1ccc(F)c(Cl)c1)N1CCN(c2ccc3nncn3n2)CC1] | Brown solid, 130 mg, 65% | (DMSO-d₆) δ 3.56 (br.s., 4H), 3.59-3.71 (m, 4H), 3.80 (s, 2H), 7.21-7.28 (m, 1H), 7.35 (s, 1H), 7.39 (d, J=10.3, 1H), 7.43-7.48 (m, 1H), 8.11 (d, J=10.3, 1H), 9.23 (s, 1H) | 375, 377 |
| 2764 | 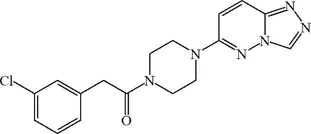 [SMILES: O=C(Cc1cccc(Cl)c1)N1CCN(c2ccc3nncn3n2)CC1] | Cream solid, 123 mg, 59% | (DMSO-d₆) δ 3.55 (d, J=5.4, 4H), 3.65 (d, J=18.1, 4H), 3.81 (s, 2H), 7.18-7.24 (m, 1H), 7.29-7.35 (m, 3H), 7.40 (s, 1H), 8.08 - 8.14 (m, 1H), 9.23 (s, 1H) | 357, 359 |
| 2767 | 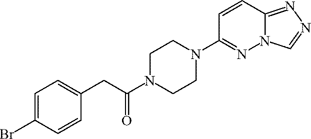 [SMILES: O=C(Cc1ccc(Br)cc1)N1CCN(c2ccc3nncn3n2)CC1] | Yellow solid, 95 mg, 51% | (DMSO-d₆) δ 3.53 (br.s., 4H), 3.58 - 3.68 (m, 4H), 3.77 (s, 2H), 7.21 (d, J=8.2, 2H), 7.38 (d, J=10.0, 1H), 7.50 (d, J=8.3, 2H), 8.11 (d, J=10.2, 1H), 9.23 (s, 1H) | 401, 403 |
| 10090 | 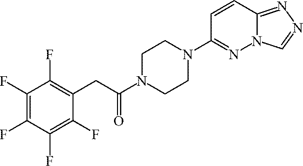 [SMILES: O=C(Cc1c(F)c(F)c(F)c(F)c1F)N1CCN(c2ccc3nncn3n2)CC1] | Yellow solid, 155 mg, 28% | (DMSO-d₆) δ 3.60 (d, J=9.54 Hz, 4 H), 3.66 - 3.80 (m, 4 H), 5.75 (s, 2 H), 7.42 (d, J=10.03 Hz, 1H), 8.13 (d, J=10.03 Hz, 1 H), 9.24 (s, 1 H) ¹⁹F NMR (376 MHz, DMSO-d6) δ -164.08 - -163.45 (m, 2 F), -157.08 - -156.68 (m, 2 F), -142.82 - -142.13 (m, 1 F) | 412 |
| 10238 | 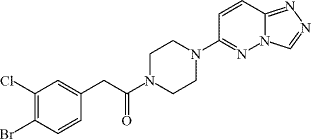 [SMILES: O=C(Cc1ccc(Br)c(Cl)c1)N1CCN(c2ccc3nncn3n2)CC1] | Yellow solid, 17 mg, 19% | (CDCl₃) δ ppm 3.65 (d, J=13.57 Hz, 6H), 3.73 (s, 2 H), 3.82 (br. s., 2 H), 6.93 (d, J=10.15 Hz, 1 H), 7.39 (s, 1 H) 7.54-7.63 (m, 2H), 7.94 (d, J=10.03 Hz, 1H), 8.80 (s, 1H) | 434, 436, 438 |
| 10239 | 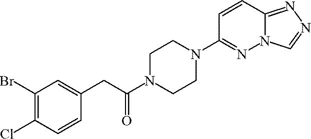 [SMILES: O=C(Cc1ccc(Cl)c(Br)c1)N1CCN(c2ccc3nncn3n2)CC1] | Yellow solid, 34 mg, 37% | (CDCl₃) δ ppm 3.55 (d, J=8.07 Hz, 2 H), 3.66 (d, J=19.07 Hz, 4 H), 3.74 (s, 2 H), 3.82 (br. s., 2 H), 6.93 (d, J=10.15 Hz, 1H), 7.17(d, J=8.56 Hz, 1H), 7.39-7.43(m, 1H), 7.55(s, 1H), 7.94 (d, J=10.03 Hz, 1H), 8.80 (s, 1H) | 434, 436, 438 |
| 10240 | 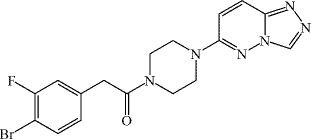 [SMILES: O=C(Cc1ccc(Br)c(F)c1)N1CCN(c2ccc3nncn3n2)CC1] | Yellow solid, 15 mg, 17% | (CDCl₃) δ ppm 3.69 (br. s., 6 H), 3.75 (s, 2 H), 3.79 - 3.86 (m, 2 H), 6.94 - 6.98 (m, 1 H), 7.07 - 7.13 (m, 1 H), 7.53 -7.58 (m, 1 H), 7.93 - 7.99 (m, 1 H), 8.00 - 8.06 (m, 1 H), 8.80 (s, 1 H) | 418, 420 |
| 10241 | 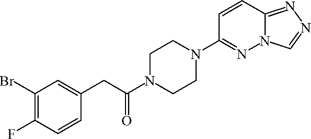 [SMILES: O=C(Cc1ccc(F)c(Br)c1)N1CCN(c2ccc3nncn3n2)CC1] | White solid, 14 mg, 16% | (CDCl₃) δ ppm 3.68 (br. s., 6 H), 3.74 (s, 2 H), 3.82 (br. s., 2 H), 6.94 (d, J=10.27 Hz, 1 H), 7.45 (d, J=5.62 Hz, 2 H), 7.49 (br. s., 2 H), 7.95 (d, J=9.90 Hz, 1 H), 8.80 (s, 1 H) | 418, 420 |
| 10442 | 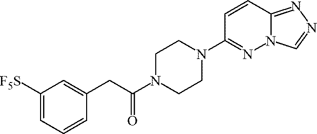 | Yellow solid, 76 mg, 45% | (CDC1₃) δ ppm 3.49 - 3.60 (m, 4 H), 3.66 (d, J=5.26 Hz, 2 H), 3.80 - 3.87 (m, 4 H), 6.94 (d, J=10.15 Hz, 1 H), 7.44 - 7.48 (m, 1H), 7.66 (s, 1H), 7.69 (d, J=6.85 Hz, 1H), 7.94 (d, J=10.15 Hz, 1H), 8.80 (s, 1H) | 448 |
| 10441 | 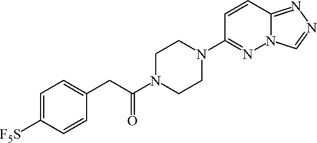 | Yellow solid, 45 mg, 26% | (CDCl₃) δ ppm 3.51 - 3.61 (m, 4 H), 3.62 - 3.69 (m, 2 H), 3.84 (s, 4 H), 6.94 (d, J=10.15 Hz, 1 H), 7.38 (d, J=8.56 Hz, 2H), 7.75 (d, J=8.68 Hz, 2H), 7.94 (s, 1H), 8.80 (s, 1H) | 448 |
| 10502 | 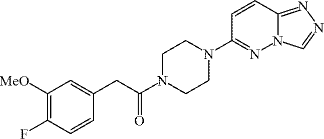 [SMILES: COc1cc(CC(=O)N2CCN(c3ccc4nncn4n3)CC2)ccc1F] | Yellow oil, 61 mg, 34% | (DMSO-d₆) δ 3.48 - 3.57 (m, 3 H), 3.59 - 3.69 (m, 4 H), 3.76 (s, 2 H), 3.81 (s, 3H), 6.98 -7.17 (m, 3 H), 7.38 - 7.46 (m, 1 H), 8.12 (d, J=10.15 Hz, 1 H), 9.27 (s, 1 H) | 370 |
| 10503 | 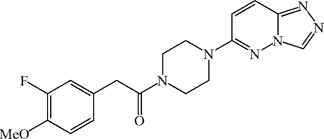 [SMILES: COc1ccc(CC(=O)N2CCN(c3ccc4nncn4n3)CC2)cc1F] | Yellow oil, 36 mg, 20% | (DMSO-d₆) δ 3.53 (d, J=3.30 Hz, 4 H), 3.58 - 3.68 (m, 4 H), 3.72 (s, 2 H), 3.81 (s, 3 H), 7.04 -7.13 (m, 3 H), 7.37 -7.45 (m, 1H), 8.12 (d, J=10.15 Hz, 1 H), 9.27 (s, 1 H) | 370 |
| 10504 | 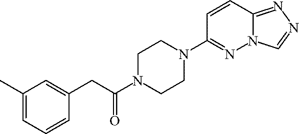 [SMILES: Cc1cccc(CC(=O)N2CCN(c3ccc4nncn4n3)CC2)c1] | Yellow oil, 19 mg, 12% | (DMSO-d₆) δ 2.28 (s, 3 H), 3.48 - 3.56 (m, 4 H), 3.58 -3.66 (m, 4 H), 3.72 - 3.75 (m, 2 H), 7.01 - 7.07 (m, 2 H), 7.16 - 7.22 (m, 1 H), 7.35 -7.40 (m, 1H), 8.07 - 8.13 (m, 1 H), 9.21 - 9.25 (m, 1 H) | 336 |
| 10505 | 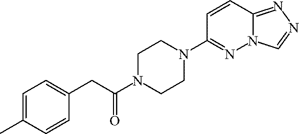 [SMILES: Cc1ccc(CC(=O)N2CCN(c3ccc4nncn4n3)CC2)cc1] | White solid, 14 mg, 9% | (DMSO-d₆) δ 2.27 (s, 3H), 3.43-3.55 (m, 4H), 3.61 (br.s., 4H), 3.72 (s, 2H), 7.12 (s, 4H), 7.39 (s, 1H), 8.10 (d, J=10.15 Hz, 1 H), 9.24 (s, 1 H) | 336 |

### Preparation of 2-{[1,1′-biphenyl]-4-yl}-1-(4-{[1,2,4]triazolo[4,3-b]pyridazin-6-yl}piperazin-1-yl)ethan-1-one (Example 2809).

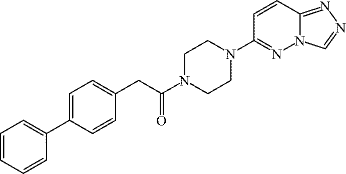 [SMILES: O=C(Cc1ccc(-c2ccccc2)cc1)N1CCN(c2ccc3nncn3n2)CC1]

Compound 2767 (50 mg, 0.13 mmol) was added to a microwave vial along with phenyl boronic acid (122 mg, 0.25 mmol), potassium carbonate (69 mg, 0.50 mmol), and Palladium tetrakis(triphenyl phosphine) (14 mg, 0.01 mmol). The mixture was dissolved in 4 mL of dioxane and 2 mL of water, sealed, degassed with nitrogen for twenty minutes, and microwaved at 100° C. for 2 hours. The reaction mixture was filtered through celite, rinsing with methanol. Most of the solvent was removed in vacuo. The remaining oil was extracted in ethyl acetate, rinsing two times with water and one time with brine. The organic layer was dried over magnesium sulfate, filtered, and concentrated. The remaining oil was purified via Reverse Phase Flash Chromatography (5➙95% CH₃CN/H₂O) to give the title compound. LC-MS: m/z 398.2 (MH)⁺. ¹H NMR (400 MHz, DMSO-d6) δ ppm 3.54 (br. s., 4 H), 3.66 (d, J=17.12 Hz, 4 H), 3.83 (s, 2 H), 7.30 - 7.41 (m, 4 H), 7.63 (dd, J=14.06, 7.83 Hz, 5 H), 8.10 (d, J=10.15 Hz, 1 H), 9.22 (s, 1 H).

### Preparation of Ethyl 2-[4-(3-{[(tert-butoxy)carbonyl]amino}prop-1-yn-1-yl)phenyl]acetate.

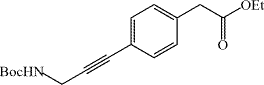 [SMILES: CCOC(=O)Cc1ccc(C#CCNC(=O)OC(C)(C)C)cc1]

Ethyl 2-(4-iodophenyl)acetate (1.25 mL, 6.90 mmol), tert-butyl N-(prop-2-yn-1-yl)carbamate (1.17 g, 7.59 mmol), bis(triphenylphosphine) palladium dichloride (245 mg, 0.35 mmol), and copper iodide (66 mg, 0.35 mmol) was dissolved in 10 mL of anhydrous THF, sealed with a septum, and degassed with nitrogen for 20 minutes. To the reaction mixture was added 5 mL of di-isopropyl amine. The mixture was allowed to stir at room temp overnight. The solvent was removed in vacuo, and the reaction mixture was filtered through celite, rinsing with methanol. The methanol was removed in vacuo and the remaining oil was extracted with ethyl acetate, rinsing twice with water and once with brine. The organic layer was dried over magnesium sulfate, filtered and concentrated in vacuo. The remaining oil was purified via Normal Phase flash chromatography (0-35% Ethyl Acetate/Hexanes) to give the title compound (1.00 g, 46%). LC-MS: m/z 317.2 (MH)⁺.

### Preparation of 2-[4-(3-{[(tert-butoxy)carbonyl]amino}prop-1-yn-1-yl)phenyl]acetic acid.

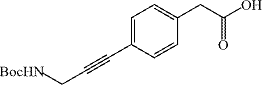 [SMILES: CC(C)(C)OC(=O)NCC#Cc1ccc(CC(=O)O)cc1]

Ethyl 2-[4-(3-{[(tert-butoxy)carbonyl]amino}prop-1-yn-1-yl)phenyl]acetate was dissolved in 10 mL of 1 M LiOH and 5 mL of ethanol. The reaction mixture was allowed to stir for two hours at room temp. The ethanol was removed in vacuo and the remaining solution was neutralized with 6 M HCl, then extracted in DCM three times. The organic layer was dried over magnesium sulfate, filtered, and concentrated to give the title compound (720 mg, 79%).

### Preparation of Tert-butyl N-(3-{4-[2-oxo-2-(4-{[1,2,4]triazolo[4,3-b]pyridazin-6-yl}piperazin-1-yl)ethyl]phenyl}prop-2-yn-1-yl)carbamate (Example 2811).

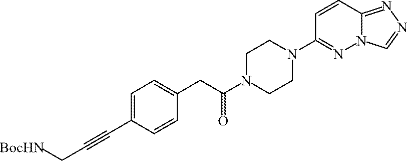 [SMILES: CC(C)(C)OC(=O)NCC#Cc1ccc(CC(=O)N2CCN(c3ccc4nncn4n3)CC2)cc1]

General Method B was followed using 2-[4-(3-{[(tert-butoxy)carbonyl]amino}prop-l-yn-l-yl)phenyl]acetic acid (200 mg, 0.70 mmol) to give the title compound (220 mg, 66%). LC-MS: m/z 475.2 (MH)⁺. ¹H NMR (400 MHz, DMSO-d6) δ ppm 1.39 (s, 9H), 3.46-3.57 (m, 4H), 3.57-3.67 (m, 4H), 3.80 (s, 1H), 3.94-3.98 (m, 1H), 7.25 (d, J=8.31 Hz, 2H), 7.34 (d, J=8.19 Hz, 2H), 7.38(d, J=10.03 Hz, 1H), 8.08-8.13 (m, 1H), 9.23 (s, 1H).

### Preparation of 2-[4-(3-aminoprop-1-yn-1-yl)phenyl]-1-(4- { [1,2,4]triazolo[4,3-b]pyridazin-6-yl}piperazin-1-yl)ethan-1-one (Example 10037).

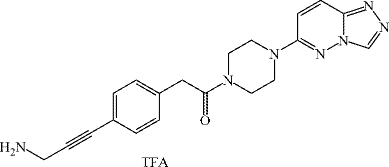 [SMILES: NCC#Cc1ccc(CC(=O)N2CCN(c3ccc4nncn4n3)CC2)cc1 ; "TFA"]

Tert-butyl N-(3-{4-[2-oxo-2-(4-{[1,2,4]triazolo[4,3-b]pyridazin-6-yl}piperazin-1-yl)ethyl]phenyl}prop-2-yn-1-yl)carbamate (Example 2811) (200 mg, 0.42 mmol) was dissolved in 5 mL of acetonitrile, and 5 mL of trifluoroacetic acid was added. The mixture was allowed to stir at room temperature for two hours. The solvent and excess TFA was removed in vacuo to give the title compound as the TFA salt (323 mg, quant.). LC-MS: m/z 375.2 (MH)⁺. ¹H NMR (400 MHz, DMSO-d6) δ ppm 3.54 (d, J=3.42 Hz, 4 H), 3.58 - 3.63 (m, 4 H), 3.66 (d, J=5.38 Hz, 2 H), 3.83 (s, 2 H), 7.29 (d, J=8.19 Hz, 2 H), 7.39-7.42 (m, 2H), 7.42-7.45 (m, 1H), 8.14 (d, J=10.27 Hz, 1H), 8.22-8.37 (m, 2H), 9.28 (s, 1H).

### Preparation of 2-[4-(3- { [(tert-butoxy)carbonyl]amino}propyl)phenyl]acetic acid.

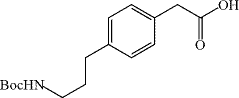 [SMILES: CC(C)(C)OC(=O)NCCCc1ccc(CC(=O)O)cc1]

2-(3-{[(tert-butoxy)carbonyl]amino}prop-1-yn-1-yl)phenyl]acetic acid (100 mg, 0.35 mmol) was dissolved in methanol, along with 10 mg of 10% Pd on Carbon. The reaction flask was pressurized with H₂ gas and shaken in a Paar Hydrogenator for three days. The reaction mixture was filtered through celite, rinsing with methanol. The solvent was removed in vacuo to give the title compound (71 mg, 70%).

### Preparation of Tert-butyl N-(3-{4-[2-oxo-2-(4-{[1,2,4]triazolo[4,3-b]pyridazin-6-yl}piperazin-1-yl)ethyl]phenyl}propyl)carbamate (Example 10242)

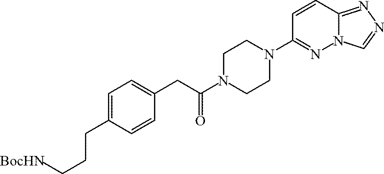 [SMILES: CC(C)(C)OC(=O)NCCCc1ccc(CC(=O)N2CCN(c3ccc4nncn4n3)CC2)cc1]

General Method B was followed using 2-[4-(3-{[(tertbutoxy)carbonyl]amino}propyl)phenyl]acetic acid (70 mg, 0.24 mmol) to give the title compound (13 mg, 11%). LC-MS: m/z 479.3 (MH)⁺. ¹H NMR (400 MHz, DMSO-d6) δ ppm 1.36 (s, 9H), 1.59-1.69 (m, 2H), 2.91 (d, J=5.87 Hz, 2H), 3.43-3.56 (m, 4H), 3.62 (br.s., 4H), 3.73 (s, 2 H), 6.85 (br.s., 1H), 7.07-7.18 (m, 4H), 7.40 (d, J=10.27 Hz, 1H), 8.11 (d, J=10.15 Hz, 1H), 9.26 (s, 1H).

### Preparation of 1-(4-{3-benzyl-[1,2,4]triazolo[4,3-b]pyridazin-6-yl}piperazin-1-yl)-2-(3,4-dichlorophenyl)ethan-1-one (Example 10249).

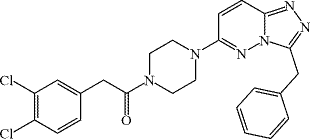 [SMILES: O=C(Cc1ccc(Cl)c(Cl)c1)N1CCN(c2ccc3nnc(Cc4ccccc4)n3n2)CC1]

General Method B was followed to couple Intermediate 5e (228 mg, 0.58 mmol) with 2-(3,4-dichlorophenyl)acetic acid (143 mg, 0.70 mmol) to give the title compound (80 mg, 29%). LC-MS: m/z 480 (MH)⁺, 482, 484. ¹H NMR (400 MHz, DMSO-d6) δ ppm 3.58 (d, J=8.56 Hz, 4 H) 3.62 - 3.68 (m, 4 H) 3.80 - 3.85 (m, 2 H) 4.37 (s, 2 H) 7.18 - 7.31 (m, 5 H) 7.51 -7.55 (m, 1H), 7.55-7.59 (m, 2H), 8.08 (d, J=10.15 Hz, 1H).

### General Procedure for Amide Couplings to Prepare Examples 2631-2634, 2720-2721, 2746-2747, 2479, 2752, 2755, 2765-2766, 2797-2800, 2817-2819, 10262, 10422, 10479-10485, 10495, 10549-10551 (Scheme 1, Step 5) (General Method C).

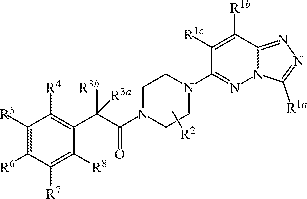

A suspension of Intermediate 5a or 5b or 5f (0.42 mmol, 1 eq) and the requisite aryl acetic acid 6 (0.68 mmol, 1.6 eq) in DMF (5 mL) was treated with DIEA (0.05 mL, 1 eq) and HBTU (0.68 mmol, 1.6 eq) at room temp. After 2 h, the mixture was then concentrated under vacuum and extracted using ethyl acetate (30 mL) three times and washed with satd NaHCO₃. The organic fraction was then dried over Na₂SO₄, filtered, and concentrated under vacuum. The crude product was first purified by reverse phase HPLC (5➙95% CH₃CN/H₂O/0.05% TFA), followed by a second purification using silica gel chromatography (0➙30% methanol/ethyl acetate) to give the final product. See Table 3 for MS and NMR characterization.

TABLE 3

Characterization of Examples 2631-2634, 2720-2721, 2746-2747, 2479, 2752, 2755, 2765-2766, 2797-2800, 2817-2819, 10262, 10422, 10479-10485, 10495, 10549-10551.

| Ex. | Structure | Yield | ¹H NMR (400 MHz) | LC-MS m/z (MH)⁺ |
|---|---|---|---|---|
| 2631 | 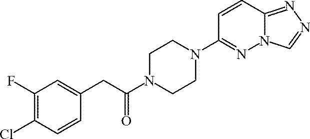 [SMILES: O=C(Cc1ccc(Cl)c(F)c1)N1CCN(c2ccc3nncn3n2)CC1] | light yellow solid, 33.5 mg, 90% | (CDCl₃) □ 3.46 - 3.53 (m, 2H), 3.54 - 3.60 (m, 2H), 3.61 - 3.66 (m, 2H), 3.76 (s, 2H), 3.80 - 3.85 (m, 2H), 6.92 (d, J=10.0, 1H), 6.98 - 7.05 (m, 1H), 7.07 -7.14 (m, 1H), 7.34 - 7.41 (m, 1H), 7.91 - 7.96 (m, 1H), 8.80 (d, J=0.7, 1H) | 375 |
| 2632 | 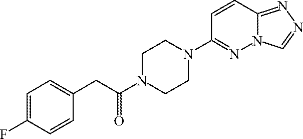 [SMILES: O=C(Cc1ccc(F)cc1)N1CCN(c2ccc3nncn3n2)CC1] | yellow solid, 22.8 mg, 35% | (CDCl₃) □ 3.48 - 3.60 (m, 4H), 3.64 (br.s., 2H), 3.74 (s, 2H), 3.82 (d, J=4.9, 2H), 6.93 (d, J=10.0, 1H), 7.10 -7.16 (m, 1H), 7.36 -7.46 (m, 2H), 7.93 (d, J=10.5, 1H), 8.80 (s, 1H) | 341 |
| 2633 | 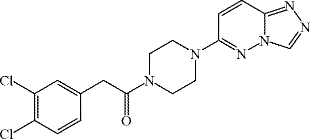 [SMILES: O=C(Cc1ccc(Cl)c(Cl)c1)N1CCN(c2ccc3nncn3n2)CC1] | 78 mg, yellow white solid, 30% | (CDCl₃) □ 8.80 (d, J= 0.7, 1H), 7.91 - 7.96 (m, 1H), 7.35 - 7.40 (m, 1H), 7.07 - 7.12 (m, 1H), 6.99 -7.03 (m, 1H), 6.92 (d,J= 10.0, 1H), 3.76 (s, 4H), 3.61 - 3.65 (m, 2H), 3.53 -3.58 (m, 2H), 3.48 - 3.52 (m, 2H) | 391 |
| 2634 | 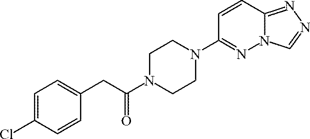 [SMILES: O=C(Cc1ccc(Cl)cc1)N1CCN(c2ccc3nncn3n2)CC1] | yellow-white solid, 8 mg, 18% | (CDCl₃) □ 8.79 (d, J = 0.7, 1H), 7.92 (dd, J = 0.6, 10.1, 1H), 7.30 - 7.36 (m, 1H), 7.18 - 7.24 (m, 1H), 6.91 (d, J= 10.0, 1H), 3.78 - 3.85 (m, 1H), 3.77 (s, 1H), 3.58 - 3.65 (m, 1H), 3.50 - 3.57 (m, 1H), 3.41 -3.47 (m, 1H) | 357 |
| 2720 | 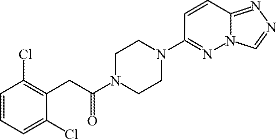 [SMILES: O=C(Cc1c(Cl)cccc1Cl)N1CCN(c2ccc3nncn3n2)CC1] | white solid, 16 mg, 10% | (DMSO-d₆) δ 3.52 - 3.73 (m, 6H), 3.77 - 3.88 (m, 2H), 4.06 (s, 2H), 7.26 -7.38 (m, 1H), 7.46 (d, J=8.1, 3H), 8.04 - 8.20 (m, 1H), 9.25 (s, 1H) | 391 |
| 2721 | 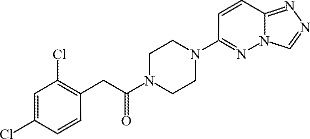 [SMILES: O=C(Cc1ccc(Cl)cc1Cl)N1CCN(c2ccc3nncn3n2)CC1] | yellow solid, 20.2 mg, 13% | (DMSO-d₆) δ 3.54 - 3.75 (m, 8H), 3.89 (s, 2H), 7.32 - 7.46 (m, 3H), 7.60 (dd, J=6.4, 1.7, 1H), 8.13 (d, J=10.0, 1H), 9.27 (s, 1H) | 391 |
| 2746 | 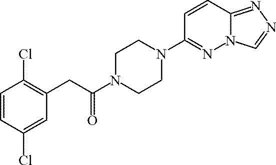 [SMILES: O=C(Cc1cc(Cl)ccc1Cl)N1CCN(c2ccc3nncn3n2)CC1] | white solid, 20.2 mg, 12% | (DMSO-d₆) δ 9.25 (s, 1 H), 8.06 - 8.27 (m, 1 H), 7.20 - 7.64 (m, 4 H), 3.91 (s, 2 H), 3.49 - 3.79 (m, 8 H) | 391 |
| 2747 | 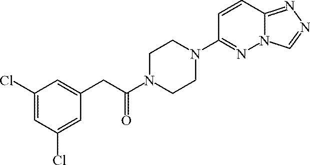 [SMILES: O=C(Cc1cc(Cl)cc(Cl)c1)N1CCN(c2ccc3nncn3n2)CC1] | white solid, 97.7 mg, 60% | (DMSO-d₆) δ 3.56 (d, J=5.9, 2H), 3.60 (br.s., 4H), 3.68 (d, J=4.7, 2H), 3.84 (s, 2H), 7.31 (s, 2H), 7.40 (d, J=10.3, 1H), 7.48 (s, 1H), 8.12 (d, J=10.3, 1H), 9.24 (s, 1H) | 391 |
| 2749 | 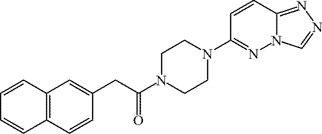 [SMILES: O=C(Cc1ccc2ccccc2c1)N1CCN(c2ccc3nncn3n2)CC1] | white solid, 72.8 mg, 50% | (DMSO-d₆) δ 3.47 - 3.58 (m, 4H), 3.62 - 3.73 (m, 4H), 3.96 (s, 2H), 7.37 (d, J=10.0, 2H), 7.48 (s, 2H), 7.76 (s, 1H), 7.86 (d, J=8.3, 3H), 8.09 (d, J=10.0, 1H), 9.22 (s, 1H) | 373 |
| 2752 | 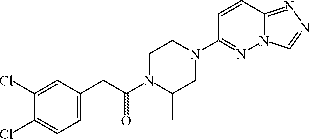 [SMILES: CC1CN(c2ccc3nncn3n2)CCN1C(=O)Cc1ccc(Cl)c(Cl)c1] | white solid, 42.3 mg, 27% | (DMSO-d₆) δ 1.09 (d, J=6.4, 3H), 2.82 - 3.13 (m, 2H), 3.17 - 3.28 (m, 1H), 3.38 - 3.51 (m, 1H), 3.80 (br.s., 2H), 3.86- 4.12 (m, 2H), 4.13 - 4.30 (m, 1H), 4.32 - 4.72 (m, 1H), 7.17 -7.29 (m, 1H), 7.34 - 7.43 (m, 1H), 7.56 (d, J--8.3, 2H), 8.10 (d, J= 10.0, 1H), 9.21 (s, 1H) | 405 |
| 2755 | 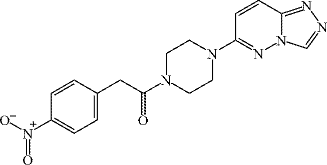 [SMILES: O=C(Cc1ccc([N+](=O)[O-])cc1)N1CCN(c2ccc3nncn3n2)CC1] | white solid, 20.2 mg, 14% | (700 MHz, DMSO-d₆) δ 3.51 - 3.75 (m, 8H), 3.98 (s, 2H), 5.75 (s, 1H), 7.34 - 7.44 (m, 1H), 7.48 - 7.57 (m, 1H), 8.08 - 8.14 (m, 1H), 8.16 - 8.22 (m, 2H), 9.23 (s, 1H) | 367 |
| 2765 | 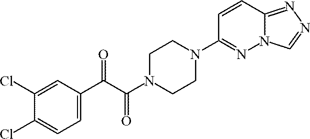 [SMILES: O=C(C(=O)N1CCN(c2ccc3nncn3n2)CC1)c1ccc(Cl)c(Cl)c1] | white solid, 32 mg, 35% | (DMSO-d₆) δ 3.45 - 3.61 (m, 4H), 3.77 (d, J=9.3, 4H), 7.40 (d, J=10.3, 1H), 7.91 (d, J=1.0, 1H), 8.08 -8.18 (m, 3H), 9.25 (s, 1H) | 405 |
| 2766 | 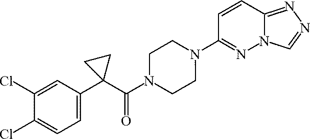 [SMILES: O=C(N1CCN(c2ccc3nncn3n2)CC1)C1(c2ccc(Cl)c(Cl)c2)CC1] | yellow solid, 93 mg, 57% | (DMSO-d₆) δ 1.24 - 1.42 (m, 4H), 3.55 (br.s., 8H), 7.20 (dd, J=8.6, 2.2, 1H), 7.32 (d, J=10.3, 1H), 7.38 (d, J=2.2, 1H), 7.56 (d, J=8.3, 1H), 8.09 (d, J=10.3, 1H), 9.20 (s, 1H) | 417 |
| 2797 | 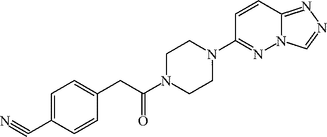 [SMILES: N#Cc1ccc(CC(=O)N2CCN(c3ccc4nncn4n3)CC2)cc1] | light yellow solid, 24.6 mg, 26% | (DMSO-d₆) δ 3.51 - 3.72 (m, 8H), 3.92 (s, 2H), 7.36 - 7.42 (m, 1H), 7.42 - 7.48 (m, 2H), 7.77 (s, 2H), 8.01 - 8.17 (m, 1H), 9.17 - 9.29 (m, 1H) | 348 |
| 2798 | 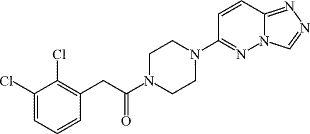 [SMILES: O=C(Cc1cccc(Cl)c1Cl)N1CCN(c2ccc3nncn3n2)CC1] | light brown solid, 36.7 mg, 24% | (DMSO-d₆) δ 3.51 - 3.77 (m, 8H), 3.96 (s, 2H), 7.31 (s, 2H), 7.39 - 7.44 (m, 1H), 7.51 - 7.57 (m, 1H), 8.09 - 8.15 (m, 1H), 9.24 (s, 1H) | 391 |
| 2799 | 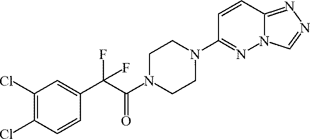 [SMILES: O=C(N1CCN(c2ccc3nncn3n2)CC1)C(F)(F)c1ccc(Cl)c(Cl)c1] | White solid, 64.3 mg, 13% | (DMSO-d₆) δ 3.45 - 3.61 (m, 4H) , 3.77 (d, J=9.8, 4H), 7.39 (d, J=10.0, 1H), 7.91 (d, J=0.7, 2H), 8.09 -8.19 (m, 2H), 9.24 (d, J=0.5, 1H). ¹⁹F NMR (376 MHz, DMSO-d₆) δ -93.22 (s, 2F) | 427 |
| 2800 | 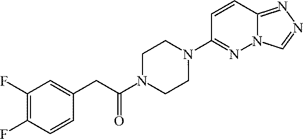 [SMILES: O=C(Cc1ccc(F)c(F)c1)N1CCN(c2ccc3nncn3n2)CC1] | Light yellow solid, 84.3 mg, 47% | (DMSO-d₆) δ 3.49 - 3.71 (m, 8H), 3.80 (s, 2H), 7.08 (br.s., 1H), 7.26 - 7.31 (m, 1H), 7.31 - 7.36 (m, 1H), 7.36 - 7.43 (m, 1H), 8.11 (d, J=10.0, 1H), 9.23 (s, 1H) | 359 |
| 2818 | 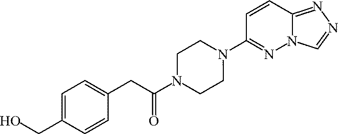 [SMILES: O=C(Cc1ccc(CO)cc1)N1CCN(c2ccc3nncn3n2)CC1] | Yellow solid, 100 mg, 27% | (DMSO-d₆) δ 3.34 - 3.89 (m, 11 H) 4.46 (br. s., 2 H) 7.22 (d, J=14.92 Hz, 4 H) 7.43 (d, J=9.29 Hz, 1 H) 8.12 (d, J=9.54 Hz, 1 H) 9.31 (br. s., 1 H) | 353 |
| 2819 | 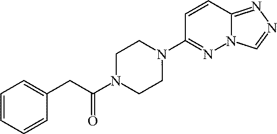 [SMILES: O=C(Cc1ccccc1)N1CCN(c2ccc3nncn3n2)CC1] | Light yellow solid, 18 mg, 13% | (DMSO-d₆) δ 9.22 (s, 1 H) , 8.10 (d, J=10.03 Hz, 1 H), 7.37 (d, J=10.27 Hz, 1 H), 7.28 - 7.35 (m, 2 H), 7.19 - 7.27 (m, 2 H), 3.78 (s, 1 H), 3.49 - 3.69 (m, 8 H) | 323 |
| 10262 | 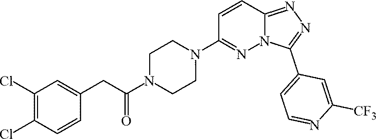 [SMILES: O=C(Cc1ccc(Cl)c(Cl)c1)N1CCN(c2ccc3nnc(-c4ccnc(C(F)(F)F)c4)n3n2)CC1] | White solid, 29 mg, 25 % | (DMSO-d₆) δ 3.62 - 3.77 (m, 8 H) 3.86 (s, 2 H) 7.22 - 7.29 (m, 1H) 7.52 - 7.61 (m, 3 H) 8.30 (s, 1 H) 8.66 - 8.71 (m, 1 H) 8.87 (s, 1 H) 8.97 - 9.03 (m, 1 H) | 536 |
| 10422 | 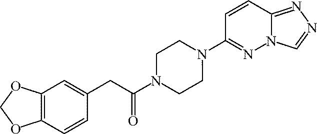 [SMILES: O=C(Cc1ccc2c(c1)OCO2)N1CCN(c2ccc3nncn3n2)CC1] | White solid, 101 mg, 33 % | (DMSO-d₆) δ 3.51 (d, J=4.89 Hz, 4 H) 3.57 -3.66 (m, 4 H) 3.68 (s, 2 H) 5.98 (s, 2 H) 6.71 (d, J=8.07 Hz, 1 H) 6.77 -6.89 (m, 2 H) 7.38 (d, J=10.03 Hz, 1 H) 8.10 (d, J=10.03 Hz, 1 H) 9.23 (d, J=0.73 Hz, 1 H) | 367 |
| 10479 | 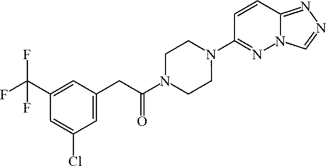 [SMILES: O=C(Cc1cc(Cl)cc(C(F)(F)F)c1)N1CCN(c2ccc3nncn3n2)CC1] | 200 mg, white solid, 48 % | (DMSO-d₆) δ 9.25 (d, J=0.7 Hz, 1 H), 8.13 (d, J=10.3 Hz, 1 H), 7.73 (s, 1 H), 7.62 (d, J=18.1 Hz, 2 H), 7.41 (d, J=10.0 Hz, 1 H), 3.95 (s, 2 H), 3.51 -3.75 (m, 8 H) | 425 |
| 10480 | 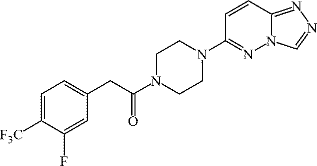 [SMILES: O=C(Cc1ccc(C(F)(F)F)c(F)c1)N1CCN(c2ccc3nncn3n2)CC1] | white solid , 118 mg, 30% | (DMSO-d₆) δ 9.26 (d, J=0.7 Hz, 1 H), 8.13 (dd, J=10.0, 0.7 Hz, 1 H), 7.72 (t, J=8.1 Hz, 1 H), 7.42 (d, J=10.3 Hz, 1 H), 7.37 (s, 1 H), 7.29 (d, J=7.8 Hz, 1 H), 3.94 (s, 2 H), 3.52 -3.79 (m, 8 H) | 409 |
| 10481 | 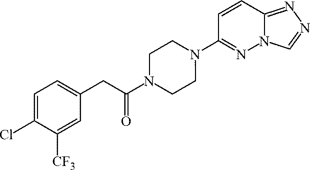 [SMILES: O=C(Cc1ccc(Cl)c(C(F)(F)F)c1)N1CCN(c2ccc3nncn3n2)CC1] | Cream white solid, 170 mg, 48 % | (DMSO-d₆) δ 9.23 (d, J=0.7 Hz, 1 H), 8.12 (dd, J=10.0, 0.7 Hz, 1 H), 7.40 (d, J=10.0 Hz, 1 H), 7.26 -7.33 (m, 1 H), 7.19 (s, 1 H), 7.05 - 7.13 (m, 1 H), 3.84 (s, 2 H), 3.57 (d, J=5.1 Hz, 8 H) | 425 |
| 10482 | 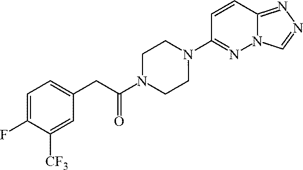 [SMILES: O=C(Cc1ccc(F)c(C(F)(F)F)c1)N1CCN(c2ccc3nncn3n2)CC1] | White solid, 228 mg, 67% | (DMSO-d₆) δ 9.23 (d, J=0.7 Hz, 1 H), 8.12 (dd, J=10.0, 0.7 Hz, 1 H), 7.40 (d, J=10.0 Hz, 1 H), 7.26 -7.33 (m, 1 H), 7.19 (s, 1 H), 7.05 - 7.13 (m, 1 H), 3.84 (s, 2 H), 3.57 (d, J=5.1 Hz, 8 H) | 409 |
| 10483 | 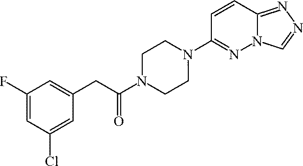 [SMILES: O=C(Cc1cc(F)cc(Cl)c1)N1CCN(c2ccc3nncn3n2)CC1] | White solid, 87 mg, 28 % | (DMSO-d₆) δ 9.26 (d, J=0.7 Hz, 1 H), 8.13 (dd, J=10.0, 0.7 Hz, 1 H), 7.72 (t, J=8.1 Hz, 1 H), 7.42 (d, J=10.3 Hz, 1 H), 7.37 (s, 1 H), 7.29 (d, J=7.8 Hz, 1 H), 3.94 (s, 2 H), 3.52 -3.79 (m, 8 H) | 375 |
| 10484 | 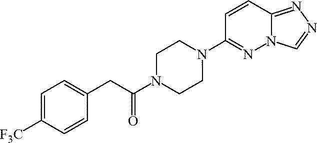 [SMILES: O=C(Cc1ccc(C(F)(F)F)cc1)N1CCN(c2ccc3nncn3n2)CC1] | White solid, 55 mg, 17 % | (DMSO-d₆) δ 9.26 (d, J=0.7 Hz, 1 H), 8.13 (dd, J=10.0, 0.7 Hz, 1 H), 7.72 (t, J=8.1 Hz, 1 H), 7.42 (d, J=10.3 Hz, 1 H), 7.37 (s, 1 H), 7.29 (d, J=7.8 Hz, 1 H), 3.94 (s, 2 H), 3.52 -3.79 (m, 8 H) | 391 |
| 10485 | 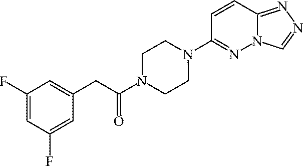 [SMILES: O=C(Cc1cc(F)cc(F)c1)N1CCN(c2ccc3nncn3n2)CC1] | White solid, 59 mg, 20 % | (DMSO-d₆) δ 9.26 (d, J=0.7 Hz, 1 H), 8.13 (dd, J=10.0, 0.7 Hz, 1 H), 7.72 (t, J=8.1 Hz, 1 H), 7.42 (d, J=10.3 Hz, 1 H), 7.37 (s, 1 H), 7.29 (d, J=7.8 Hz, 1 H), 3.94 (s, 2 H), 3.52 -3.79 (m, 8 H) | 359 |
| 10495 | 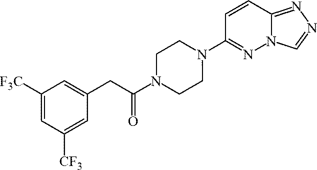 [SMILES: O=C(Cc1cc(C(F)(F)F)cc(C(F)(F)F)c1)N1CCN(c2ccc3nncn3n2)CC1] | White solid, 150 mg, 39% | (DMSO-d₆) δ 9.26 (d, J=0.7 Hz, 1 H), 8.13 (dd, J=10.0, 0.7 Hz, 1 H), 7.72 (t, J=8.1 Hz, 1 H), 7.42 (d, J=10.3 Hz, 1 H), 7.37 (s, 1 H), 7.29 (d, J=7.8 Hz, 1 H), 3.94 (s, 2 H), 3.52 -3.79 (m, 8 H) | 459 |
| 10549 | 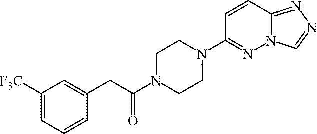 [SMILES: O=C(Cc1cccc(C(F)(F)F)c1)N1CCN(c2ccc3nncn3n2)CC1] | White solid, 12 mg, 4% | (DMSO-d₆) δ 9.22 - 9.25 (m, 1H), 8.07 - 8.16 (m, 1H), 7.52 - 7.64 (m, 3H), 7.34 - 7.44 (m, 1H), 3.89 -3.95 (m, 2H), 3.52 - 3.73 (m, 8H) | 391 |
| 10550 | 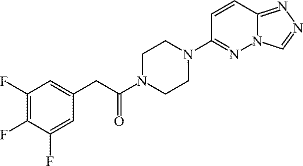 [SMILES: O=C(Cc1cc(F)c(F)c(F)c1)N1CCN(c2ccc3nncn3n2)CC1] | White solid, 92 mg, 29 % | (DMSO-d₆) δ 9.23 (s, 1H), 8.00 - 8.16 (m, 1H), 7.35 -7.45 (m, 1H), 7.14 - 7.26 (m, 2H), 3.81 (s, 2H), 3.50 - 3.72 (m, 8H) | 377 |
| 10551 | 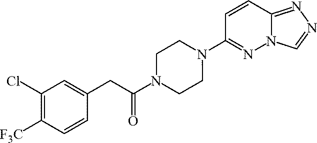 [SMILES: O=C(Cc1ccc(C(F)(F)F)c(Cl)c1)N1CCN(c2ccc3nncn3n2)CC1] | White solid, 16 mg, 5 % | (DMSO-d₆) δ 9.12 - 9.31 (m, 1H), 8.04 - 8.17 (m, 1H), 7.72 - 7.85 (m, 1H), 7.55 - 7.65 (m, 1H), 7.33 -7.48 (m, 2H), 3.93 (s, 2H), 3.50 - 3.76 (m, 8H) | 425 |

### Preparation of 2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one. (Example 2796)

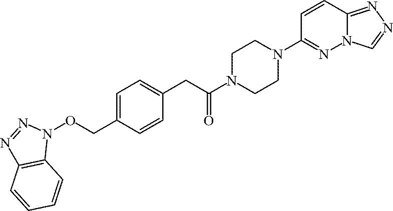 [SMILES: O=C(Cc1ccc(COn2nnc3ccccc32)cc1)N1CCN(c2ccc3nncn3n2)CC1]

A suspension of Intermediate 5a (50 mg, 0.20 mmol) and 4-bromomethylphenyl acetic acid, (68 mg, 3.0 mmol) was treated with DIEA (34 µL, 0.20 mmol) and HBTU (129.8 mg,0.34 mmol) at room temperature. After reaction period of 2 hours, the mixture was then concentrated under vacuum and extracted using ethyl acetate (30 mL) and wash with NaHCO₃. The extraction was done three times. The organic fraction was then dried over Na₂SO₄ under vacuum. The crude compound was purified by flash chromatography (0➔30% methanol/ethyl acetate) followed by reverse phase HPLC (5➔95% CH₃CN/H₂O/0.05% TFA) to give the title compound as a white solid compound (10 mg). ¹H NMR (400 MHz, D₂O) δ 3.59 (br. s., 8 H) 3.78 (s, 2 H) 5.57 (s, 2 H) 7.21 - 7.28 (m, 1 H) 7.32 - 7.44 (m, 2 H) 7.45 -7.57 (m, 1 H) 7.94 - 8.01 (m, 1 H) 8.06 - 8.15 (m, 1 H) 9.12 - 9.26 (m, 1 H). LC-MS: m/z 470 (MH)⁺.

### Preparation of 4-(2-(4-([1,2,4]triazolo[4,3-b]pyridazin-6-yl)piperazin-1-yl)-2-oxoethyl)benzyl 4-methylbenzenesulfonate.

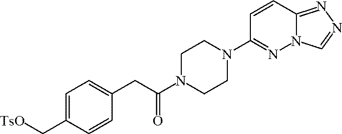 [SMILES: Cc1ccc(S(=O)(=O)OCc2ccc(CC(=O)N3CCN(c4ccc5nncn5n4)CC3)cc2)cc1]

2-(Hydroxymethyl)phenyl]-1-(4-{[1,2,4]triazolo[4,3-b]pyridazin-6-yl}piperazin-1-yl)ethan-1-one (Example 2818; 100 mg, 0.28 mmol) was weighed into an oven-dried round bottom flask with a stirring bar. 4 mL of DCM was added to dissolve the compound and the flask was cooled to 0° C. 79 µL (0.57 mmol) of triethylamine was added and stirred for 20 mins after which 65.1 mg (0.57 mmol) of TsCl was added and allowed to stir overnight. The mixture was then extracted using with water and EtOAc while washed with NH₄Cl, dried over Na₂SO₄, and concentrated to furnish the title compound as brown sticky solid (120 mg, 100%). LCMS m/z 431 (MH)⁺.

### Preparation of 2-{4-[(Morpholin-4-yl)methyl]phenyl}-1-(4-{[1,2,4]triazolo[4,3-b]pyridazin-6-yl}piperazin-1-yl)ethan-1-one (Example 2817).

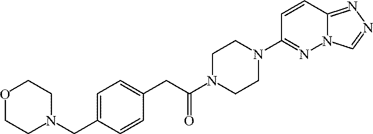 [SMILES: O=C(Cc1ccc(CN2CCOCC2)cc1)N1CCN(c2ccc3nncn3n2)CC1]

4-(4-([1,2,4]triazolo[4,3-b]pyridazin-6-yl)piperazin-1-yl)-2-oxoethyl)benzyl 4-methylbenzenesulfonate (125 mg, 0.29 mmol) was weighed into an oven-dried round bottom flask with a stirring bar. 4 mL of DCM was added to dissolve the compound with the flask was cooled to 0° C. in an ice bath. 82 µL (0.58 mmol) of triethylamine was added and stirred for 20 mins after which 50 µL (0.58 mmol) of morphine was added and allowed to stir overnight. The mixture is concentrated and purified by silica gel chromatography with 0➔30% methanol/ethyl acetate and further purified by reverse-phase HPLC (5➔95% CH₃CN/H₂O) to furnish the title compound as a white solid (20 mg, 0.048 mmol, 16%). ¹H NMR (400 MHz, DMSO-d₆) δ 9.22 (d, J = 0.73 Hz, 1H), 8.09 (s, 1H), 7.37 (d, J = 10.27 Hz, 1H), 7.22 (d, J = 9.54 Hz, 4H), 3.76 (s, 2H), 3.62 (br. s., 4H), 3.49 - 3.58 (m, 6H), 3.44 - 3.49 (m, 2H), 3.42 (s, 2H), 2.33 (d, J = 3.91 Hz, 4H). LC-MS: m/z 422 (MH)⁺.

### Preparation of Tert-butyl 4-[2-(3,4-dichlorophenyl)acetyl]piperazine-1-carboxylate.

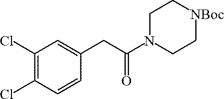 [SMILES: CC(C)(C)OC(=O)N1CCN(C(=O)Cc2ccc(Cl)c(Cl)c2)CC1]

General Method B was followed using 2-(3,4-dichlorophenyl)acetic acid (1.00 g, 4.90 mmol) and N-Boc-piperazine (1.09 g, 5.88 mmol) to give the title compound (1.57 g, 86%).

### Preparation of 2-(3,4-dichlorophenyl)-1-(piperazin-1-yl)ethan-1-one hydrochloride.

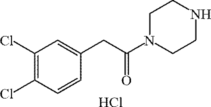 [SMILES: O=C(Cc1ccc(Cl)c(Cl)c1)N1CCNCC1 ; Cl]

tert-butyl 4-[2-(3,4-dichlorophenyl)acetyl]piperazine-1-carboxylate (1.50 g, 4.03 mmol) was dissolved in 5 mL of 4 M HCl in dioxane and stirred at room temperature for two hours. Dioxane and excess HCl were removed in vacuo to give the crude title compound.

### Preparation of 2-(3,4-dichlorophenyl)-1-(4-{3-phenyl-[1,2,4]triazolo[4,3-b]pyridazin-6-yl}piperazin-1-yl)ethan-1-one (Example 2807).

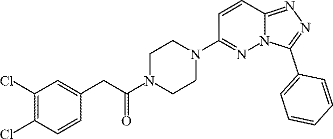 [SMILES: O=C(Cc1ccc(Cl)c(Cl)c1)N1CCN(c2ccc3nnc(-c4ccccc4)n3n2)CC1]

Intermediate 3d (65 mg, 0.28 mmol) and 2-(3,4-dichlorophenyl)-1-(piperazin-1-yl)ethan-1-one hydrochloride (85 mg, 0.31 mmol) was dissolved in ethanol and placed in a microwave vial, to which DIEA (0.08 mL, 0.42 mmol) was added. The vial was sealed and heated to 100° C. for 4 hours. Solvent was removed in vacuo, and the crude product was extracted in ethyl acetate, rinsing twice with water and once with brine. The organic layer was dried over magnesium sulfate, filtered, and concentrated. The crude product was purified via Normal Phase flash chromatography (0-20% MeOH/EtOAc) to give the title compound (45 mg, 31%). LC-MS: m/z 466.1 (MH)⁺, 468.1, 470.1. ¹H NMR (400 MHz, CHLOROFORM-d) δ ppm 3.59 (dd, J=11.25, 4.77 Hz, 4H), 3.68 (br.s., 2H), 3.76 (s, 2H), 3.87 (br.s., 2H), 6.95(d, J=10.03 Hz, 1H), 7.14 (d, J=8.31 Hz, 1H), 7.40 (s, 1H), 7.43 (d, J=8.31 Hz, 1H), 7.47-7.59 (m, 3H), 8.00 (d, J=10.03 Hz, 1H), 8.44 (d, J=7.34 Hz, 2H).

### Preparation of 2-(3,4-dichlorophenyl)-1-(4-{3-methyl-[1,2,4]triazolo[4,3-b]pyridazin-6-yl}piperazin-1-yl)ethan-1-one (Example 2808).

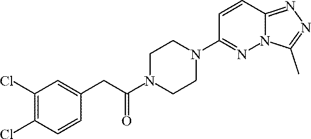 [SMILES: Cc1nnc2ccc(N3CCN(C(=O)Cc4ccc(Cl)c(Cl)c4)CC3)nn12]

Intermediate 3b (90 mg, 0.61 mmol) and 2-(3,4-dichlorophenyl)-1-(piperazin-1-yl)ethan-1-one hydrochloride (179 mg, 0.66 mmol) was dissolved in ethanol and placed in a microwave vial, to which DIEA (0.16 mL, 0.91 mmol) was added. The vial was sealed and heated to 100° C. for 4 hours. Solvent was removed in vacuo, and the crude product was extracted in ethyl acetate, rinsing twice with water and once with brine. The organic layer was dried over magnesium sulfate, filtered, and concentrated. The crude product was purified via Normal Phase flash chromatography (0-20% MeOH/EtOAc) to give the title compound (32 mg, 13%). LC-MS: m/z 404.1 (MH)⁺, 406.1, 408.1. ¹H NMR (400 MHz, CHLOROFORM-d) δ ppm 2.69 (s, 3H), 3.51-3.59 (m, 4H), 3.65 (br.s., 2H), 3.75 (s, 2H), 3.83 (br.s., 2H), 6.88 (d, J=10.15 Hz, 1 H), 7.13 (dd, J=6.24, 1.83 Hz, 1 H), 7.39 (d, J=1.83 Hz, 1 H), 7.43 (d, J=8.19 Hz, 1 H), 7.88 (d, J=10.03 Hz, 1 H).

### Preparation of 2-(3,4-dichlorophenyl)-1-{4-[3-(trifluoromethyl)-[1,2,4]triazolo[4,3-b]pyridazin-6-yl]piperazin-1-yl}ethan-1-one (Example 2810).

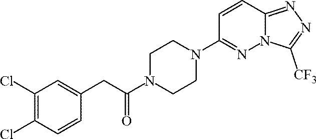 [SMILES: O=C(Cc1ccc(Cl)c(Cl)c1)N1CCN(c2ccc3nnc(C(F)(F)F)n3n2)CC1]

Intermediate 3c (100 mg, 0.50 mmol) and 2-(3,4-dichlorophenyl)-1-(piperazin-1-yl)ethan-1-one hydrochloride (147 mg, 0.54 mmol) was dissolved in ethanol and placed in a microwave vial, to which DIEA (0.13 mL, 0.74 mmol) was added. The vial was sealed and heated to 100° C. for 4 hours. Solvent was removed in vacuo, and the crude product was extracted in ethyl acetate, rinsing twice with water and once with brine. The organic layer was dried over magnesium sulfate, filtered, and concentrated. The crude product was purified via Normal Phase flash chromatography (0-20% MeOH/EtOAc) to give the title compound (64 mg, 28%). LC-MS: m/z 458.1 (MH)⁺, 460.1, 462.1. ¹H NMR (400 MHz, CHLOROFORM-d) δ ppm 3.59 - 3.69 (m, 6 H) 3.74 (s, 2 H) 3.83 (d, J=5.26 Hz, 2 H) 7.07 (d, J=10.15 Hz, 1 H) 7.12 (dd, J=8.25, 2.02 Hz, 1 H) 7.39 (d, J=1.96 Hz, 1 H) 7.43 (d, J=8.31 Hz, 1 H) 8.01 (d, J=10.27 Hz, 1 H).

### Preparation of Ethyl 2-(4-ethynylphenyl)acetate

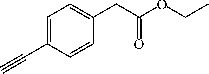

To a round bottom flask was added ethyl 2-(4-iodophenyl)acetate (1.25 mL, 6.90 mmol), bis(triphenylphosphine) Palladium dichloride (28 mg, 0.40 mmol), copper(I) iodide (7.5 mg, 0.40 mmol) and 10 mL of di-isopropyl amine. The mixture was then dissolved in 20 mL of THF and degassed with nitrogen. TMS-acetylene (0.94 mL, 7.65 mmol) was added to the solution, and stirred overnight at room temperature. The solvent and excess base was removed in vacuo and filtered through celite, rinsing with methanol. The methanol was removed in vacuo and the crude oil was dissolved in THF. To the solution was added 7 mL of 1 M TBAF in THF. The reaction mixture was stirred overnight to give the title compound (633 mg, 49%). LC-MS: m/z 188.1 (MH)⁺.

### Preparation of 2-(4-ethynylphenyl)acetic Acid

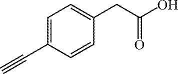

Ethyl 2-(4-ethynylphenyl)acetate was dissolved in 5 mL of ethanol. To the solution was added 5 mL of 1 M LiOH in water. The reaction mixture was stirred at room temperature for two hours. The ethanol was removed in vacuo and the remaining solution was neutralized with 6 M HCl, then extracted in DCM three times. The organic layer was dried over magnesium sulfate, filtered, and concentrated to give the title compound (607 mg, quant.).

### Preparation of Tert-butyl 4-[2-(4-ethynylphenyl)acetyl]piperazine-1-carboxylate.

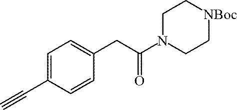 [SMILES: C#Cc1ccc(CC(=O)N2CCN(C(=O)OC(C)(C)C)CC2)cc1]

General Method B was followed using ethyl 2-(4-ethynylphenyl)acetic acid (281 mg, 1.76 mmol) and N-Boc-piperazine (392 mg, 2.11 mmol) to give the title compound (529 mg, 91%). LC-MS: m/z 328.2 (MH)⁺.

### Preparation of 2-(4-ethynylphenyl)-1-(piperazin-1-yl)ethan-1-one trifluoroacetate.

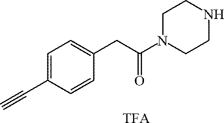 [SMILES: C#Cc1ccc(CC(=O)N2CCNCC2)cc1 ; "TFA"]

Tert-butyl 4-[2-(4-ethynylphenyl)acetyl]piperazine-1-carboxylate (529 mg, 1.62 mmol) was dissolved in 5 mL of trifluoroacetic acid and 5 mL of acetonitrile and stirred at room temperature for two hours. Solvent and excess TFA were removed in vacuo. The crude product was rinsed with DCM. The DCM was removed in vacuo, then the crude product was rinsed with diethyl ether, which was subsequently removed in vacuo. The crude product was rinsed again with diethyl ether. The diethyl ether was decanted, giving the title compound (318 mg, 60%). LC-MS: m/z 228.2 (MH)⁺.

### Preparation of 2-(4-ethynylphenyl)-1-(4-{[1,2,4]triazolo[4,3-b]pyridazin-6-yl}piperazin-1-yl)ethan-1-one (Example 10046).

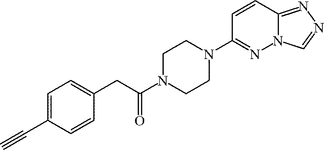 [SMILES: C#Cc1ccc(CC(=O)N2CCN(c3ccc4nncn4n3)CC2)cc1]

Intermediate 3a (130 mg, 0.84 mmol) and 2-(4-ethynylphenyl)-1-(piperazin-1-yl)ethan-1-one trifluoroacetate (318 mg, 0.93 mmol) was dissolved in ethanol and placed in a microwave vial, to which DIEA (0.30 mL, 1.69 mmol) was added. The vial was sealed and heated to 100° C. for 4 hours. Solvent was removed in vacuo, and the crude product was extracted in ethyl acetate, rinsing twice with water and once with brine. The organic layer was dried over magnesium sulfate, filtered, and concentrated. The crude oil was purified via Reverse Phase Flash Chromatography (5➔95% CH₃CN/H₂O) to give the title compound (16 mg, 5%). LC-MS: m/z 346.2 (MH)⁺. ¹H NMR (400 MHz, DMSO-d6) δ ppm 3.47-3.57 (m, 4H), 3.63 (d, J=4.40 Hz, 4H), 3.81 (s, 2H), 4.14 (s, 1H), 7.26 (d, J=8.31 Hz, 2H), 7.38 (d, J=10.27 Hz, 1 H), 7.42 (d, J=8.19 Hz, 2 H), 8.10 (d, J=10.15 Hz, 1 H), 9.23 (d, J=0.73 Hz, 1 H).

### Preparation of Tert-butyl N-[3-(5-bromo-2-fluorophenyl)prop-2-yn-1-yl]carbamate.

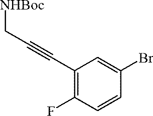 [SMILES: CC(C)(C)OC(=O)NCC#Cc1cc(Br)ccc1F]

4-bromofluoro-2-iodobenzene (900 mg, 3.0 mmol), tert-butyl N-(prop-2-yn-1-yl)carbamate (512 mg, 3.3 mmol), bis(triphenylphosphine) palladium dichloride (106 mg, 0.15 mmol), and copper(I) iodide (29 mg, 0.15 mmol) was dissolved in 5 mL of THF. To the solution was added 2.5 mL of di-isopropyl amine, which was then degassed with nitrogen. The mixture was allowed to stir overnight at room temp. The crude reaction mixture was filtered through celite, rinsing with methanol. The solvent was removed in vacuo and the crude oil was extracted in ethyl acetate, rinsing twice with water and once with brine. The organic layer was then dried over magnesium sulfate, filtered and concentrated to give the crude title compound (1.14 g).

### Preparation of Tert-butyl N-{3-[2-fluoro-5-(4,4,5,5-tetramethyl-1,3,2-dioxaborolan-2-yl)phenyl]prop-2-yn-1-yl} Carbamate.

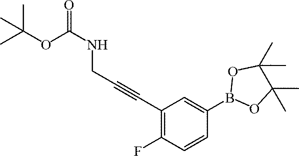 [SMILES: CC(C)(C)OC(=O)NCC#Cc1cc(B2OC(C)(C)C(C)(C)O2)ccc1F]

tert-butyl N-[3-(5-bromo-2-fluorophenyl)prop-2-yn-1-yl]carbamate (100 mg, 0.31 mmol), B₂Pin₂ (155 mg, 0.61 mmol), Pd(dppf)Cl₂-DCM (18 mg, 0.02 mmol), and Potassium Acetate (60 mg, 0.61 mmol) was dissolved in 6 mL of dioxane in a microwave vial, sealed, and degassed with nitrogen. The mixture was microwaved at 100° C. for 2 hours. The reaction mixture was filtered through a celite, rinsing with methanol. The solvent was removed in vacuo and the crude oil was extracted in ethyl acetate, rinsing twice with water and once with brine. The organic layer was dried over magnesium sulfate, filtered, and concentrated to give the crude title compound (228 mg). LC-MS: m/z 375.2 (MH)⁺.

### Preparation of 2-[3-(3-{[(tert-butoxy)carbonyl]amino}prop-1-yn-1-yl)-4-fluorophenyl]acetic Acid.

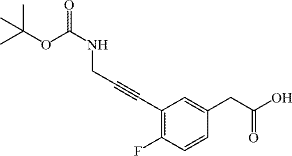 [SMILES: CC(C)(C)OC(=O)NCC#Cc1cc(CC(=O)O)ccc1F]

General Method A was followed using tert-butyl N-{3-[2-fluoro-5-(4,4,5,5-tetramethyl-1,3,2-dioxaborolan-2-yl)phenyl]prop-2-yn-1-yl}carbamate (228 mg, 0.31 mmol) to give the title compound (60 mg, 63%) as an off-white solid.

### Preparation of Tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate (Example 10443).

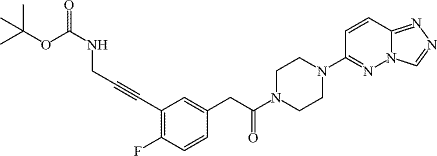 [SMILES: CC(C)(C)OC(=O)NCC#Cc1cc(CC(=O)N2CCN(c3ccc4nncn4n3)CC2)ccc1F]

General Method B was followed using 2-[3-(3-{[(tert-butoxy)carbonyl]amino}prop-1-yn-1-yl)-4-fluorophenyl]acetic acid to give the title compound (7 mg, 7%). LC-MS: m/z 493.2 (MH)⁺. ¹H NMR (400 MHz, CHLOROFORM-d) δ ppm 1.48 (s, 9H), 3.42-3.58 (m, 4H), 3.62 (br.s., 2H), 3.69-3.77 (m, 2H), 3.81 (d, J=5.14 Hz, 2H), 6.89-6.95 (m, 1H), 7.00-7.15 (m, 2H), 7.29-7.35 (m, 1H), 7.91 (s, 1H), 8.79 (d, J=1.83 Hz, 1H).

### Preparation of 4-(4-hydroxybenzoyl)phenyl Trifluoromethanesulfonate

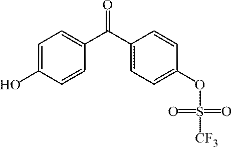 [SMILES: O=C(c1ccc(O)cc1)c1ccc(OS(=O)(=O)C(F)(F)F)cc1]

4,4′-dihydroxybenzophenone (3.00 g, 14.0 mmol) was dissolved in 100 mL of anhydrous DCM and pyridine was added. The solution was chilled to 0° C. and stirred for 10 minutes. To the chilled solution was added 1 M Triflic Anhydride in DCM (15.4 mL, 15.4 mmol). The solution was stirred and allowed to come to room temp overnight. The reaction mixture was poured into a separatory funnel and quenched with water. The organic layer was rinsed with water several times, dried over magnesium sulfate, filtered, and concentrated to give the crude title compound (1.83 g, 38%). LC-MS: m/z 346 (MH)⁺.

### Preparation of 4-[4-(4,4,5,5-tetramethyl-1,3,2-dioxaborolan-2-yl)benzoyl]phenol.

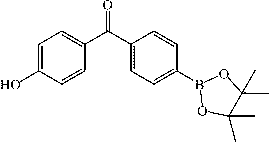 [SMILES: CC1(C)OB(c2ccc(C(=O)c3ccc(O)cc3)cc2)OC1(C)C]

4-hydroxybenzoyl)phenyl trifluoromethanesulfonate (1.83 g, 5.29 mmol), B₂Pin₂ (2.69 g, 10.59 mmol), Pd(dppf)Cl₂-DCM (212 mg, 0.26 mmol), and Potassium Acetate (828 mg, 10.59 mmol) was dissolved in 15 mL of dioxane in a microwave vial, sealed, and degassed with nitrogen. The mixture was microwaved at 100° C. for 2 hours. The reaction mixture was filtered through a celite, rinsing with methanol. The solvent was removed in vacuo and the crude oil was extracted in ethyl acetate, rinsing twice with water and once with brine. The organic layer was dried over magnesium sulfate, filtered, and concentrated to give the crude title compound (1.307 g, 76%). LC-MS: m/z 324.2 (MH)⁺.

### Preparation of Ethyl 2-[4-(4-hydroxybenzoyl)phenyl] Acetate

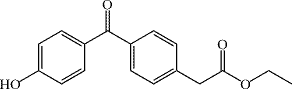 [SMILES: CCOC(=O)Cc1ccc(C(=O)c2ccc(O)cc2)cc1]

General Method A was followed, excluding the addition of LiOH, with 4-[4-(4,4,5,5-tetramethyl-1,3,2-dioxaborolan-2-yl)benzoyl]phenol (1.31 g, 1.04 mmol) to give the title compound (456 mg, 40%). LC-MS: m/z 284.1 (MH)⁺.

### Preparation of 2-[4-(4-hydroxybenzoyl)phenyl]acetic Acid

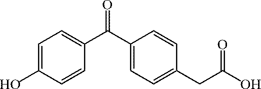 [SMILES: O=C(O)Cc1ccc(C(=O)c2ccc(O)cc2)cc1]

Ethyl 2-[4-(4-hydroxybenzoyl)phenyl] acetate (456 mg, 1.61 mmol) was dissolved in 5 mL of ethanol and 5 mL of 1 M LiOH. The solution was allowed to stir at room temp for 2 hours. The ethanol was removed in vacuo and the Acid/Base extraction procedure from General Method A to give the crude title compound (463 mg). LC-MS: m/z 256.1 (MH)⁺.

### Preparation of 2-[4-(4-hydroxybenzoyl)phenyl]-1-(4-{[1,2,4]triazolo[4,3-b]pyridazin-6-yl}piperazin-1-yl)ethan-1-one (Example 10500)

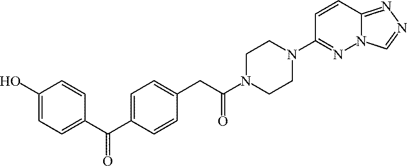 [SMILES: O=C(c1ccc(O)cc1)c1ccc(CC(=O)N2CCN(c3ccc4nncn4n3)CC2)cc1]

General Method B was followed using 2-[4-(4-hydroxybenzoyl)phenyl]acetic acid (463 mg, 1.81 mmol) to give the title compound (312 mg, 39%). LC-MS: m/z 442 (MH)⁺. ¹H NMR (400 MHz, DMSO-d6) δ ppm 3.55-3.69 (m, 8H), 5.06 (s, 2H), 7.07-7.12 (m, 2H), 7.42 (d, J=10.15 Hz, 1H), 7.52-7.58 (m, 2H), 7.62- 7.66 (m, 1H), 7.66-7.71 (m, 2H), 7.71-7.77 (m, 2H), 8.13 (d, J=10.15 Hz, 1H), 9.24 (d, J=0.73 Hz, 1H).

### Biological Assays.

In vitro EC₅₀ values for inhibition of C. parvum development in HCT-8 cells are given in Table 4.

TABLE 4

In vitro EC₅₀ values for inhibition of C. parvum development in HCT-8 cells.

| Example | EC₅₀ (µM) |
|---|---|
| 2178 | > 25.0 |
| 2257 | 17.1 |
| 2631 | 0.53 |
| 2632 | 1.18 |
| 2633 | 0.174 |
| 2634 | 0.88 |
| 2720 | > 25.0 |
| 2721 | > 25.0 |
| 2746 | 33 |
| 2747 | 0.99 |
| 2749 | 12.4 |
| 2752 | 7.44 |
| 2755 | 2.58 |
| 2756 | 1.26 |
| 2757 | 5.56 |
| 2758 | 0.292 |
| 2759 | 0.335 |
| 2760 | 2.29 |
| 2761 | 16.4 |
| 2762 | 16.4 |
| 2763 | 0.24 |
| 2764 | 3.61 |
| 2765 | 2.25 |
| 2766 | 8.33 |
| 2767 | 1.12 |
| 2796 | > 25.0 |
| 2797 | 11 |
| 2798 | > 25.0 |
| 2799 | 2.3 |
| 2800 | 0.76 |
| 2807 | 15 |
| 2808 | 30 |
| 2809 | > 25.0 |
| 2810 | 29 |
| 2811 | 12.8 |
| 2817 | > 25.0 |
| 2818 | > 25.0 |
| 2819 | 22 |
| 10037 | |
| 10046 | 0.883 |
| 10090 | > 50.0 |
| 10238 | 0.251 |
| 10239 | 0.121 |
| 10240 | 0.773 |
| 10241 | 0.232 |
| 10242 | > 50.0 |
| 10249 | 16 |
| 10262 | 6.11 |
| 10422 | 10.4 |
| 10441 | 11.5 |
| 10442 | 1.12 |
| 10443 | |
| 10479 | 0.407 |
| 10480 | 2 |
| 10481 | 0.272 |
| 10482 | 0.0687 |
| 10483 | 0.88 |
| 10484 | 5.45 |
| 10485 | 5.66 |

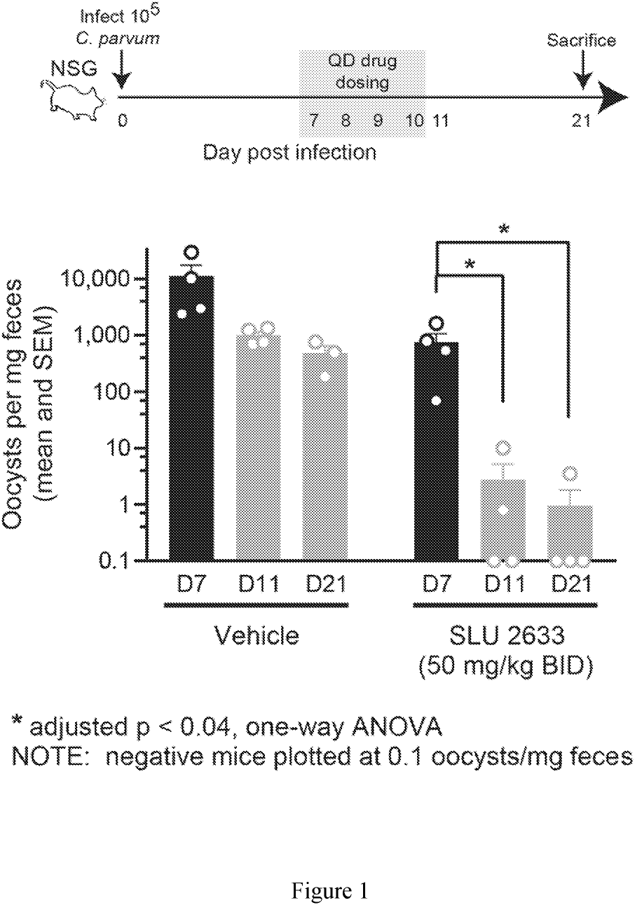

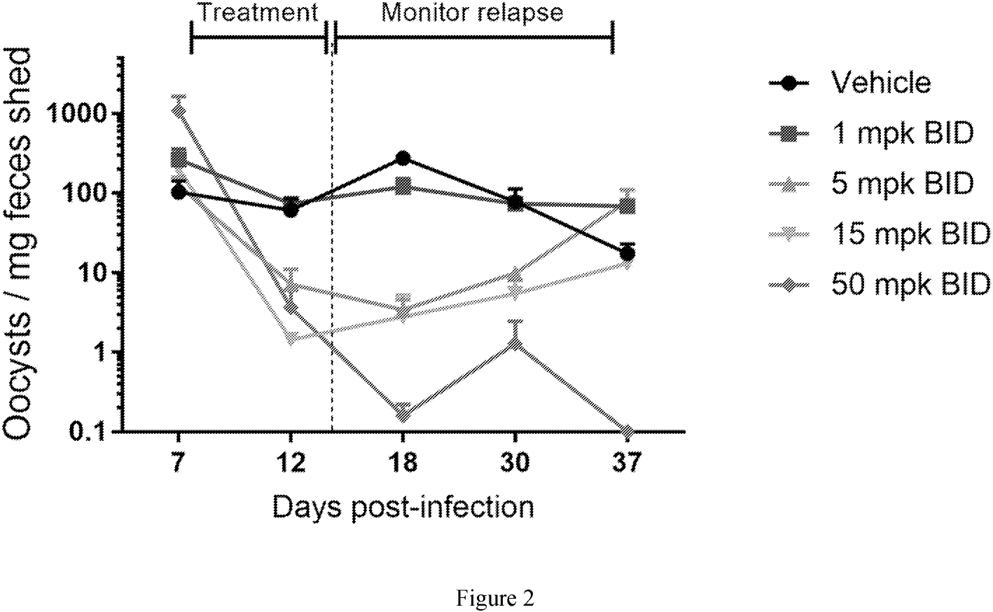

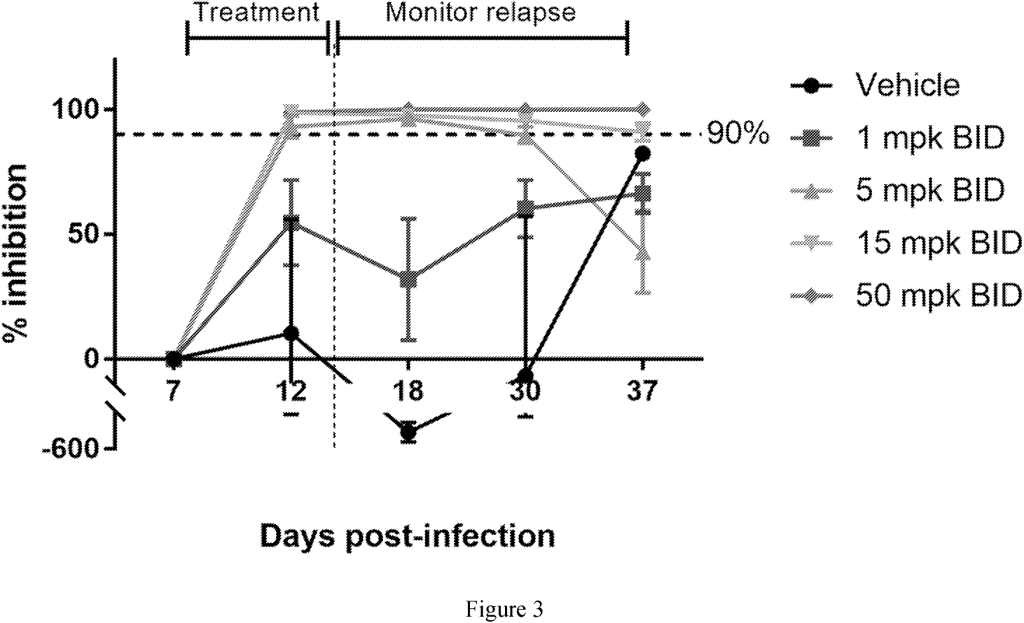

NOD SCID gamma immunocompromised mouse model of cryptosporidiosis. Results for treatment of C. parvum infected NOD SCID gamma mice with Example 2633 (SLU-0002633) by oral gavage at 50 mg/kg twice daily are shown in FIG. 1. Results for treatment of C. parvum infected NOD SCID gamma mice with Example 2633 by oral gavage at 1, 5, 15, and 50 mg/kg twice daily are shown in FIGS. 2 and 3.

TABLE 5

EC₅₀ (µM) of various compounds of the present disclosure.

| Name | Structure | IUPAC | Cp HTC-8 Avg EC50 (uM) |
|---|---|---|---|
| SLU-0010482 | 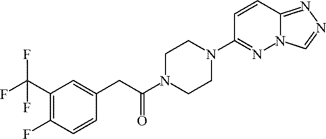 [SMILES: O=C(Cc1ccc(F)c(C(F)(F)F)c1)N1CCN(c2ccc3nncn3n2)CC1] | 2-[4-fluoro-3-(trifluoromethyl)phenyl] -1-(4-{[1,2,4]triazolo[4,3-b]pyridazin-6-yl}piperazin-1-yl)ethan-1-one | 0.0686 |
| SLU-0010239 | 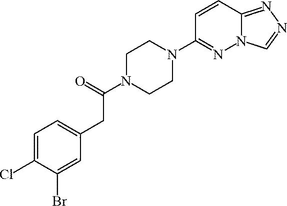 [SMILES: O=C(Cc1ccc(Cl)c(Br)c1)N1CCN(c2ccc3nncn3n2)CC1] | 2-(3-bromo-4-chlorophenyl)-1-(4-{[1,2,4]triazolo[4,3-b]pyridazin-6-yl}piperazin-1-yl)ethan-1-one | 0.121 |
| SLU-0002633 | 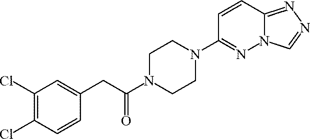 [SMILES: O=C(Cc1ccc(Cl)c(Cl)c1)N1CCN(c2ccc3nncn3n2)CC1] | 2-(3,4-dichlorophenyl)-1-(4-{[1,2,4]triazolo[4,3-b]pyridazin-6-yl}piperazin-1-yl)ethan-1-one | 0.174 |
| SLU-0010241 | 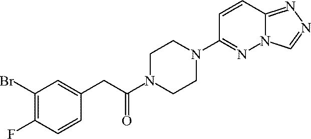 [SMILES: O=C(Cc1ccc(F)c(Br)c1)N1CCN(c2ccc3nncn3n2)CC1] | 2-(3-bromo-4-fluorophenyl)-1-(4-{[1,2,4]triazolo[4,3-b]pyridazin-6-yl}piperazin-1-yl)ethan-1-one | 0.232 |
| SLU-0002763 | 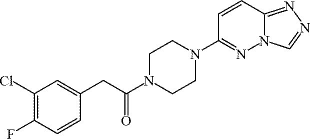 [SMILES: O=C(Cc1ccc(F)c(Cl)c1)N1CCN(c2ccc3nncn3n2)CC1] | 2-(3-chloro-4-fluorophenyl)-1-(4-{[1,2,4]triazolo[4,3-b]pyridazin-6-yl}piperazin-1-yl)ethan-1-one | 0.238 |
| SLU-0010238 | 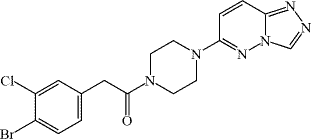 [SMILES: O=C(Cc1ccc(Br)c(Cl)c1)N1CCN(c2ccc3nncn3n2)CC1] | 2-(4-bromo-3-chlorophenyl)-1-(4-{[1,2,4]triazolo[4,3-b]pyridazin-6-yl}piperazin-1-yl)ethan-1-one | 0.251 |
| SLU-0010481 | 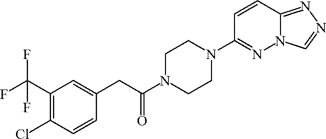 [SMILES: O=C(Cc1ccc(Cl)c(C(F)(F)F)c1)N1CCN(c2ccc3nncn3n2)CC1] | 2-[4-chloro-3-(trifluoromethyl)phenyl] -1-(4-{[1,2,4]triazolo[4,3-b]pyridazin-6-yl}piperazin-1-yl)ethan-1-one | 0.272 |
| SLU-0002758 | 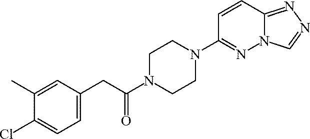 [SMILES: Cc1cc(CC(=O)N2CCN(c3ccc4nncn4n3)CC2)ccc1Cl] | 2-(4-chloro-3-methylphenyl)-1-(4-{[1,2,4]triazolo[4,3-b]pyridazin-6-yl}piperazin-1-yl)ethan-1-one | 0.279 |
| SLU-0010495 | 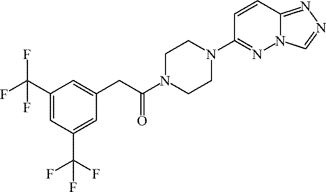 [SMILES: O=C(Cc1cc(C(F)(F)F)cc(C(F)(F)F)c1)N1CCN(c2ccc3nncn3n2)CC1] | 2-[3,5-bis(trifluoromethyl)phen yl]-1-(4-{[1,2,4]triazolo[4,3-b]pyridazin-6-yl}piperazin-1-yl)ethan-1-one | 0.319 |
| SLU- 0002759 | 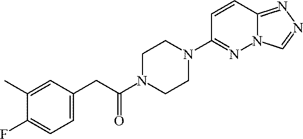 [SMILES: Cc1cc(CC(=O)N2CCN(c3ccc4nncn4n3)CC2)ccc1F] | 2-(4-fluoro-3-methylphenyl)-1-(4-{[1,2,4]triazolo[4,3-b]pyridazin-6-yl}piperazin-1-yl)ethan-1-one | 0.353 |
| SLU- 0010479 | 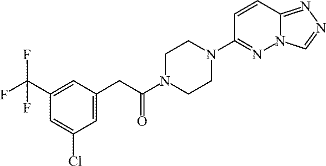 [SMILES: O=C(Cc1cc(Cl)cc(C(F)(F)F)c1)N1CCN(c2ccc3nncn3n2)CC1] | 2-[3-chloro-5-(trifluoromethyl)phenyl] -1-(4-{[1,2,4]triazolo[4,3-b]pyridazin-6-yl}piperazin-1-yl)ethan-1-one | 0.407 |
| SLU- 0002631 | 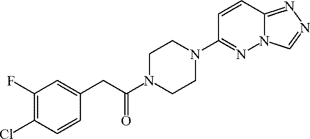 [SMILES: O=C(Cc1ccc(Cl)c(F)c1)N1CCN(c2ccc3nncn3n2)CC1] | 2-(4-chloro-3-fluorophenyl)-1-(4-{[1,2,4]triazolo[4,3-b]pyridazin-6-yl}piperazin-1-yl)ethan-1-one | 0.53 |
| SLU- 0002800 | 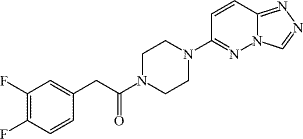 [SMILES: O=C(Cc1ccc(F)c(F)c1)N1CCN(c2ccc3nncn3n2)CC1] | 2-(3,4-difluorophenyl)-1-(4-{[1,2,4]triazolo[4,3-b]pyridazin-6-yl}piperazin-1-yl)ethan-1-one | 0.76 |
| SLU- 0010240 | 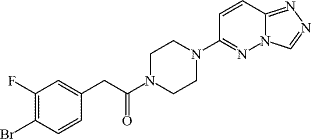 [SMILES: O=C(Cc1ccc(Br)c(F)c1)N1CCN(c2ccc3nncn3n2)CC1] | 2-(4-bromo-3-fluorophenyl)-1-(4-{[1,2,4]triazolo[4,3-b]pyridazin-6-yl}piperazin-1-yl)ethan-1-one | 0.773 |
| SLU-0002634 | 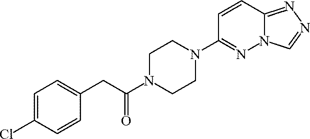 [SMILES: O=C(Cc1ccc(Cl)cc1)N1CCN(c2ccc3nncn3n2)CC1] | 2-(4-chlorophenyl)-1-(4-{[1,2,4]triazolo[4,3-b]pyridazin-6-yl}piperazin-1-yl)ethan-1-one | 0.88 |
| SLU-0010483 | 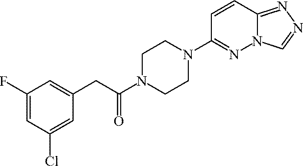 [SMILES: O=C(Cc1cc(F)cc(Cl)c1)N1CCN(c2ccc3nncn3n2)CC1] | 2-(3-chloro-5-fluorophenyl)-1-(4-{[1,2,4]triazolo[4,3-b]pyridazin-6-yl}piperazin-1-yl)ethan-1-one | 0.88 |
| SLU-0010046 | 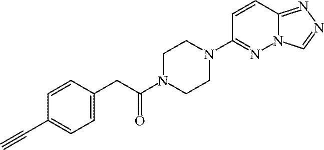 [SMILES: C#Cc1ccc(CC(=O)N2CCN(c3ccc4nncn4n3)CC2)cc1] | 2-(4-ethynylphenyl)-1 -(4-{[1,2,4]triazolo[4,3-b]pyridazin-6-yl}piperazin-1-yl)ethan-1-one | 0.883 |
| SLU-0002747 | 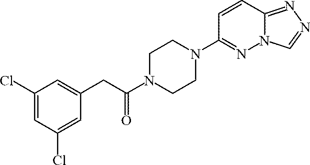 [SMILES: O=C(Cc1cc(Cl)cc(Cl)c1)N1CCN(c2ccc3nncn3n2)CC1] | 2-(3,5-dichlorophenyl)-1-(4-{[1,2,4]triazolo[4,3-b]pyridazin-6-yl}piperazin-1-yl)ethan-1-one | 0.99 |

Although the present disclosure has been described with respect to one or more particular embodiments and examples, it will be understood that other embodiments and examples of the present disclosure may be made without departing from the scope of the present disclosure.

1. A compound having the following structure: 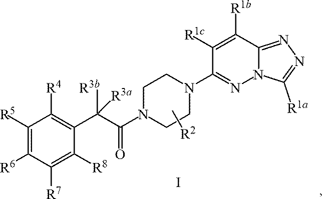
wherein
R^{1a}, R^{1b}, and R^{1c} are independently chosen from H, substituted alkyl groups, unsubstituted alkyl groups, substituted aryl groups, unsubstituted aryl groups, substituted aralkyl groups, and unsubstituted aralkyl groups;
R² is chosen from H, substituted alkyl groups, and unsubstituted alkyl groups;
R^{3a} and R^{3b} are independently chosen from H, substituted alkyl groups, unsubstituted alkyl groups, halogen, OH, NH₂, and R^{3a} and R^{3b} may combine to form a ring or a ═O;
R⁴ and R⁸ are independently chosen from H, halogens, substituted alkyl groups, and unsubstituted alkyl groups;
R⁵, R⁶, and R⁷ are independently chosen from H, halogens, substituted alkyl groups, unsubstituted alkyl groups, substituted cycloalkyl groups, unsubstituted cycloalkyl groups, substituted alkoxy groups, unsubstituted alkoxy groups, substituted aryl groups, unsubstituted aryl groups, substituted aralkyl groups, unsubstituted aralkyl groups, SO₂NR′R″, (CH₂)ₙ-alkoxy groups (wherein n is 1, 2, 3, or 4), (CH₂)ₙNHCO-alkyl (wherein alkyl is substituted or unsubstituted, and n is 1, 2, 3, or 4), nitro groups, alcohol groups, —SFs groups, CONR′R″, R⁵ is joined with R⁴ to make a ring, R⁵ is joined with R⁶ to make a ring, R⁶ is joined with R⁷ to make a ring, and R⁷ is joined with R⁸ to make a ring, wherein R′ and R″ are independently chosen from H, substituted alkyl groups, and unsubstituted alkyl group and, wherein with the proviso compound is not:
1-(4-([1,2,4]triazolo[4,3-b]pyridazin-6-yl)piperazin-1-yl)-2-(2-chlorophenyl)ethan-1-one;
1-(4-([ 1,2,4]triazolo[4,3-b]pyridazin-6-yl)piperazin-1-yl)-2-phenylethan-1-one;
1-(4-([1,2,4]triazolo[4,3-b]pyridazin-6-yl)piperazin-1-yl)-2-(p-tolyl)ethan-1-one;
1-(4-([1,2,4]triazolo[4,3-b]pyridazin-6-yl)piperazin-1-yl)-2-(4-aminophenyl)ethan-1-one;
1-(4-([1,2,4]triazolo[4,3-b]pyridazin-6-yl)piperazin-1-yl)-2-amino-2-phenylethan-1-one;
1-(4-([1,2,4]triazolo[4,3-b]pyridazin-6-yl)piperazin-1-yl)-2-(m-tolyl)ethan-1-one;
1-(4-([1,2,4]triazolo[4,3-b]pyridazin-6-yl)piperazin-1-yl)-2-(o-tolyl)ethan-1-one;
1-(4-([1,2,4]triazolo[4,3-b]pyridazin-6-yl)piperazin-1-yl)-2-(4-bromophenyl)ethan-1-one;
1-(4-([1,2,4]triazolo[4,3-b]pyridazin-6-yl)piperazin-1-yl)-2-(4-chlorophenyl)ethan-1-one;
1-(4-([1,2,4]triazolo[4,3-b]pyridazin-6-yl)piperazin-1-yl)-2-(4-fluorophenyl)ethan-1-one;
1-(4-([ 1,2,4]triazolo[4,3 -b]pyridazin-6-yl)piperazin-1 -yl)-2-phenylbutan-1 -one;
1-(4-([1,2,4]triazolo[4,3-b]pyridazin-6-yl)piperazin-1-yl)-2-(4-methoxyphenyl)ethan-1-one;
1-(4-([1,2,4]triazolo[4,3 -b]pyridazin-6-yl)piperazin-1-yl)-2-amino-2-phenylpropan- 1 -one;
1-(4-([1,2,4]triazolo[4,3-b]pyridazin-6-yl)piperazin-1-yl)-2-amino-2-(p-tolyl)ethan-1-one;
1 -(4-([1,2,4]triazolo[4,3 -b]pyridazin-6-yl)piperazin-1-yl)-2-(4-ethoxyphenyl)ethan-1-one;
1-(4-([1,2,4]triazolo[4,3-b]pyridazin-6-yl)piperazin-1-yl)-2-(3-hydroxyphenyl)propan-1-one;
1-(4-([1,2,4]triazolo[4,3 -b]pyridazin-6-yl)piperazin-1-yl)-2-(naphthalen-1-yl)ethan-1-one;
1-(4-([1,2,4]triazolo[4,3-b]pyridazin-6-yl)piperazin-1-yl)-2-(naphthalen-2-yl)ethan-1-one;
1-(4-([1,2,4]triazolo[4,3-b]pyridazin-6-yl)piperazin-1-yl)-2-(2-fluorophenyl)ethan-1-one;
1-(4-([1,2,4]triazolo[4,3-b]pyridazin-6-yl)piperazin-1-yl)-2-(2-chlorophenyl)ethan-1-one;
1-(4-(3-methyl-[1,2,4]triazolo[4,3-b]pyridazin-6-yl)piperazin-1-yl)-2-(p-tolyl)ethan-1-one;
1-(4-([1,2,4]triazolo[4,3-b]pyridazin-6-yl)piperazin-1-yl)-2-(4-nitrophenyl)ethan-1-one;
2-([1,1′-biphenyl]-4-yl)-1-(4-([1,2,4]triazolo[4,3-b]pyridazin-6-yl)piperazin-1-yl)ethan-1-one;
2-(4-aminophenyl)-1-(4-(3-methyl-[1,2,4]triazolo[4,3-b]pyridazin-6-yl)piperazin-1-yl)ethan-1-one;
1-(4-(3-ethyl-[1,2,4]triazolo[4,3-b]pyridazin-6-yl)piperazin-1-yl)-2-(p-tolyl)ethan-1-one;
2-amino-1-(4-(3-methyl-[1,2,4]triazolo[4,3-b]pyridazin-6-yl)piperazin-1-yl)-2-phenylethan-1-one;
1-(4-(3-methyl-[1,2,4]triazolo[4,3-b]pyridazin-6-yl)piperazin-1-yl)-2-(o-tolyl)ethan-1-one;
1-(4-([1,2,4]triazolo[4,3-b]pyridazin-6-yl)piperazin-1-yl)-2-(2-methoxyphenyl)ethan-1-one;
1-(4-(3-cyclopropyl-[1,2,4]triazolo[4,3-b]pyridazin-6-yl)piperazin-1-yl)-2-phenylethan-1-one;
2-(2-aminophenyl)-1-(4-(3-methyl-[1,2,4]triazolo[4,3-b]pyridazin-6-yl)piperazin-1-yl)ethan-1-one;
3-amino-1-(4-(3-methyl-[1,2,4]triazolo[4,3-b]pyridazin-6-yl)piperazin-1-yl)-2-phenylpropan-1-one;
2-methyl-1-(4-(3-methyl-[1,2,4]triazolo[4,3-b]pyridazin-6-yl)piperazin-1-yl)-2-(p-tolyl)propan-1-one;
1-(4-(3-ethyl-[ 1,2,4]triazolo[4,3 -b]pyridazin-6-yl)piperazin-1 -yl)-2-(o-tolyl)ethan-1 -one;
1-(4-([1,2,4]triazolo[4,3-b]pyridazin-6-yl)piperazin-1-yl)-2-(2-chloro-6-fluorophenyl)ethan-1 -one;
1 -(4-(3 -methyl-[1,2,4]triazolo[4,3-b]pyridazin-6-yl)piperazin-1-yl)-2-(naphthalen-1-yl)ethan-1-one;
1-(4-(3-cyclopropyl-[1,2,4]triazolo[4,3-b]pyridazin-6-yl)piperazin-1-yl)-2-(p-tolyl)ethan-1-one;
(4-([1,2,4]triazolo[4,3-b]pyridazin-6-yl)piperazin-1-yl)(4-phenylpiperidin-4-yl)methanone;
1-(4-(3-ethyl-[1,2,4]triazolo[4,3-b]pyridazin-6-yl)piperazin-1-yl)-2-(naphthalen-1-yl)ethan-1-one;
2-(2-chlorophenyl)-1-(4-(3-methyl-[1,2,4]triazolo[4,3-b]pyridazin-6-yl)piperazin-1-yl)ethan-1-one;
2-(2-fluorophenyl)-1-(4-(3-methyl-[1,2,4]triazolo[4,3-b]pyridazin-6-yl)piperazin-1-yl)ethan-1-one;
3 -(1 -(4-([ 1,2,4]triazolo[4,3 -b]pyridazin-6-yl)piperazine-1-carbonyl)cyclopropyl)benzonitrile;
1-(4-(3-cyclopropyl-[1,2,4]triazolo[4,3-b]pyridazin-6-yl)piperazin-1-yl)-2-(m-tolyl)ethan-1-one;
1 -(4-(3 -ethyl-[1,2,4]triazolo[4,3-b]pyridazin-6-yl)piperazin-1-yl)-2-(2-fluorophenyl)ethan-1-one;
2-(2-chlorophenyl)-1-(4-(3-ethyl-[1,2,4]triazolo[4,3-b]pyridazin-6-yl)piperazin-1-yl)ethan-1-one;
(4-([1,2,4]triazolo[4,3 -b]pyridazin-6-yl)piperazin-1-yl)(1-(4-fluorophenyl)cyclopropyl)methanone;
1-(4-(3-cyclopropyl-[1,2,4]triazolo[4,3-b]pyridazin-6-yl)piperazin-1-yl)-2-(o-tolyl)ethan-1-one;
2-(2-methoxyphenyl)-1-(4-(3-methyl-[1,2,4]triazolo[4,3-b]pyridazin-6-yl)piperazin-1-yl)ethan-1-one;
1-(4-([1,2,4]triazolo[4,3-b]pyridazin-6-yl)piperazin-1-yl)-2-(3,4-dimethoxyphenyl)ethan-1-one;
1-(4-(3-cyclopropyl-[1,2,4]triazolo[4,3-b]pyridazin-6-yl)piperazin-1-yl)-2-phenylbutan-1 -one;
(4-([1,2,4]triazolo[4,3-b]pyridazin-6-yl)piperazin-1-yl)(1-(o-tolyl)cyclopropyl)methanone;
1-(4-(3-cyclopropyl-[1,2,4]triazolo[4,3-b]pyridazin-6-yl)piperazin-1-yl)-2-(4-fluorophenyl)ethan-1-one;
2-(4-bromophenyl)-1-(4-(3-cyclopropyl-[1,2,4]triazolo[4,3-b]pyridazin-6-yl)piperazin-1-yl)ethan-1-one;
1-(4-(3-cyclopropyl-[1,2,4]triazolo[4,3-b]pyridazin-6-yl)piperazin-1-yl)-2-(4-methoxyphenyl)ethan-1-one;
2-(4-chlorophenyl)-1-(4-(3-cyclopropyl-[ 1,2,4]triazolo[4,3-b]pyridazin-6-yl)piperazin-1-yl)ethan-1-one;
1-(4-(3-cyclopropyl-[1,2,4]triazolo[4,3-b]pyridazin-6-yl)piperazin-1-yl)-2-(naphthalen-1-yl)ethan-1-one;
2-amino-2-(3-chlorophenyl)-1-(4-(3-methyl-[1,2,4]triazolo[4,3-b]pyridazin-6-yl)piperazin-1-yl)ethan-1-one;
2-(2-chloro-6-fluorophenyl)-1-(4-(3-methyl-[ 1,2,4]triazolo[4,3-b]pyridazin-6-yl)piperazin-1-yl)ethan-1-one;
1 -(4-(3-ethyl-[1,2,4]triazolo[4,3-b]pyridazin-6-yl)piperazin-1-yl)-2-(2-methoxyphenyl)ethan-1-one;
1-(4-(3-cyclopropyl-[1,2,4]triazolo[4,3-b]pyridazin-6-yl)piperazin-1-yl)-2-(4-ethoxyphenyl)ethan-1-one;
2-(4-fluorophenyl)-1-(4-(3-phenyl-[1,2,4]triazolo[4,3-b]pyridazin-6-yl)piperazin-1-yl)ethan-1-one;
(4-([1,2,4]triazolo[4,3 -b]pyridazin-6-yl)piperazin-1-yl)((1 s,3 s)-3-hydroxy-1 -phenylcyclobutyl)methanone;
2-([1,1′-biphenyl]-4-yl)-1-(4-(3 -cyclopropyl-[1,2,4]triazolo[4,3-b]pyridazin-6-yl)piperazin-1-yl)ethan-1-one;
2-(2-chlorophenyl)-1-(4-(3-cyclopropyl-[1,2,4]triazolo[4,3-b]pyridazin-6-yl)piperazin-1-yl)ethan-1-one;
2-(2-chloro-6-fluorophenyl)-1-(4-(3-ethyl-[1,2,4]triazolo[4,3-b]pyridazin-6-yl)piperazin-1-yl)ethan-1-one;
1-(4-(3-cyclopropyl-[1,2,4]triazolo[4,3-b]pyridazin-6-yl)piperazin-1-yl)-2-(2-fluorophenyl)ethan-1-one;
(4-(3-methyl-[1,2,4]triazolo[4,3-b]pyridazin-6-yl)piperazin-1-yl)(4-phenylpiperidin-4-yl)methanone;
1-(4-(3-cyclopropyl-[1,2,4]triazolo[4,3-b]pyridazin-6-yl)piperazin-1-yl)-2-(4-nitrophenyl)ethan-1-one;
6-(2-(4-(3-cyclopropyl-[1,2,4]triazolo[4,3-b]pyridazin-6-yl)piperazin-1-yl)-2-oxoethyl)indolin-2-one;
2-(2-fluorophenyl)-1-(4-(3-phenyl-[1,2,4]triazolo[4,3-b]pyridazin-6-yl)piperazin-1-yl)ethan-1-one;
1-(4-(3-cyclopropyl-[1,2,4]triazolo[4,3-b]pyridazin-6-yl)piperazin-1-yl)-2-(2-methoxyphenyl)ethan-1-one;
2-amino-2-(2-methoxyphenyl)-1-(4-(3-methyl-[1,2,4]triazolo[4,3-b]pyridazin-6-yl)piperazin-1-yl)ethan-1-one;
2-(2-chloro-6-fluorophenyl)-1-(4-(3-cyclopropyl-[1,2,4]triazolo[4,3-b]pyridazin-6-yl)piperazin-1-yl)ethan-1-one;
2-(4-chlorophenyl)-1-(4-(3-(trifluoromethyl)-[1,2,4]triazolo[4,3-b]pyridazin-6-yl)piperazin-1-yl)ethan-1-one;
1 -(4-(3 -(2-fluorophenyl)-[1,2,4]triazolo[4,3-b]pyridazin-6-yl)piperazin-1-yl)-2-methyl-2-phenylpropan-1-one;
(4-([1,2,4]triazolo[4,3-b]pyridazin-6-yl)piperazin-1-yl)(1-(3-methoxy-4-methylphenyl)cyclopropyl)methanone;
2-(2-chlorophenyl)-1-(4-(3-(2-fluorophenyl)-[1,2,4]triazolo[4,3-b]pyridazin-6-yl)piperazin-1-yl)ethan-1-one;
2-(2-chlorophenyl)-1-(4-(3-(trifluoromethyl)-[1,2,4]triazolo[4,3-b]pyridazin-6-yl)piperazin-1-yl)ethan-1-one;
1-(4-(3-cyclopropyl-[1,2,4]triazolo[4,3-b]pyridazin-6-yl)piperazin-1-yl)-2-(3,4-dimethoxyphenyl)ethan-1 -one;
2-(2-chloro-6-fluorophenyl)-1-(4-(3-(thiophen-2-ylmethyl)-[1,2,4]triazolo[4,3-b]pyridazin-6-yl)piperazin-1 -yl)ethan-1 -one;
or salts thereof.

2. The compound of claim 1, wherein R^{1a} is chosen from —H, methyl, trifluoromethyl, phenyl, benzyl, cyclopropyl, and chosen from —H and methyl. 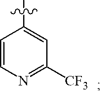
R^{1b} is chosen from —H and methyl; and R^{1c} is.

3. The compound of claim 1, wherein each R² is independently chosen from —H and methyl.

4. The compound of claim 1, wherein R⁴ is chosen from —H, —Cl, and —F.

5. The compound of claim 1, wherein R⁴ is joined with R⁵ to form a ring.

6. The compound of claim 1, wherein R⁵, R⁶, and R⁷ are independently chosen from —H, —F, —Cl, —Br, —OCH₃, —SF₅, —CF₃, —CH₃, t-butyl, ethyl, isopropyl, cyclopropyl, —CONH₂, —SO₂NH₂, —SO₂CH₃, phenyl, benzyl, phenoxy, —CH₂OH, —CCH, —CHF₂, —CH₂F, —CN, —NO₂, —OCF₃, —CF₂CH₃, —CF₂CF₃, —CCCH₃, —CH₂OCH₃, —CO₂H (and salts and deprotonated forms thereof), —CONHCH₃, —SO₂NHCH₃, 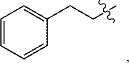 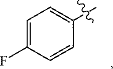 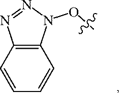 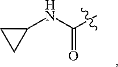 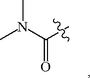 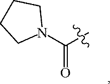 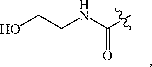 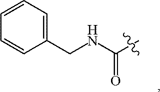 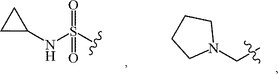 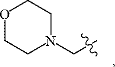 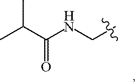 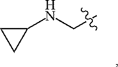 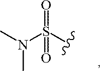 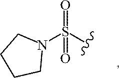 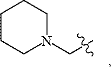 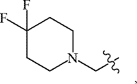 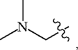 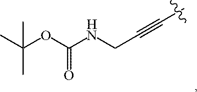 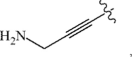 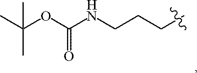 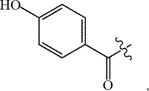
and combinations thereof.

7. The compound of claim 1, wherein R⁴ and R⁵ are joined to form a ring, R⁵ and R⁶ are joined to form a ring, R⁶ and R⁷ are joined to form a ring, or R⁷ and R⁸ are joined to form a ring.

8. The compound of claim 1, wherein R⁸ is chosen from —H, —Cl, and —F.

9. The compound of claim 1, wherein the compound is:
2-(2-chlorophenyl)-1 -(4- {[1,2,4]triazolo[4,3-b]pyridazin-6-yl }piperazin-1-yl)ethan-1 -one;
2-(4-chloro-3-fluorophenyl)-1-(4-{[1,2,4]triazolo[4,3-b]pyridazin-6-yl}piperazin-1-yl)ethan-1-one;
2-(3,4-dichlorophenyl)-1-(4-{[1,2,4]triazolo[4,3-b]pyridazin-6-y}piperazin-1-yl)ethan-1-one;
2-(2,6-dichlorophenyl)-1-(4-{[1,2,4]triazolo[4,3-b]pyridazin-6-yl}piperazin-1-yl)ethan-1-one;
2-(2,4-dichlorophenyl)-1-(4-{[1,2,4]triazolo[4,3-b]pyridazin-6-yl}piperazin-1-yl)ethan-1-one;
2-(2,5-dichlorophenyl)-1-(4-{[1,2,4]triazolo[4,3-b]pyridazin-6-yl}piperazin-1-yl)ethan-1-one;
2-(3,5-dichlorophenyl)-1-(4-{[1,2,4]triazolo[4,3-b]pyridazin-6-yl}piperazin-1-yl)ethan-1-one;
2-(3,4-dichlorophenyl)-1-(2-methyl-4-{[1,2,4]triazolo[4,3-b]pyridazin-6-yl}piperazin-1-yl)ethan-1-one;
2-(3-chloro-4-methylphenyl)-1-(4-{[1,2,4]triazolo[4,3-b]pyridazin-6-yl}piperazin-1-yl)ethan-1-one;
2-(3-fluoro-4-methylphenyl)-1-(4-{[1,2,4]triazolo[4,3-b]pyridazin-6-yl}piperazin-1-yl)ethan-1-one;
2-(4-chloro-3-methylphenyl)-1-(4-{[1,2,4]triazolo[4,3-b]pyridazin-6-yl}piperazin-1-yl)ethan-1-one;
2-(4-fluoro-3-methylphenyl)-1-(4-{[1,2,4]triazolo[4,3-b]pyridazin-6-yl}piperazin-1-yl)ethan-1-one;
2-(3 -bromophenyl)-1-(4- {[1,2,4]triazolo[4,3-b]pyridazin-6-yl }piperazin-1-yl)ethan-1 -one;
2-(3-methoxyphenyl)-1-(4-{[1,2,4]triazolo[4,3-b]pyridazin-6-yl}piperazin-1-yl)ethan-1-one;
2-(3-chloro-4-fluorophenyl)-1-(4-{[1,2,4]triazolo[4,3-b]pyridazin-6-yl}piperazin-1-yl)ethan-1-one;
2-(3-chlorophenyl)-1-(4-{[1,2,4]triazolo[4,3-b]pyridazin-6-yl}piperazin-1-yl)ethan-1-one;
1-(3,4-dichlorophenyl)-2-(4-{[1,2,4]triazolo[4,3-b]pyridazin-6-yl}piperazin-1-yl)ethane-1,2-dione;
1-[1-(3,4-dichlorophenyl)cyclopropanecarbonyl]-4-{[1,2,4]triazolo[4,3-b]pyridazin-6-yl}piperazine;
2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one;
4-[2-oxo-2-(4-{[1,2,4]triazolo[4,3-b]pyridazin-6-yl}piperazin-1-yl)ethyl]benzonitrile;
2-(2,3-dichlorophenyl)-1-(4-{[1,2,4]triazolo[4,3-b]pyridazin-6-yl}piperazin-1-yl)ethan-1-one;
2-(3,4-dichlorophenyl)-2,2-difluoro-1-(4-{[1,2,4]triazolo[4,3-b]pyridazin-6-yl }piperazin-1 -yl)ethan-1 -one;
2-(3,4-difluorophenyl)-1-(4-{[1,2,4]triazolo[4,3 -b]pyridazin-6-yl }piperazin-1-yl)ethan-1-one;
2-(3,4-dichlorophenyl)-1-(4-3-methyl-[1,2,4]triazolo[4,3-b]pyridazin-6-yl}piperazin-1-yl)ethan-1-one;
2-(3,4-dichlorophenyl)-1-(4-{3-phenyl-[1,2,4]triazolo[4,3-b]pyridazin-6-yl}piperazin-1-yl)ethan-1-one;
1-(4-{3-benzyl-[1,2,4]triazolo[4,3-b]pyridazin-6-yl}piperazin-1-yl)-2-(3,4-dichlorophenyl)ethan-1-one;
2-(3,4-dichlorophenyl)-1-{4-[3-(trifluoromethyl)-[1,2,4]triazolo[4,3-b]pyridazin-6-yl]piperazin-1-yl}ethan-1-one;
1-(4-{3-cyclopropyl-[1,2,4]triazolo[4,3-b]pyridazin-6-yl}piperazin-1-yl)-2-(3,4-dichlorophenyl)ethan-1-one;
2-(3,4-dichlorophenyl)-1-(4-{[1,2,4]triazolo[4,3-b]pyridazin-6-yl}piperazin-1-yl)propan-1-one;
2-(3,4-dichlorophenyl)-2-methyl-1-(4-{[1,2,4]triazolo[4,3-b]pyridazin-6-yl}piperazin-1-yl)propan-1-one;
2-(3,4-dichlorophenyl)-1-(3-methyl-4-{[1,2,4]triazolo[4,3-b]pyridazin-6-yl}piperazin-1-yl)ethan-1-one;
2-(3,4-dichlorophenyl)-1-(3,5-dimethyl-4-{[1,2,4]triazolo[4,3-b]pyridazin-6-yl}piperazin-1-yl)ethan-1-one;
2-(3,4-dichlorophenyl)-1-(2,6-dimethyl-4-{[1,2,4]triazolo[4,3-b]pyridazin-6-yl}piperazin-1-yl)ethan-1-one;
2-(3-chloro-5-fluorophenyl)-1-(4-{[1,2,4]triazolo[4,3-b]pyridazin-6-yl}piperazin-1-yl)ethan-1-one;
2-[4-(pentafluoro-λ⁶-sulfanyl)phenyl]-1-(4-{[1,2,4]triazolo[4,3-b]pyridazin-6-yl}piperazin-1-yl)ethan-1-one;
2-[3-(pentafluoro-λ⁶-sulfanyl)phenyl]-1-(4-{[1,2,4]triazolo[4,3-b]pyridazin-6-yl}piperazin-1-yl)ethan-1-one;
2-[4-chloro-3-(trifluoromethyl)phenyl]-1-(4-{[1,2,4]triazolo[4,3-b]pyridazin-6-yl}piperazin-1-yl)ethan-1-one;
2-[3-chloro-4-(trifluoromethyl)phenyl]-1-(4-{[1,2,4]triazolo[4,3-b]pyridazin-6-yl}piperazin-1-yl)ethan-1-one;
2-[4-fluoro-3-(trifluoromethyl)phenyl]-1-(4-{[1,2,4]triazolo[4,3-b]pyridazin-6-yl}piperazin-1-yl)ethan-1-one;
2-[3-fluoro-4-(trifluoromethyl)phenyl]-1-(4-{[1,2,4]triazolo[4,3-b]pyridazin-6-yl}piperazin-1-yl)ethan-1-one;
1-(4-{[1,2,4]triazolo[4,3-b]pyridazin-6-yl}piperazin-1-yl)-2-[3-(trifluoromethyl)phenyl]ethan-1-one;
1-(4-{[1,2,4]triazolo[4,3-b]pyridazin-6-yl}piperazin-1yl)-2-[4-(trifluoromethyl)phenyl]ethan-1-one;
2-(2,3-dihydro-1H-inden-5-yl)-1-(4-{[1,2,4]triazolo[4,3-b]pyridazin-6-yl}piperazin-1-yl)ethan-1-one;
2-(3-tert-butylphenyl)-1-(4-{[1,2,4]triazolo[4,3-b]pyridazin-6-yl}piperazin-1-yl)ethan-1-one;
2-(4-tert-butylphenyl)-1-(4-{[1,2,4]triazolo[4,3-b]pyridazin-6-yl}piperazin-1-yl)ethan-1-one;
2-(4-chloro-3-ethylphenyl)-1-(4-{[1,2,4]triazolo[4,3-b]pyridazin-6-yl}piperazin-1-yl)ethan-1-one;
2-(3-ethyl-4-fluorophenyl)-1-(4-{[1,2,4]triazolo[4,3-b]pyridazin-6-yl}piperazin-1-yl)ethan-1-one;
2-[4-chloro-3-(propan-2-yl)phenyl]-1-(4-{[1,2,4]triazolo[4,3-b]pyridazin-6-yl}piperazin-1-yl)ethan-1-one;
2-(4-chloro-3-cyclopropylphenyl)-1-(4-{[1,2,4]triazolo[4,3-b]pyridazin-6-yl}piperazin-1-yl)ethan-1-one;
2-(4-methylphenyl)-1-(4-{[1,2,4]triazolo[4,3-b]pyridazin-6-yl}piperazin-1-yl)ethan-1-one;
2-(3-methylphenyl)-1-(4-{[1,2,4]triazolo[4,3-b]pyridazin-6-yl}piperazin-1-yl)ethan-1-one;
4-[2-oxo-2-(4-{[1,2,4]triazolo[4,3-b]pyridazin-6-yl}piperazin-1-yl)ethyl]benzamide;
3-[2-oxo-2-(4-{[1,2,4]triazolo[4,3-b]pyridazin-6-yl}piperazin-1-yl)ethyl]benzamide;
N-cyclopropyl-4-[2-oxo-2-(4-{[1,2,4]triazolo[4,3-b]pyridazin-6-yl}piperazin-1-yl)ethyl]benzamide;
N-cyclopropyl-3-[2-oxo-2-(4-{[1,2,4]triazolo[4,3-b]pyridazin-6-yl}piperazin-1-yl)ethyl]benzamide;
N,N-dimethyl-4-[2-oxo-2-(4-{[1,2,4]triazolo[4,3-b]pyridazin-6-yl}piperazin-1-yl)ethyl]benzamide;
N,N-dimethyl-3-[2-oxo-2-(4-{[1,2,4]triazolo[4,3-b]pyridazin-6-yl}piperazin-1-yl)ethyl]benzamide;
2-[4-(pyrrolidine-1-carbonyl)phenyl]-1-(4-{[1,2,4]triazolo[4,3-b]pyridazin-6-yl}piperazin-1-yl)ethan-1-one;
2-[3-(pyrrolidine-1-carbonyl)phenyl]-1-(4-{[1,2,4]triazolo[4,3-b]pyridazin-6-yl}piperazin-1-yl)ethan-1-one;
N-(2-hydroxyethyl)-4-[2-oxo-2-(4-{[1,2,4]triazolo[4,3-b]pyridazin-6-yl}piperazin-1-yl)ethyl]benzamide;
N-(2-hydroxyethyl)-3-[2-oxo-2-(4-{[1,2,4]triazolo[4,3-b]pyridazin-6-yl}piperazin-1-yl)ethyl]benzamide;
N-benzyl-4-[2-oxo-2-(4-{[1,2,4]triazolo[4,3-b]pyridazin-6-yl}piperazin-1-yl)ethyl]benzamide;
N-benzyl-3-[2-oxo-2-(4-{[1,2,4]triazolo[4,3-b]pyridazin-6-yl}piperazin-1-yl)ethyl]benzamide;
4-[2-oxo-2-(4-{[1,2,4]triazolo[4,3-b]pyridazin-6-yl}piperazin-1-yl)ethyl]benzene-1-sulfonamide;
3-[2-oxo-2-(4-{[1,2,4]triazolo[4,3-b]pyridazin-6-yl}piperazin-1-yl)ethyl]benzene-1-sulfonamide;
N-cyclopropyl-4-[2-oxo-2-(4-{[1,2,4]triazolo[4,3-b]pyridazin-6-yl}piperazin-1-yl)ethyl]benzene-1-sulfonamide;
N-cyclopropyl-3-[2-oxo-2-(4-}[1,2,4]triazolo[4,3-b]pyridazin-6-yl}piperazin-1-yl)ethyl]benzene-1-sulfonamide;
2-(4-methanesulfonylphenyl)-1-(4-{[1,2,4]triazolo[4,3-b]pyridazin-6-yl}piperazin-1-yl)ethan-1-one;
2-(3-methanesulfonylphenyl)-1-(4-{[1,2,4]triazolo[4,3-b]pyridazin-6-yl}piperazin-1-yl)ethan-1-one;
2-{[1,1′-biphenyl]-3-yl}-1-(4-{[1,2,4]triazolo[4,3-b]pyridazin-6-yl}piperazin-1-yl)ethan-1-one;
2-(4-benzylphenyl)-1-(4-{[1,2,4]triazolo[4,3-b]pyridazin-6-yl}piperazin-1-yl)ethan-1-one;
2-(3-benzylphenyl)-1-(4-{[1,2,4]triazolo[4,3-b]pyridazin-6-yl}piperazin-1-yl)ethan-1-one;
2-(4-phenoxyphenyl)-1-(4-{[1,2,4]triazolo[4,3-b]pyridazin-6-yl}piperazin-1-yl)ethan-1-one;
2-(3-phenoxyphenyl)-1-(4-{[1,2,4]triazolo[4,3-b]pyridazin-6-yl}piperazin-1-yl)ethan-1-one;
2-[4-(2-phenylethyl)phenyl]-1-(4-{[1,2,4]triazolo[4,3-b]pyridazin-6-yl}piperazin-1-yl)ethan-1-one;
2-[3-(2-phenylethyl)phenyl]-1-(4-{[1,2,4]triazolo[4,3-b]pyridazin-6-yl}piperazin-1-yl)ethan-1-one;
2-[3-(hydroxymethyl)phenyl]-1-(4-{[1,2,4]triazolo[4,3-b]pyridazin-6-yl}piperazin-1-yl)ethan-1-one;
2-[3-chloro-4-(hydroxymethyl)phenyl]-1-(4-{[1,2,4]triazolo[4,3-b]pyridazin-6-yl}piperazin-1-yl)ethan-1-one;
2-[4-chloro-3-(hydroxymethyl)phenyl]-1-(4-{[1,2,4]triazolo[4,3-b]pyridazin-6-yl}piperazin-1-yl)ethan-1-one;
2-{4-[(pyrrolidin-1-yl)methyl]phenyl}-1-(4-{[1,2,4]triazolo[4,3-b]pyridazin-6-yl}piperazin-1-yl)ethan-1-one;
2-{3-[(pyrrolidin-1-yl)methyl]phenyl}-1-(4-{[1,2,4]triazolo[4,3-b]pyridazin-6-yl}piperazin-1-yl)ethan-1-one;
2-{3-chloro-4-[(pyrrolidin-1-yl)methyl]phenyl}-1-(4-{[1,2,4]triazolo[4,3-b]pyridazin-6-yl}piperazin-1-yl)ethan-1-one;
2-{4-chloro-3-[(pyrrolidin-1-yl)methyl]phenyl}-1-(4-{[1,2,4]triazolo[4,3-b]pyridazin-6-yl}piperazin-1-yl)ethan-1-one;
2-{4-[(morpholin-4-yl)methyl]phenyl}-1-(4-{[1,2,4]triazolo[4,3-b]pyridazin-6-yl}piperazin-1-yl)ethan-1-one;
2-{3-[(morpholin-4-yl)methyl]phenyl}-1-(4-{[1,2,4]triazolo[4,3-b]pyridazin-6-yl}piperazin-1-yl)ethan-1-one;
2-{3-chloro-4-[(morpholin-4-yl)methyl]phenyl}-1-(4-{[1,2,4]triazolo[4,3-b]pyridazin-6-yl}piperazin-1-yl)ethan-1-one;
2-{4-chloro-3-[(morpholin-4-yl)methyl]phenyl}-1-(4-{[1,2,4]triazolo[4,3-b]pyridazin-6-yl}piperazin-1-yl)ethan-1-one;
2-methyl-N-({4-[2-oxo-2-(4-{[1,2,4]triazolo[4,3-b]pyridazin-6-yl}piperazin-1-yl)ethyl]phenyl}methyl)propanamide;
2-methyl-N-({3-[2-oxo-2-(4-{[1,2,4]triazolo[4,3-b]pyridazin-6-yl}piperazin-1-yl)ethyl]phenyl}methyl)propanamide;
N-({2-chloro-4-[2-oxo-2-(4-{[1,2,4]triazolo[4,3-b]pyridazin-6-yl}piperazin-1-yl)ethyl]phenyl}methyl)-2-methylpropanamide;
N-({2-chloro-5-[2-oxo-2-(4-{[1,2,4]triazolo[4,3-b]pyridazin-6-yl}piperazin-1-yl)ethyl]phenyl}methyl)-2-methylpropanamide;
2-{3-chloro-4-[(cyclopropylamino)methyl]phenyl}-1-(4-{[1,2,4]triazolo[4,3-b]pyridazin-6-yl}piperazin-1-yl)ethan-1-one;
2-{4-chloro-3-[(cyclopropylamino)methyl]phenyl}-1-(4-{[1,2,4]triazolo[4,3-b]pyridazin-6-yl}piperazin-1-yl)ethan-1-one;
N,N-dimethyl-4-[2-oxo-2-(4-{[1,2,4]triazolo[4,3-b]pyridazin-6-yl}piperazin-1-yl)ethyl]benzene-1-sulfonamide;
N,N-dimethyl-3-[2-oxo-2-(4-{[1,2,4]triazolo[4,3-b]pyridazin-6-yl}piperazin-1-yl)ethyl]benzene-1-sulfonamide;
2-[4-(pyrrolidine-1-sulfonyl)phenyl]-1-(4-{[1,2,4]triazolo[4,3-b]pyridazin-6-yl}piperazin-1 -yl)ethan-1 -one;
2-[3-(pyrrolidine-1-sulfonyl)phenyl]-1-(4-{[1,2,4]triazolo[4,3-b]pyridazin-6-yl}piperazin-1 -yl)ethan-1 -one;
2-(3-ethynylphenyl)-1-(4-{[1,2,4]triazolo[4,3-b]pyridazin-6-yl}piperazin-1-yl)ethan-1-one;
2-{4-[(piperidin-1-yl)methyl]phenyl}-1-(4-{[1,2,4]triazolo[4,3-b]pyridazin-6-yl}piperazin-1 -yl)ethan-1 -one;
2-{4-[(4,4-difluoropiperidin-1-yl)methyl]phenyl}-1-(4-{[1,2,4]triazolo[4,3-b]pyridazin-6-yl}piperazin-1-yl)ethan-1-one;
2-{4-[(dimethylamino)methyl]phenyl}-1-(4-{[1,2,4]triazolo[4,3-b]pyridazin-6-yl}piperazin-1 -yl)ethan-1 -one;
2-[4-chloro-3-(difluoromethyl)phenyl]-1-(4-{[1,2,4]triazolo[4,3-b]pyridazin-6-yl}piperazin-1 -yl)ethan-1 -one;
2-[4-chloro-3-(fluoromethyl)phenyl]-1-(4-{[1,2,4]triazolo[4,3-b]pyridazin-6-yl}piperazin-1 -yl)ethan-1 -one;
2-(3,4-dichlorophenyl)-1-(4-{8-methyl-[1,2,4]triazolo[4,3-b]pyridazin-6-yl }piperazin-1-yl)ethan-1-one;
2-(3,4-dichlorophenyl)-1-(4-{7-methyl-[1,2,4]triazolo[4,3-b]pyridazin-6-yl}piperazin-1-yl)ethan-1-one;
2-(3,4-dichlorophenyl)-1-{4-[3-(trifluoromethyl)-[1,2,4]triazolo[4,3-b]pyridazin-6-yl]piperazin-1 -yl} ethan-1 -one;
tert-butyl N-(3-{4-[2-oxo-2-(4-{[1,2,4]triazolo[4,3-b]pyridazin-6-y}piperazin-1-yl)ethyl]phenyl }prop-2-yn-1 -yl)carbamate;
2-[4-(hydroxymethyl)phenyl]-1-(4-{[1,2,4]triazolo[4,3-b]pyridazin-6-yl }piperazin-1 -yl)ethan-1-one;
2-[4-(3-aminoprop-1-yn-1-yl)phenyl]-1-(4-{[1,2,4]triazolo[4,3-b]pyridazin-6-yl}piperazin-1 -yl)ethan-1 -one;
2-(4-ethynylphenyl)-1-(4-{ [1,2,4]triazolo[4,3-b]pyridazin-6-yl}piperazin-1-yl)ethan-1-one;
2-(2,3,4,5,6-pentafluorophenyl)-1-(4-{[1,2,4]triazolo[4,3-b]pyridazin-6-yl}piperazin-1-yl)ethan-1-one;
2-(4-bromo-3-chlorophenyl)-1-(4-{[1,2,4]triazolo[4,3-b]pyridazin-6-yl}piperazin-1-yl)ethan-1-one;
2-(3-bromo-4-chlorophenyl)-1-(4-{[1,2,4]triazolo[4,3-b]pyridazin-6-yl}piperazin-1-yl)ethan-1-one;
2-(4-bromo-3-fluorophenyl)-1-(4-{[1,2,4]triazolo[4,3-b]pyridazin-6-yl}piperazin-1-yl)ethan-1-one;
2-(3-bromo-4-fluorophenyl)-1-(4-{[1,2,4]triazolo[4,3-b]pyridazin-6-yl}piperazin-1-yl)ethan-1-one;
tert-butylN-(3-{4-[2-oxo-2-(4-{[1,2,4]triazolo[4,3-b]pyridazin-6-yl}piperazin-1-yl)ethyl]phenyl}propyl)carbamate;
2-(3,4-dichlorophenyl)-1-(4-{3-[2-(trifluoromethyl)pyridin-4-yl]-[1,2,4]triazolo[4,3-b]pyridazin-6-yl}piperazin-1-yl)ethan-1-one;
2-(2H-1,3-benzodioxol-5-yl)-1-(4-{[1,2,4]triazolo[4,3-b]pyridazin-6-yl}piperazin-1-yl)ethan-1-one;
tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate;
2-[3-chloro-5-(trifluoromethyl)phenyl]-1-(4-{[1,2,4]triazolo[4,3-b]pyridazin-6-yl}piperazin-1-yl)ethan-1-one;
2-(3,5-difluorophenyl)-1-(4-{[1,2,4]triazolo[4,3-b]pyridazin-6-yl}piperazin-1-yl)ethan-1-one;
2-[4-(4-hydroxybenzoyl)phenyl]-1-(4-{[1,2,4]triazolo[4,3-b]pyridazin-6-yl}piperazin-1-yl)ethan-1-one;
2-(4-fluoro-3-methoxyphenyl)-1-(4-{[1,2,4]triazolo[4,3-b]pyridazin-6-yl}piperazin-1-yl)ethan-1-one;
2-[3,5-bis(trifluoromethyl)phenyl]-1-(4-{[1,2,4]triazolo[4,3-b]pyridazin-6-yl}piperazin-1-yl)ethan-1-one;
2-(3-fluoro-4-methoxyphenyl)-1-(4-{[1,2,4]triazolo[4,3-b]pyridazin-6-yl}piperazin-1-yl)ethan-1-one;
1-(4-{[1,2,4]triazolo[4,3-b]pyridazin-6-yl}piperazin-1-yl)-2-(3,4,5-trifluorophenyl)ethan-1-one;
2-[4-fluoro-3-(propan-2-yl)phenyl]-1-(4-{[1,2,4]triazolo[4,3-b]pyridazin-6-yl}piperazin-1-yl)ethan-1-one;
2-(3-cyclopropyl-4-fluorophenyl)-1-(4-{[1,2,4]triazolo[4,3-b]pyridazin-6-yl}piperazin-1-yl)ethan-1-one;
2-[3-(difluoromethyl)-4-fluorophenyl]-1-(4-{[1,2,4]triazolo[4,3-b]pyridazin-6-yl}piperazin-1-yl)ethan-1-one;
2-[4-fluoro-3-(fluoromethyl)phenyl]-1-(4-{[1,2,4]triazolo[4,3-b]pyridazin-6-yl}piperazin-1-yl)ethan-1-one;
2-[3,4-bis(trifluoromethyl)phenyl]-1-(4-{[1,2,4]triazolo[4,3-b]pyridazin-6-yl}piperazin-1-yl)ethan-1-one;
2-[3-fluoro-5-(trifluoromethyl)phenyl]-1-(4-{[1,2,4]triazolo[4,3-b]pyridazin-6-yl}piperazin-1-yl)ethan-1-one;
2-[2-fluoro-3-(trifluoromethyl)phenyl]-1-(4-{[1,2,4]triazolo[4,3-b]pyridazin-6-yl}piperazin-1-yl)ethan-1-one;
2-fluoro-5-[2-oxo-2-(4-{[1,2,4]triazolo[4,3-b]pyridazin-6-yl}piperazin-1-yl)ethyl]benzonitrile;
2-(4-fluoro-3-nitrophenyl)-1-(4-{[1,2,4]triazolo[4,3-b]pyridazin-6-yl}piperazin-1-yl)ethan-1-one;
2-[4-fluoro-3-(trifluoromethoxy)phenyl]-1-(4-{[1,2,4]triazolo[4,3-b]pyridazin-6-yl}piperazin-1-yl)ethan-1-one;
2-{6-fluoro-[1,1′-biphenyl]-3-yl}-1-(4-{[1,2,4]triazolo[4,3-b]pyridazin-6-yl}piperazin-1-yl)ethan-1-one;
2-{4′,6-difluoro-[1,1′-biphenyl]-3-yl}-1-(4-{[1,2,4]triazolo[4,3-b]pyridazin-6-yl}piperazin-1-yl)ethan-1-one;
2-[3-(1,1-difluoroethyl)-4-fluorophenyl]-1-(4-{[1,2,4]triazolo[4,3-b]pyridazin-6-yl}piperazin-1-yl)ethan-1-one;
2-[4-fluoro-3-(1,1,2,2,2-pentafluoroethyl)phenyl]-1-(4-{[1,2,4]triazolo[4,3-b]pyridazin-6-yl}piperazin-1-yl)ethan-1-one;
2-(3-ethynyl-4-fluorophenyl)-1-(4-{[1,2,4]triazolo[4,3-b]pyridazin-6-yl}piperazin-1-yl)ethan-1-one;
2-[4-fluoro-3-(prop-1-yn-1-yl)phenyl]-1-(4-{[1,2,4]triazolo[4,3-b]pyridazin-6-yl}piperazin-1-yl)ethan-1-one;
2-(3-tert-butyl-4-fluorophenyl)-1-(4-{[1,2,4]triazolo[4,3-b]pyridazin-6-yl}piperazin-1-yl)ethan-1-one;
2-[4-fluoro-3-(hydroxymethyl)phenyl]-1-(4-{[1,2,4]triazolo[4,3-b]pyridazin-6-yl}piperazin-1-yl)ethan-1-one;
2-[4-fluoro-3-(methoxymethyl)phenyl]-1-(4-{[1,2,4]triazolo[4,3-b]pyridazin-6-yl}piperazin-1-yl)ethan-1-one;
2-fluoro-5-[2-oxo-2-(4-{[1,2,4]triazolo[4,3-b]pyridazin-6-yl}piperazin-1-yl)ethyl]benzoicacid;
2-fluoro-5-[2-oxo-2-(4-{[1,2,4]triazolo[4,3-b]pyridazin-6-yl}piperazin-1-yl)ethyl]benzamide;
2-fluoro-N-methyl-5-[2-oxo-2-(4-{[1,2,4]triazolo[4,3-b]pyridazin-6-yl}piperazin-1-yl)ethyl]benzamide;
2-fluoro-N,N-dimethyl-5-[2-oxo-2-(4-{[1,2,4]triazolo[4,3-b]pyridazin-6-yl}piperazin-1-yl)ethyl]benzamide;
2-fluoro-5-[2-oxo-2-(4-{[1,2,4]triazolo[4,3-b]pyridazin-6-yl}piperazin-1-yl)ethyl]benzene-1-sulfonamide;
2-fluoro-N-methyl-5-[2-oxo-2-(4-{[1,2,4]triazolo[4,3-b]pyridazin-6-yl}piperazin-1-yl)ethyl]benzene-1-sulfonamide;
2-fluoro-N,N-dimethyl-5-[2-oxo-2-(4-{[1,2,4]triazolo[4,3-b]pyridazin-6-yl}piperazin-1-yl)ethyl]benzene-1-sulfonamide;or
2-(4-fluoro-3-methanesulfonylphenyl)-1-(4-{[1,2,4]triazolo[4,3-b]pyridazin-6-yl}piperazin-1-yl)ethan-1-one.

10. Thecompoundofclaim9,whereinthecompoundis:
2-[4-fluoro-3-(trifluoromethyl)phenyl]-1-(4-{[1,2,4]triazolo[4,3-b]pyridazin-6-yl}piperazin-1-yl)ethan-1-one;
2-(3-bromo-4-chlorophenyl)-1-(4-{[1,2,4]triazolo[4,3-b]pyridazin-6-yl}piperazin-1-yl)ethan-1-one;
2-(3,4-dichlorophenyl)-1-(4-{[1,2,4]triazolo[4,3-b]pyridazin-6-yl}piperazin-1-yl)ethan-1-one;
2-(3-bromo-4-fluorophenyl)-1-(4-{[1,2,4]triazolo[4,3-b]pyridazin-6-yl}piperazin-1-yl)ethan-1-one;
2-(3-chloro-4-fluorophenyl)-1-(4-[1,2,4]triazolo[4,3-b]pyridazin-6-yl}piperazin-1-yl)ethan-1-one;
2-(4-bromo-3-chlorophenyl)-1-(4-{[1,2,4]triazolo[4,3-b]pyridazin-6-yl}piperazin-1-yl)ethan-1-one;
2-[4-chloro-3-(trifluoromethyl)phenyl]-1-(4-{[1,2,4]triazolo[4,3-b]pyridazin-6-yl}piperazin-1-yl)ethan-1-one;
2-(4-chloro-3-methylphenyl)-1-(4-{[1,2,4]triazolo[4,3-b]pyridazin-6-yl}piperazin-1-yl)ethan-1-one;
2-[3,5-bis(trifluoromethyl)phenyl]-1-(4-{[1,2,4]triazolo[4,3-b]pyridazin-6-yl}piperazin-1-yl)ethan-1-one;
2-(4-fluoro-3-methylphenyl)-1-(4-{[1,2,4]triazolo[4,3-b]pyridazin-6-yl}piperazin-1-yl)ethan-1-one;
2-[3-chloro-5-(trifluoromethyl)phenyl]-1-(4-{[1,2,4]triazolo[4,3-b]pyridazin-6-yl}piperazin-1-yl)ethan-1-one;
2-(4-chloro-3-fluorophenyl)-1-(4-{[1,2,4]triazolo[4,3-b]pyridazin-6-yl}piperazin-1-yl)ethan-1-one;
2-(3,4-difluorophenyl)-1-(4-{[1,2,4]triazolo[4,3-b]pyridazin-6-yl}piperazin-1-yl)ethan-1-one;
2-(4-bromo-3-fluorophenyl)-1-(4-{[1,2,4]triazolo[4,3-b]pyridazin-6-yl}piperazin-1-yl)ethan-1-one;
2-(3-chloro-5-fluorophenyl)-1-(4-{[1,2,4]triazolo[4,3-b]pyridazin-6-yl}piperazin-1-yl)ethan-1-one;
2-(4-ethynylphenyl)-1-(4-{[1,2,4]triazolo[4,3-b]pyridazin-6-yl}piperazin-1-yl)ethan-1-one;or
2-(3,5-dichlorophenyl)-1-(4-{[1,2,4]triazolo[4,3-b]pyridazin-6-yl}piperazin-1-yl)ethan-1-one.

11. A composition comprising a pharmaceutically acceptable carrier and one or more compounds having the following structure: 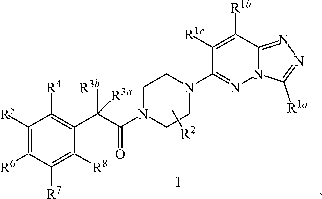
wherein
R^{1a}, R^{1b}, and R^{1c} are independently chosen from H, substituted alkyl groups, unsubstituted alkyl groups, substituted aryl groups, unsubstituted aryl groups, substituted aralkyl groups, and unsubstituted aralkyl groups;
R² is chosen from H, substituted alkyl groups, and unsubstituted alkyl groups;
R^{3a} and R^{3b} are independently chosen from H, substituted alkyl groups, unsubstituted alkyl groups, halogen, OH, NH₂, and R^{3a} and R^{3b} may combine to form a ring or a ═O;
R⁴ and R⁸ are independently chosen from H, halogens, substituted alkyl groups, and unsubstituted alkyl groups;
R⁵, R⁶, and R⁷ are independently chosen from H, halogens, substituted alkyl groups, unsubstituted alkyl groups, substituted cycloalkyl groups, unsubstituted cycloalkyl groups, substituted alkoxy groups, unsubstituted alkoxy groups, substituted aryl groups, unsubstituted aryl groups, substituted aralkyl groups, unsubstituted aralkyl groups, SO₂NR′R″, (CH₂)ₙ-alkoxy groups (wherein n is 1, 2, 3, or 4), (CH₂)ₙNHCO-alkyl (wherein alkyl is substituted or unsubstituted, and n is 1, 2, 3, or 4), nitro groups, alcohol groups, —SFs groups, CONR′R″, R⁵ is joined with R⁴ to make a ring, R⁵ is joined with R⁶ to make a ring, R⁶ is joined with R⁷ to make a ring, and R⁷ is joined with R⁸ to make a ring, wherein R′ and R″ are independently chosen from H, substituted alkyl groups, and unsubstituted alkyl group.

12. The composition of claim 11, where the composition is for oral administration and the one or more compounds are encapsulated in a pH sensitive polymer suitable for release of the compound in the small intestines, distal small intestine, or colon.

13. The composition of claim 11, wherein the one or more compounds are chosen from:
2-(2-chlorophenyl)-1-(4-{[1,2,4]triazolo[4,3-b]pyridazin-6-yl}piperazin-1-yl)ethan-1-one;
2-(naphthalen-1-yl)-1-(4-{[1,2,4]triazolo[4,3-b]pyridazin-6-yl}piperazin-1-yl)ethan-1-one;
2-(4-chloro-3-fluorophenyl)-1-(4-{[1,2,4]triazolo[4,3-b]pyridazin-6-yl}piperazin-1-yl)ethan-1-one;
2-(4-fluorophenyl)-1-(4-{[1,2,4]triazolo[4,3-b]pyridazin-6-yl}piperazin-1-yl)ethan-1-one;
2-(3,4-dichlorophenyl)-1-(4-{[1,2,4]triazolo[4,3-b]pyridazin-6-yl}piperazin-1-yl)ethan-1-one;
2-(4-chlorophenyl)-1-(4-{[1,2,4]triazolo[4,3-b]pyridazin-6-yl}piperazin-1-yl)ethan-1-one;
2-(2,6-dichlorophenyl)-1-(4-{[1,2,4]triazolo[4,3-b]pyridazin-6-yl}piperazin-1-yl)ethan-1-one;
2-(2,4-dichlorophenyl)-1-(4-{[1,2,4]triazolo[4,3-b]pyridazin-6-yl}piperazin-1-yl)ethan-1-one;
2-(2,5-dichlorophenyl)-1-(4-{[1,2,4]triazolo[4,3-b]pyridazin-6-yl}piperazin-1-yl)ethan-1-one;
2-(3,5-dichlorophenyl)-1-(4-{[1,2,4]triazolo[4,3-b]pyridazin-6-yl}piperazin-1-yl)ethan-1-one;
2-(naphthalen-2-yl)-1-(4-{[1,2,4]triazolo[4,3-b]pyridazin-6-yl}piperazin-1-yl)ethan-1-one;
2-(3,4-dichlorophenyl)-1-(2-methyl-4-{[1,2,4]triazolo[4,3-b]pyridazin-6-yl}piperazin-1-yl)ethan-1-one;
2-(4-nitrophenyl)-1-(4-{[1,2,4]triazolo[4,3-b]pyridazin-6-yl}piperazin-1-yl)ethan-1-one;
2-(3-chloro-4-methylphenyl)-1-(4-{[1,2,4]triazolo[4,3-b]pyridazin-6-yl}piperazin-1-yl)ethan-1-one;
2-(3-fluoro-4-methylphenyl)-1-(4-{[1,2,4]triazolo[4,3-b]pyridazin-6-yl}piperazin-1-yl)ethan-1-one;
2-(4-chloro-3-methylphenyl)-1-(4-{[1,2,4]triazolo[4,3-b]pyridazin-6-yl}piperazin-1-yl)ethan-1-one;
2-(4-fluoro-3-methylphenyl)-1-(4-{[1,2,4]triazolo[4,3-b]pyridazin-6-yl}piperazin-1-yl)ethan-1-one;
2-(3-bromophenyl)-1-(4-{[1,2,4]triazolo[4,3-b]pyridazin-6-yl}piperazin-1-yl)ethan-1-one;
2-(4-methoxyphenyl)-1-(4-{[1,2,4]triazolo[4,3-b]pyridazin-6-yl}piperazin-1-yl)ethan-1-one;
2-(3-methoxyphenyl)-1-(4-{[1,2,4]triazolo[4,3-b]pyridazin-6-yl}piperazin-1-yl)ethan-1-one;
2-(3-chloro-4-fluorophenyl)-1-(4-{[1,2,4]triazolo[4,3-b]pyridazin-6-yl}piperazin-1-yl)ethan-1-one;
2-(3-chlorophenyl)-1-(4-{[1,2,4]triazolo[4,3-b]pyridazin-6-yl}piperazin-1-yl)ethan-1-one;
1-(3,4-dichlorophenyl)-2-(4-{[1,2,4]triazolo[4,3-b]pyridazin-6-yl}piperazin-1-yl)ethane-1,2-dione;
1-[1-(3,4-dichlorophenyl)cyclopropanecarbonyl]-4-{[1,2,4]triazolo[4,3-b]pyridazin-6-yl}piperazine;
2-(4-bromophenyl)-1-(4-{[1,2,4]triazolo[4,3-b]pyridazin-6-yl}piperazin-1-yl)ethan-1-one;
2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one;
4-[2-oxo-2-(4-{[1,2,4]triazolo[4,3-b]pyridazin-6-yl}piperazin-1-yl)ethyl]benzonitrile;
2-(2,3-dichlorophenyl)-1-(4-{[1,2,4]triazolo[4,3-b]pyridazin-6-yl}piperazin-1-yl)ethan-1-one;
2-(3,4-dichlorophenyl)-2,2-difluoro-1-(4-{[1,2,4]triazolo[4,3-b]pyridazin-6-yl}piperazin-1-yl)ethan-1-one;
2-(3,4-difluorophenyl)-1-(4-{[1,2,4]triazolo[4,3-b]pyridazin-6-yl}piperazin-1-yl)ethan-1-one;
2-(3,4-dichlorophenyl)-1-(4-{3-methyl-[1,2,4]triazolo[4,3-b]pyridazin-6-yl}piperazin-1-yl)ethan-1-one;
2-(3,4-dichlorophenyl)-1-(4-{3-phenyl-[1,2,4]triazolo[4,3-b]pyridazin-6-yl}piperazin-1-yl)ethan-1-one;
1-(4-{3-benzyl-[1,2,4]triazolo[4,3-b]pyridazin-6-yl}piperazin-1-yl)-2-(3,4-dichlorophenyl)ethan-1-one;
2-(3,4-dichlorophenyl)-1-{4-[3-(trifluoromethyl)-[1,2,4]triazolo[4,3-b]pyridazin-6-yl]piperazin-1-yl}ethan-1-one;
1-(4-{3-cyclopropyl-[1,2,4]triazolo[4,3-b]pyridazin-6-yl}piperazin-1-yl)-2-(3,4-dichlorophenyl)ethan-1-one;
2-(3,4-dichlorophenyl)-1-(4-{[1,2,4]triazolo[4,3-b]pyridazin-6-yl}piperazin-1-yl)propan-1-one;
2-(3,4-dichlorophenyl)-2-methyl-1-(4-{[1,2,4]triazolo[4,3-b]pyridazin-6-yl}piperazin-1-yl)propan-1-one;
2-(3,4-dichlorophenyl)-1-(3-methyl-4-{[1,2,4]triazolo[4,3-b]pyridazin-6-yl}piperazin-1-yl)ethan-1-one;
2-(3,4-dichlorophenyl)-1-(3,5-dimethyl-4-{[1,2,4]triazolo[4,3-b]pyridazin-6-yl}piperazin-1-yl)ethan-1-one;
2-(3,4-dichlorophenyl)-1-(2,6-dimethyl-4-{[1,2,4]triazolo[4,3-b]pyridazin-6-yl}piperazin-1-yl)ethan-1-one;
2-(3-chloro-5-fluorophenyl)-1-(4-{[1,2,4]triazolo[4,3-b]pyridazin-6-yl}piperazin-1-yl)ethan-1-one;
2-[4-(pentafluoro-λ⁶-sulfanyl)phenyl]-1-(4-{[1,2,4]triazolo[4,3-b]pyridazin-6-yl}piperazin-1-yl)ethan-1-one;
2-[3-(pentafluoro-λ⁶-sulfanyl)phenyl]-1-(4-{[1,2,4]triazolo[4,3-b]pyridazin-6-yl}piperazin-1-yl)ethan-1-one;
2-[4-chloro-3-(trifluoromethyl)phenyl]-1-(4-{[1,2,4]triazolo[4,3-b]pyridazin-6-yl}piperazin-1-yl)ethan-1-one;
2-[3-chloro-4-(trifluoromethyl)phenyl]-1-(4-{[1,2,4]triazolo[4,3-b]pyridazin-6-yl}piperazin-1-yl)ethan-1-one;
2-[4-fluoro-3-(trifluoromethyl)phenyl]-1-(4-{[1,2,4]triazolo[4,3-b]pyridazin-6-yl}piperazin-1-yl)ethan-1-one;
2-[3-fluoro-4-(trifluoromethyl)phenyl]-1-(4-{[1,2,4]triazolo[4,3-b]pyridazin-6-yl}piperazin-1-yl)ethan-1-one;
1-(4-{[1,2,4]triazolo[4,3-b]pyridazin-6-yl}piperazin-1-yl)-2-[3-(trifluoromethyl)phenyl]ethan-1-one;
1-(4-{[1,2,4]triazolo[4,3-b]pyridazin-6-yl}piperazin-1-yl)-2-[4-(trifluoromethyl)phenyl]ethan-1-one;
2-(2,3-dihydro-1H-inden-5-yl)-1-(4-{[1,2,4]triazolo[4,3-b]pyridazin-6-yl}piperazin-1-yl)ethan-1-one;
2-(3-tert-butylphenyl)-1-(4-{[1,2,4]triazolo[4,3-b]pyridazin-6-yl}piperazin-1-yl)ethan-1-one;
2-(4-tert-butylphenyl)-1-(4-{[1,2,4]triazolo[4,3-b]pyridazin-6-yl}piperazin-1-yl)ethan-1-one;
2-(4-chloro-3-ethylphenyl)-1-(4-{[1,2,4]triazolo[4,3-b]pyridazin-6-yl}piperazin-1-yl)ethan-1-one;
2-(3-ethyl-4-fluorophenyl)-1-(4-1,2,4]triazolo[4,3-b]pyridazin-6-yl}piperazin-1-yl)ethan-1-one;
2-[4-chloro-3-(propan-2-yl)phenyl]-1-(4-{[1,2,4]triazolo[4,3-b]pyridazin-6-yl}piperazin-1-yl)ethan-1-one;
2-(4-chloro-3-cyclopropylphenyl)-1-(4-{[1,2,4]triazolo[4,3-b]pyridazin-6-yl}piperazin-1-yl)ethan-1-one;
2-(4-methylphenyl)-1-(4-{[1,2,4]triazolo[4,3-b]pyridazin-6-yl}piperazin-1-yl)ethan-1-one;
2-(3-methylphenyl)-1-(4-{[1,2,4]triazolo[4,3-b]pyridazin-6-yl}piperazin-1-yl)ethan-1-one;
4-[2-oxo-2-(4-{[1,2,4]triazolo[4,3-b]pyridazin-6-yl}piperazin-1-yl)ethyl]benzamide;
3-[2-oxo-2-(4-{[1,2,4]triazolo[4,3-b]pyridazin-6-yl}piperazin-1-yl)ethyl]benzamide;
N-cyclopropyl-4-[2-oxo-2-(4-{[1,2,4]triazolo[4,3-b]pyridazin-6-yl}piperazin-1-yl)ethyl]benzamide;
N-cyclopropyl-3-[2-oxo-2-(4-{[1,2,4]triazolo[4,3-b]pyridazin-6-yl}piperazin-1-yl)ethyl]benzamide;
N,N-dimethyl-4-[2-oxo-2-(4-{[1,2,4]triazolo[4,3-b]pyridazin-6-yl}piperazin-1-yl)ethyl]benzamide;
N,N-dimethyl-3-[2-oxo-2-(4-{[1,2,4]triazolo[4,3-b]pyridazin-6-yl}piperazin-1-yl)ethyl]benzamide;
2-[4-(pyrrolidine-1-carbonyl)phenyl]-1-(4-{[1,2,4]triazolo[4,3-b]pyridazin-6-yl}piperazin-1-yl)ethan-1-one;
2-[3-(pyrrolidine-1-carbonyl)pheny]-1-(4-{[1,2,4]triazolo[4,3-b]pyridazin-6-yl}piperazin-1-yl)ethan-1-one;
N-(2-hydroxyethyl)-4-[2-oxo-2-(4-{[1,2,4]triazolo[4,3-b]pyridazin-6-yl}piperazin-1-yl)ethyl]benzamide;
N-(2-hydroxyethyl)-3-[2-oxo-2-(4-{[1,2,4]triazolo[4,3-b]pyridazin-6-yl}piperazin-1-yl)ethyl]benzamide;
N-benzyl-4-[2-oxo-2-(4-{[1,2,4]triazolo[4,3-b]pyridazin-6-yl}piperazin-1-yl)ethyl]benzamide;
N-benzyl-3-[2-oxo-2-(4-{[1,2,4]triazolo[4,3-b]pyridazin-6-yl}piperazin-1-yl)ethyl]benzamide;
4-[2-oxo-2-(4-{[1,2,4]triazolo[4,3-b]pyridazin-6-yl}piperazin-1-yl)ethyl]benzene-1-sulfonamide;
3-[2-oxo-2-(4-{[1,2,4]triazolo[4,3-b]pyridazin-6-yl}piperazin-1-yl)ethyl]benzene-1-sulfonamide;
N-cyclopropyl-4-[2-oxo-2-(4-{[1,2,4]triazolo[4,3-b]pyridazin-6-yl}piperazin-1-yl)ethyl]benzene-1-sulfonamide;
N-cyclopropyl-3-[2-oxo-2-(4-{[1,2,4]triazolo[4,3-b]pyridazin-6-yl}piperazin-1-yl)ethyl]benzene-1-sulfonamide;
2-(4-methanesulfonylphenyl)-1-(4-{[1,2,4]triazolo[4,3-b]pyridazin-6-yl}piperazin-1-yl)ethan-1-one;
2-(3-methanesulfonylphenyl)-1-(4-{[1,2,4]triazolo[4,3-b]pyridazin-6-yl}piperazin-1-yl)ethan-1-one;
2-{[1,1′-biphenyl]-4-yl}-1-(4-{[1,2,4]triazolo[4,3-b]pyridazin-6-yl}piperazin-1-yl)ethan-1-one;
2-{[1,1′-biphenyl]-3-yl}-1-(4-{[1,2,4]triazolo[4,3-b]pyridazin-6-yl}piperazin-1-yl)ethan-1-one;
2-(4-benzylphenyl)-1-(4-{[1,2,4]triazolo[4,3-b]pyridazin-6-yl}piperazin-1-yl)ethan-1-one;
2-(3-benzylphenyl)-1-(4-{[1,2,4]triazolo[4,3-b]pyridazin-6-yl}piperazin-1-yl)ethan-1-one;
2-(4-phenoxyphenyl)-1-(4-{[1,2,4]triazolo[4,3-b]pyridazin-6-yl}piperazin-1-yl)ethan-1-one;
2-(3-phenoxyphenyl)-1-(4-{[1,2,4]triazolo[4,3-b]pyridazin-6-yl}piperazin-1-yl)ethan-1-one;
2-[4-(2-phenylethyl)phenyl]-1-(4-{[1,2,4]triazolo[4,3-b]pyridazin-6-yl}piperazin-1-yl)ethan-1-one;
2-[3-(2-phenylethyl)phenyl]-1-(4-{[1,2,4]triazolo[4,3-b]pyridazin-6-yl}piperazin-1-yl)ethan-1-one;
2-[3-(hydroxymethyl)phenyl]-1-(4-{[1,2,4]triazolo[4,3-b]pyridazin-6-yl}piperazin-1-yl)ethan-1-one;
2-[3-chloro-4-(hydroxymethyl)phenyl]-1-(4-{[1,2,4]triazolo[4,3-b]pyridazin-6-yl}piperazin-1-yl)ethan-1-one;
2-[4-chloro-3-(hydroxymethyl)phenyl]-1-(4-{[1,2,4]triazolo[4,3-b]pyridazin-6-yl}piperazin-1-yl)ethan-1-one;
2-{4-[(pyrrolidin-1-yl)methyl]phenyl}-1-(4-{[1,2,4]triazolo[4,3-b]pyridazin-6-yl}piperazin-1-yl)ethan-1-one;
2-{3-[(pyrrolidin-1-yl)methyl]phenyl}-1-(4-{[1,2,4]triazolo[4,3-b]pyridazin-6-yl}piperazin-1-yl)ethan-1-one;
2-{3-chloro-4-[(pyrrolidin-1-yl)methyl]phenyl}-1-(4-{[1,2,4]triazolo[4,3-b]pyridazin-6-yl}piperazin-1-yl)ethan-1-one;
2-{4-chloro-3-[(pyrrolidin-1-yl)methyl]phenyl}-1-(4-{[1,2,4]triazolo[4,3-b]pyridazin-6-yl}piperazin-1-yl)ethan-1-one;
2-{4-[(morpholin-4-yl)methyl]phenyl}-1-(4-{[1,2,4]triazolo[4,3-b]pyridazin-6-yl}piperazin-1-yl)ethan-1-one;
2-{3-[(morpholin-4-yl)methyl]phenyl}-1-(4-{[1,2,4]triazolo[4,3-b]pyridazin-6-yl}piperazin-1-yl)ethan-1-one;
2-{3-chloro-4-[(morpholin-4-yl)methyl]phenyl}-1-(4-{[1,2,4]triazolo[4,3-b]pyridazin-6-yl}piperazin-1-yl)ethan-1-one;
2-{4-chloro-3-[(morpholin-4-yl)methyl]phenyl}-1-(4-{[1,2,4]triazolo[4,3-b]pyridazin-6-yl}piperazin-1-yl)ethan-1-one;
2-methyl-N-({4-[2-oxo-2-(4-{[1,2,4]triazolo[4,3-b]pyridazin-6-yl}piperazin-1-yl)ethyl]phenyl}methyl)propanamide;
2-methyl-N-({3-[2-oxo-2-(4-{[1,2,4]triazolo[4,3-b]pyridazin-6-yl}piperazin-1-yl)ethyl]phenyl}methyl)propanamide;
N-({2-chloro-4-[2-oxo-2-(4-{[1,2,4]triazolo[4,3-b]pyridazin-6-yl}piperazin-1-yl)ethyl]phenyl}methyl)-2-methylpropanamide;
N-({2-chloro-5-[2-oxo-2-(4-{[1,2,4]triazolo[4,3-b]pyridazin-6-yl}piperazin-1-yl)ethyl]phenyl}methyl)-2-methylpropanamide;
2-{3-chloro-4-[(cyclopropylamino)methyl]phenyl}-1-(4-{[1,2,4]triazolo[4,3-b]pyridazin-6-yl}piperazin-1-yl)ethan-1-one;
2-{4-chloro-3-[(cyclopropylamino)methyl]phenyl}-1-(4-{[1,2,4]triazolo[4,3-b]pyridazin-6-yl}piperazin-1-yl)ethan-1-one;
N,N-dimethyl-4-[2-oxo-2-(4-{[1,2,4]triazolo[4,3-b]pyridazin-6-yl}piperazin-1-yl)ethyl]benzene-1-sulfonamide;
N,N-dimethyl-3-[2-oxo-2-(4-{[1,2,4]triazolo[4,3-b]pyridazin-6-yl}piperazin-1-yl)ethyl]benzene-1-sulfonamide;
2-[4-(pyrrolidine-1-sulfonyl)phenyl]-1-(4-{[1,2,4]triazolo[4,3-b]pyridazin-6-yl}piperazin-1-yl)ethan-1-one;
2-[3-(pyrrolidine-1-sulfonyl)phenyl]-1-(4-{[1,2,4]triazolo[4,3-b]pyridazin-6-yl}piperazin-1-yl)ethan-1-one;
2-(3-ethynylphenyl)-1-(4-{[1,2,4]triazolo[4,3-b]pyridazin-6-yl}piperazin-1-yl)ethan-1-one;
2-{4-[(piperidin-1-yl)methyl]phenyl}-1-(4-{[1,2,4]triazolo[4,3-b]pyridazin-6-yl}piperazin-1-yl)ethan-1-one;
2-{4-[(4,4-difluoropiperidin-1-yl)methyl]phenyl}-1-(4-{[1,2,4]triazolo[4,3-b]pyridazin-6-yl}piperazin-1-yl)ethan-1-one;
2-{4-[(dimethylamino)methyl]phenyl}-1-(4-{[1,2,4]triazolo[4,3-b]pyridazin-6-yl}piperazin-1-yl)ethan-1-one;
2-[4-chloro-3-(difluoromethyl)phenyl]-1-(4-{[1,2,4]triazolo[4,3-b]pyridazin-6-yl}piperazin-1-yl)ethan-1-one;
2-[4-chloro-3-(fluoromethyl)phenyl]-1-(4-{[1,2,4]triazolo[4,3-b]pyridazin-6-yl}piperazin-1-yl)ethan-1-one;
2-(3,4-dichlorophenyl)-1-(4-{8-methyl-[1,2,4]triazolo[4,3-b]pyridazin-6-yl}piperazin-1-yl)ethan-1-one;
2-(3,4-dichlorophenyl)-1-(4-7-methyl-[1,2,4]triazolo[4,3-b]pyridazin-6-yl}piperazin-1-yl)ethan-1-one;
2-(3,4-dichlorophenyl)-1-{4-[3-(trifluoromethyl)-[1,2,4]triazolo[4,3-b]pyridazin-6-yl]piperazin-1-yl}ethan-1-one;
tert-butyl N-(3-{4-[2-oxo-2-(4-{[1,2,4]triazolo[4,3-b]pyridazin-6-yl}piperazin-1-yl)ethyl]phenyl}prop-2-yn-1-yl)carbamate;
2-[4-(hydroxymethyl)phenyl]-1-(4-{[1,2,4]triazolo[4,3-b]pyridazin-6-yl}piperazin-1-yl)ethan-1-one;
2-phenyl-1-(4-{[1,2,4]triazolo[4,3-b]pyridazin-6-yl}piperazin-1-yl)ethan-1-one;
2-[4-(3-aminoprop-1-yn-1-yl)phenyl]-1-(4-{[1,2,4]triazolo[4,3-b]pyridazin-6-yl}piperazin-1-yl)ethan-1-one;
2-(4-ethynylphenyl)-1-(4-{[1,2,4]triazolo[4,3-b]pyridazin-6-yl}piperazin-1-yl)ethan-1-one;
2-(2,3,4,5,6-pentafluorophenyl)-1-(4-{[1,2,4]triazolo[4,3-b]pyridazin-6-yl}piperazin-1-yl)ethan-1-one;
2-(4-bromo-3-chlorophenyl)-1-(4-{[1,2,4]triazolo[4,3-b]pyridazin-6-yl}piperazin-1-yl)ethan-1-one;
2-(3-bromo-4-chlorophenyl)-1-(4-{[1,2,4]triazolo[4,3-b]pyridazin-6-yl}piperazin-1-yl)ethan-1-one;
2-(4-bromo-3-fluorophenyl)-1-(4-{[1,2,4]triazolo[4,3-b]pyridazin-6-yl}piperazin-1-yl)ethan-1-one;
2-(3-bromo-4-fluorophenyl)-1-(4-{[1,2,4]triazolo[4,3-b]pyridazin-6-yl}piperazin-1-yl)ethan-1-one;
tert-butyl N-(3-{4-[2-oxo-2-(4-{[1,2,4]triazolo[4,3-b]pyridazin-6-yl}piperazin-1-yl)ethyl]phenyl}propyl)carbamate;
2-(3,4-dichlorophenyl)-1-(4-{3-[2-(trifluoromethyl)pyridin-4-yl]-[1,2,4]triazolo[4,3-b]pyridazin-6-yl}piperazin-1-yl)ethan-1-one;
2-(2H-1,3-benzodioxol-5-yl)-1-(4-{[1,2,4]triazolo[4,3-b]pyridazin-6-yl}piperazin-1-yl)ethan-1-one;
tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate;
2-[3-chloro-5-(trifluoromethyl)phenyl]-1-(4-{[1,2,4]triazolo[4,3-b]pyridazin-6-yl}piperazin-1-yl)ethan-1-one;
2-(3,5-difluorophenyl)-1-(4-{[1,2,4]triazolo[4,3-b]pyridazin-6-yl}piperazin-1-yl)ethan-1-one;
2-[4-(4-hydroxybenzoyl)phenyl]-1-(4-{[1,2,4]triazolo[4,3-b]pyridazin-6-yl}piperazin-1-yl)ethan-1-one;
2-(4-fluoro-3-methoxyphenyl)-1-(4-{[1,2,4]triazolo[4,3-b]pyridazin-6-yl}piperazin-1-yl)ethan-1-one;
2-[3,5-bis(trifluoromethyl)phenyl]-1-(4-{[1,2,4]triazolo[4,3-b]pyridazin-6-yl}piperazin-1-yl)ethan-1-one;
2-(3-fluoro-4-methoxyphenyl)-1-(4-{[1,2,4]triazolo[4,3-b]pyridazin-6-yl}piperazin-1-yl)ethan-1-one;
2-(3-methylphenyl)-1-(4-{[1,2,4]triazolo[4,3-b]pyridazin-6-yl}piperazin-1-yl)ethan-1-one;
2-(4-methylphenyl)-1-(4-{[1,2,4]triazolo[4,3-b]pyridazin-6-yl}piperazin-1-yl)ethan-1-one;
1-(4-{[1,2,4]triazolo[4,3-b]pyridazin-6-yl}piperazin-1-yl)-2-(3,4,5-trifluorophenyl)ethan-1-one;
2-[4-fluoro-3-(propan-2-yl)phenyl]-1-(4-{[1,2,4]triazolo[4,3-b]pyridazin-6-yl}piperazin-1-yl)ethan-1-one;
2-(3-cyclopropyl-4-fluorophenyl)-1-(4-{[1,2,4]triazolo[4,3-b]pyridazin-6-yl}piperazin-1-yl)ethan-1-one;
2-[3-(difluoromethyl)-4-fluorophenyl]-l-(4-{[l,2,4]triazolo[4,3-b]pyridazin-6-yl}piperazin-1-yl)ethan-1-one;
2-[4-fluoro-3-(fluoromethyl)phenyl]-1-(4-{[1,2,4]triazolo[4,3-b]pyridazin-6-yl}piperazin-1-yl)ethan-1-one;
2-[3,4-bis(trifluoromethyl)phenyl]-1-(4-{[1,2,4]triazolo[4,3-b]pyridazin-6-yl}piperazin-1-yl)ethan-1-one;
2-[3-fluoro-5-(trifluoromethyl)phenyl]-1-(4-{[1,2,4]triazolo[4,3-b]pyridazin-6-yl}piperazin-1-yl)ethan-1-one;
2-[2-fluoro-3-(trifluoromethyl)phenyl]-1-(4-{[1,2,4]triazolo[4,3-b]pyridazin-6-yl}piperazin-1-yl)ethan-1-one;
2-fluoro-5-[2-oxo-2-(4-{[1,2,4]triazolo[4,3-b]pyridazin-6-yl}piperazin-1-yl)ethyl]benzonitrile;
2-(4-fluoro-3-nitrophenyl)-1-(4-{[1,2,4]triazolo[4,3 -b]pyridazin-6-yl}piperazin-1-yl)ethan-1-one;
2-[4-fluoro-3-(trifluoromethoxy)phenyl]-1-(4-{[1,2,4]triazolo[4,3-b]pyridazin-6-yl}piperazin-1-yl)ethan-1-one;
2-{6-fluoro-[1,1′-biphenyl]-3-yl}-1-(4-{[1,2,4]triazolo[4,3-b]pyridazin-6-yl}piperazin-1-yl)ethan-1-one;
2-{4′,6-difluoro-[1,1′-biphenyl]-3-yl}-1-(4-{[1,2,4]triazolo[4,3-b]pyridazin-6-yl}piperazin-1-yl)ethan-1-one;
2-[3-(1,1-difluoroethyl)-4-fluorophenyl]-1-(4-{[1,2,4]triazolo[4,3-b]pyridazin-6-yl}piperazin-1-yl)ethan-1-one;
2-[4-fluoro-3-(1,1,2,2,2-pentafluoroethyl)phenyl]-1-(4-{[1,2,4]triazolo[4,3-b]pyridazin-6-yl}piperazin-1-yl)ethan-1-one;
2-(3-ethynyl-4-fluorophenyl)-1-(4-{[1,2,4]triazolo[4,3-b]pyridazin-6-yl}piperazin-1-yl)ethan-1-one;
2-[4-fluoro-3-(prop-1-yn-1-yl)phenyl]-1-(4-{[1,2,4]triazolo[4,3-b]pyridazin-6-yl}piperazin-1-yl)ethan-1-one;
2-(3-tert-butyl-4-fluorophenyl)-1-(4-{[1,2,4]triazolo[4,3-b]pyridazin-6-yl}piperazin-1-yl)ethan-1-one;
2-[4-fluoro-3-(hydroxymethyl)phenyl]-1-(4-{[1,2,4]triazolo[4,3-b]pyridazin-6-yl}piperazin-1-yl)ethan-1-one;
2-[4-fluoro-3-(methoxymethyl)phenyl]-1-(4-{[1,2,4]triazolo[4,3-b]pyridazin-6-yl}piperazin-1-yl)ethan-1-one;
2-fluoro-5-[2-oxo-2-(4-{[1,2,4]triazolo[4,3-b]pyridazin-6-yl}piperazin-1-yl)ethyl]benzoic acid;
2-fluoro-5-[2-oxo-2-(4-{[1,2,4]triazolo[4,3-b]pyridazin-6-yl}piperazin-1-yl)ethyl]benzamide;
2-fluoro-N-methyl-5-[2-oxo-2-(4-{[1,2,4]triazolo[4,3-b]pyridazin-6-yl}piperazin-1-yl)ethyl]benzamide;
2-fluoro-N,N-dimethyl-5-[2-oxo-2-(4-{[1,2,4]triazolo[4,3-b]pyridazin-6-yl}piperazin-1-yl)ethyl]benzamide;
2-fluoro-5-[2-oxo-2-(4-{[1,2,4]triazolo[4,3-b]pyridazin-6-yl}piperazin-1-yl)ethyl]benzene-1-sulfonamide;
2-fluoro-N-methyl-5-[2-oxo-2-(4-{[1,2,4]triazolo[4,3-b]pyridazin-6-yl}piperazin-1-yl)ethyl]benzene-1-sulfonamide;
2-fluoro-N,N-dimethyl-5-[2-oxo-2-(4-{[1,2,4]triazolo[4,3-b]pyridazin-6-yl}piperazin-1-yl)ethyl]benzene-1-sulfonamide;
2-(4-fluoro-3-methanesulfonylphenyl)-1-(4-{[1,2,4]triazolo[4,3-b]pyridazin-6-yl}piperazin-1-yl)ethan-1-one;
and combinations thereof.

14. The composition of claim 13, wherein the one or more compounds are chosen from:
2-[4-fluoro-3-(trifluoromethyl)phenyl]-1-(4-{[1,2,4]triazolo[4,3-b]pyridazin-6-yl}piperazin-1-yl)ethan-1-one;
2-(3-bromo-4-chlorophenyl)-1-(4-{[1,2,4]triazolo[4,3-b]pyridazin-6-yl}piperazin-1-yl)ethan-1-one;
2-(3,4-dichlorophenyl)-1-(4-{[1,2,4]triazolo[4,3-b]pyridazin-6-yl}piperazin-1-yl)ethan-1-one;
2-(3-bromo-4-fluorophenyl)-1-(4-{[1,2,4]triazolo[4,3-b]pyridazin-6-yl}piperazin-1-yl)ethan-1-one;
2-(3-chloro-4-fluorophenyl)-1-(4-{[1,2,4]triazolo[4,3-b]pyridazin-6-yl}piperazin-1-yl)ethan-1-one;
2-(4-bromo-3-chlorophenyl)-1-(4-{[1,2,4]triazolo[4,3-b]pyridazin-6-yl}piperazin-1-yl)ethan-1-one;
2-[4-chloro-3-(trifluoromethyl)phenyl]-1-(4-{[1,2,4]triazolo[4,3-b]pyridazin-6-yl}piperazin-1-yl)ethan-1-one;
2-(4-chloro-3-methylphenyl)-1-(4-{[1,2,4]triazolo[4,3-b]pyridazin-6-yl}piperazin-1-yl)ethan-1-one;
2-[3,5-bis(trifluoromethyl)phenyl]-1-(4-{[1,2,4]triazolo[4,3-b]pyridazin-6-yl}piperazin-1-yl)ethan-1-one;
2-(4-fluoro-3-methylphenyl)-1-(4-{[1,2,4]triazolo[4,3-b]pyridazin-6-yl}piperazin-1-yl)ethan-1-one;
2-[3-chloro-5-(trifluoromethyl)phenyl]-1-(4-{[1,2,4]triazolo[4,3-b]pyridazin-6-yl}piperazin-1-yl)ethan-1-one;
2-(4-chloro-3-fluorophenyl)-1-(4-{[1,2,4]triazolo[4,3-b]pyridazin-6-yl}piperazin-1-yl)ethan-1-one;
2-(3,4-difluorophenyl)-1-(4-{[1,2,4]triazolo[4,3-b]pyridazin-6-yl}piperazin-1-yl)ethan-1-one;
2-(4-bromo-3-fluorophenyl)-1-(4-{[1,2,4]triazolo[4,3-b]pyridazin-6-yl}piperazin-1-yl)ethan-1-one;
2-(4-chlorophenyl)-1-(4-{[1,2,4]triazolo[4,3-b]pyridazin-6-yl}piperazin-1-yl)ethan-1-one;
2-(3-chloro-5-fluorophenyl)-1-(4-{[1,2,4]triazolo[4,3-b]pyridazin-6-yl}piperazin-1-yl)ethan-1-one;
2-(4-ethynylphenyl)-1-(4-{ [1,2,4]triazolo[4,3 -b]pyridazin-6-yl } piperazin-1 -yl)ethan-1-one;
2-(3,5-dichlorophenyl)-1-(4-{[1,2,4]triazolo[4,3-b]pyridazin-6-yl}piperazin-1-yl)ethan-1-one; and
and combinations thereof.

15. A method for treating an individual diagnosed with or suspected of having a Cryptosporidium infection or prophylaxis in an individual who is at risk of having a Cryptosporidium infection comprising administering one or more compounds having the following structure: 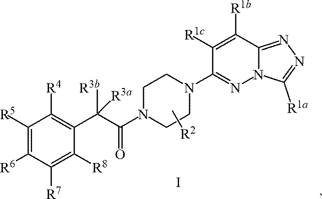
wherein
R^{1a}, R^{1b}, and R^{1c} are independently chosen from H, substituted alkyl groups, unsubstituted alkyl groups, substituted aryl groups, unsubstituted aryl groups, substituted aralkyl groups, and unsubstituted aralkyl groups;
R² is chosen from H, substituted alkyl groups, and unsubstituted alkyl groups;
R^{3a} and R^{3b} are independently chosen from H, substituted alkyl groups, unsubstituted alkyl groups, halogen, OH, NH₂, and R^{3a} and R^{3b} may combine to form a ring or a ═O;
R⁴ and R⁸ are independently chosen from H, halogens, substituted alkyl groups, and unsubstituted alkyl groups;
R⁵, R⁶, and R⁷ are independently chosen from H, halogens, substituted alkyl groups, unsubstituted alkyl groups, substituted cycloalkyl groups, unsubstituted cycloalkyl groups, substituted alkoxy groups, unsubstituted alkoxy groups, substituted aryl groups, unsubstituted aryl groups, substituted aralkyl groups, unsubstituted aralkyl groups, SO₂NR′R″, (CH₂)ₙ-alkoxy groups (wherein n is 1, 2, 3, or 4), (CH₂)ₙNHCO-alkyl (wherein alkyl is substituted or unsubstituted, and n is 1, 2, 3, or 4), nitro groups, alcohol groups, —SFs groups, CONR′R″, R⁵ is joined with R⁴ to make a ring, R⁵ is joined with R⁶ to make a ring, R⁶ is joined with R⁷ to make a ring, and R⁷ is joined with R⁸ to make a ring, wherein R′ and R″ are independently chosen from H, substituted alkyl groups, and unsubstituted alkyl group,
or a composition comprising the one or more compounds in a therapeutically effective or prophylactically effective amount.

16. The method of claim 15, wherein the individual is a human or non-human animal.

17. The method of claim 15, wherein the one or more compounds are chosen from:
2-(2-chlorophenyl)-1-(4-{[1,2,4]triazolo[4,3-b]pyridazin-6-yl}piperazin-1-yl)ethan-1-one;
2-(naphthalen-1-yl)-1-(4-{[1,2,4]triazolo[4,3-b]pyridazin-6-yl}piperazin-1-yl)ethan-1-one;
2-(4-chloro-3-fluorophenyl)-1-(4-{[1,2,4]triazolo[4,3-b]pyridazin-6-yl}piperazin-1-yl)ethan-1-one;
2-(4-fluorophenyl)-1-(4-{[1,2,4]triazolo[4,3-b]pyridazin-6-yl}piperazin-1-yl)ethan-1-one
2-(3,4-dichlorophenyl)-1-(4-{[1,2,4]triazolo[4,3-b]pyridazin-6-yl}piperazin-1-yl)ethan-1-one;
2-(4-chlorophenyl)-1-(4-{[1,2,4]triazolo[4,3-b]pyridazin-6-yl}piperazin-1-yl)ethan-1-one;
2-(2,6-dichlorophenyl)-1-(4-{[1,2,4]triazolo[4,3-b]pyridazin-6-yl}piperazin-1-yl)ethan-1-one;
2-(2,4-dichlorophenyl)-1-(4-{[1,2,4]triazolo[4,3-b]pyridazin-6-yl}piperazin-1-yl)ethan-1-one;
2-(2,5-dichlorophenyl)-1-(4-{[1,2,4]triazolo[4,3-b]pyridazin-6-yl}piperazin-1-yl)ethan-1-one;
2-(3,5-dichlorophenyl)-1-(4-{[1,2,4]triazolo[4,3-b]pyridazin-6-yl}piperazin-1-yl)ethan-1-one;
2-(naphthalen-2-yl)-1-(4-{[1,2,4]triazolo[4,3-b]pyridazin-6-yl}piperazin-1-yl)ethan-1-one
2-(3,4-dichlorophenyl)-1-(2-methyl-4-{[1,2,4]triazolo[4,3-b]pyridazin-6-yl}piperazin-1-yl)ethan-1-one;
2-(4-nitrophenyl)-1-(4-{[1,2,4]triazolo[4,3-b]pyridazin-6-yl}piperazin-1-yl)ethan-1-one;
2-(3-chloro-4-methylphenyl)-1-(4-{[1,2,4]triazolo[4,3-b]pyridazin-6-yl}piperazin-1-yl)ethan-1-one;
2-(3-fluoro-4-methylphenyl)-1-(4-{[1,2,4]triazolo[4,3-b]pyridazin-6-yl}piperazin-1-yl)ethan-1-one;
2-(4-chloro-3-methylphenyl)-1-(4-{[1,2,4]triazolo[4,3-b]pyridazin-6-yl}piperazin-1-yl)ethan-1-one;
2-(4-fluoro-3-methylphenyl)-1-(4-{[1,2,4]triazolo[4,3-b]pyridazin-6-yl}piperazin-1-yl)ethan-1-one;
2-(3-bromophenyl)-1-(4-{[1,2,4]triazolo[4,3-b]pyridazin-6-yl}piperazin-1-yl)ethan-1-one;
2-(4-methoxyphenyl)-1-(4-{[1,2,4]triazolo[4,3-b]pyridazin-6-yl}piperazin-1-yl)ethan-1-one;
2-(3-methoxyphenyl)-1-(4-{[1,2,4]triazolo[4,3-b]pyridazin-6-yl}piperazin-1-yl)ethan-1-one;
2-(3-chloro-4-fluorophenyl)-1-(4-{[1,2,4]triazolo[4,3-b]pyridazin-6-yl}piperazin-1-yl)ethan-1-one;
2-(3-chlorophenyl)-1-(4-{[1,2,4]triazolo[4,3-b]pyridazin-6-yl}piperazin-1-yl)ethan-1-one;
1-(3,4-dichlorophenyl)-2-(4-{[1,2,4]triazolo[4,3-b]pyridazin-6-yl}piperazin-1-yl)ethane-1,2-dione;
1-[1-(3,4-dichlorophenyl)cyclopropanecarbonyl]-4-{[1,2,4]triazolo[4,3-b]pyridazin-6-yl}piperazine;
2-(4-bromophenyl)-1-(4-{[1,2,4]triazolo[4,3-b]pyridazin-6-yl}piperazin-1-yl)ethan-1-one;
2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one;
4-[2-oxo-2-(4-{[1,2,4]triazolo[4,3-b]pyridazin-6-yl}piperazin-1-yl)ethyl]benzonitrile;
2-(2,3-dichlorophenyl)-1-(4-{[1,2,4]triazolo[4,3-b]pyridazin-6-yl}piperazin-1-yl)ethan-1-one;
2-(3,4-dichlorophenyl)-2,2-difluoro-1-(4-{[1,2,4]triazolo[4,3-b]pyridazin-6-yl}piperazin-1-yl)ethan-1-one;
2-(3,4-difluorophenyl)-1-(4-{[1,2,4]triazolo[4,3-b]pyridazin-6-yl}piperazin-1-yl)ethan-1-one;
2-(3,4-dichlorophenyl)-1-(4-{3-methyl-[1,2,4]triazolo[4,3-b]pyridazin-6-yl}piperazin-1-yl)ethan-1-one;
2-(3,4-dichlorophenyl)-1-(4-{3-phenyl-[1,2,4]triazolo[4,3-b]pyridazin-6-yl}piperazin-1-yl)ethan-1-one;
1-(4-{3-benzyl-[1,2,4]triazolo[4,3-b]pyridazin-6-yl}piperazin-1-yl)-2-(3,4-dichlorophenyl)ethan-1-one;
2-(3,4-dichlorophenyl)-1-{4-[3-(trifluoromethyl)-[1,2,4]triazolo[4,3-b]pyridazin-6-yl]piperazin-1-yl}ethan-1-one;
1-(4-{3-cyclopropyl-[1,2,4]triazolo[4,3-b]pyridazin-6-yl}piperazin-1-yl)-2-(3,4-dichlorophenyl)ethan-1-one;
2-(3,4-dichlorophenyl)-1-(4-{[1,2,4]triazolo[4,3-b]pyridazin-6-yl}piperazin-1-yl)propan-1-one;
2-(3,4-dichlorophenyl)-2-methyl-1-(4-{[1,2,4]triazolo[4,3-b]pyridazin-6-yl}piperazin-1-yl)propan-1-one;
2-(3,4-dichlorophenyl)-1-(3-methyl-4-{[1,2,4]triazolo[4,3-b]pyridazin-6-yl}piperazin-1-yl)ethan-1-one;
2-(3,4-dichlorophenyl)-1-(3,5-dimethyl-4-{[1,2,4]triazolo[4,3-b]pyridazin-6-yl}piperazin-1-yl)ethan-1-one;
2-(3,4-dichlorophenyl)-1-(2,6-dimethyl-4-{[1,2,4]triazolo[4,3-b]pyridazin-6-yl}piperazin-1-yl)ethan-1-one;
2-(3-chloro-5-fluorophenyl)-1-(4-{[1,2,4]triazolo[4,3-b]pyridazin-6-yl}piperazin-1-yl)ethan-1-one;
2-[4-(pentafluoro-λ⁶-sulfanyl)phenyl]-1-(4-{[1,2,4]triazolo[4,3-b]pyridazin-6-yl}piperazin-1-yl)ethan-1-one;
2-[3-(pentafluoro-λ⁶-sulfanyl)phenyl]-1-(4-{[1,2,4]triazolo[4,3-b]pyridazin-6-yl}piperazin-1-yl)ethan-1-one;
2-[4-chloro-3-(trifluoromethyl)phenyl]-1-(4-{[1,2,4]triazolo[4,3-b]pyridazin-6-yl}piperazin-1-yl)ethan-1-one;
2-[3-chloro-4-(trifluoromethyl)phenyl]-1-(4-{[1,2,4]triazolo[4,3-b]pyridazin-6-yl}piperazin-1-yl)ethan-1-one;
2-[4-fluoro-3-(trifluoromethyl)phenyl]-1-(4-{[1,2,4]triazolo[4,3-b]pyridazin-6-yl}piperazin-1-yl)ethan-1-one;
2-[3-fluoro-4-(trifluoromethyl)phenyl]-1-(4-{[1,2,4]triazolo[4,3-b]pyridazin-6-yl}piperazin-1-yl)ethan-1-one;
1-(4-{[1,2,4]triazolo[4,3-b]pyridazin-6-yl}piperazin-1-yl)-2-[3-(trifluoromethyl)phenyl]ethan-1-one;
1-(4-{[1,2,4]triazolo[4,3-b]pyridazin-6-yl}piperazin-1-yl)-2-[4-(trifluoromethyl)phenyl]ethan-1-one;
2-(2,3-dihydro-1H-inden-5-yl)-1-(4-{[1,2,4]triazolo[4,3-b]pyridazin-6-yl}piperazin-1-yl)ethan-1-one;
2-(3-tert-butylphenyl)-1-(4-{[1,2,4]triazolo[4,3-b]pyridazin-6-yl}piperazin-1-yl)ethan-1-one;
2-(4-tert-butylphenyl)-1-(4-{[1,2,4]triazolo[4,3-b]pyridazin-6-yl}piperazin-1-yl)ethan-1-one;
2-(4-chloro-3-ethylphenyl)-1-(4-{[1,2,4]triazolo[4,3-b]pyridazin-6-yl}piperazin-1-yl)ethan-1-one;
2-(3-ethyl-4-fluorophenyl)-1-(4-1,2,4]triazolo[4,3-b]pyridazin-6-ylpiperazin-1-yl)ethan-1-one;
2-[4-chloro-3-(propan-2-yl)phenyl]-1-(4-{[1,2,4]triazolo[4,3-b]pyridazin-6-yl}piperazin-1-yl)ethan-1-one;
2-(4-chloro-3-cyclopropylphenyl)-1-(4-{[1,2,4]triazolo[4,3-b]pyridazin-6-yl}piperazin-1-yl)ethan-1-one;
2-(4-methylphenyl)-1-(4-{[1,2,4]triazolo[4,3-b]pyridazin-6-yl}piperazin-1-yl)ethan-1-one;
2-(3-methylphenyl)-1-(4-{[1,2,4]triazolo[4,3-b]pyridazin-6-yl}piperazin-1-yl)ethan-1-one;
4-[2-oxo-2-(4-{[1,2,4]triazolo[4,3-b]pyridazin-6-yl}piperazin-1-yl)ethyl]benzamide;
3-[2-oxo-2-(4-{[1,2,4]triazolo[4,3-b]pyridazin-6-yl}piperazin-1-yl)ethyl]benzamide;
N-cyclopropyl-4-[2-oxo-2-(4-{[1,2,4]triazolo[4,3-b]pyridazin-6-yl}piperazin-1-yl)ethyl]benzamide;
N-cyclopropyl-3-[2-oxo-2-(4-{[1,2,4]triazolo[4,3-b]pyridazin-6-yl}piperazin-1-yl)ethyl]benzamide;
N,N-dimethyl-4-[2-oxo-2-(4-{[1,2,4]triazolo[4,3-b]pyridazin-6-yl}piperazin-1-yl)ethyl]benzamide;
N,N-dimethyl-3-[2-oxo-2-(4-{[1,2,4]triazolo[4,3-b]pyridazin-6-yl}piperazin-1-yl)ethyl]benzamide;
2-[4-(pyrrolidine-1-carbonyl)phenyl]-1-(4-{[1,2,4]triazolo[4,3-b]pyridazin-6-yl}piperazin-1-yl)ethan-1-one;
2-[3-(pyrrolidine-1-carbonyl)phenyl]-1-(4-{[1,2,4]triazolo[4,3-b]pyridazin-6-yl}piperazin-1-yl)ethan-1-one;
N-(2-hydroxyethyl)-4-[2-oxo-2-(4-{[1,2,4]triazolo[4,3-b]pyridazin-6-yl}piperazin-1-yl)ethyl]benzamide;
N-(2-hydroxyethyl)-3-[2-oxo-2-(4-{[1,2,4]triazolo[4,3-b]pyridazin-6-yl}piperazin-1-yl)ethyl]benzamide;
N-benzyl-4-[2-oxo-2-(4-{[1,2,4]triazolo[4,3-b]pyridazin-6-yl}piperazin-1-yl)ethyl]benzamide;
N-benzyl-3-[2-oxo-2-(4-{[1,2,4]triazolo[4,3-b]pyridazin-6-yl}piperazin-1-yl)ethyl]benzamide;
4-[2-oxo-2-(4-{[1,2,4]triazolo[4,3-b]pyridazin-6-yl}piperazin-1-yl)ethyl]benzene-1-sulfonamide;
3-[2-oxo-2-(4-{[1,2,4]triazolo[4,3-b]pyridazin-6-yl}piperazin-1-yl)ethyl]benzene-1-sulfonamide;
N-cyclopropyl-4-[2-oxo-2-(4-{[1,2,4]triazolo[4,3-b]pyridazin-6-yl}piperazin-1-yl)ethyl]benzene-1-sulfonamide;
N-cyclopropyl-3-[2-oxo-2-(4-{[1,2,4]triazolo[4,3-b]pyridazin-6-yl}piperazin-1-yl)ethyl]benzene-1-sulfonamide;
2-(4-methanesulfonylphenyl)-1-(4-{[1,2,4]triazolo[4,3-b]pyridazin-6-yl}piperazin-1-yl)ethan-1-one;
2-(3-methanesulfonylphenyl)-1-(4-{[1,2,4]triazolo[4,3-b]pyridazin-6-yl}piperazin-1-yl)ethan-1-one;
2-{[1,1′-biphenyl]-4-yl}-1-(4-{[1,2,4]triazolo[4,3-b]pyridazin-6-yl}piperazin-1-yl)ethan-1-one;
2-{[1,1′-biphenyl]-3-yl}-1-(4-{[1,2,4]triazolo[4,3-b]pyridazin-6-yl}piperazin-1-yl)ethan-1-one;
2-(4-benzylphenyl)-1-(4-{[1,2,4]triazolo[4,3-b]pyridazin-6-yl}piperazin-1-yl)ethan-1-one;
2-(3-benzylphenyl)-1-(4-{[1,2,4]triazolo[4,3-b]pyridazin-6-yl}piperazin-1-yl)ethan-1-one;
2-(4-phenoxyphenyl)-1-(4-{[1,2,4]triazolo[4,3-b]pyridazin-6-yl}piperazin-1-yl)ethan-1-one;
2-(3-phenoxyphenyl)-1-(4-{[1,2,4]triazolo[4,3-b]pyridazin-6-yl}piperazin-1-yl)ethan-1-one;
2-[4-(2-phenylethyl)phenyl]-1-(4-{[1,2,4]triazolo[4,3-b]pyridazin-6-yl}piperazin-1-yl)ethan-1-one;
2-[3-(2-phenylethyl)phenyl]-1-(4-{[1,2,4]triazolo[4,3-b]pyridazin-6-yl}piperazin-1-yl)ethan-1-one;
2-[3-(hydroxymethyl)phenyl]-1-(4-{[1,2,4]triazolo[4,3-b]pyridazin-6-yl}piperazin-1-yl)ethan-1-one;
2-[3-chloro-4-(hydroxymethyl)phenyl]-1-(4-{[1,2,4]triazolo[4,3-b]pyridazin-6-yl}piperazin-1-yl)ethan-1-one;
2-[4-chloro-3-(hydroxymethyl)phenyl]-1-(4-{[1,2,4]triazolo[4,3-b]pyridazin-6-yl}piperazin-1-yl)ethan-1-one;
2-{4-[(pyrrolidin-1-yl)methyl]phenyl}-1-(4-{[1,2,4]triazolo[4,3-b]pyridazin-6-yl}piperazin-1-yl)ethan-1-one;
2-{3-[(pyrrolidin-1-yl)methyl]phenyl}-1-(4-{[1,2,4]triazolo[4,3-b]pyridazin-6-yl}piperazin-1-yl)ethan-1-one;
2-{3-chloro-4-[(pyrrolidin-1-yl)methyl]phenyl}-1-(4-{[1,2,4]triazolo[4,3-b]pyridazin-6-yl}piperazin-1-yl)ethan-1-one;
2-{4-chloro-3-[(pyrrolidin-1-yl)methyl]phenyl}-1-(4-{[1,2,4]triazolo[4,3-b]pyridazin-6-yl}piperazin-1-yl)ethan-1-one;
2-{4-[(morpholin-4-yl)methyl]phenyl}-1-(4-{[1,2,4]triazolo[4,3-b]pyridazin-6-yl}piperazin-1-yl)ethan-1-one;
2-{3-[(morpholin-4-yl)methyl]phenyl}-1-(4-{[1,2,4]triazolo[4,3-b]pyridazin-6-yl}piperazin-1-yl)ethan-1-one;
2-{3-chloro-4-[(morpholin-4-yl)methyl]phenyl}-1-(4-{[1,2,4]triazolo[4,3-b]pyridazin-6-yl}piperazin-1-yl)ethan-1-one;
2-{4-chloro-3-[(morpholin-4-yl)methyl]phenyl}-1-(4-{[1,2,4]triazolo[4,3-b]pyridazin-6-yl}piperazin-1-yl)ethan-1-one;
2-methyl-N-({4-[2-oxo-2-(4-{[1,2,4]triazolo[4,3-b]pyridazin-6-yl}piperazin-1-yl)ethyl]phenyl}methyl)propanamide;
2-methyl-N-({3-[2-oxo-2-(4-{[1,2,4]triazolo[4,3-b]pyridazin-6-yl}piperazin-1-yl)ethyl]phenyl}methyl)propanamide;
N-({2-chloro-4-[2-oxo-2-(4-{[1,2,4]triazolo[4,3-b]pyridazin-6-yl}piperazin-1-yl)ethyl]phenyl}methyl)-2-methylpropanamide;
N-({2-chloro-5-[2-oxo-2-(4-{[1,2,4]triazolo[4,3-b]pyridazin-6-yl}piperazin-1-yl)ethyl]phenyl}methyl)-2-methylpropanamide;
2-{3-chloro-4-[(cyclopropylamino)methyl]phenyl}-1-(4-{[1,2,4]triazolo[4,3-b]pyridazin-6-yl}piperazin-1-yl)ethan-1-one;
2-{4-chloro-3-[(cyclopropylamino)methyl]phenyl}-1-(4-{[1,2,4]triazolo[4,3-b]pyridazin-6-yl}piperazin-1-yl)ethan-1-one;
N,N-dimethyl-4-[2-oxo-2-(4-{[1,2,4]triazolo[4,3-b]pyridazin-6-yl}piperazin-1-yl)ethyl]benzene-1-sulfonamide;
N,N-dimethyl-3-[2-oxo-2-(4-{[1,2,4]triazolo[4,3-b]pyridazin-6-yl}piperazin-1-yl)ethyl]benzene-1-sulfonamide;
2-[4-(pyrrolidine-1-sulfonyl)phenyl]-1-(4-{[1,2,4]triazolo[4,3-b]pyridazin-6-yl}piperazin-1-yl)ethan-1-one;
2-[3-(pyrrolidine-1-sulfonyl)phenyl]-1-(4-{[1,2,4]triazolo[4,3-b]pyridazin-6-yl}piperazin-1-yl)ethan-1-one;
2-(3-ethynylphenyl)-1-(4-{[1,2,4]triazolo[4,3-b]pyridazin-6-yl}piperazin-1-yl)ethan-1-one;
2-{4-[(piperidin-1-yl)methyl]phenyl}-1-(4-{[1,2,4]triazolo[4,3-b]pyridazin-6-yl}piperazin-1-yl)ethan-1-one;
2-{4-[(4,4-difluoropiperidin-1-yl)methyl]phenyl}-1-(4-{[1,2,4]triazolo[4,3-b]pyridazin-6-yl}piperazin-1-yl)ethan-1-one;
2-{4-[(dimethylamino)methyl]phenyl}-1-(4-{[1,2,4]triazolo[4,3-b]pyridazin-6-yl}piperazin-1-yl)ethan-1-one;
2-[4-chloro-3-(difluoromethyl)phenyl]-1-(4-{[1,2,4]triazolo[4,3-b]pyridazin-6-yl}piperazin-1-yl)ethan-1-one;
2-[4-chloro-3-(fluoromethyl)phenyl]-1-(4-{[1,2,4]triazolo[4,3-b]pyridazin-6-yl}piperazin-1-yl)ethan-1-one;
2-(3,4-dichlorophenyl)-1-(4-{8-methyl-[1,2,4]triazolo[4,3-b]pyridazin-6-yl}piperazin-1-yl)ethan-1-one;
2-(3,4-dichlorophenyl)-1-(4-{7-methyl-[1,2,4]triazolo[4,3-b]pyridazin-6-yl}piperazin-1-yl)ethan-1-one;
2-(3,4-dichlorophenyl)-1-{4-[3-(trifluoromethyl)-[1,2,4]triazolo[4,3-b]pyridazin-6-yl]piperazin-1-yl}ethan-1-one;
tert-butyl N-(3-{4-[2-oxo-2-(4-{[1,2,4]triazolo[4,3-b]pyridazin-6-yl}piperazin-1-yl)ethyl]phenyl}prop-2-yn-1-yl)carbamate;
2-[4-(hydroxymethyl)phenyl]-1-(4-{[1,2,4]triazolo[4,3-b]pyridazin-6-yl}piperazin-1-yl)ethan-1-one;
2-phenyl-1-(4-{[1,2,4]triazolo[4,3-b]pyridazin-6-yl}piperazin-1-yl)ethan-1-one;
2-[4-(3-aminoprop-1-yn-1-yl)phenyl]-1-(4-{[1,2,4]triazolo[4,3-b]pyridazin-6-yl}piperazin-1-yl)ethan-1-one;
2-(4-ethynylphenyl)-1-(4-{[1,2,4]triazolo[4,3-b]pyridazin-6-yl}piperazin-1-yl)ethan-1-one;
2-(2,3,4,5,6-pentafluorophenyl)-1-(4-{[1,2,4]triazolo[4,3-b]pyridazin-6-yl}piperazin-1-yl)ethan-1-one;
2-(4-bromo-3-chlorophenyl)-1-(4-{[1,2,4]triazolo[4,3-b]pyridazin-6-yl}piperazin-1-yl)ethan-1-one;
2-(3-bromo-4-chlorophenyl)-1-(4-{[1,2,4]triazolo[4,3-b]pyridazin-6-yl}piperazin-1-yl)ethan-1-one;
2-(4-bromo-3-fluorophenyl)-1-(4-{[1,2,4]triazolo[4,3-b]pyridazin-6-yl}piperazin-1-yl)ethan-1-one;
2-(3-bromo-4-fluorophenyl)-1-(4-{[1,2,4]triazolo[4,3-b]pyridazin-6-yl}piperazin-1-yl)ethan-1-one;
tert-butyl N-(3-{4-[2-oxo-2-(4-{[1,2,4]triazolo[4,3-b]pyridazin-6-yl}piperazin-1-yl)ethyl]phenyl}propyl)carbamate;
2-(3,4-dichlorophenyl)-1-(4-{3-[2-(trifluoromethyl)pyridin-4-yl]-[1,2,4]triazolo[4,3-b]pyridazin-6-yl}piperazin-1-yl)ethan-1-one;
2-(2H-1,3-benzodioxol-5-yl)-1-(4-{[1,2,4]triazolo[4,3-b]pyridazin-6-yl}piperazin-1-yl)ethan-1-one;
tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate;
2-[3-chloro-5-(trifluoromethyl)phenyl]-1-(4-{[1,2,4]triazolo[4,3-b]pyridazin-6-yl}piperazin-1-yl)ethan-1-one;
2-(3,5-difluorophenyl)-1-(4-{[1,2,4]triazolo[4,3-b]pyridazin-6-yl}piperazin-1-yl)ethan-1-one;
2-[4-(4-hydroxybenzoyl)phenyl]-1-(4-{[1,2,4]triazolo[4,3-b]pyridazin-6-yl}piperazin-1-yl)ethan-1-one;
2-(4-fluoro-3-methoxyphenyl)-1-(4-{[1,2,4]triazolo[4,3-b]pyridazin-6-yl}piperazin-1-yl)ethan-1-one;
2-[3,5-bis(trifluoromethyl)phenyl]-1-(4-{[1,2,4]triazolo[4,3-b]pyridazin-6-yl}piperazin-1-yl)ethan-1-one;
2-(3-fluoro-4-methoxyphenyl)-1-(4-{[1,2,4]triazolo[4,3-b]pyridazin-6-yl}piperazin-1-yl)ethan-1-one;
2-(3-methylphenyl)-1-(4-{[1,2,4]triazolo[4,3-b]pyridazin-6-yl}piperazin-1-yl)ethan-1-one;
2-(4-methylphenyl)-1-(4-{[1,2,4]triazolo[4,3-b]pyridazin-6-yl}piperazin-1-yl)ethan-1-one;
1-(4-{[1,2,4]triazolo[4,3-b]pyridazin-6-yl}piperazin-1-yl)-2-(3,4,5-trifluorophenyl)ethan-1-one;
2-[4-fluoro-3-(propan-2-yl)phenyl]-1-(4-{[1,2,4]triazolo[4,3-b]pyridazin-6-yl}piperazin-1-yl)ethan-1-one;
2-(3-cyclopropyl-4-fluorophenyl)-1-(4-{[1,2,4]triazolo[4,3-b]pyridazin-6-yl}piperazin-1-yl)ethan-1-one;
2-[3-(difluoromethyl)-4-fluorophenyl]-1-(4-{[1,2,4]triazolo[4,3-b]pyridazin-6-yl}piperazin-1-yl)ethan-1-one;
2-[4-fluoro-3-(fluoromethyl)phenyl]-1-(4-{[1,2,4]triazolo[4,3-b]pyridazin-6-yl}piperazin-1-yl)ethan-1-one;
2-[3,4-bis(trifluoromethyl)phenyl]-1-(4-{[1,2,4]triazolo[4,3-b]pyridazin-6-yl}piperazin-1-yl)ethan-1-one;
2-[3-fluoro-5-(trifluoromethyl)phenyl]-1-(4-{[1,2,4]triazolo[4,3-b]pyridazin-6-yl}piperazin-1-yl)ethan-1-one;
2-[2-fluoro-3-(trifluoromethyl)phenyl]-1-(4-{[1,2,4]triazolo[4,3-b]pyridazin-6-yl}piperazin-1-yl)ethan-1-one;
2-fluoro-5-[2-oxo-2-(4-{[1,2,4]triazolo[4,3-b]pyridazin-6-yl}piperazin-1-yl)ethyl]benzonitrile;
2-(4-fluoro-3-nitrophenyl)-1-(4-{[1,2,4]triazolo[4,3-b]pyridazin-6-yl}piperazin-1-yl)ethan-1-one;
2-[4-fluoro-3-(trifluoromethoxy)phenyl]-1-(4-{[1,2,4]triazolo[4,3-b]pyridazin-6-yl}piperazin-1-yl)ethan-1-one;
2-{6-fluoro-[1,1′-biphenyl]-3-yl}-1-(4-{[1,2,4]triazolo[4,3-b]pyridazin-6-yl}piperazin-1-yl)ethan-1-one;
2-{4′,6-difluoro-[1,1′-biphenyl]-3-yl}-1-(4-{[1,2,4]triazolo[4,3-b]pyridazin-6-yl}piperazin-1-yl)ethan-1-one;
2-[3-(1,1-difluoroethyl)-4-fluorophenyl]-1-(4-{[1,2,4]triazolo[4,3-b]pyridazin-6-yl}piperazin-1-yl)ethan-1-one;
2-[4-fluoro-3-(1,1,2,2,2-pentafluoroethyl)phenyl]-1-(4-{[1,2,4]triazolo[4,3-b]pyridazin-6-yl}piperazin-1-yl)ethan-1-one;
2-(3-ethynyl-4-fluorophenyl)-1-(4-{[1,2,4]triazolo[4,3-b]pyridazin-6-yl}piperazin-1-yl)ethan-1-one;
2-[4-fluoro-3-(prop-1-yn-1-yl)phenyl]-1-(4-{[1,2,4]triazolo[4,3-b]pyridazin-6-yl}piperazin-1-yl)ethan-1-one;
2-(3-tert-butyl-4-fluorophenyl)-1-(4-{[1,2,4]triazolo[4,3-b]pyridazin-6-yl}piperazin-1-yl)ethan-1-one;
2-[4-fluoro-3-(hydroxymethyl)phenyl]-1-(4-{[1,2,4]triazolo[4,3-b]pyridazin-6-yl}piperazin-1-yl)ethan-1-one;
2-[4-fluoro-3-(methoxymethyl)phenyl]-1-(4-{[1,2,4]triazolo[4,3-b]pyridazin-6-yl}piperazin-1-yl)ethan-1-one;
2-fluoro-5-[2-oxo-2-(4-{[1,2,4]triazolo[4,3-b]pyridazin-6-yl}piperazin-1-yl)ethyl]benzoic acid;
2-fluoro-5-[2-oxo-2-(4-{[1,2,4]triazolo[4,3-b]pyridazin-6-yl}piperazin-1-yl)ethyl]benzamide;
2-fluoro-N-methyl-5-[2-oxo-2-(4-{[1,2,4]triazolo[4,3-b]pyridazin-6-yl}piperazin-1-yl)ethyl]benzamide;
2-fluoro-N,N-dimethyl-5-[2-oxo-2-(4-{[1,2,4]triazolo[4,3-b]pyridazin-6-yl}piperazin-1-yl)ethyl]benzamide;
2-fluoro-5-[2-oxo-2-(4-{[1,2,4]triazolo[4,3-b]pyridazin-6-yl}piperazin-1-yl)ethyl]benzene-1-sulfonamide;
2-fluoro-N-methyl-5-[2-oxo-2-(4-{[1,2,4]triazolo[4,3-b]pyridazin-6-yl}piperazin-1-yl)ethyl]benzene-1-sulfonamide;
2-fluoro-N,N-dimethyl-5-[2-oxo-2-(4-{[1,2,4]triazolo[4,3-b]pyridazin-6-yl}piperazin-1-yl)ethyl]benzene-1-sulfonamide;
2-(4-fluoro-3-methanesulfonylphenyl)-1-(4-{[1,2,4]triazolo[4,3-b]pyridazin-6-yl}piperazin-1-yl)ethan-1-one;
and combinations thereof.
2-(2-chlorophenyl)-1-(4-{[1,2,4]triazolo[4,3-b]pyridazin-6-yl}piperazin-1-yl)ethan-1-one;
2-(naphthalen-1-yl)-1-(4-{[1,2,4]triazolo[4,3-b]pyridazin-6-yl}piperazin-1-yl)ethan-1-one;
2-(4-chloro-3-fluorophenyl)-1-(4-{[1,2,4]triazolo[4,3-b]pyridazin-6-yl}piperazin-1-yl)ethan-1-one;
2-(4-fluorophenyl)-1-(4- {[1,2,4]triazolo[4,3-b]pyridazin-6-yl}piperazin-1-yl)ethan-1-one
2-(3,4-dichlorophenyl)-1-(4-{[1,2,4]triazolo[4,3-b]pyridazin-6-yl}piperazin-1-yl)ethan-1-one;
2-(4-chlorophenyl)-1-(4- {[1,2,4]triazolo[4,3-b]pyridazin-6-yl}piperazin-1-yl)ethan-1-one;
2-(2,6-dichlorophenyl)-1-(4-{[1,2,4]triazolo[4,3-b]pyridazin-6-yl}piperazin-1-yl)ethan-1-one;
2-(2,4-dichlorophenyl)-1-(4-{[1,2,4]triazolo[4,3-b]pyridazin-6-yl}piperazin-1-yl)ethan-1-one;
2-(2,5-dichlorophenyl)-1-(4-{[1,2,4]triazolo[4,3-b]pyridazin-6-yl}piperazin-1-yl)ethan-1-one;
2-(3,5-dichlorophenyl)-1-(4-{[1,2,4]triazolo[4,3-b]pyridazin-6-yl}piperazin-1-yl)ethan-1-one;
2-(naphthalen-2-yl)-1-(4-{[1,2,4]triazolo[4,3-b]pyridazin-6-yl}piperazin-1-yl)ethan-1-one
2-(3,4-dichlorophenyl)-1-(2-methyl-4-{[1,2,4]triazolo[4,3-b]pyridazin-6-yl}piperazin-1-yl)ethan-1-one;
2-(4-nitrophenyl)-1-(4-{[1,2,4]triazolo[4,3-b]pyridazin-6-yl}piperazin-1-yl)ethan-1-one;
2-(3-chloro-4-methylphenyl)-1-(4-{[1,2,4]triazolo[4,3-b]pyridazin-6-yl}piperazin-1-yl)ethan-1-one;
2-(3-fluoro-4-methylphenyl)-1-(4-{[1,2,4]triazolo[4,3-b]pyridazin-6-yl}piperazin-1-yl)ethan-1-one;
2-(4-chloro-3-methylphenyl)-1-(4-{[1,2,4]triazolo[4,3-b]pyridazin-6-yl}piperazin-1-yl)ethan-1-one;
2-(4-fluoro-3-methylphenyl)-1-(4-{[1,2,4]triazolo[4,3-b]pyridazin-6-yl}piperazin-1-yl)ethan-1-one;
2-(3-bromophenyl)-1-(4-{[1,2,4]triazolo[4,3-b]pyridazin-6-yl}piperazin-1-yl)ethan-1-one;
2-(4-methoxyphenyl)-1-(4-{[1,2,4]triazolo[4,3-b]pyridazin-6-yl}piperazin-1-yl)ethan-1-one;
2-(3-methoxyphenyl)-1-(4-{[1,2,4]triazolo[4,3-b]pyridazin-6-yl}piperazin-1-yl)ethan-1-one;
2-(3-chloro-4-fluorophenyl)-1-(4-{[1,2,4]triazolo[4,3-b]pyridazin-6-yl}piperazin-1-yl)ethan-1-one;
2-(3-chlorophenyl)-1-(4-{[1,2,4]triazolo[4,3-b]pyridazin-6-yl}piperazin-1-yl)ethan-1-one;
1-(3,4-dichlorophenyl)-2-(4-{[1,2,4]triazolo[4,3-b]pyridazin-6-yl}piperazin-1-yl)ethane-1,2-dione;
1-[1-(3,4-dichlorophenyl)cyclopropanecarbonyl]-4-{[1,2,4]triazolo[4,3-b]pyridazin-6-yl}piperazine;
2-(4-bromophenyl)-1-(4-{[1,2,4]triazolo[4,3-b]pyridazin-6-yl}piperazin-1-yl)ethan-1-one;
2-{4-[(1H-1,2,3-benzotriazol-1-yloxy)methyl]phenyl}-1-(4-{[1,2,4]triazolo[4,3-b]pyridazin-6-yl}piperazin-1-yl)ethan-1-one;
4-[2-oxo-2-(4-{[1,2,4]triazolo[4,3-b]pyridazin-6-yl}piperazin-1-yl)ethyl]benzonitrile;
2-(2,3-dichlorophenyl)-1-(4-{[1,2,4]triazolo[4,3-b]pyridazin-6-yl}piperazin-1-yl)ethan-1-one;
2-(3,4-dichlorophenyl)-2,2-difluoro-1-(4-{[1,2,4]triazolo[4,3-b]pyridazin-6-yl}piperazin-1-yl)ethan-1-one;
2-(3,4-difluorophenyl)-1-(4-{[1,2,4]triazolo[4,3-b]pyridazin-6-yl}piperazin-1-yl)ethan-1-one;
2-(3,4-dichlorophenyl)-1-(4-{3-methyl-[1,2,4]triazolo[4,3-b]pyridazin-6-yl}piperazin-1-yl)ethan-1-one;
2-(3,4-dichlorophenyl)-1-(4-{3-phenyl-[1,2,4]triazolo[4,3-b]pyridazin-6-yl}piperazin-1-yl)ethan-1-one;
1-(4-{3-benzyl-[1,2,4]triazolo[4,3-b]pyridazin-6-yl}piperazin-1-yl)-2-(3,4-dichlorophenyl)ethan-1-one;
2-(3,4-dichlorophenyl)-1-{4-[3-(trifluoromethyl)-[1,2,4]triazolo[4,3-b]pyridazin-6-yl]piperazin-1-yl}ethan-1-one;
1-(4-{3-cyclopropyl-[1,2,4]triazolo[4,3-b]pyridazin-6-yl}piperazin-1-yl)-2-(3,4-dichlorophenyl)ethan-1-one;
2-(3,4-dichlorophenyl)-1-(4-{[1,2,4]triazolo[4,3-b]pyridazin-6-yl}piperazin-1-yl)propan-1-one;
2-(3,4-dichlorophenyl)-2-methyl-1-(4-{[1,2,4]triazolo[4,3-b]pyridazin-6-yl}piperazin-1-yl)propan-1-one;
2-(3,4-dichlorophenyl)-1-(3-methyl-4-{[1,2,4]triazolo[4,3-b]pyridazin-6-yl}piperazin-1-yl)ethan-1-one;
2-(3,4-dichlorophenyl)-1-(3,5-dimethyl-4-{[1,2,4]triazolo[4,3-b]pyridazin-6-yl}piperazin-1-yl)ethan-1-one;
2-(3,4-dichlorophenyl)-1-(2,6-dimethyl-4-{[1,2,4]triazolo[4,3-b]pyridazin-6-yl}piperazin-1-yl)ethan-1-one;
2-(3-chloro-5-fluorophenyl)-1-(4-{[1,2,4]triazolo[4,3-b]pyridazin-6-yl}piperazin-1-yl)ethan-1-one;
2-[4-(pentafluoro-λ⁶-sulfanyl)phenyl]-1-(4-{[1,2,4]triazolo[4,3-b]pyridazin-6-yl}piperazin-1-yl)ethan-1-one;
2-[3-(pentafluoro-λ⁶-sulfanyl)phenyl]-1-(4-{[1,2,4]triazolo[4,3-b]pyridazin-6-yl}piperazin-1-yl)ethan-1-one;
2-[4-chloro-3-(trifluoromethyl)phenyl]-1-(4-{[1,2,4]triazolo[4,3-b]pyridazin-6-yl}piperazin-1-yl)ethan-1-one;
2-[3-chloro-4-(trifluoromethyl)phenyl]-1-(4-{[1,2,4]triazolo[4,3-b]pyridazin-6-yl}piperazin-1-yl)ethan-1-one;
2-[4-fluoro-3-(trifluoromethyl)phenyl]-1-(4-{[1,2,4]triazolo[4,3-b]pyridazin-6-yl}piperazin-1-yl)ethan-1-one;
2-[3-fluoro-4-(trifluoromethyl)phenyl]-1-(4-{[1,2,4]triazolo[4,3-b]pyridazin-6-yl}piperazin-1-yl)ethan-1-one;
1-(4-{[1,2,4]triazolo[4,3-b]pyridazin-6-yl}piperazin-1-yl)-2-[3-(trifluoromethyl)phenyl]ethan-1-one;
1-(4-{[1,2,4]triazolo[4,3-b]pyridazin-6-yl}piperazin-1-yl)-2-[4-(trifluoromethyl)phenyl]ethan-1-one;
2-(2,3-dihydro-1H-inden-5-yl)-1-(4-{[1,2,4]triazolo[4,3-b]pyridazin-6-yl}piperazin-1-yl)ethan-1-one;
2-(3 -tert-butylphenyl)-1 -(4-{ [1,2,4]triazolo[4,3 -b]pyridazin-6-yl}piperazin-1 -yl)ethan-1-one;
2-(4-tert-butylphenyl)-1 -(4-{ [1,2,4]triazolo[4,3 -b]pyridazin-6-yl}piperazin-1 -yl)ethan-1-one;
2-(4-chloro-3-ethylphenyl)-1-(4-{[1,2,4]triazolo[4,3-b]pyridazin-6-yl}piperazin-1-yl)ethan-1-one;
2-(3-ethyl-4-fluorophenyl)-1-(4-{[1,2,4]triazolo[4,3-b]pyridazin-6-yl}piperazin-1-yl)ethan-1-one;
2-[4-chloro-3-(propan-2-yl)phenyl]-1-(4-{[1,2,4]triazolo[4,3-b]pyridazin-6-yl}piperazin-1-yl)ethan-1-one;
2-(4-chloro-3-cyclopropylphenyl)-1-(4-{[1,2,4]triazolo[4,3-b]pyridazin-6-yl}piperazin-1-yl)ethan-1-one;
2-(4-methylphenyl)-1-(4-{[1,2,4]triazolo[4,3-b]pyridazin-6-yl}piperazin-1-yl)ethan-1-one;
2-(3-methylphenyl)-1-(4-{[1,2,4]triazolo[4,3-b]pyridazin-6-yl}piperazin-1-yl)ethan-1-one;
4-[2-oxo-2-(4-{[1,2,4]triazolo[4,3-b]pyridazin-6-yl}piperazin-1-yl)ethyl]benzamide;
3-[2-oxo-2-(4-{[1,2,4]triazolo[4,3-b]pyridazin-6-yl}piperazin-1-yl)ethyl]benzamide;
N-cyclopropyl-4-[2-oxo-2-(4-{[1,2,4]triazolo[4,3-b]pyridazin-6-yl}piperazin-1-yl)ethyl]benzamide;
N-cyclopropyl-3-[2-oxo-2-(4-{[1,2,4]triazolo[4,3-b]pyridazin-6-yl}piperazin-1-yl)ethyl]benzamide;
N,N-dimethyl-4-[2-oxo-2-(4-{[1,2,4]triazolo[4,3-b]pyridazin-6-yl}piperazin-1-yl)ethyl]benzamide;
N,N-dimethyl-3-[2-oxo-2-(4-{[1,2,4]triazolo[4,3-b]pyridazin-6-yl}piperazin-1-yl)ethyl]benzamide;
2-[4-(pyrrolidine-1-carbonyl)phenyl]-1-(4-{[1,2,4]triazolo[4,3-b]pyridazin-6-yl}piperazin-1-yl)ethan-1-one;
2-[3-(pyrrolidine-1-carbonyl)phenyl]-1-(4-{[1,2,4]triazolo[4,3-b]pyridazin-6-yl}piperazin-1-yl)ethan-1-one;
N-(2-hydroxyethyl)-4-[2-oxo-2-(4-{[1,2,4]triazolo[4,3-b]pyridazin-6-yl}piperazin-1-yl)ethyl]benzamide;
N-(2-hydroxyethyl)-3-[2-oxo-2-(4-{[1,2,4]triazolo[4,3-b]pyridazin-6-yl}piperazin-1-yl)ethyl]benzamide;
N-benzyl-4-[2-oxo-2-(4-{[1,2,4]triazolo[4,3-b]pyridazin-6-yl}piperazin-1-yl)ethyl]benzamide;
N-benzyl-3-[2-oxo-2-(4-{[1,2,4]triazolo[4,3-b]pyridazin-6-yl}piperazin-1-yl)ethyl]benzamide;
4-[2-oxo-2-(4-{[1,2,4]triazolo[4,3-b]pyridazin-6-yl}piperazin-1-yl)ethyl]benzene-1-sulfonamide;
3-[2-oxo-2-(4-{[1,2,4]triazolo[4,3-b]pyridazin-6-yl}piperazin-1-yl)ethyl]benzene-1-sulfonamide;
N-cyclopropyl-4-[2-oxo-2-(4-{[1,2,4]triazolo[4,3-b]pyridazin-6-yl}piperazin-1-yl)ethyl]benzene-1-sulfonamide;
N-cyclopropyl-3-[2-oxo-2-(4-{[1,2,4]triazolo[4,3-b]pyridazin-6-yl}piperazin-1-yl)ethyl]benzene-1-sulfonamide;
2-(4-methanesulfonylphenyl)-1-(4-{[1,2,4]triazolo[4,3-b]pyridazin-6-yl}piperazin-1-yl)ethan-1-one;
2-(3-methanesulfonylphenyl)-1-(4-{[1,2,4]triazolo[4,3-b]pyridazin-6-yl}piperazin-1-yl)ethan-1-one;
2-{[1,1′-biphenyl]-4-yl}-1-(4-{[1,2,4]triazolo[4,3-b]pyridazin-6-yl}piperazin-1-yl)ethan-1-one;
2-{[1,1′-biphenyl]-3-yl}-1-(4-{[1,2,4]triazolo[4,3-b]pyridazin-6-yl}piperazin-1-yl)ethan-1-one;
2-(4-benzylphenyl)-1-(4-{[1,2,4]triazolo[4,3-b]pyridazin-6-yl}piperazin-1-yl)ethan-1-one;
2-(3-benzylphenyl)-1-(4-{[1,2,4]triazolo[4,3-b]pyridazin-6-yl}piperazin-1-yl)ethan-1-one;
2-(4-phenoxyphenyl)-1-(4-{[1,2,4]triazolo[4,3-b]pyridazin-6-yl}piperazin-1-yl)ethan-1-one;
2-(3-phenoxyphenyl)-1-(4-{[1,2,4]triazolo[4,3-b]pyridazin-6-yl}piperazin-1-yl)ethan-1-one;
2-[4-(2-phenylethyl)phenyl]-1-(4-{[1,2,4]triazolo[4,3-b]pyridazin-6-yl}piperazin-1-yl)ethan-1-one;
2-[3-(2-phenylethyl)phenyl]-1-(4-{[1,2,4]triazolo[4,3-b]pyridazin-6-yl}piperazin-1-yl)ethan-1-one;
2-[3-(hydroxymethyl)phenyl]-1-(4-{[1,2,4]triazolo[4,3-b]pyridazin-6-yl}piperazin-1-yl)ethan-1-one;
2-[3-chloro-4-(hydroxymethyl)phenyl]-1-(4-{[1,2,4]triazolo[4,3-b]pyridazin-6-yl}piperazin-1-yl)ethan-1-one;
2-[4-chloro-3-(hydroxymethyl)phenyl]-1-(4-{[1,2,4]triazolo[4,3-b]pyridazin-6-yl}piperazin-1-yl)ethan-1-one;
2-{4-[(pyrrolidin-1-yl)methyl]phenyl}-1-(4-{[1,2,4]triazolo[4,3-b]pyridazin-6-yl}piperazin-1-yl)ethan-1-one;
2-{3-[(pyrrolidin-1-yl)methyl]phenyl}-1-(4-{[1,2,4]triazolo[4,3-b]pyridazin-6-yl}piperazin-1-yl)ethan-1-one;
2-{3-chloro-4-[(pyrrolidin-1-yl)methyl]phenyl}-1-(4-{[1,2,4]triazolo[4,3-b]pyridazin-6-yl}piperazin-1-yl)ethan-1-one;
2-{4-chloro-3-[(pyrrolidin-1-yl)methyl]phenyl}-1-(4-{[1,2,4]triazolo[4,3-b]pyridazin-6-yl}piperazin-1-yl)ethan-1-one;
2-{4-[(morpholin-4-yl)methyl]phenyl}-1-(4-{[1,2,4]triazolo[4,3-b]pyridazin-6-yl}piperazin-1-yl)ethan-1-one;
2-{3-[(morpholin-4-yl)methyl]phenyl}-1-(4-{[1,2,4]triazolo[4,3-b]pyridazin-6-yl}piperazin-1-yl)ethan-1-one;
2-{3-chloro-4-[(morpholin-4-yl)methyl]phenyl}-1-(4-{[1,2,4]triazolo[4,3-b]pyridazin-6-yl}piperazin-1-yl)ethan-1-one;
2-{4-chloro-3-[(morpholin-4-yl)methyl]phenyl}-1-(4-{[1,2,4]triazolo[4,3-b]pyridazin-6-yl}piperazin-1-yl)ethan-1-one;
2-methyl-N-({4-[2-oxo-2-(4-{[1,2,4]triazolo[4,3-b]pyridazin-6-yl}piperazin-1-yl)ethyl]phenyl}methyl)propanamide;
2-methyl-N-({3-[2-oxo-2-(4-{[1,2,4]triazolo[4,3-b]pyridazin-6-yl}piperazin-1-yl)ethyl]phenyl}methyl)propanamide;
N-({2-chloro-4-[2-oxo-2-(4-{[1,2,4]triazolo[4,3-b]pyridazin-6-yl}piperazin-1-yl)ethyl]phenyl}methyl)-2-methylpropanamide;
N-({2-chloro-5-[2-oxo-2-(4-{[1,2,4]triazolo[4,3-b]pyridazin-6-yl}piperazin-1-yl)ethyl]phenyl}methyl)-2-methylpropanamide;
2-{3-chloro-4-[(cyclopropylamino)methyl]phenyl}-1-(4-{[1,2,4]triazolo[4,3-b]pyridazin-6-yl}piperazin-1-yl)ethan-1-one;
2-{4-chloro-3-[(cyclopropylamino)methyl]phenyl}-1-(4-{[1,2,4]triazolo[4,3-b]pyridazin-6-yl}piperazin-1-yl)ethan-1-one;
N,N-dimethyl-4-[2-oxo-2-(4-{[1,2,4]triazolo[4,3-b]pyridazin-6-yl}piperazin-1-yl)ethyl]benzene-1-sulfonamide;
N,N-dimethyl-3-[2-oxo-2-(4-{[1,2,4]triazolo[4,3-b]pyridazin-6-yl}piperazin-1-yl)ethyl]benzene-1-sulfonamide;
2-[4-(pyrrolidine-1-sulfonyl)phenyl]-1-(4-{[1,2,4]triazolo[4,3-b]pyridazin-6-yl}piperazin-1-yl)ethan-1-one;
2-[3-(pyrrolidine-1-sulfonyl)phenyl]-1-(4-{[1,2,4]triazolo[4,3-b]pyridazin-6-yl}piperazin-1-yl)ethan-1-one;
2-(3-ethynylphenyl)-1-(4-{[1,2,4]triazolo[4,3-b]pyridazin-6-yl}piperazin-1-yl)ethan-1-one;
2-{4-[(piperidin-1-yl)methyl]phenyl}-1-(4-{[1,2,4]triazolo[4,3-b]pyridazin-6-yl}piperazin-1-yl)ethan-1-one;
2-{4-[(4,4-difluoropiperidin-1-yl)methyl]phenyl}-1-(4-{[1,2,4]triazolo[4,3-b]pyridazin-6-yl}piperazin-1-yl)ethan-1-one;
2-{4-[(dimethylamino)methyl]phenyl}-1-(4-{[1,2,4]triazolo[4,3-b]pyridazin-6-yl}piperazin-1-yl)ethan-1-one;
2-[4-chloro-3-(difluoromethyl)phenyl]-1-(4-{[1,2,4]triazolo[4,3-b]pyridazin-6-yl}piperazin-1-yl)ethan-1-one;
2-[4-chloro-3-(fluoromethyl)phenyl]-1-(4-{[1,2,4]triazolo[4,3-b]pyridazin-6-yl}piperazin-1-yl)ethan-1-one;
2-(3,4-dichlorophenyl)-1-(4-{8-methyl-[1,2,4]triazolo[4,3-b]pyridazin-6-yl}piperazin-1-yl)ethan-1-one;
2-(3,4-dichlorophenyl)-1-(4-{7-methyl-[1,2,4]triazolo[4,3-b]pyridazin-6-yl}piperazin-1-yl)ethan-1-one;
2-(3,4-dichlorophenyl)-1-{4-[3-(trifluoromethyl)-[1,2,4]triazolo[4,3-b]pyridazin-6-yl]piperazin-1-yl}ethan-1-one;
tert-butyl N-(3-{4-[2-oxo-2-(4-{[1,2,4]triazolo[4,3-b]pyridazin-6-yl}piperazin-1-yl)ethyl]phenyl}prop-2-yn-1-yl)carbamate;
2-[4-(hydroxymethyl)phenyl]-1-(4-{[1,2,4]triazolo[4,3-b]pyridazin-6-yl}piperazin-1-yl)ethan-1-one;
2-phenyl-1-(4-{[1,2,4]triazolo[4,3-b]pyridazin-6-yl}piperazin-1-yl)ethan-1-one;
2-[4-(3-aminoprop-1-yn-1-yl)phenyl]-1-(4-{[1,2,4]triazolo[4,3-b]pyridazin-6-yl}piperazin-1-yl)ethan-1-one;
2-(4-ethynylphenyl)-1-(4-{[1,2,4]triazolo[4,3-b]pyridazin-6-yl}piperazin-1-yl)ethan-1-one;
2-(2,3,4,5,6-pentafluorophenyl)-1-(4-{[1,2,4]triazolo[4,3-b]pyridazin-6-yl}piperazin-1-yl)ethan-1-one;
2-(4-bromo-3-chlorophenyl)-1-(4-{[1,2,4]triazolo[4,3-b]pyridazin-6-yl}piperazin-1-yl)ethan-1-one;
2-(3-bromo-4-chlorophenyl)-1-(4-{[1,2,4]triazolo[4,3-b]pyridazin-6-yl}piperazin-1-yl)ethan-1-one;
2-(4-bromo-3-fluorophenyl)-1-(4-{[1,2,4]triazolo[4,3-b]pyridazin-6-yl}piperazin-1-yl)ethan-1-one;
2-(3-bromo-4-fluorophenyl)-1-(4-{[1,2,4]triazolo[4,3-b]pyridazin-6-yl}piperazin-1-yl)ethan-1-one;
tert-butyl N-(3-{4-[2-oxo-2-(4-{[1,2,4]triazolo[4,3-b]pyridazin-6-yl}piperazin-1-yl)ethyl]phenyl}propyl)carbamate;
2-(3,4-dichlorophenyl)-1-(4-{3-[2-(trifluoromethyl)pyridin-4-yl]-[1,2,4]triazolo[4,3-b]pyridazin-6-yl}piperazin-1-yl)ethan-1-one;
2-(2H-1,3-benzodioxol-5-yl)-1-(4-{[1,2,4]triazolo[4,3-b]pyridazin-6-yl}piperazin-1-yl)ethan-1-one;
tert-butyl N-(3-{2-fluoro-5-[2-oxo-2-(4-{[1,2,4]triazolo[4,3-b]pyridazin-6-yl}piperazin-1-yl)ethyl]phenyl}prop-2-yn-1-yl)carbamate;
2-[3-chloro-5-(trifluoromethyl)phenyl]-1-(4-{[1,2,4]triazolo[4,3-b]pyridazin-6-yl}piperazin-1-yl)ethan-1-one;
2-(3,5-difluorophenyl)-1-(4-{[1,2,4]triazolo[4,3-b]pyridazin-6-yl}piperazin-1-yl)ethan-1-one;
2-[4-(4-hydroxybenzoyl)phenyl]-1-(4-{[1,2,4]triazolo[4,3-b]pyridazin-6-yl}piperazin-1-yl)ethan-1-one;
2-(4-fluoro-3-methoxyphenyl)-1-(4-{[1,2,4]triazolo[4,3-b]pyridazin-6-yl}piperazin-1-yl)ethan-1-one;
2-[3,5-bis(trifluoromethyl)phenyl]-1-(4-{[1,2,4]triazolo[4,3-b]pyridazin-6-yl}piperazin-1-yl)ethan-1-one;
2-(3-fluoro-4-methoxyphenyl)-1-(4-{[1,2,4]triazolo[4,3-b]pyridazin-6-yl}piperazin-1-yl)ethan-1-one;
2-(3-methylphenyl)-1-(4-{[1,2,4]triazolo[4,3-b]pyridazin-6-yl}piperazin-1-yl)ethan-1-one;
2-(4-methylphenyl)-1-(4-{[1,2,4]triazolo[4,3-b]pyridazin-6-yl}piperazin-1-yl)ethan-1-one;
1-(4-{[1,2,4]triazolo[4,3-b]pyridazin-6-yl}piperazin-1-yl)-2-(3,4,5-trifluorophenyl)ethan-1-one;
2-[4-fluoro-3-(propan-2-yl)phenyl]-1-(4-{[1,2,4]triazolo[4,3-b]pyridazin-6-yl}piperazin-1-yl)ethan-1-one;
2-(3-cyclopropyl-4-fluorophenyl)-1-(4-{[1,2,4]triazolo[4,3-b]pyridazin-6-yl}piperazin-1-yl)ethan-1-one;
2-[3-(difluoromethyl)-4-fluorophenyl]-1-(4-{[1,2,4]triazolo[4,3-b]pyridazin-6-yl}piperazin-1-yl)ethan-1-one;
2-[4-fluoro-3-(fluoromethyl)phenyl]-1-(4-{[1,2,4]triazolo[4,3-b]pyridazin-6-yl}piperazin-1-yl)ethan-1-one;
2-[3,4-bis(trifluoromethyl)phenyl]-1-(4-{[1,2,4]triazolo[4,3-b]pyridazin-6-yl}piperazin-1-yl)ethan-1-one;
2-[3-fluoro-5-(trifluoromethyl)phenyl]-1-(4-{[1,2,4]triazolo[4,3-b]pyridazin-6-yl}piperazin-1-yl)ethan-1-one;
2-[2-fluoro-3-(trifluoromethyl)phenyl]-1-(4-{[1,2,4]triazolo[4,3-b]pyridazin-6-yl}piperazin-1-yl)ethan-1-one;
2-fluoro-5-[2-oxo-2-(4-{[1,2,4]triazolo[4,3-b]pyridazin-6-yl}piperazin-1-yl)ethyl]benzonitrile;
2-(4-fluoro-3-nitrophenyl)-1-(4-{[1,2,4]triazolo[4,3-b]pyridazin-6-yl}piperazin-1-yl)ethan-1-one;
2-[4-fluoro-3-(trifluoromethoxy)phenyl]-1-(4-{[1,2,4]triazolo[4,3-b]pyridazin-6-yl}piperazin-1-yl)ethan-1-one;
2-{6-fluoro-[1,1′-biphenyl]-3-yl}-1-(4-{[1,2,4]triazolo[4,3-b]pyridazin-6-yl}piperazin-1-yl)ethan-1-one;
2-{4′,6-difluoro-[1,1′-biphenyl]-3-yl}-1-(4-{[1,2,4]triazolo[4,3-b]pyridazin-6-yl}piperazin-1-yl)ethan-1-one;
2-[3-(1,1-difluoroethyl)-4-fluorophenyl]-1-(4-{[1,2,4]triazolo[4,3-b]pyridazin-6-yl}piperazin-1-yl)ethan-1-one;
2-[4-fluoro-3-(1,1,2,2,2-pentafluoroethyl)phenyl]-1-(4-{[1,2,4]triazolo[4,3-b]pyridazin-6-yl}piperazin-1-yl)ethan-1-one;
2-(3-ethynyl-4-fluorophenyl)-1-(4-{[1,2,4]triazolo[4,3-b]pyridazin-6-yl}piperazin-1-yl)ethan-1-one;
2-[4-fluoro-3-(prop-1-yn-1-yl)phenyl]-1-(4-{[1,2,4]triazolo[4,3-b]pyridazin-6-yl}piperazin-1-yl)ethan-1-one;
2-(3-tert-butyl-4-fluorophenyl)-1-(4-{[1,2,4]triazolo[4,3-b]pyridazin-6-yl}piperazin-1-yl)ethan-1-one;
2-[4-fluoro-3-(hydroxymethyl)phenyl]-1-(4-{[1,2,4]triazolo[4,3-b]pyridazin-6-yl}piperazin-1-yl)ethan-1-one;
2-[4-fluoro-3-(methoxymethyl)phenyl]-1-(4-{[1,2,4]triazolo[4,3-b]pyridazin-6-yl}piperazin-1-yl)ethan-1-one;
2-fluoro-5-[2-oxo-2-(4-{[1,2,4]triazolo[4,3-b]pyridazin-6-yl}piperazin-1-yl)ethyl]benzoic acid;
2-fluoro-5-[2-oxo-2-(4-{[1,2,4]triazolo[4,3-b]pyridazin-6-yl}piperazin-1-yl)ethyl]benzamide;
2-fluoro-N-methyl-5-[2-oxo-2-(4-{[1,2,4]triazolo[4,3-b]pyridazin-6-yl}piperazin-1-yl)ethyl]benzamide;
2-fluoro-N,N-dimethyl-5-[2-oxo-2-(4-{[1,2,4]triazolo[4,3-b]pyridazin-6-yl}piperazin-1-yl)ethyl]benzamide;
2-fluoro-5-[2-oxo-2-(4-{[1,2,4]triazolo[4,3-b]pyridazin-6-yl}piperazin-1-yl)ethyl]benzene-1-sulfonamide;
2-fluoro-N-methyl-5-[2-oxo-2-(4-{[1,2,4]triazolo[4,3-b]pyridazin-6-yl}piperazin-1-yl)ethyl]benzene-1-sulfonamide;
2-fluoro-N,N-dimethyl-5-[2-oxo-2-(4-{[1,2,4]triazolo[4,3-b]pyridazin-6-yl}piperazin-1-yl)ethyl]benzene-1-sulfonamide;
2-(4-fluoro-3-methanesulfonylphenyl)-1-(4-{[1,2,4]triazolo[4,3-b]pyridazin-6-yl}piperazin-1-yl)ethan-1-one;
and combinations thereof.

18. The method of claim 17, wherein the one or more compounds are chosen from:
2-[4-fluoro-3-(trifluoromethyl)phenyl]-1-(4-{[1,2,4]triazolo[4,3-b]pyridazin-6-yl}piperazin-1-yl)ethan-1-one;
2-(3-bromo-4-chlorophenyl)-1-(4-{[1,2,4]triazolo[4,3-b]pyridazin-6-yl}piperazin-1-yl)ethan-1-one;
2-(3,4-dichlorophenyl)-1-(4-{[1,2,4]triazolo[4,3-b]pyridazin-6-yl}piperazin-1-yl)ethan-1-one;
2-(3-bromo-4-fluorophenyl)-1-(4-{[1,2,4]triazolo[4,3-b]pyridazin-6-yl}piperazin-1-yl)ethan-1-one;
2-(3-chloro-4-fluorophenyl)-1-(4-{[1,2,4]triazolo[4,3-b]pyridazin-6-yl}piperazin-1-yl)ethan-1-one;
2-(4-bromo-3-chlorophenyl)-1-(4-{[1,2,4]triazolo[4,3-b]pyridazin-6-yl}piperazin-1-yl)ethan-1-one;
2-[4-chloro-3-(trifluoromethyl)phenyl]-1-(4-{[1,2,4]triazolo[4,3-b]pyridazin-6-yl}piperazin-1-yl)ethan-1-one;
2-(4-chloro-3-methylphenyl)-1-(4-{[1,2,4]triazolo[4,3-b]pyridazin-6-yl}piperazin-1-yl)ethan-1-one;
2-[3,5-bis(trifluoromethyl)phenyl]-1-(4-{[1,2,4 ]triazolo[4,3-b]pyridazin-6-yl}piperazin-1-yl)ethan-1-one;
2-(4-fluoro-3-methylphenyl)-1-(4-{[1,2,4]triazolo[4,3-b]pyridazin-6-yl}piperazin-1-yl)ethan-1-one;
2-[3-chloro-5-(trifluoromethyl)phenyl]-1-(4-{[1,2,4]triazolo[4,3-b]pyridazin-6-yl}piperazin-1-yl)ethan-1-one;
2-(4-chloro-3-fluorophenyl)-1-(4-{[1,2,4]triazolo[4,3-b]pyridazin-6-yl}piperazin-1-yl)ethan-1-one;
2-(3,4-difluorophenyl)-1-(4-{[1,2,4]triazolo[4,3-b]pyridazin-6-yl}piperazin-1-yl)ethan-1-one;
2-(4-bromo-3-fluorophenyl)-1-(4-{[1,2,4]triazolo[4,3-b]pyridazin-6-yl}piperazin-1-yl)ethan-1-one;
2-(4-chlorophenyl)-1-(4-{[1,2,4]triazolo[4,3-b]pyridazin-6-yl}piperazin-1-yl)ethan-1-one;
2-(3-chloro-5-fluorophenyl)-1-(4-{[1,2,4]triazolo[4,3-b]pyridazin-6-yl}piperazin-1-yl)ethan-1-one;
2-(4-ethynylphenyl)-1-(4-{[1,2,4]triazolo[4,3-b]pyridazin-6-yl}piperazin-1-yl)ethan-1-one;
2-(3,5-dichlorophenyl)-1-(4-{[1,2,4]triazolo[4,3-b]pyridazin-6-yl}piperazin-1-yl)ethan-1-one; and
and combinations thereof.

19. A kit comprising one or more compounds of claim 1 or a composition comprising the one or more compounds or materials to prepare the composition and instructions on administration details to an individual who has been diagnosed with or who is at risk of getting Cryptosporidium infection wherein the details comprise one or more of the following: dosage, frequency, and length of time for administration of the compound or the composition.
